(19) United States
(12) Patent Application Publication
(10) Pub. No.: US 2020/0331925 A1
(43) Pub. Date: Oct. 22, 2020

(54) HETEROCYCLIC COMPOUNDS FOR THE TREATMENT OF ABNORMAL CELLULAR PROLIFERATION

(71) Applicant: G1 Therapeutics, Inc., Research Triangle Park, NC (US)

(72) Inventors: Jay Copeland Strum, Hillsborough, NC (US); David Jung, Durham, NC (US)

(73) Assignee: G1 Therapeutics, Inc., Research Triangle Park, NC (US)

(21) Appl. No.: 16/918,985

(22) Filed: Jul. 1, 2020

Related U.S. Application Data
(63) Continuation of application No. PCT/US2019/012343, filed on Jan. 4, 2019.
(60) Provisional application No. 62/613,727, filed on Jan. 4, 2018.

(51) Int. Cl. C07D 487/14; A61K 35/00

(52) U.S. Cl. CPC C07D 487/14; A61K 35/00

(57) ABSTRACT

This invention is in the area of heterocyclic-based compounds for the treatment of disorders involving abnormal cellular proliferation, including but not limited to tumors and cancers.

### CROSS-REFERENCE TO RELATED APPLICATIONS

This application is a continuation of International Patent Application No. PCT/US2019/012343, filed in the U.S. Receiving Office on Jan. 4, 2019, which claims the benefit of U.S. Provisional Application 62/613,727, filed on Jan. 4, 2018. The entirety of this application is hereby incorporated by reference herein for all purposes.

### FIELD OF THE INVENTION

This invention is in the area of heterocyclic-based compounds for the treatment of disorders involving abnormal cellular proliferation, including but not limited to tumors and cancers.

### BACKGROUND

In normal tissue, cellular proliferation is generally restricted to cells that are required to replenish the tissue. Once cells have terminally differentiated, they have a specialized function and no longer divide. Most tissues are made up of non-dividing cells. Thus, normal cell proliferation is tightly controlled to ensure that only the necessary cells divide. There is also a careful balance between cell division and programmed cell death (apoptosis).

Cell division, sometimes referred to as the cell cycle, has four phases: G₁ phase (synthesis of various enzymes required for DNA replication), S phase (DNA replication producing two identical sets of chromosomes), G₂ (significant protein synthesis, including production of microtubules) and M phase (nuclear division, cytoplasmic division and formation of new cell membrane). Cell division also includes a complex system of cell signaling networks that allow cells to interpret information from numerous extracellular signals, including through receptor proteins, inflammatory factors and pro-apoptotic and anti-apoptotic signals. Dysfunctional signals include those from genetic mutation, infection, exposure to environmental factors including toxins, system stress, autoimmune disorders, and inflammation.

Cyclin dependent kinases (CDKs) play an important role in modulating cellular division and are thus therapeutic targets for treating a variety of disorders, including benign growths, neoplasms, tumorigenesis, cancerogenesis, autoimmune disorders, inflammatory disorders graft-versus-host rejection, and fibrotic disorders.

Over the past 20 years, a number of CDK inhibitors have been developed as potential agents for the targeting of the cell-cycle pathway. CDK inhibitors can be broad (“pan”), specific or multiple target inhibitors. Some CDK inhibitors are non-specific in their CDK targeting and often referred to as pan-CDK inhibitors. For example, flavopiridol has been shown to inhibit CDK1, CDK2, CDK4, CDK6, CDK7, and CDK9 and can induce cell cycle arrest in both G1 and G2. Additional pan-CDK inhibitors include roscovitine, staurosporine, genistein, and dinaciclib, a potent inhibitor of CDK1, CDK2, CDK5 and CDK9, as well as CDK 4, CDK6 and CDK7. In contrast, Cortistatin derivatives are potent inhibitors of CDK8.

Compounds with at least some selectivity for CDK-9 include SNS-032 and LDC000067. Pan CDK inhibitors in clinical trials include Dinaciclib, AT7519, P276-00, AZD5438, and PHA-793887. Compounds with selectivy for CDK4/6 include Palbociclib, Abemaciclib, and Ribociclib. Tricyclic lactams Trilaciclib and Lerociclib demonstrate advantageous selectivity for CDK4/6 inhibition.

Various other tricyclic lactam compounds have also been identified for the treatment of hyperproliferative diseases, including those in U.S. Pat. Nos. 8,822,683; 8,598,197; 8,598,186; 8,691,830; 8,829,102; 8,822,683; 9,102,682; 9,260,442; 9,481,591; 9,499,564; 9,717,735; 9,745,316; 9,856,268; and U.S. Pat. No. 9,957,276 filed by Tavares and Strum and assigned to G1 Therapeutics describing a class of N-(heteroaryl)-pyrrolo[3,2-d]pyrimidin-2-amine cyclin dependent kinase inhibitors such as those of the formula (with variables as defined therein):

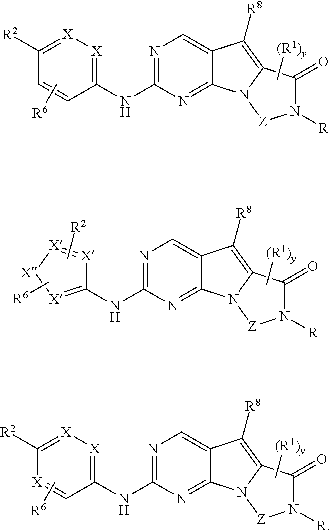

Other publications include the following: WO 2014/144326, WO 2014/144596, WO 2014/144847, WO 2014/144740, WO 2015/161285, WO 2015/161287, WO 2015/161283, WO 2015/161288, WO 2016/040858, and WO 2016/040848.

Additional CDK inhibitors include those described in WO 2018/005533, WO 2018/005860, and WO 2018/005863 filed by Strum et al. and assigned to G1 Therapeutics.

There remains a need for additional compounds to treat disorders associated with abnormal cellular proliferation, including a tumor or cancer.

### SUMMARY

The invention includes compounds of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, and Formula XVII or a pharmaceutically acceptable salt or composition thereof. In one embodiment, an active compound or its salt, composition, or prodrug thereof is used in an effective amount to treat a medical disorder involving abnormal cellular proliferation in a host in need thereof.

Based on this discovery, compounds, compositions, and their uses are presented for the treatment of a patient with a proliferative disorder including a tumor or cancer that includes administering an effective amount of one or a combination of the compounds described herein to a patient in need thereof, optionally in a pharmaceutically acceptable carrier. In certain embodiments, the antiproliferative disorder is selected from a benign growth, neoplasm, tumor, cancer, autoimmune disorder, inflammatory disorder, graft-versus-host rejection and a fibrotic disorder. In a typical embodiment, the patient is a human.

In one aspect of the present invention a compound of Formula I, Formula II, Formula III, Formula IV, Formula V, Formula VI, Formula VII, Formula VIII, Formula IX, Formula X, Formula XI, Formula XII, Formula XIII, or Formula XIV is provided:

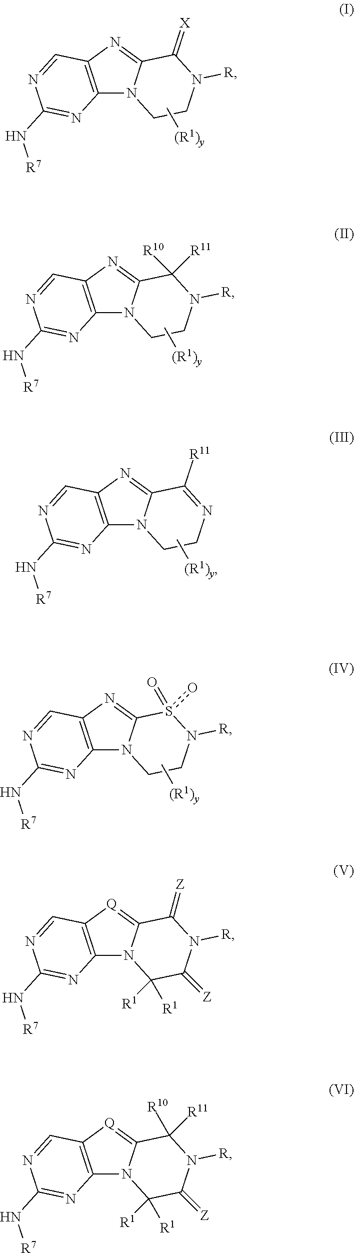

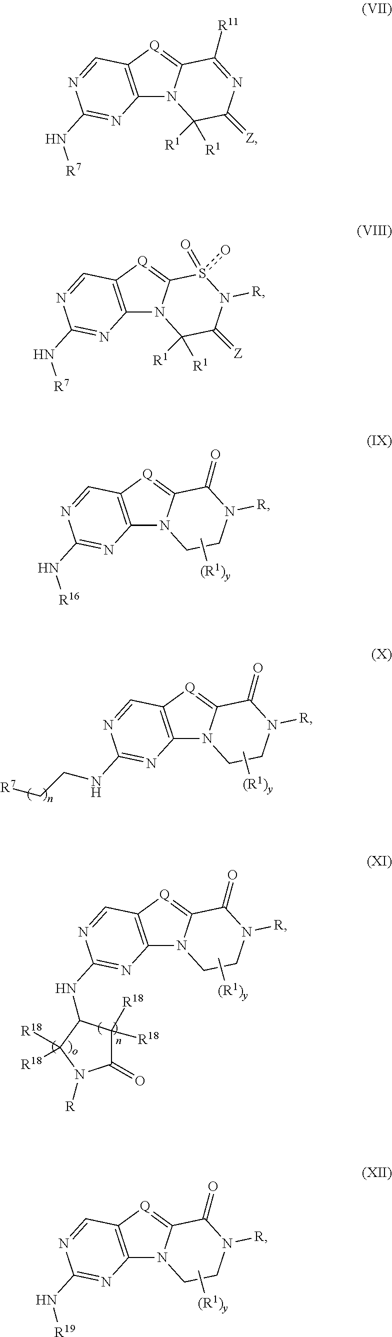

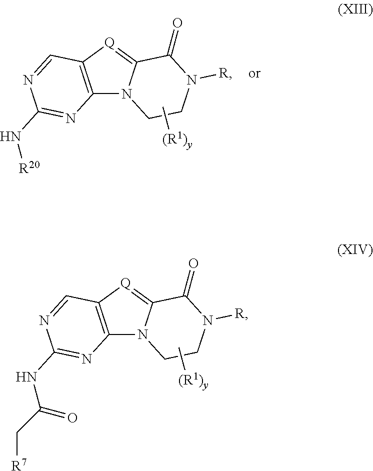

or a pharmaceutically acceptable salt, N-oxide, isotopic derivative, prodrug, and/or a pharmaceutically acceptable composition thereof;
wherein:

each y is independently 0, 1, 2, 3, or 4, and typically 0, 1, or 2;

X is S, CH₂, CHR¹², CR¹²R¹³, NH, or NR¹²;

Z is independently O, S, CH₂, CHR¹², CR¹²R¹³, NH, or NR¹²;

Q is CH or N;

[symbol] represents the presence or absence of a double bond;

each R is independently hydrogen, C₁-C₆alkyl, —(C₀-C₂alkyl)(C₃-C₈cycloalkyl), —(C₀-C₂alkyl)(C₃-C₈ heterocycle), —(C₀-C₂alkyl)(aryl), —(C₀-C₂alkyl)(heteroaryl), —COOalkyl, —COOalkyl-aryl, or —COOH;

each R¹ is independently hydrogen, alkyl, aryl, cycloalkyl, haloalkyl, heteroaryl, or heterocycle, wherein two R¹s on adjacent ring atom(s) or on the same ring atom may come together with the ring atom(s) to which they are attached to optionally constitute a 3, 4, 5, 6, 7, or 8-membered cycloalkyl or heterocycle that has 1, 2, or 3 heteroatoms selected from N, O, and S;

wherein the 3, 4, 5, 6, 7, or 8-membered cycloalkyl or heterocycle formed by combining two R¹s with the atom(s) to which they are attached can be optionally substituted with 1, 2, 3, or 4 substituents independently selected from R⁵⁰;

R⁵⁰ is selected from hydrogen, amino, —NHR¹⁴, —NR¹⁴R¹⁵, hydroxyl, OR¹⁴, R⁶, and R²;

R⁷ is selected from aryl, heteroaryl, cycloalkyl, heterocycle, alkyl, —C(O)aryl, —C(O)heteroaryl, —C(O)cycloalkyl, —C(O)heterocycle, —C(O)alkyl, and —C(O)heterocycle; each of which R⁷ is optionally substituted with 1, 2, 3, or 4 substituents independently selected from amino, halogen, alkyl, —NHR¹⁴, —NR¹⁴R¹⁵, hydroxyl, OR¹⁴, R⁶, and R²;

R² is independently selected from -(alkylene)ₘ-heterocycle, -(alkylene)ₘ-heteroaryl, -(alkylene)ₘ-NR³R⁴, -(alkylene)ₘ-C(O)—NR³R⁴; -(alkylene)ₘ-C(O)—O-alkyl; -(alkylene)ₘ-O—R⁵, -(alkylene)ₘ-S(O)ₙ—R⁵, and -(alkylene)ₘ-S(O)ₙ—NR³R⁴; any of which may be optionally independently substituted with 1, 2, 3, or 4 R^{x} groups as allowed by valance, and wherein two R^{x} groups bound to the same or adjacent atom may optionally combine to form a ring;

m is 0 or 1;

n is independently 0, 1, or 2;

o is independently 0, 1, 2, or 3;

wherein for compounds of Formula XI n and o cannot both be 0;

R³ and R⁴ at each occurrence are independently selected from hydrogen, alkyl, cycloalkyl, heterocycle, aryl, heteroaryl, alkyl-cycloalkyl, alkyl-heterocycle, alkyl-aryl, and alkyl-heteroaryl; each of which R³ and R⁴ except hydrogen may be optionally independently substituted with 1, 2, 3, or 4 R^{x} groups as allowed by valance; or

R³ and R⁴ together with the nitrogen atom to which they are attached may combine to form a heterocycle ring optionally independently substituted with 1, 2, 3, or 4 R^{x} groups as allowed by valance, and wherein two R^{x}s bound to the same or adjacent atom(s) may optionally combine to form a 3, 4, 5, 6, 7, or 8-membered cycloalkyl or heterocycle that has 1, 2, or 3 heteroatoms selected from N, O, and S;

R⁵ is independently selected at each occurrence from hydrogen, alkyl, alkenyl, alkynyl, cycloalkyl, heterocycle, aryl, heteroaryl, alkyl-cycloalkyl, alkyl-heterocycle, alkyl-aryl, and alkyl-heteroaryl; each of which R⁵ except hydrogen may be optionally independently substituted with 1, 2, 3, or 4 R^{x} groups as allowed by valance;

R^{x} at each occurrence is independently selected from hydrogen, hydroxy, —O-alkyl, halogen, cyano, nitro, oxo, alkyl, haloalkyl, alkenyl, alkynyl, cycloalkyl, heterocycle, aryl, heteroaryl, alkyl-aryl, alkyl-heteroaryl, alkyl-cycloalkyl, amino, —C(O)N(R⁶)₂, —C(O)OR⁶, and alkyl-heterocycle; each of which R^{x} groups except hydrogen, halogen, cyano, nitro, and oxo may be further independently substituted with 1, 2, 3, or 4 substituents independently selected from hydroxy, —O-alkyl, halo, cyano, nitro, oxo, alkyl, haloalkyl, alkenyl, alkynyl, cycloalkyl, heterocycle, aryl, heteroaryl, alkyl-aryl, alkyl-heteroaryl, alkyl-cycloalkyl, and alkyl-heterocycle;

In one embodiment, R^{x} is selected from —C(O)alkyl and —C(O)cycloalkyl;

R⁶ is selected independently at each instance from hydrogen, alkyl, alkenyl, alkynyl, cycloalkyl, heterocycle, aryl, heteroaryl, alkyl-cycloalkyl, alkyl-heterocycle, alkyl-aryl, and alkyl-heteroaryl;

R¹⁰ and R¹¹ are independently selected from hydrogen, alkyl, —NH₂, —NHR¹², —NR¹²R¹³, —S(O)alkyl, —SO₂alkyl, cycloalkyl, heterocycle, aryl, heteroaryl, alkyl-aryl, and alkyl-heteroaryl; each of which R¹⁰ and R¹¹ except hydrogen is optionally substituted with 1, 2, 3, or 4 substituents selected from amino, —NHR¹⁴, —NR¹⁴R¹⁵, hydroxyl, OR¹⁴, R⁶, and R²;

R¹² is selected from hydrogen, alkyl, alkenyl, alkynyl, —C(O)R⁶, —C(O)alkyl, —C(S)alkyl, aryl, —SO₂alkyl, heteroaryl, alkyl-aryl, cycloalkyl, heterocycle, and alkyl-heteroaryl; each of which R¹² except hydrogen is optionally substituted with 1, 2, 3, or 4 substituents selected from amino, —NHR¹⁴, —NR¹⁴R¹⁵, hydroxyl, OR¹⁴, R⁶, and R²;

R¹³ is independently selected from hydrogen, alkyl, alkenyl, alkynyl, —C(O)R⁶, —C(O)alkyl, —C(S)alkyl, aryl, —SO₂alkyl, heteroaryl, alkyl-aryl, cycloalkyl, heterocycle, and alkyl-heteroaryl; each of which R¹³ except hydrogen is optionally substituted with 1, 2, 3, or 4 substituents selected from amino, —NHR¹⁴, —NR¹⁴R¹⁵, hydroxyl, OR¹⁴, R⁶, and R²;

R¹⁴ and R¹⁵ are independently selected from hydrogen, alkyl, alkenyl, alkynyl, —C(O)R⁶, —C(O)alkyl, —C(S)alkyl, aryl, —SO₂alkyl, heteroaryl, heterocycle, alkyl-aryl, and alkyl-heteroaryl;

R¹⁶ is cycloalkyl substituted with at least one substituent selected from R¹⁷ and optionally substituted with 1, 2, 3, or 4 substituents independently selected from R^{x}; or

R¹⁶ is cycloalkyl-heterocycle-R optionally substituted with 1, 2, 3, or 4 additional substituents independently selected from R^{x};

R¹⁷ is independently selected from —NR—S(O)alkyl, —NR—S(O)₂alkyl, —NR-heterocycle, —NR-heteroaryl, —NR-aryl, —NR-alkyl-heteroaryl, and —NR-alkyl-aryl; each of which R¹⁷ is optionally substituted with 1, 2, 3, or 4 substituents selected from R^{x};

each R¹⁸ is independently hydrogen, alkyl, aryl, cycloalkyl, haloalkyl, heteroaryl, —NHR¹⁴, —NR¹⁴R¹⁵ hydroxyl, OR¹⁴, R⁶, or R²;

wherein two R¹⁸s on the same ring atom together with the ring atom to which they are attached optionally form an oxo group and wherein in this embodiment, typically two oxo groups are not adjacent and typically there are not more than two oxo groups in the N-heterocyle; and

wherein two R¹⁸s on adjacent ring atoms together with the ring atoms to which they are attached optionally form a 6-membered aryl or heteroaryl group;

R¹⁹ is hydrogen or a heterocycle substituted with at least one substituent independently selected from amino, halogen, alkyl, —NHR¹⁴, —NR¹⁴R¹⁵, hydroxyl, OR¹⁴, R⁶, oxo, and R²; and

R²⁰ is selected from —C(O)alkyl, —C(O)aryl, —C(O)heteroaryl, —C(O)cycloalkyl, and —C(O)heterocycle; each of which R²⁰ is optionally substituted with 1, 2, 3, or 4 substituents independently selected from amino, halogen, alkyl, —NHR¹⁴, —NR¹⁴R¹⁵, hydroxyl, OR¹⁴, R⁶, —C(O)R⁶, and R².

In one embodiment R^{x} cannot be hydrogen.

In one embodiment R⁶ cannot be hydrogen.

In another aspect of the present invention a compound of Formula XV, Formula XVI, or Formula XVII is provided:

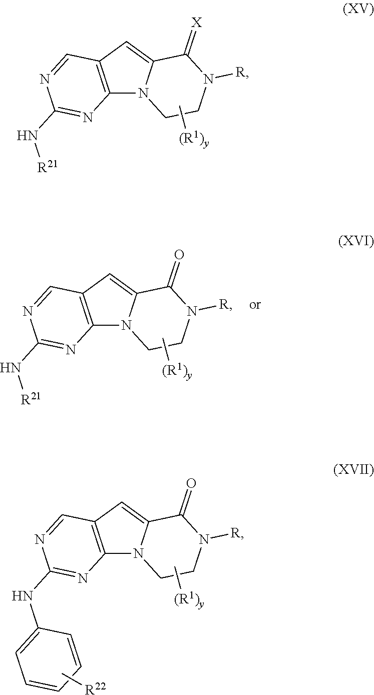

or a pharmaceutically acceptable salt, N-oxide, isotopic derivative, prodrug, and/or a pharmaceutically acceptable composition thereof;
wherein:

R²¹ is selected from

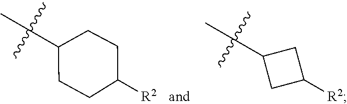

R²² is selected from

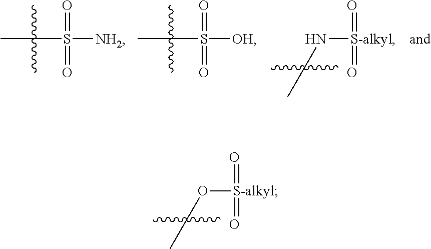

and R¹, y, X, R², and R are defined above.

These compounds can be used to treat conditions of abnormal cellular proliferation in a host in need thereof, typically a human.

In one embodiment, the active compound acts as an inhibitor of a cyclin-dependent kinase (CDK), for example CDK2, CDK4, CDK6, and/or CDK9. In one aspect, the compound is a selective inhibitor of CDK4 and/or CDK6. In one aspect, the compound is a selective inhibitor of CDK2. In one aspect the compound is a selective inhibitor of CDK9. In another embodiment, the selectivity is for CDK4 and/or CDK6 over CDK2. In another embodiment, the selectivity is for CDK4 and/or CDK6 over CDK9. In another embodiment, the selectivity is for CDK9 over CDK4 and/or CDK6. In another embodiment, the selectivity is for CDK2 over CDK4 and/or CDK6. In another embodiment, the selectivity is for CDK2 over CDK9. In another embodiment, the selectivity is for CDK4 over CDK6. In another embodiment, the selectivity is for CDK6 over CDK4. In another embodiment, the selectivity is for CDK9 over CDK2. Based on this, in one embodiment, the method for the treatment of a disorder of abnormal cellular proliferation that is mediated by CDK2, CDK4, CDK6, and/or CDK9 is provided that includes the administration of an effective amount of a compound of the present invention or a pharmaceutically acceptable salt thereof, optionally in a pharmaceutically acceptable carrier, as described in more detail below.

In an alternative embodiment, a method for the treatment of a disorder of abnormal cellular proliferation that is not mediated by CDK2, CDK4, CDK6, or CDK9 is provided that includes the administration of an effective amount of a compound of the present invention or a pharmaceutically acceptable salt thereof, optionally in a pharmaceutically acceptable carrier, as described in more detail below.

In another embodiment, a method for the treatment of a fibrotic disorder in a host is provided that includes the administration of an effective amount of a compound of the present invention or a pharmaceutically acceptable salt thereof, optionally in a pharmaceutically acceptable carrier.

In another embodiment, a method for the treatment of rheumatoid arthritis or psoriasis in a host is provided that includes the administration of an effective amount of a compound of the present invention or a pharmaceutically acceptable salt thereof, optionally in a pharmaceutically acceptable carrier.

In yet another embodiment, a method for the treatment of an autoimmune disorder in a host is provided that includes the administration of an effective amount of a compound of the present invention or a pharmaceutically acceptable salt thereof, optionally in a pharmaceutically acceptable carrier.

In a principal embodiment, a method for the treatment of a tumor or cancer in a host is provided that includes the administration of an effective amount of a compound of the present invention or a pharmaceutically acceptable salt thereof, optionally in a pharmaceutically acceptable carrier. In an aspect of this embodiment, the cancer is an Rb-positive tumor or cancer. In another aspect of this embodiment, the cancer is an Rb-negative tumor or cancer. In certain aspects, the cancer is selected from breast cancer, prostate cancer (including androgen-resistant prostate cancer), another cancer of the reproductive system such as endometrial, ovarian or testicular cancer, small cell lung carcinoma, glioblastoma, colon, and head and/or neck cancer.

In yet another embodiment, a method for the treatment of a disorder of abnormal cellular proliferation in a host such as a human is provided that includes administering an effective amount of a combination of one or more of the active compounds described herein in combination or alternation with another active compound. In certain aspects of the invention, the second compound is a chemotherapeutic agent. In another aspect of this embodiment, the second active compound is an immune modulator, including but not limited to a checkpoint inhibitor such as an anti-PD1, anti-CTLA, anti-LAG-3, anti-Tim, etc. antibody, small molecule, peptide, nucleotide or other inhibitor (including but not limited to ipilimumab (Yervoy), Pembrolizumab (Keytruda) and nivolumab (Opdivo).

In yet another embodiment, one of the active compounds described herein is administered in an effective amount for the treatment of abnormal tissue of the female reproductive system such as breast, ovarian, endometrial, or uterine cancer, in combination or alternation with an effective amount of an estrogen inhibitor including but not limited to a SERM (selective estrogen receptor modulator), a SERD (selective estrogen receptor degrader), a complete estrogen receptor degrader, or another form of partial or complete estrogen antagonist.

In one aspect, an active compound described herein is administered in an effective amount for the treatment of abnormal tissue of the male reproductive system such as prostate or testicular cancer, in combination or alternation with an effective amount of an androgen (such as testosterone) inhibitor including but not limited to a selective androgen receptor modulator, a selective androgen receptor degrader, a complete androgen receptor degrader, or another form of partial or complete androgen antagonist. In one embodiment, the prostate or testicular cancer is androgen-resistant.

In one embodiment, the compounds described herein inhibit a cyclin dependent kinase. For example, a compound described in the present invention provides G1-arresting effect on a subject's CDK replication dependent healthy cells. Non-limiting examples are hematopoetic stem progenitor cells, breast cancer cells, nonsmall cell lung cancer carcinoma, HSPCs or renal epithelial cells. The methods provided for herein are sufficient to afford chemoprotection to targeted CDK replication dependent healthy cells during chemotherapeutic agent exposure, for example, during the time period that a DNA-damaging chemotherapeutic agent is capable of DNA-damaging effects on CDK replication dependent healthy cells in the subject.

In one embodiment, the use of the compounds or methods described herein may be combined with the use of hematopoietic growth factors including, but not limited to, granulocyte colony stimulating factor (G-CSF), granulocyte-macrophage colony stimulating factor (GM-CSF), thrombopoietin, interleukin (IL)-12, steel factor, and erythropoietin (EPO), or a derivative thereof.

In one embodiment, the compound is administered prior to administration of the hematopoietic growth factor. In one embodiment, the hematopoietic growth factor administration is timed so that the compound's effect on HSPCs has dissipated.

In one embodiment, a compound described herein is administered in combination with a BTK inhibitor. In another embodiment, a compound described herein is administered in combination with an EGFR inhibitor.

In one embodiment, the compound of the present invention provides improved microsomal stability. In another embodiment, the compound of the present invention is useful in treating chemotherapy resistant cancer.

The present invention thus includes at least the following features:

(a) a compound of the present invention as described herein, and pharmaceutically acceptable salts and prodrugs thereof;

(b) a compound of the present invention as described herein, and pharmaceutically acceptable salts and prodrugs thereof that are useful in the treatment of a disorder of abnormal cellular proliferation, including a tumor or cancer;

(c) use of a compound of the present invention, or pharmaceutically acceptable salts and prodrugs thereof in the manufacture of a medicament for the treatment of a disorder of abnormal cellular proliferation, such as a tumor or cancer;

(d) a method for manufacturing a medicament intended for the therapeutic use of treating a disorder of abnormal cellular proliferation including a tumor or cancer, characterized in that a compound of the present invention as described herein is used in the manufacture;

(e) a compound of the present invention as described herein, and pharmaceutically acceptable salts and prodrugs thereof that are useful in the treatment of cancer, including any of the cancers described herein;

(f) use of a compound of the present invention, and pharmaceutically acceptable salts and prodrugs thereof in the manufacture of a medicament for the treatment of cancer, including any of the cancers described herein;

(g) a method for manufacturing a medicament intended for the therapeutic use of treating cancer, including any of the cancers described herein, characterized in that a compound of the present invention as described herein is used in the manufacture;

(h) a compound of the present invention as described herein, and pharmaceutically acceptable salts and prodrugs thereof that are useful in the treatment of a tumor, including any of the tumors described herein;

(i) use of a compound of the present invention, and pharmaceutically acceptable salts and prodrugs thereof in the manufacture of a medicament for the treatment of a tumor, including any of the tumors described herein;

(j) a method for manufacturing a medicament intended for the therapeutic use of treating a tumor, including any of the tumors described herein, characterized in that a compound of the present invention as described herein is used in the manufacture;

(k) a compound of the present invention as described herein, and pharmaceutically acceptable salts and prodrugs thereof that are useful in the treatment of a fibrotic disorder;

(l) use of a compound of the present invention, and pharmaceutically acceptable salts and prodrugs thereof in the manufacture of a medicament for the treatment of a fibrotic disorder;

(m) a method for manufacturing a medicament intended for the therapeutic use of treating a fibrotic disorder, characterized in that a compound of the present invention as described herein is used in the manufacture;

(n) a compound of the present invention as described herein, and pharmaceutically acceptable salts and prodrugs thereof that are useful in the treatment of an autoimmune or inflammatory disorder;

(o) use of a compound of the present invention, and pharmaceutically acceptable salts and prodrugs thereof in the manufacture of a medicament for the treatment of an autoimmune or inflammatory disorder;

(p) a method for manufacturing a medicament intended for the therapeutic use of treating an autoimmune or inflammatory disorder, characterized in that a compound of the present invention as described herein is used in the manufacture;

(q) a pharmaceutical formulation comprising an effective host-treating amount of the compound of the present invention or a pharmaceutically acceptable salt or prodrug thereof together with a pharmaceutically acceptable carrier or diluent;

(r) a compound of the present invention as described herein as a mixture of enantiomers or diastereomers (as relevant), including as a racemate;

(s) a compound of the present invention as described herein in enantiomerically or diastereomerically (as relevant) enriched form, including as an isolated enantiomer or diastereomer (i.e., greater than 85, 90, 95, 97 or 99% pure); and,

(t) a process for the preparation of therapeutic products that contain an effective amount of a compound of the present invention, as described herein.

(u) a compound of the present invention as described herein, and pharmaceutically acceptable salts and prodrugs thereof that are useful in chemoprotection;

(v) use of a compound of the present invention, and pharmaceutically acceptable salts and prodrugs thereof in the manufacture of a medicament for chemoprotection; and

(w) a method for manufacturing a medicament intended for the therapeutic use of chemoprotection, characterized in that a compound of the present invention as described herein is used in the manufacture.

### DETAILED DESCRIPTION

### I. Compounds

In one embodiment a compound of Formula I, Formula II, Formula III, Formula IV, Formula V, Formula VI, Formula VII, Formula VIII, Formula IX, Formula X, Formula XI, Formula XII, Formula XIII, or Formula XIV is provided:

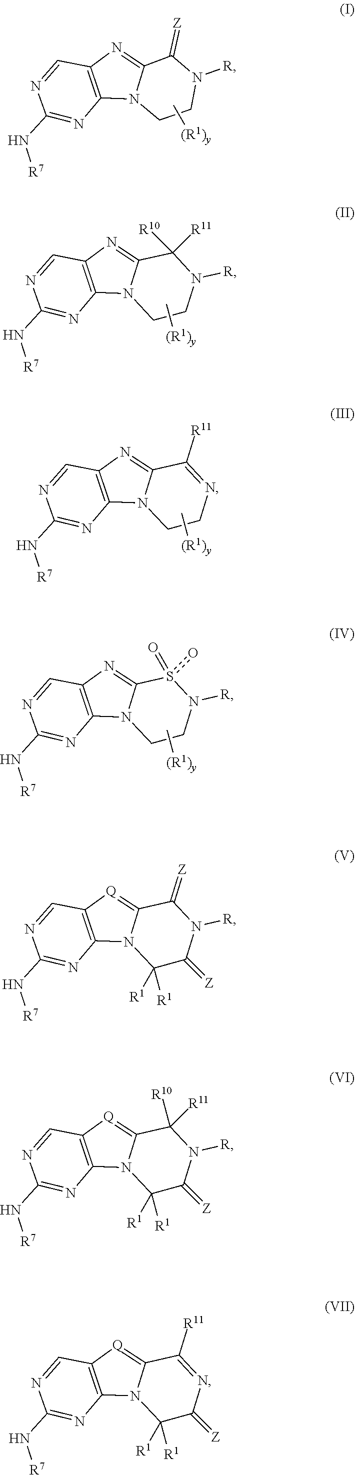

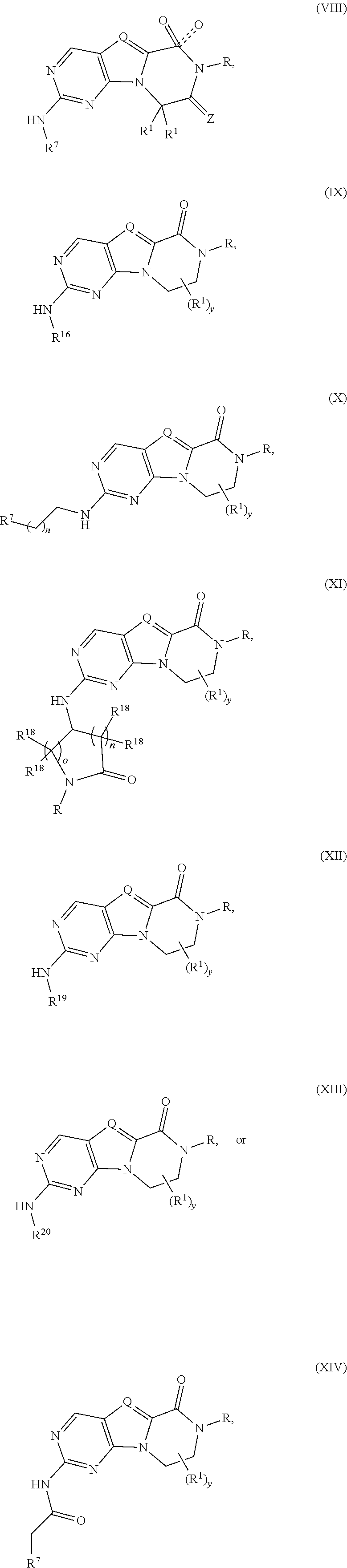

or a pharmaceutically acceptable salt, N-oxide, isotopic derivative, prodrug, and/or a pharmaceutically acceptable composition thereof; wherein the variables are as defined above in the Summary.

In one embodiment, the compound of the present invention is of formula:

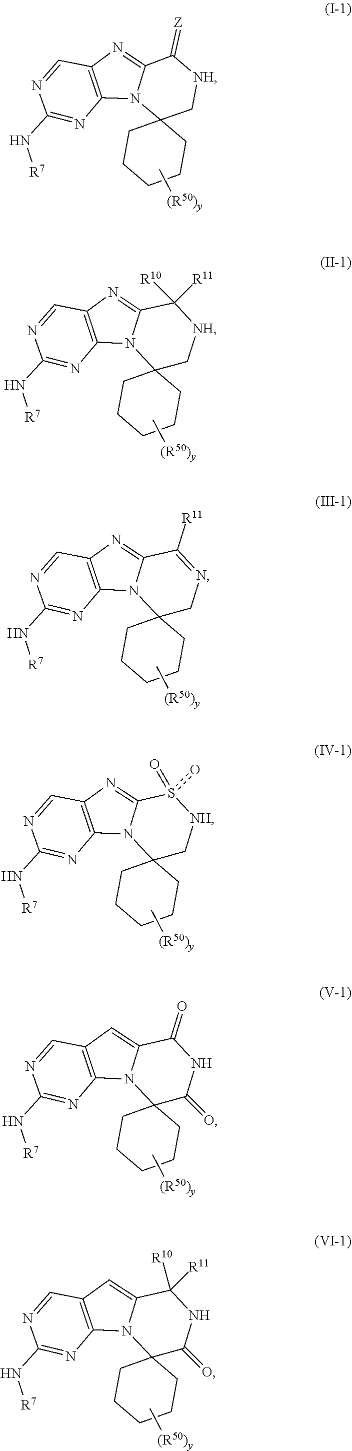

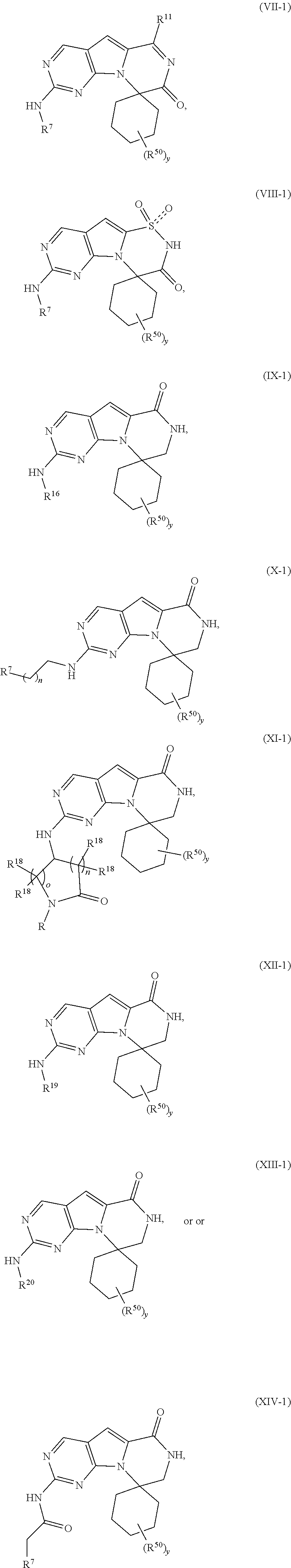

or a pharmaceutically acceptable salt, N-oxide, isotopic derivative, prodrug, and/or a pharmaceutically acceptable composition thereof; wherein the variables are as defined above in the Summary.

In another embodiment the compound is of Formula XV-1, Formula XVI-1, or Formula XVII-1:

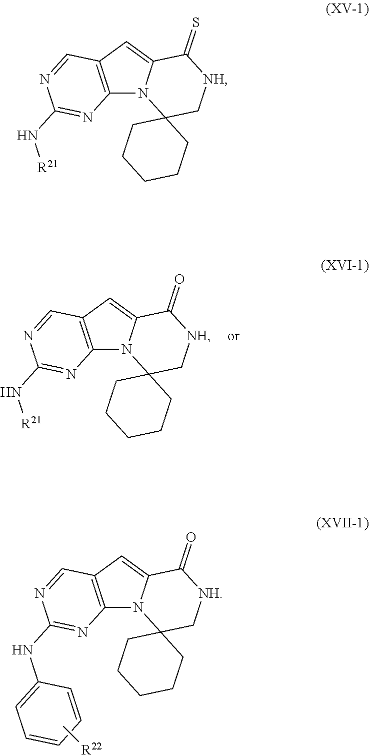

In one embodiment, the compound of the present invention is of formula:

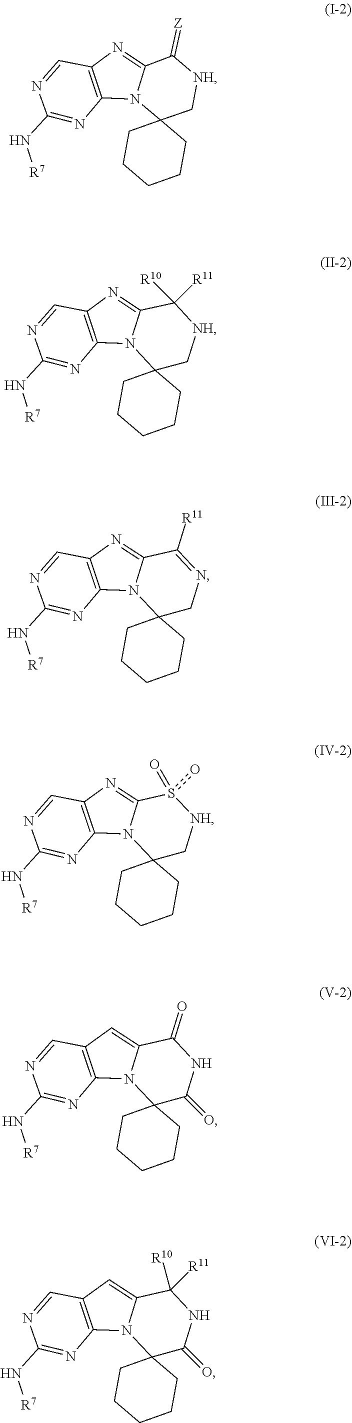

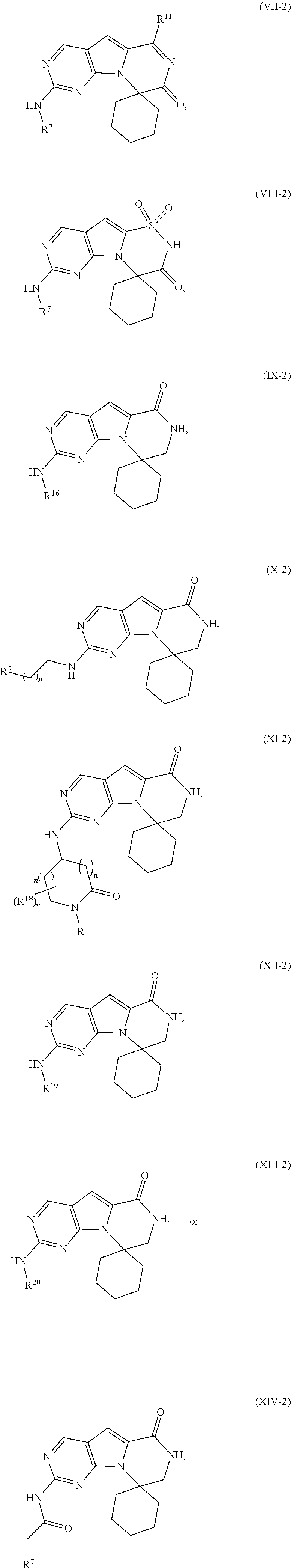

or a pharmaceutically acceptable salt, N-oxide, isotopic derivative, prodrug, and/or a pharmaceutically acceptable composition thereof; wherein the variables are as defined above in the Summary.

In one embodiment, the compound of the present invention is of formula:

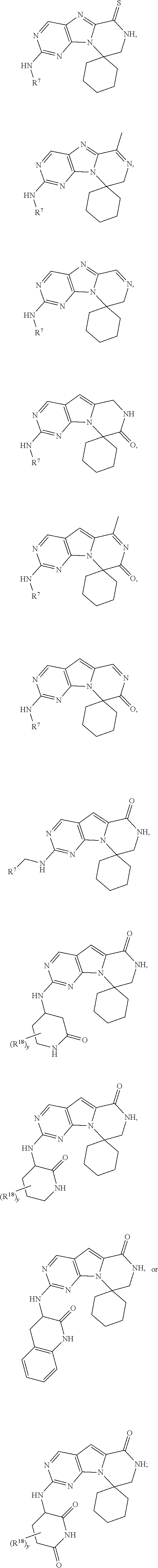

or a pharmaceutically acceptable salt, N-oxide, isotopic derivative, prodrug, and/or a pharmaceutically acceptable composition thereof; wherein the variables are as defined above in the Summary.

In one embodiment, the compound of the present invention is of formula:

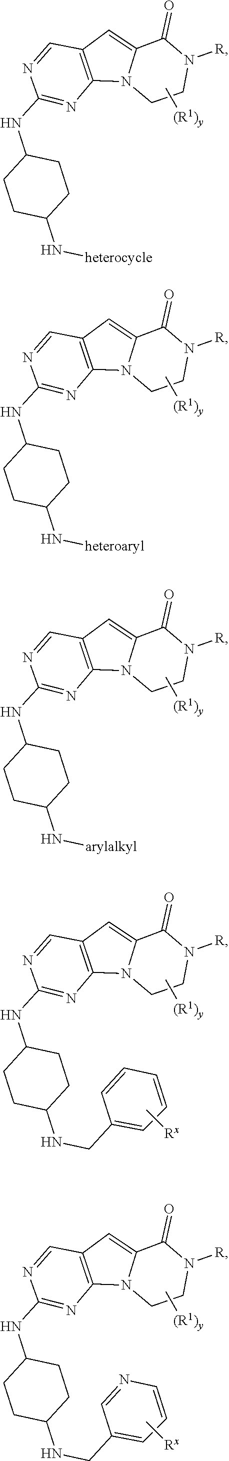

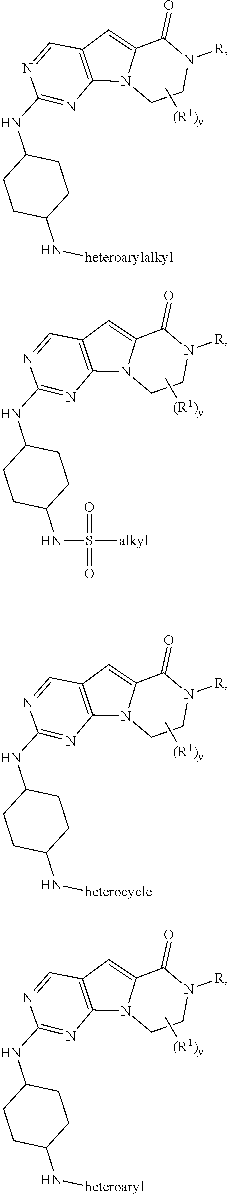

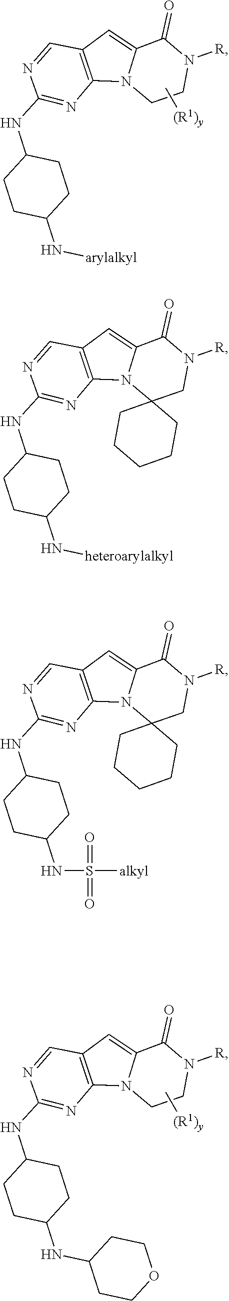

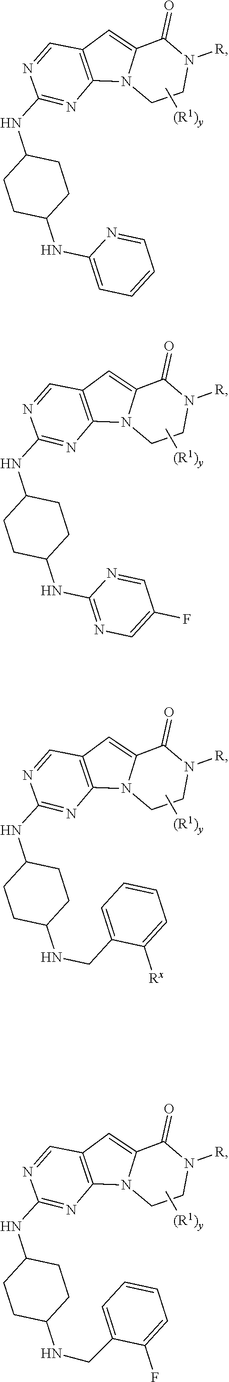

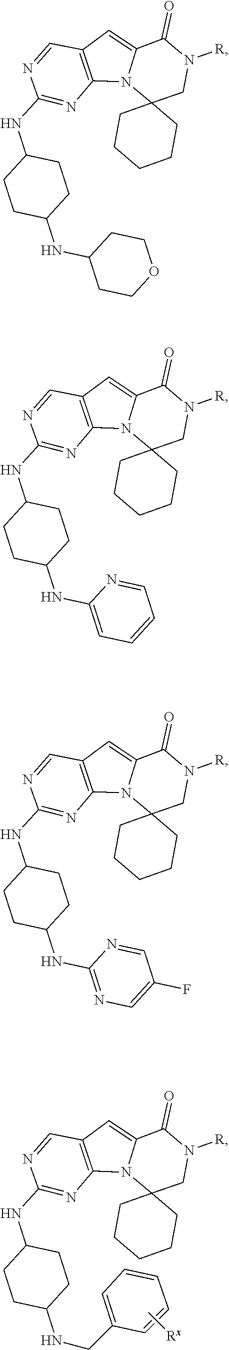

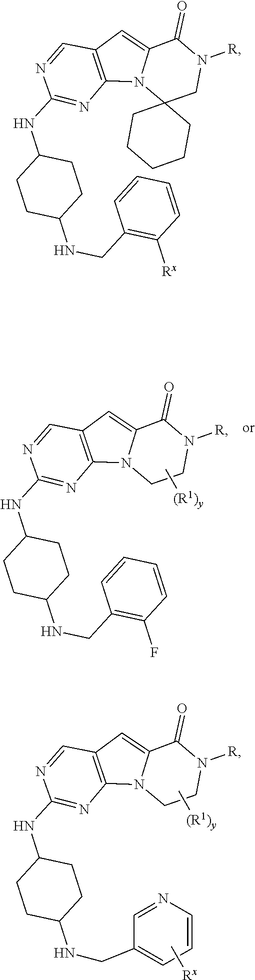

or a pharmaceutically acceptable salt, N-oxide, isotopic derivative, prodrug, and/or a pharmaceutically acceptable composition thereof; wherein the variables are as defined above in the Summary.

In one embodiment, the compound of the present invention is of formula:

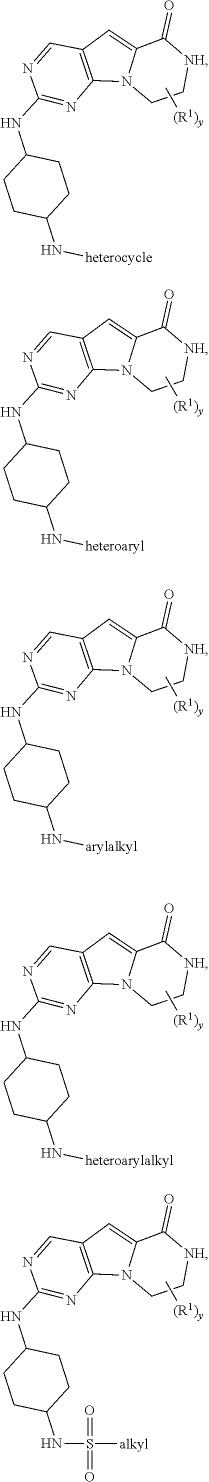

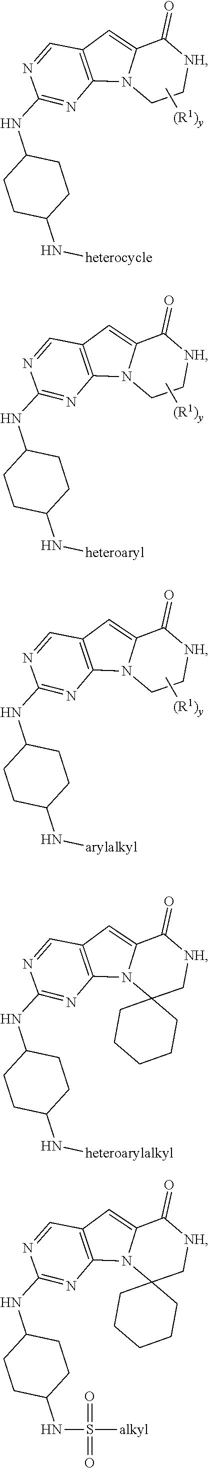

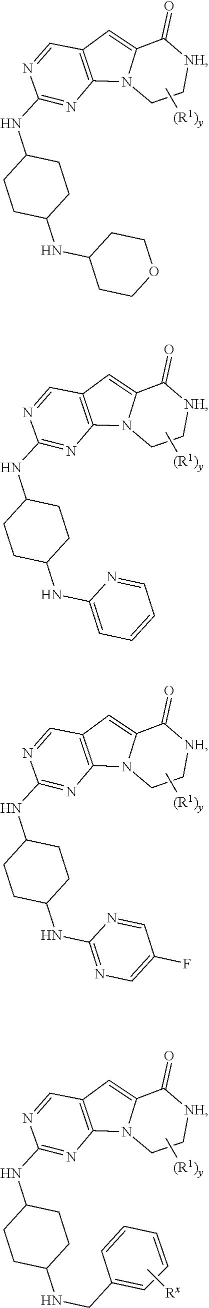

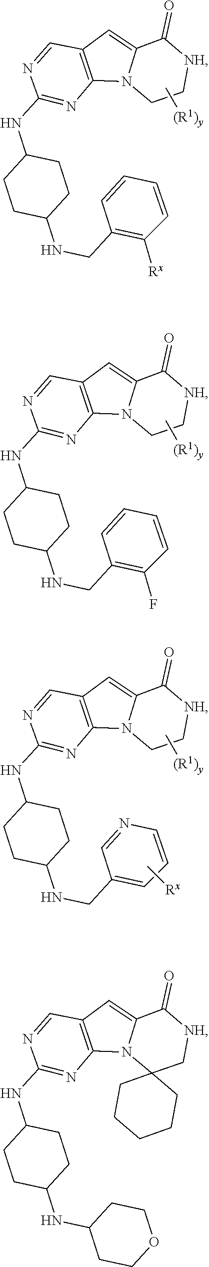

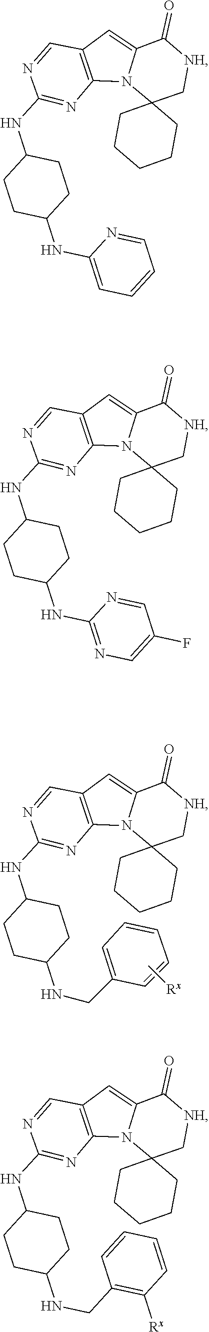

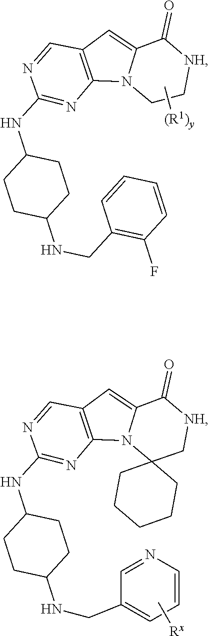

or a pharmaceutically acceptable salt, N-oxide, isotopic derivative, prodrug, and/or a pharmaceutically acceptable composition thereof, wherein the variables are as defined above in the Summary.

In some aspects, the compound is selected from:

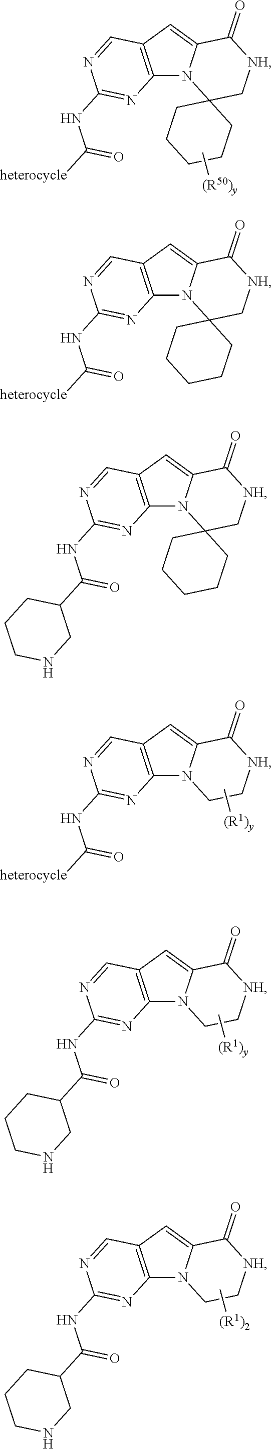

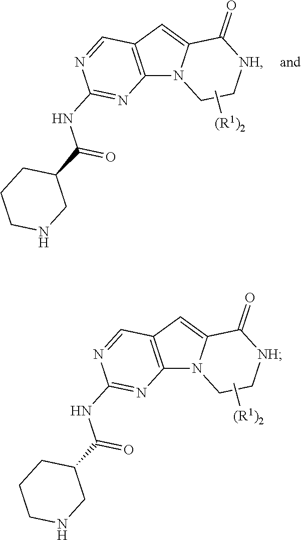

or a pharmaceutically acceptable salt, N-oxide, isotopic derivative, prodrug, and/or a pharmaceutically acceptable composition thereof, wherein the variables are as defined above in the Summary.

In some aspects, the compound is selected from:

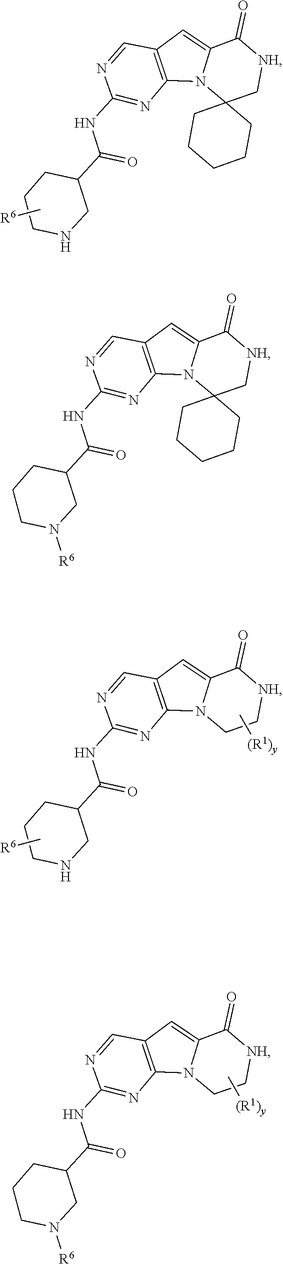

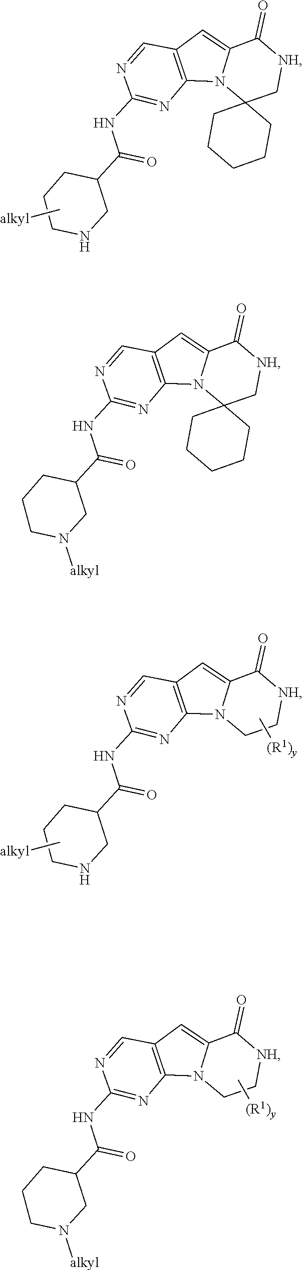

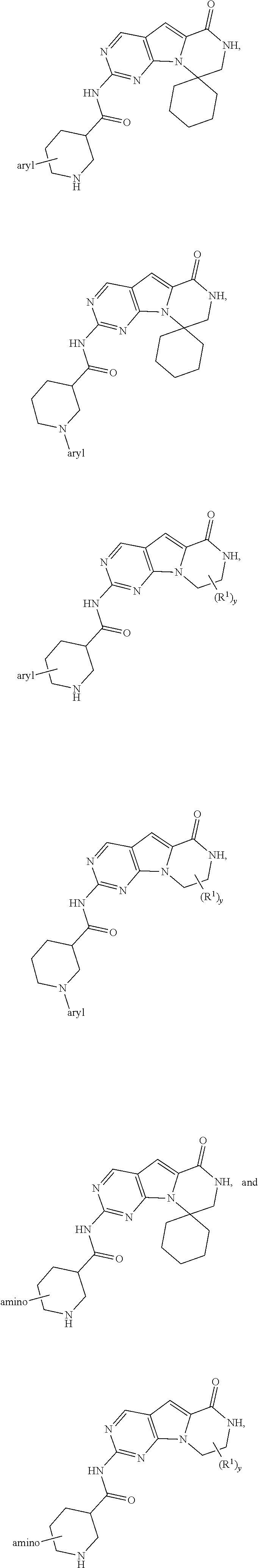

or a pharmaceutically acceptable salt, N-oxide, isotopic derivative, prodrug, and/or a pharmaceutically acceptable composition thereof; wherein the variables are as defined above in the Summary.

### Embodiments of “Alkyl”

In one embodiment “alkyl” is a C₁-C₁₀alkyl, C₁-C₉alkyl, C₁-C₅alkyl, C₁-C₇alkyl, C₁-C₆alkyl, C₁-C₅alkyl, C₁-C₄alkyl, C₁-C₃alkyl, or C₁-C₂alkyl.

In one embodiment “alkyl” has one carbon.

In one embodiment “alkyl” has two carbons.

In one embodiment “alkyl” has three carbons.

In one embodiment “alkyl” has four carbons.

In one embodiment “alkyl” has five carbons.

In one embodiment “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In one embodiment “alkyl” is “substituted alkyl”.

In one embodiment “alkenyl” is “substituted alkenyl”.

In one embodiment “alkynyl” is “substituted alkynyl”.

### Embodiments of “Haloalkyl”

In one embodiment “haloalkyl” is a C₁-C₁₀haloalkyl, C₁-C₉haloalkyl, C₁-C₅haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In one embodiment “haloalkyl” has one carbon.

In one embodiment “haloalkyl” has one carbon and one halogen.

In one embodiment “haloalkyl” has one carbon and two halogens.

In one embodiment “haloalkyl” has one carbon and three halogens.

In one embodiment “haloalkyl” has two carbons.

In one embodiment “haloalkyl” has three carbons.

In one embodiment “haloalkyl” has four carbons.

In one embodiment “haloalkyl” has five carbons.

In one embodiment “haloalkyl” has six carbons.

Non-limiting examples of “haloalkyl” include:

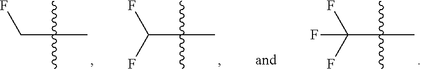

Additional non-limiting examples of “haloalkyl” include:

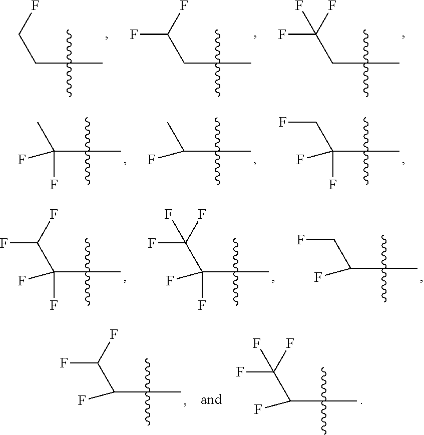

Additional non-limiting examples of “haloalkyl” include:

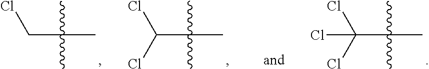

Additional non-limiting examples of “haloalkyl” include:

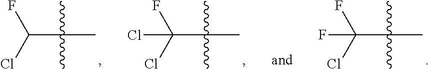

### Embodiments of “Aryl”

In one embodiment “aryl” is a 6 carbon aromatic group (phenyl).

In one embodiment “aryl” is a 10 carbon aromatic group (napthyl).

In one embodiment “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

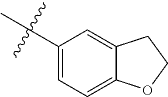 [SMILES: *c1ccc2c(c1)CCO2]

is an “aryl” group.

However,

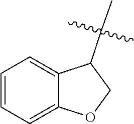 [SMILES: *C1COc2ccccc21]

is a “heterocycle” group.

In one embodiment “aryl” is a 6 carbon aromatic group fused to a cycloalkyl wherein the point of attachment is the aryl ring. Non-limiting examples of “aryl” include dihydro-indene and tetrahydronaphthalene wherein the point of attachment for each group is on the aromatic ring.

For example

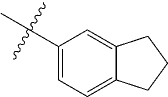 [SMILES: *c1ccc2c(c1)CCC2]

is an “aryl” group.

However,

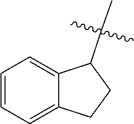 [SMILES: *C1CCc2ccccc21]

is a “cycloalkyl” group.

In one embodiment “aryl” is “substituted aryl”.

### Embodiments of “Heteroaryl”

In one embodiment “heteroaryl” is a 5 membered aromatic group containing 1, 2, 3, or 4 nitrogen atoms.

Non-limiting examples of 5 membered “heteroaryl” groups include pyrrole, furan, thiophene, pyrazole, imidazole, triazole, tetrazole, isoxazole, oxazole, oxadiazole, oxatriazole, isothiazole, thiazole, thiadiazole, and thiatriazole.

Additional non-limiting examples of 5 membered “heteroaryl” groups include:

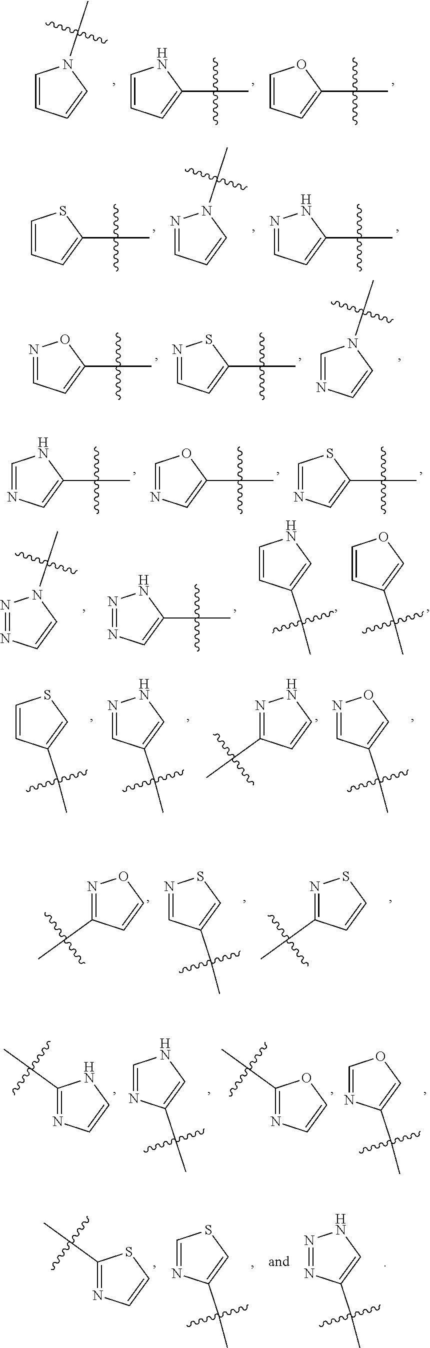

In one embodiment “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

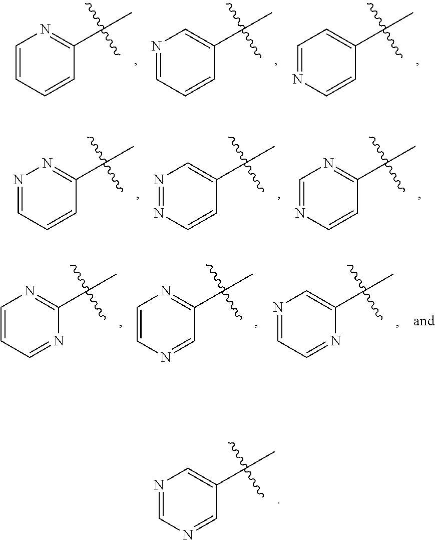

In one embodiment “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

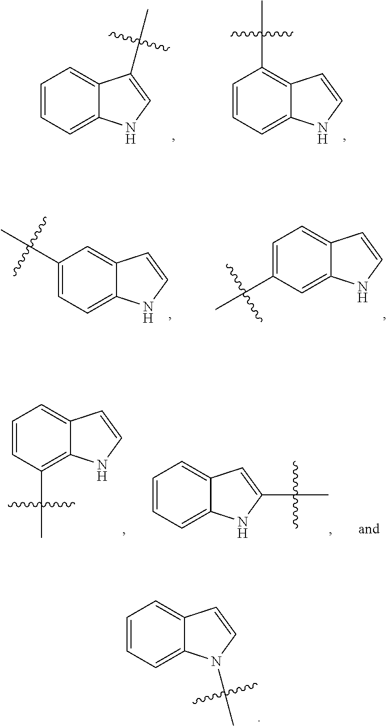

Additional non-limiting examples of “heteroaryl” groups that are bicyclic include:

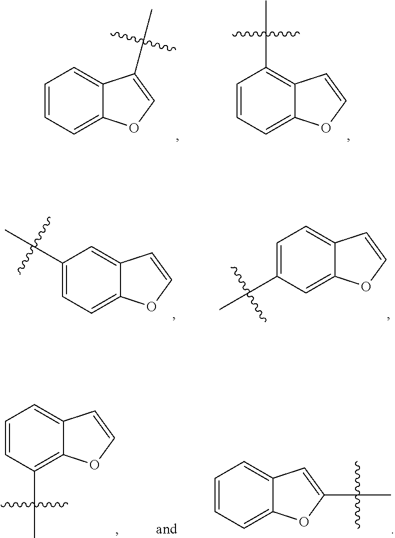 [SMILES: *c1coc2ccccc12 ; *c1cccc2occc12 ; *c1ccc2occc2c1 ; *c1ccc2ccoc2c1 ; *c1cccc2ccoc12 ; *c1cc2ccccc2o1]

Additional non-limiting examples of “heteroaryl” groups that are bicyclic include:

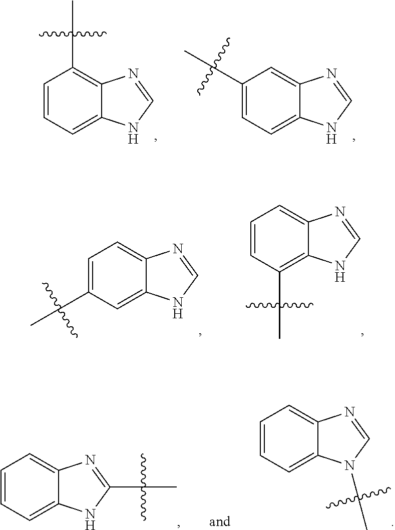 [SMILES: *c1cccc2[nH]cnc12 ; *c1ccc2[nH]cnc2c1 ; *c1ccc2nc[nH]c2c1 ; *c1cccc2nc[nH]c12 ; *c1nc2ccccc2[nH]1 ; *n1cnc2ccccc21]

In one embodiment “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

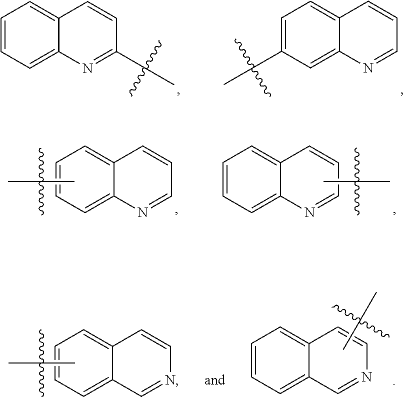

In one embodiment “heteroaryl” is “substituted heteroaryl”.

### Embodiments of “Cycloalkyl”

In one embodiment “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₅-C₈cycloalkyl, or C₆-C₈cycloalkyl.

In one embodiment “cycloalkyl” has three carbons.

In one embodiment “cycloalkyl” has four carbons.

In one embodiment “cycloalkyl” has five carbons.

In one embodiment “cycloalkyl” has six carbons.

In one embodiment “cycloalkyl” has seven carbons.

In one embodiment “cycloalkyl” has eight carbons.

In one embodiment “cycloalkyl” has nine carbons.

In one embodiment “cycloalkyl” has ten carbons.

Non-limiting examples of “cycloalkyl” include: cyclopropyl, cyclobutyl, cyclopentyl, cyclohexyl, cycloheptyl, cyclooctyl, and cyclodecyl.

Additional non-limiting examples of “cycloalkyl” include dihydro-indene and tetrahydronaphthalene wherein the point of attachment for each group is on the cycloalkyl ring.

For example,

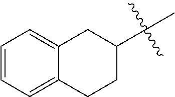 [SMILES: *C1CCc2ccccc2C1]

is an “cycloalkyl” group.

However,

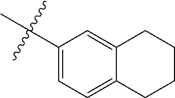 [SMILES: *c1ccc2c(c1)CCCC2]

is an “aryl” group.

In one embodiment “cycloalkyl” is a “substituted cycloalkyl”.

### Embodiments of “Heterocycle”

In one embodiment “heterocycle” refers to a cyclic ring with one nitrogen and 3, 4, 5, 6, 7, or 8 carbon atoms.

In one embodiment “heterocycle” refers to a cyclic ring with one nitrogen and one oxygen and 3, 4, 5, 6, 7, or 8 carbon atoms.

In one embodiment “heterocycle” refers to a cyclic ring with two nitrogens and 3, 4, 5, 6, 7, or 8 carbon atoms.

In one embodiment “heterocycle” refers to a cyclic ring with one oxygen and 3, 4, 5, 6, 7, or 8 carbon atoms.

In one embodiment “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

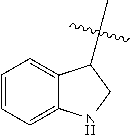 [SMILES: *C1CNc2ccccc21]

is a “heterocycle” group.

However,

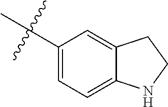 [SMILES: *c1ccc2c(c1)CCN2]

is an “aryl” group.

Non-limiting examples of “heterocycle” also include:

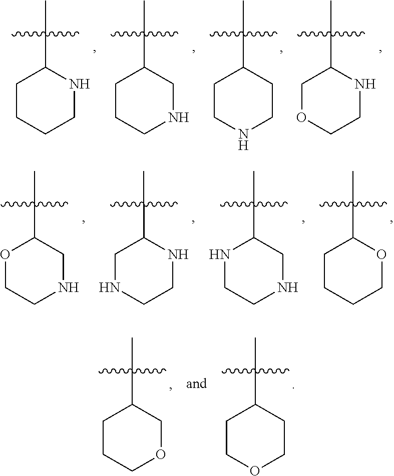

Additional non-limiting examples of “heterocycle” include:

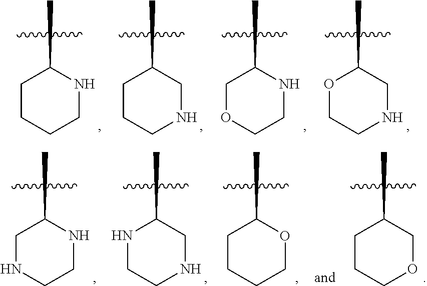

Additional non-limiting examples of “heterocycle” include:

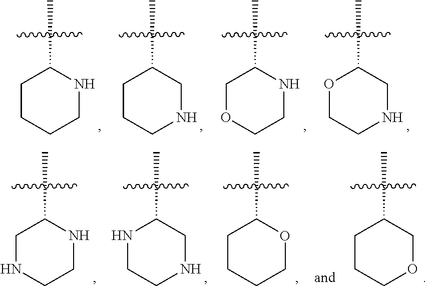

Non-limiting examples of “heterocycle” also include:

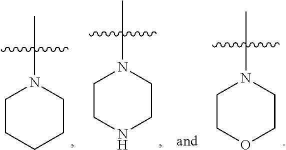

Non-limiting examples of “heterocycle” also include:

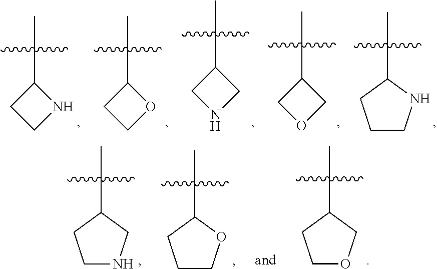

Additional non-limiting examples of “heterocycle” include:

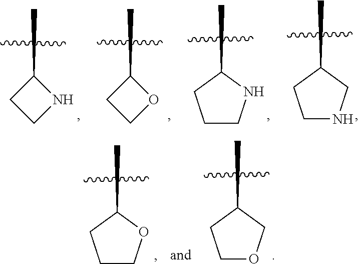

Additional non-limiting examples of “heterocycle” include:

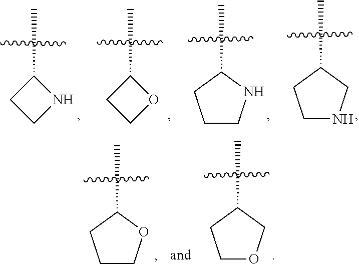

In one embodiment “heterocycle” is “substituted heterocycle”.

### Embodiments of “Alkyl-Aryl”

In one embodiment the “alkyl-aryl” refers to a 1 carbon alkyl group substituted with an aryl group.

Non-limiting examples of “alkyl-aryl” include:

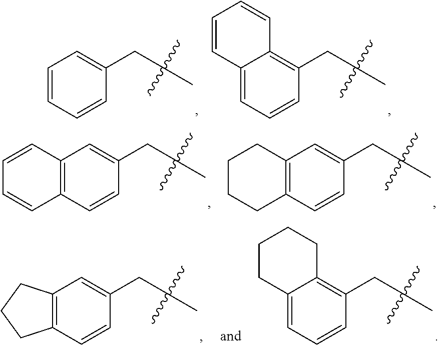

In one embodiment “alkyl-aryl” is

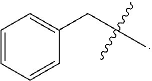 [SMILES: *Cc1ccccc1]

In one embodiment the “alkyl-aryl” refers to a 2 carbon alkyl group substituted with an aryl group.

Non-limiting examples of “alkyl-aryl” include:

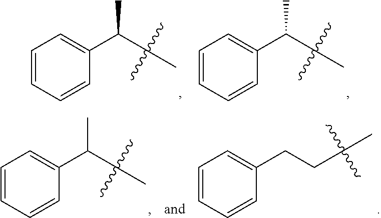

In one embodiment the “alkyl-aryl” refers to a 3 carbon alkyl group substituted with an aryl group.

### Optional Substituents

In one embodiment a group described herein that can be substituted with 1, 2, 3, or 4 substituents is substituted with one substituent.

In one embodiment a group described herein that can be substituted with 1, 2, 3, or 4 substituents is substituted with two substituents.

In one embodiment a group described herein that can be substituted with 1, 2, 3, or 4 substituents is substituted with three substituents.

In one embodiment a group described herein that can be substituted with 1, 2, 3, or 4 substituents is substituted with four substituents.

### Exemplary Compounds of the Present Invention:

In one embodiment the compound of the present invention is selected from:

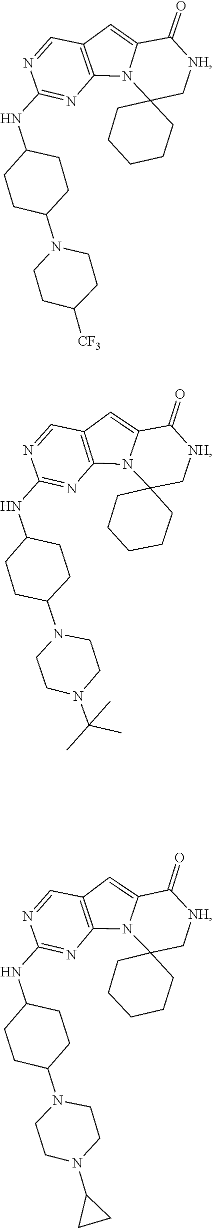 [SMILES: O=C1NCC2(CCCCC2)n2c1cc1cnc(NC3CCC(N4CCC(C(F)(F)F)CC4)CC3)nc12 ; CC(C)(C)N1CCN(C2CCC(Nc3ncc4cc5n(c4n3)C3(CCCCC3)CNC5=O)CC2)CC1 ; O=C1NCC2(CCCCC2)n2c1cc1cnc(NC3CCC(N4CCN(C5CC5)CC4)CC3)nc12]

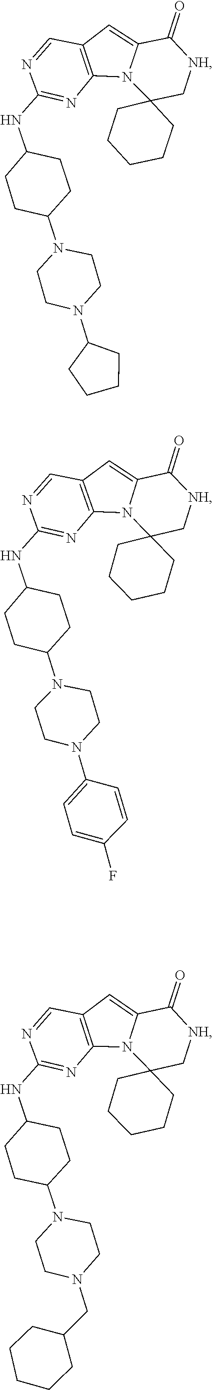 [SMILES: O=C1NCC2(CCCCC2)n2c1cc1cnc(NC3CCC(N4CCN(C5CCCC5)CC4)CC3)nc12 ; O=C1NCC2(CCCCC2)n2c1cc1cnc(NC3CCC(N4CCN(c5ccc(F)cc5)CC4)CC3)nc12 ; O=C1NCC2(CCCCC2)n2c1cc1cnc(NC3CCC(N4CCN(CC5CCCCC5)CC4)CC3)nc12]

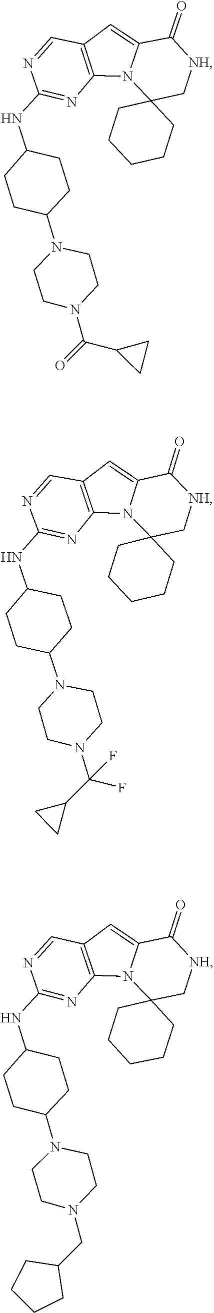 [SMILES: O=C1NCC2(CCCCC2)n2c1cc1cnc(NC3CCC(N4CCN(C(=O)C5CC5)CC4)CC3)nc12 ; O=C1NCC2(CCCCC2)n2c1cc1cnc(NC3CCC(N4CCN(C(F)(F)C5CC5)CC4)CC3)nc12 ; O=C1NCC2(CCCCC2)n2c1cc1cnc(NC3CCC(N4CCN(CC5CCCC5)CC4)CC3)nc12]

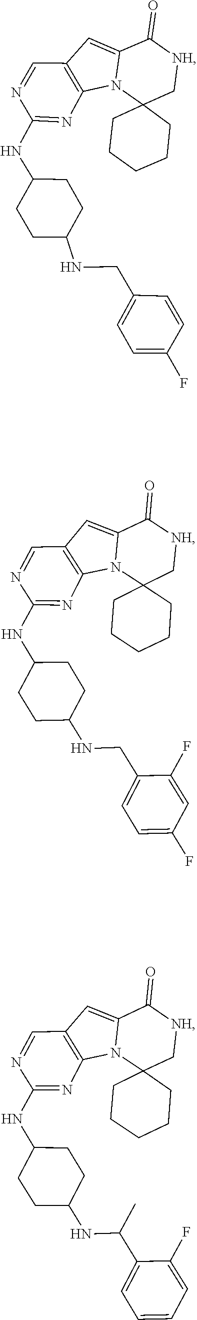 [SMILES: O=C1NCC2(CCCCC2)n2c1cc1cnc(NC3CCC(NCc4ccc(F)cc4)CC3)nc12 ; O=C1NCC2(CCCCC2)n2c1cc1cnc(NC3CCC(NCc4ccc(F)cc4F)CC3)nc12 ; CC(NC1CCC(Nc2ncc3cc4n(c3n2)C2(CCCCC2)CNC4=O)CC1)c1ccccc1F]

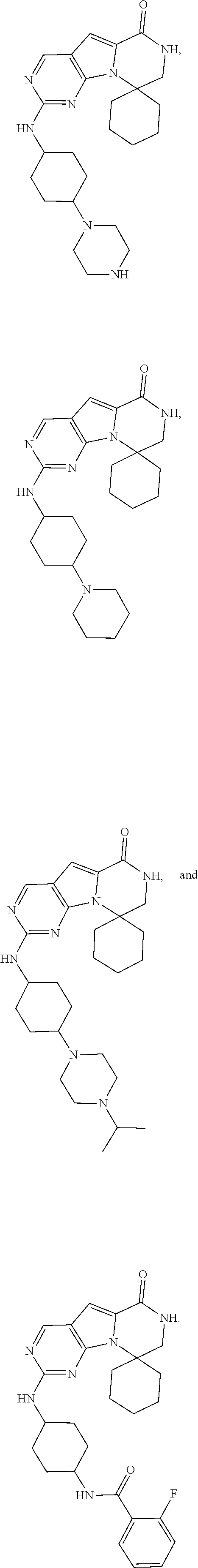 [SMILES: O=C1NCC2(CCCCC2)n2c1cc1cnc(NC3CCC(N4CCNCC4)CC3)nc12 ; O=C1NCC2(CCCCC2)n2c1cc1cnc(NC3CCC(N4CCCCC4)CC3)nc12 ; CC(C)N1CCN(C2CCC(Nc3ncc4cc5n(c4n3)C3(CCCCC3)CNC5=O)CC2)CC1 ; O=C(NC1CCC(Nc2ncc3cc4n(c3n2)C2(CCCCC2)CNC4=O)CC1)c1ccccc1F]

In one embodiment the compound of the present invention is selected from:

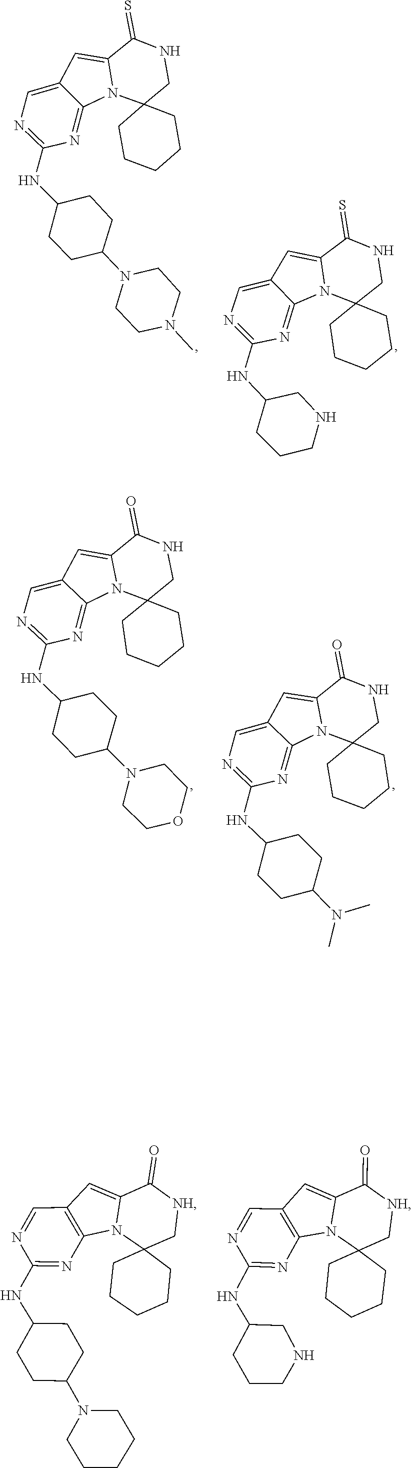

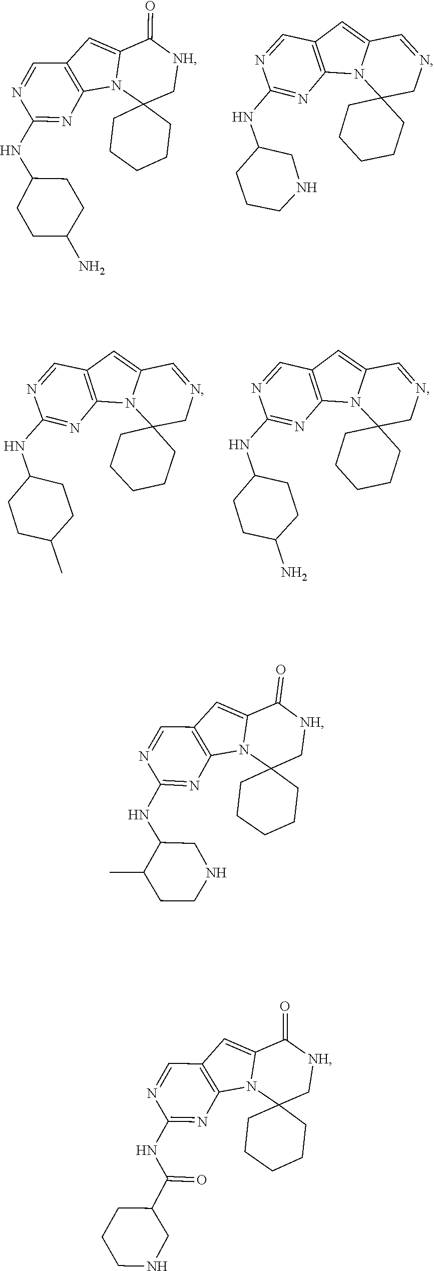 [SMILES: NC1CCC(Nc2ncc3cc4n(c3n2)C2(CCCCC2)CNC4=O)CC1 ; C1=NCC2(CCCCC2)n2c1cc1cnc(NC3CCCNC3)nc12 ; CC1CCC(Nc2ncc3cc4n(c3n2)C2(CCCCC2)CN=C4)CC1 ; NC1CCC(Nc2ncc3cc4n(c3n2)C2(CCCCC2)CN=C4)CC1 ; CC1CCNCC1Nc1ncc2cc3n(c2n1)C1(CCCCC1)CNC3=O ; O=C1NCC2(CCCCC2)n2c1cc1cnc(NC(=O)C3CCCNC3)nc12]

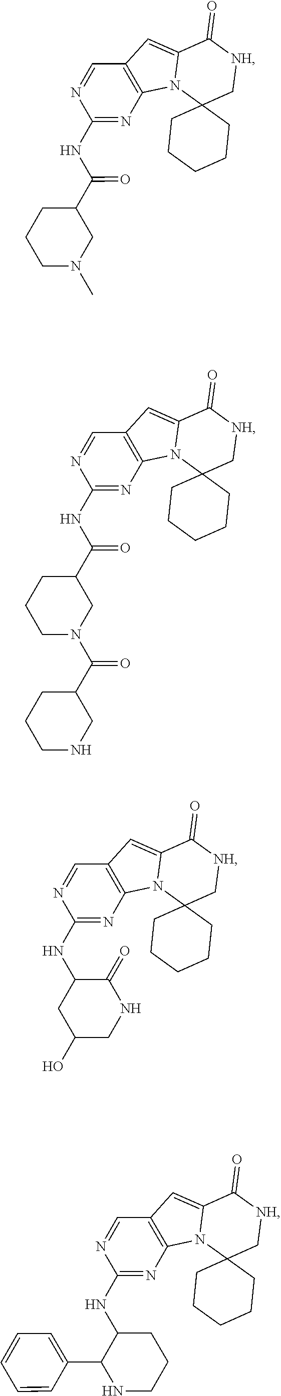 [SMILES: CN1CCCC(C(=O)Nc2ncc3cc4n(c3n2)C2(CCCCC2)CNC4=O)C1 ; O=C1NCC2(CCCCC2)n2c1cc1cnc(NC(=O)C3CCCN(C(=O)C4CCCNC4)C3)nc12 ; O=C1NCC2(CCCCC2)n2c1cc1cnc(NC3CC(O)CNC3=O)nc12 ; O=C1NCC2(CCCCC2)n2c1cc1cnc(NC3CCCNC3c3ccccc3)nc12]

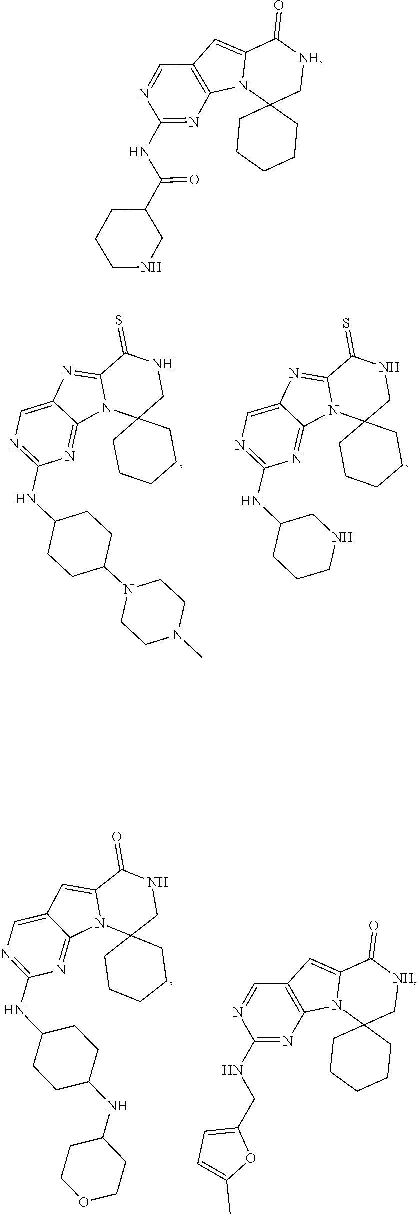 [SMILES: O=C1NCC2(CCCCC2)n2c1cc1cnc(NC(=O)C3CCCNC3)nc12 ; CN1CCN(C2CCC(Nc3ncc4nc5n(c4n3)C3(CCCCC3)CNC5=S)CC2)CC1 ; S=C1NCC2(CCCCC2)n2c1nc1cnc(NC3CCCNC3)nc12 ; O=C1NCC2(CCCCC2)n2c1cc1cnc(NC3CCC(NC4CCOCC4)CC3)nc12 ; Cc1ccc(CNc2ncc3cc4n(c3n2)C2(CCCCC2)CNC4=O)o1]

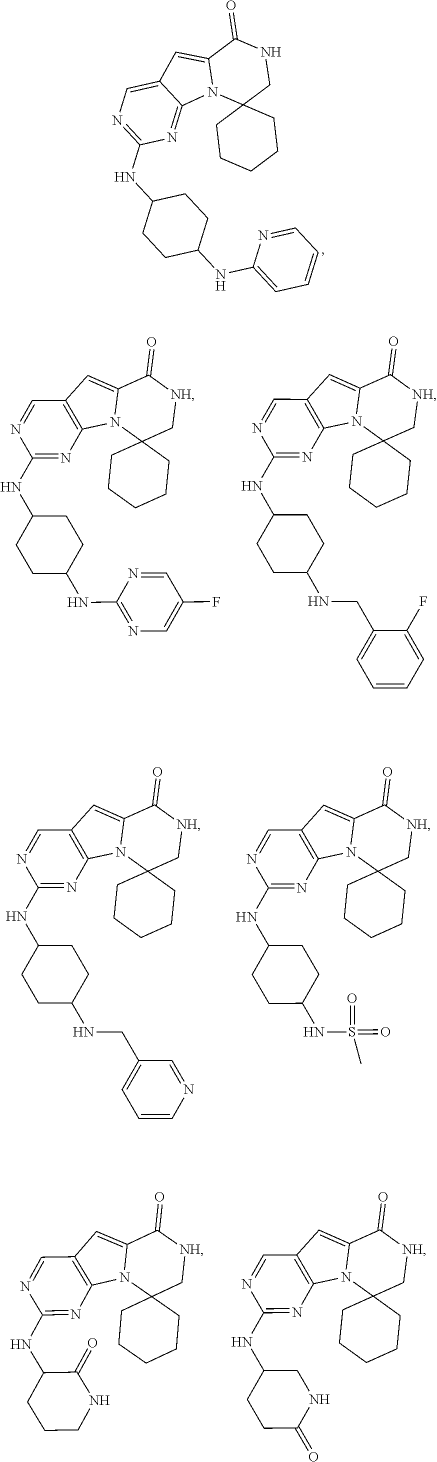

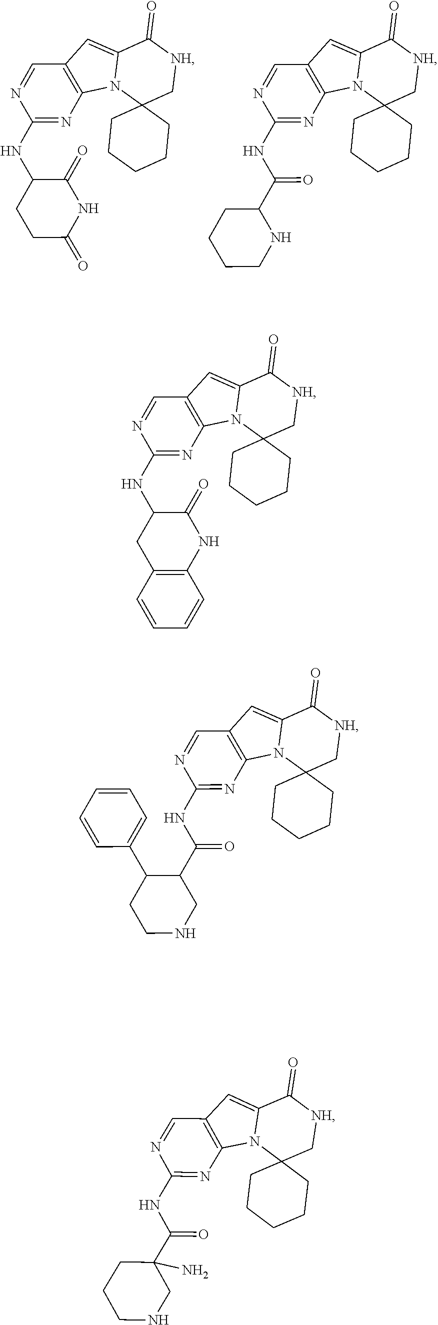 [SMILES: O=C1CCC(Nc2ncc3cc4n(c3n2)C2(CCCCC2)CNC4=O)C(=O)N1 ; O=C1NCC2(CCCCC2)n2c1cc1cnc(NC(=O)C3CCCCN3)nc12 ; O=C1NCC2(CCCCC2)n2c1cc1cnc(NC3Cc4ccccc4NC3=O)nc12 ; O=C1NCC2(CCCCC2)n2c1cc1cnc(NC(=O)C3CNCCC3c3ccccc3)nc12 ; NC1(C(=O)Nc2ncc3cc4n(c3n2)C2(CCCCC2)CNC4=O)CCCNC1]

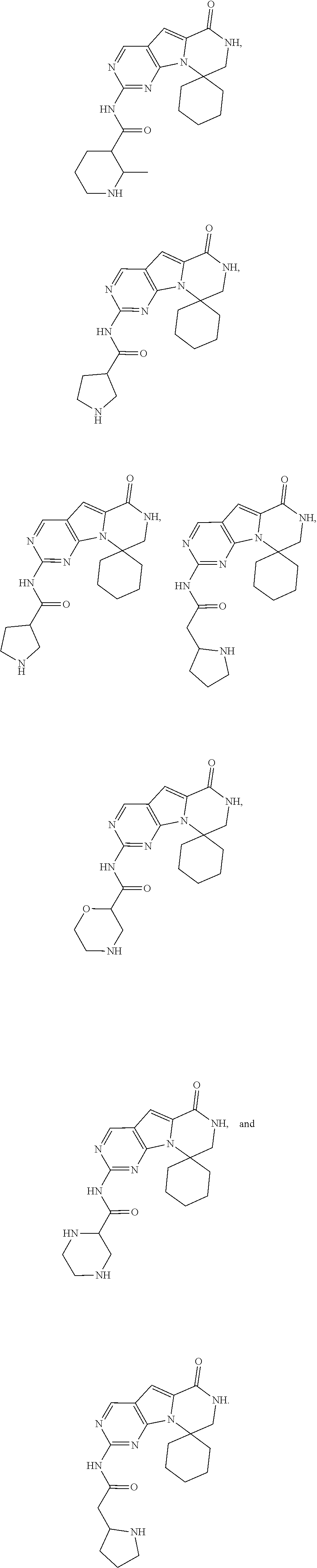

In one embodiment the compound of the present invention is selected from:

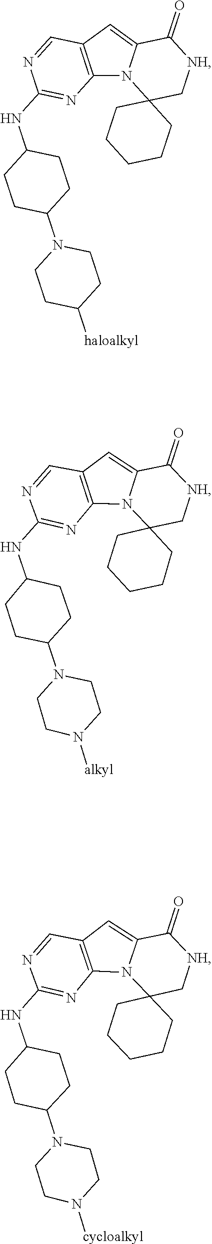

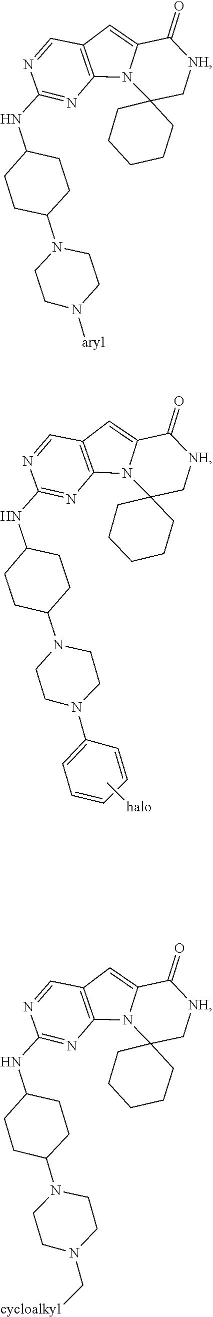

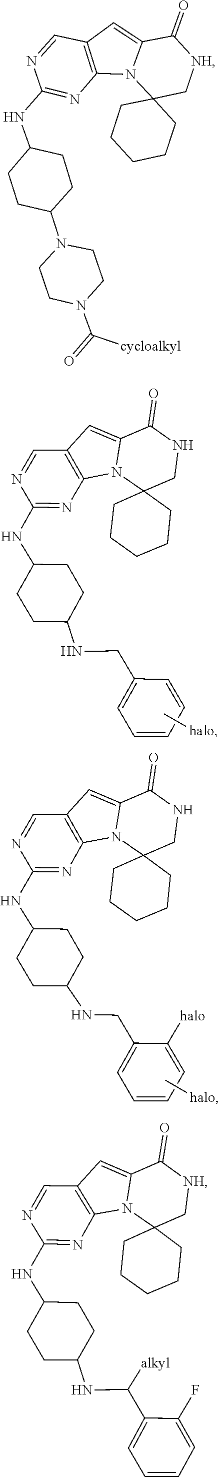

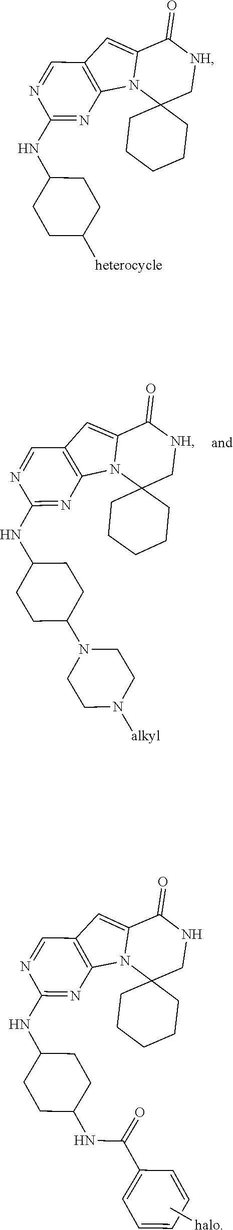

In one embodiment the compound of the present invention is selected from:

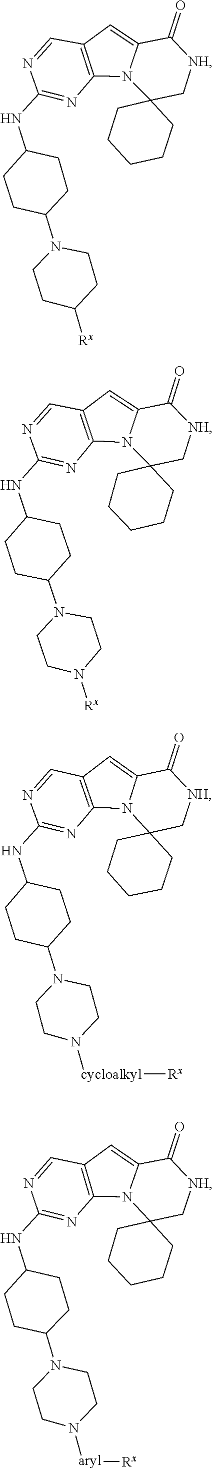

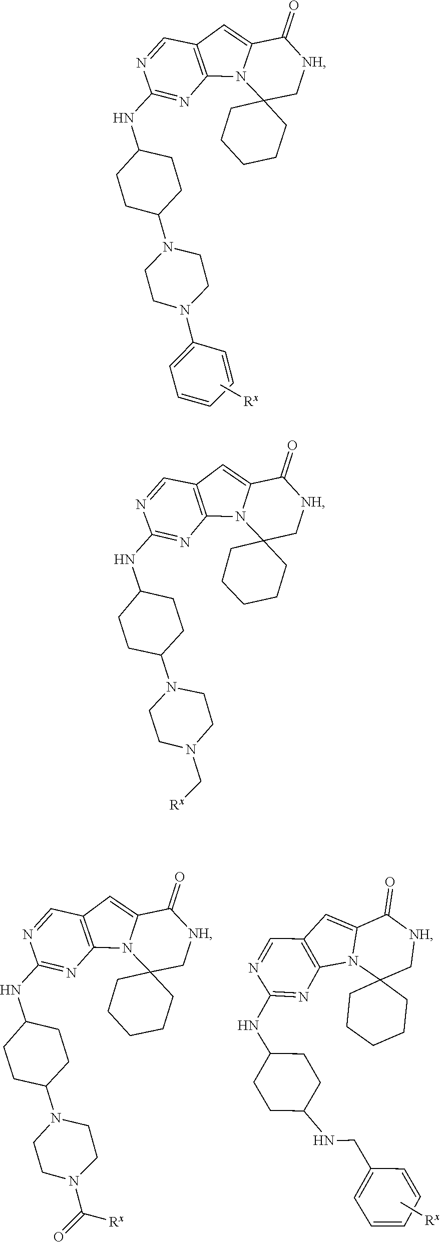

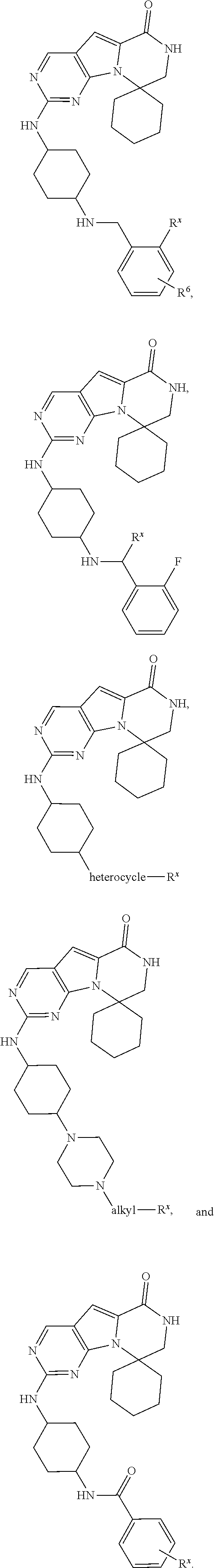

In one embodiment the compounds above are trans disasteromers.

In another embodiment the compounds above are cis diasteromers.

### Embodiments of R¹

In one embodiment y is 1 and R¹ is aryl.

In one embodiment y is 1 and R¹ is phenyl.

In one embodiment y is 1 and R¹ is alkyl.

In one embodiment y is 2 and the two R¹s are on the same ring atom and combine to form a 5 membered cycloalkyl. In one embodiment the cycloalkyl is substituted with one R⁵¹ substituent. In one embodiment the cycloalkyl is substituted with two R⁵¹ substituents. In one embodiment the cycloalkyl is substituted with NH₂. In one embodiment the cycloalkyl is substituted with OR¹⁴. In one embodiment the cycloalkyl is substituted with OH. In one embodiment the cycloalkyl is substituted with alkyl. In one embodiment the cycloalkyl is substituted with CH₃.

In one embodiment y is 2 and the two R¹s are on the same ring atom and combine to form a 6 membered cycloalkyl. In one embodiment the cycloalkyl is substituted with one R⁵⁰ substituent. In one embodiment the cycloalkyl is substituted with two R⁵⁰ substituents. In one embodiment the cycloalkyl is substituted with NH₂. In one embodiment the cycloalkyl is substituted with OR¹⁴. In one embodiment the cycloalkyl is substituted with OH. In one embodiment the cycloalkyl is substituted with alkyl. In one embodiment the cycloalkyl is substituted with CH₃.

In one embodiment y is 2 and the two R¹s are on the same ring atom and combine to form a 5 membered heterocycle. In one embodiment the heterocycle is substituted with one R⁵⁰ substituent. In one embodiment the heterocycle is substituted with two R⁵⁰ substituents. In one embodiment the heterocycle is substituted with N12. In one embodiment the heterocycle is substituted with OR¹⁴. In one embodiment the heterocycle is substituted with OH. In one embodiment the heterocycle is substituted with alkyl. In one embodiment the heterocycle is substituted with CH₃.

In one embodiment y is 2 and the two R¹s are on the same ring atom and combine to form a 6 membered heterocycle. In one embodiment the heterocycle is substituted with one R⁵⁰ substituent. In one embodiment the heterocycle is substituted with two R⁵⁰ substituents. In one embodiment the heterocycle is substituted with N12. In one embodiment the heterocycle is substituted with OR¹⁴. In one embodiment the heterocycle is substituted with OH. In one embodiment the heterocycle is substituted with alkyl. In one embodiment the heterocycle is substituted with CH₃.

### Embodiments of m, n, and o

In one embodiment m is 0.

In one embodiment m is 1.

In one embodiment n is 0.

In one embodiment n is 1.

In one embodiment n is 2.

In one embodiment o is 0.

In one embodiment o is 1.

In one embodiment o is 2.

In one embodiment o is 3.

### Embodiments of R²

In some aspects, R² is -(alkylene)ₘ-heterocycle, -(alkylene)ₘ-heteroaryl, -(alkylene)ₘ-NR³R⁴, -(alkylene)ₘ-C(O)—NR³R⁴; -(alkylene)ₘ-O—R⁵, -(alkylene)ₘ-S(O)ₙ—R, or -(alkylene)ₘ-S(O)ₙ—NR³R⁴; any of which may be optionally independently substituted with 1, 2, 3, or 4 R^{x} groups as allowed by valance, and wherein two R^{x} groups bound to the same or adjacent atom may optionally combine to form a ring and wherein m is 0 or 1 and n is 0, 1 or 2.

In some aspects, R² is -(alkylene)ₘ-heterocycle, -(alkylene)ₘ-NR³R⁴, -(alkylene)ₘ-C(O)—NR³R⁴, -(alkylene)ₘ-C(O)—O-alkyl or -(alkylene)ₘ-OR⁵; any of which may be optionally independently substituted with 1, 2, 3, or 4 R^{x} groups as allowed by valance, and wherein two R^{x} groups bound to the same or adjacent atom may optionally combine to form a ring.

In some aspects, R² is -(alkylene)ₘ-heterocycle, -(alkylene)ₘ-NR³R⁴, -(alkylene)ₘ-C(O)—NR³R⁴, -(alkylene)ₘ-C(O)—O-alkyl or -(alkylene)ₘ-OR⁵ without further substitution.

In some aspects, m in R² is 1. In a further aspect, the alkylene in R² is methylene.

In some aspects, R² is

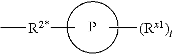

wherein:

R²* is a bond, alkylene, -(alkylene)ₘ-O-(alkylene)ₘ-, -(alkylene)ₘ-C(O)-(alkylene)ₘ-, -(alkylene)ₘ-S(O)₂-(alkylene)ₘ- or -(alkylene)ₘ-NH-(alkylene)ₘ-; wherein each m is independently 0 or 1;

P is a 4- to 8-membered mono- or bicyclic saturated heterocycle group;

each R^{x1} is independently -(alkylene)ₘ-(C(O))ₘ-(alkylene)ₘ-(N(R^{N}))ₘ-(alkyl)ₘ wherein each m is independently 0 or 1 provided at least one m is 1, —(C(O))—O-alkyl, -(alkylene)ₘ-cycloalkyl wherein m is 0 or 1, —N(R^{N})-cycloalkyl, —C(O)-cycloalkyl, -(alkylene)ₘ-heterocycle wherein m is 0 or 1, or —N(R^{N})-heterocycle, —C(O)-heterocycle, —S(O)₂-(alkylene)ₘ wherein m is 1 or 2, wherein:

R^{N} is H, C₁ to C₄ alkyl or C₁ to C₆ heteroalkyl, and
wherein two R^{x1} can, together with the atoms to which they attach on P, which may be the same atom, form a ring; and

t is 0, 1 or 2.

In some aspects, each R^{x1} is only optionally substituted by unsubstituted alkyl, halogen or hydroxy.

In some aspects, R^{x1} is hydrogen or unsubstituted C₁-C₄ alkyl.

In some aspects, at least one R^{x1} is -(alkylene)ₘ-heterocycle wherein m is 0 or 1.

In some aspects, R² is

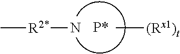

wherein P* is a 4- to 8-membered mono- or bicyclic saturated heterocycle group; and wherein R²*, R^{x1} and t are as previously defined.

In some aspects, R² is

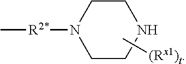

In some aspects, R² is

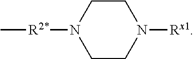

In some aspects, R² is

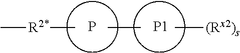

wherein:

R²* is a bond, alkylene, -(alkylene)ₘ-O-(alkylene)ₘ-, -(alkylene)ₘ-C(O)-(alkylene)ₘ-, -(alkylene)ₘ-S(O)₂-(alkylene)ₘ- or -(alkylene)ₘ-NH-(alkylene)ₘ- wherein each m is independently 0 or 1;

P is a 4- to 8-membered mono- or bicyclic saturated heterocycle group;

P1 is a 4- to 6-membered monocyclic saturated heterocycle group;

each R^{x} is independently hydrogen or alkyl; and

s is 0, 1 or 2.

In some aspects, R² is

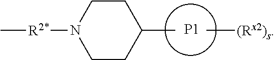

In some aspects, P1 includes at least one nitrogen.

In some embodiments, the compound is selected from a Formula presented above and

### R² is

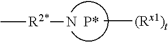

wherein P* is a 4- to 8-membered mono- or bicyclic saturated heterocycle group and R²*, R^{x1} and t are as previously defined.

In some embodiments, the compound is selected from a Formula presented above and R² is

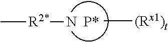

wherein P* is a 4- to 8-membered mono- or bicyclic saturated heterocycle group, R^{x1} is hydrogen or unsubstituted C₁-C₄ alkyl; and wherein R²* and t are as previously defined.

In some embodiments, the compound is selected from a Formula presented above and R² is selected from

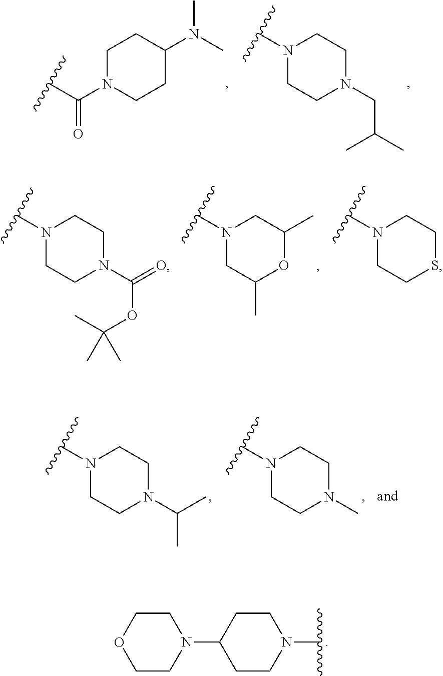

In some embodiments, the compound is selected from a Formula presented above and R² is selected from,

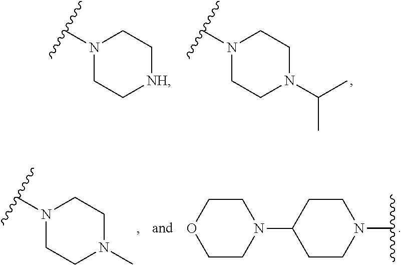

In some embodiments, the compound is selected from a Formula presented above and R is alkyl.

In some embodiments, the compound is selected from a Formula presented above and R is hydrogen.

In some aspects, any alkylene in R²* in any previous aspect is not further substituted.

In some aspects, R² is

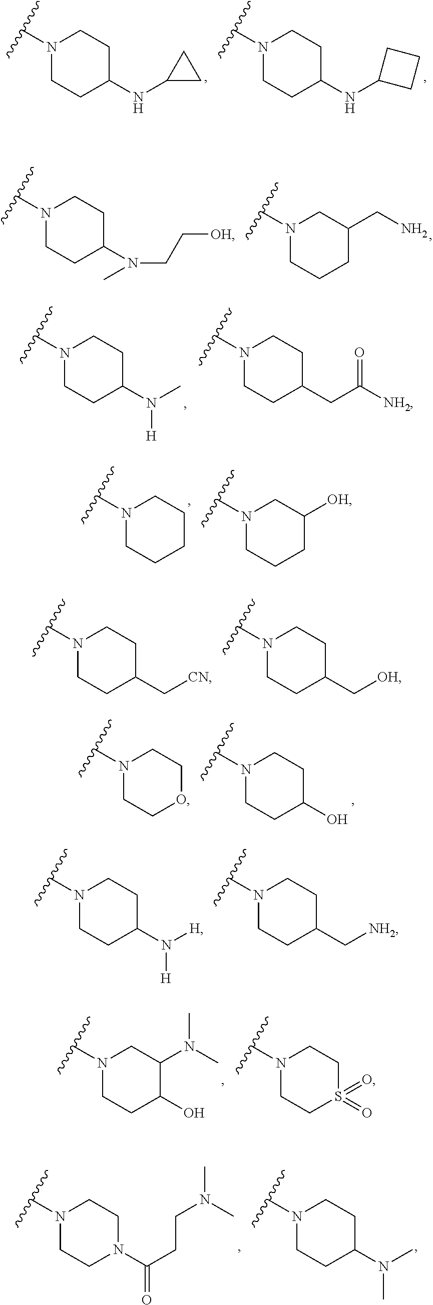

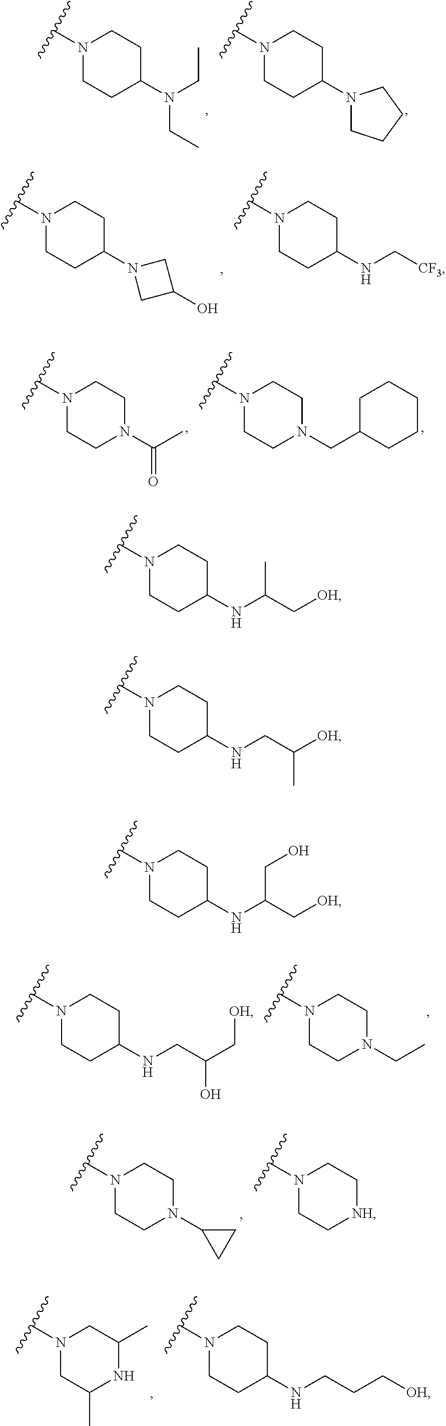

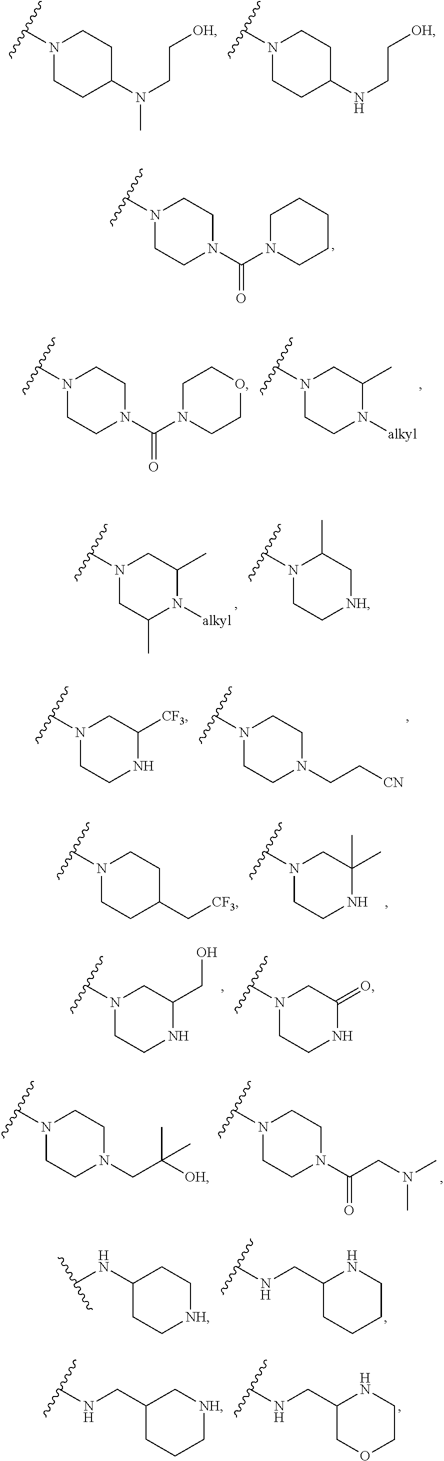

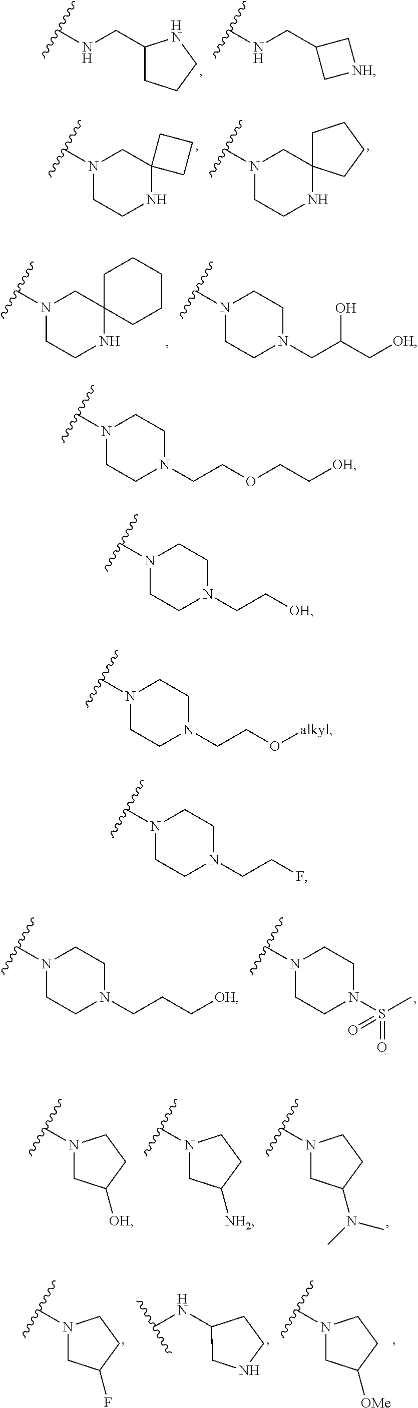

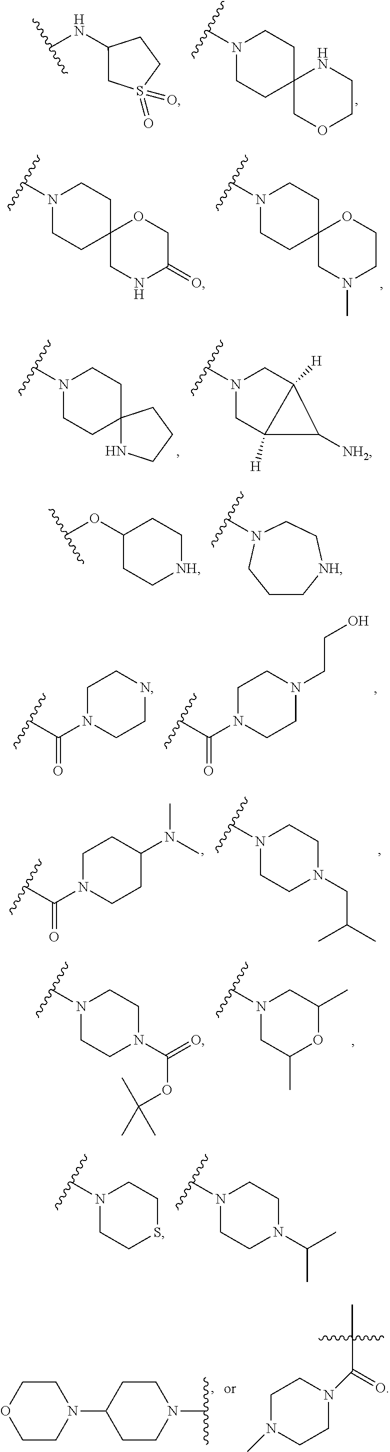

### Embodiments of R⁷

In one embodiment R⁷ is selected from:

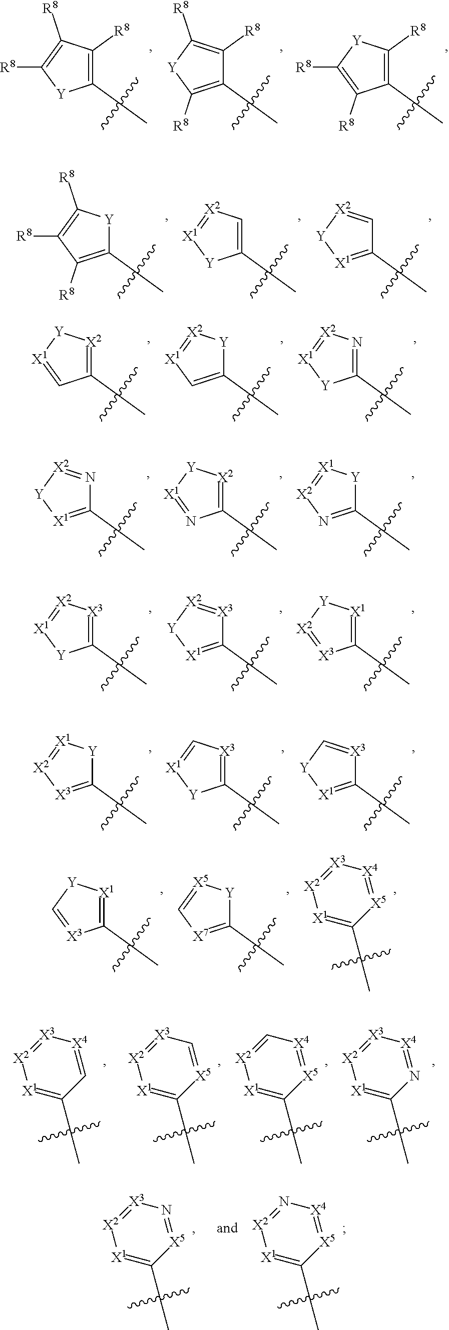

wherein

Y is NH, O, S, or NR⁹;

X¹, X², X³, X⁴, and X⁵ are independently N or CR⁸;

R⁸ is selected independently at each instance from hydrogen, amino, halogen, R⁶, and R²; and

R⁹ is selected from —C(O)H, —C(O)alkyl, —C(S)alkyl, alkyl, aryl, heteroaryl, alkyl-aryl, and alkyl-heteroaryl.

In another embodiment, R⁷ is selected from:

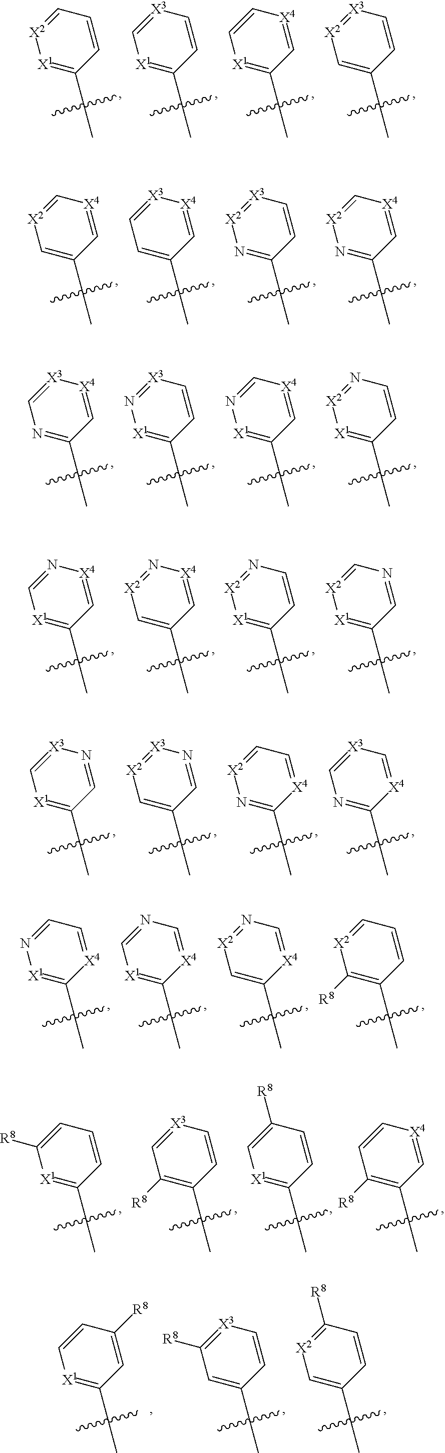

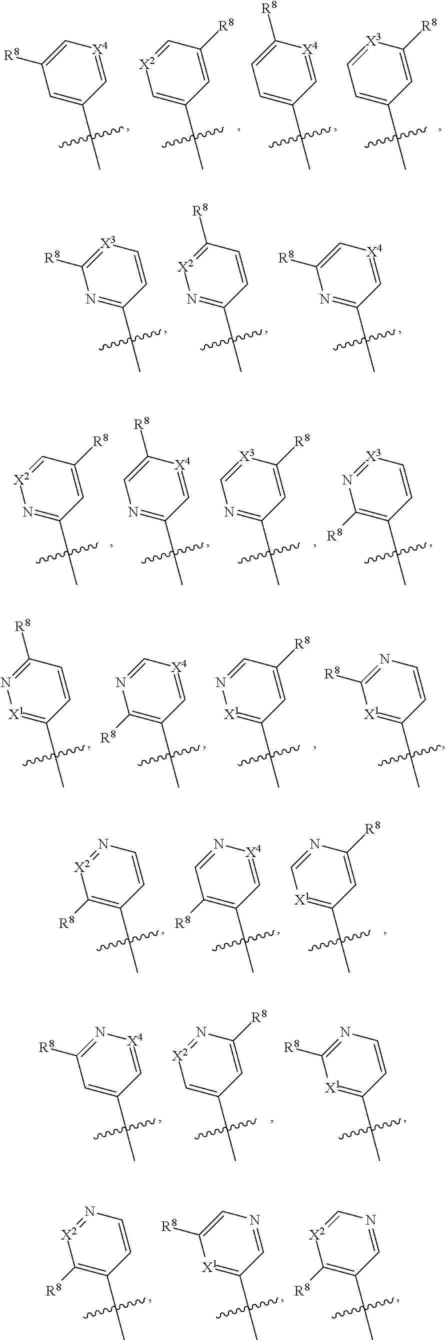

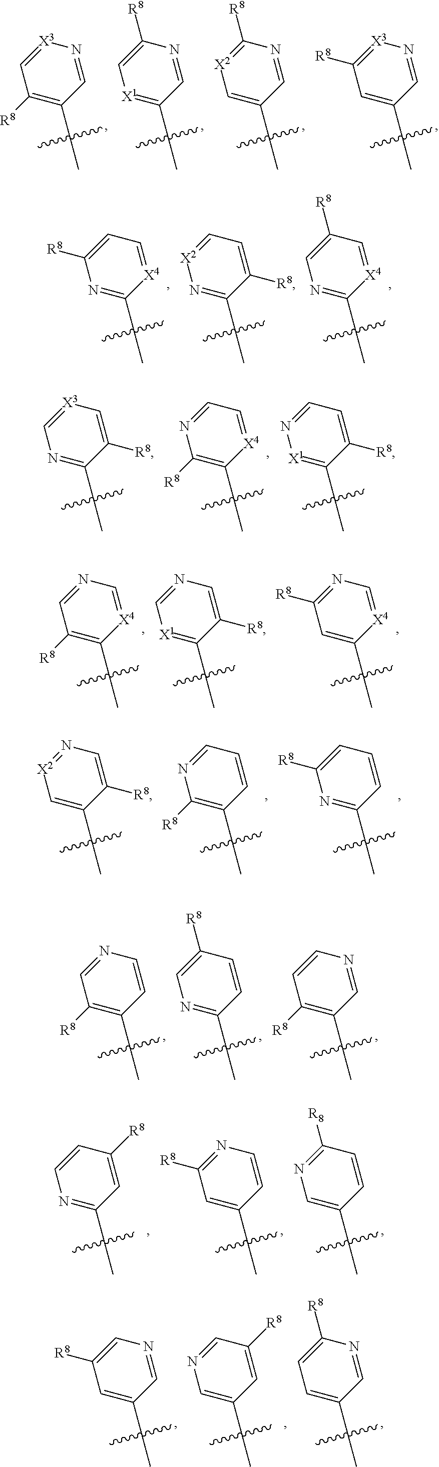

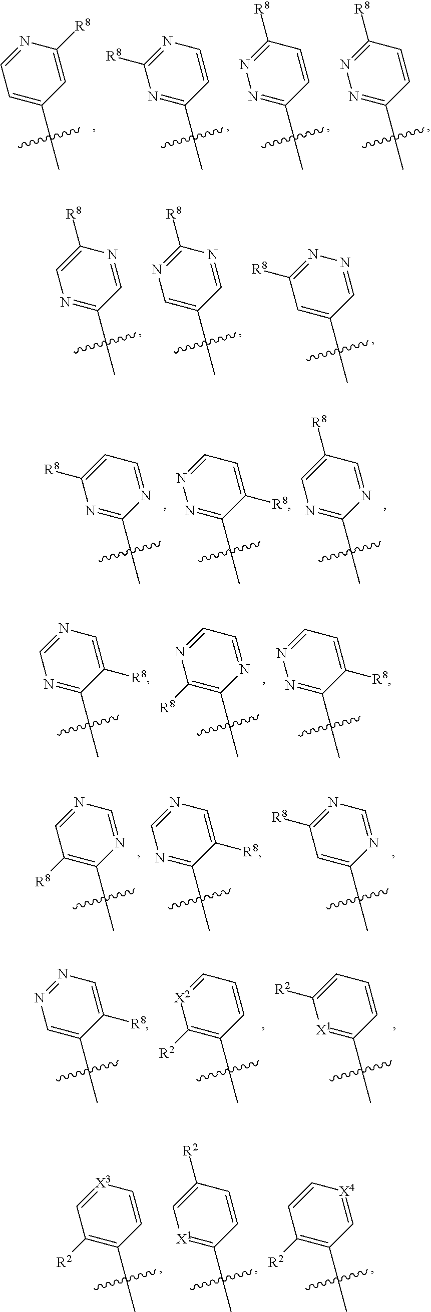

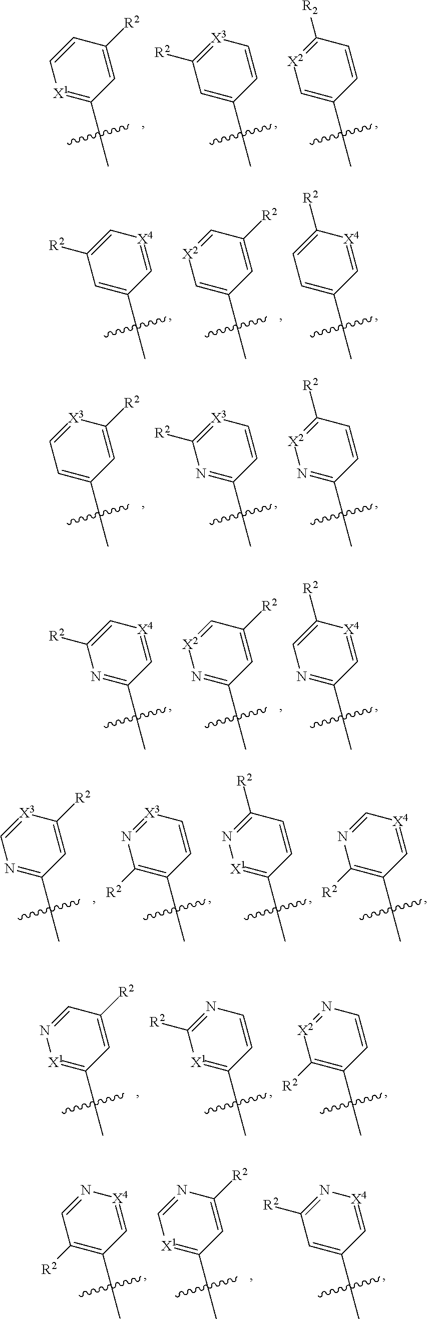

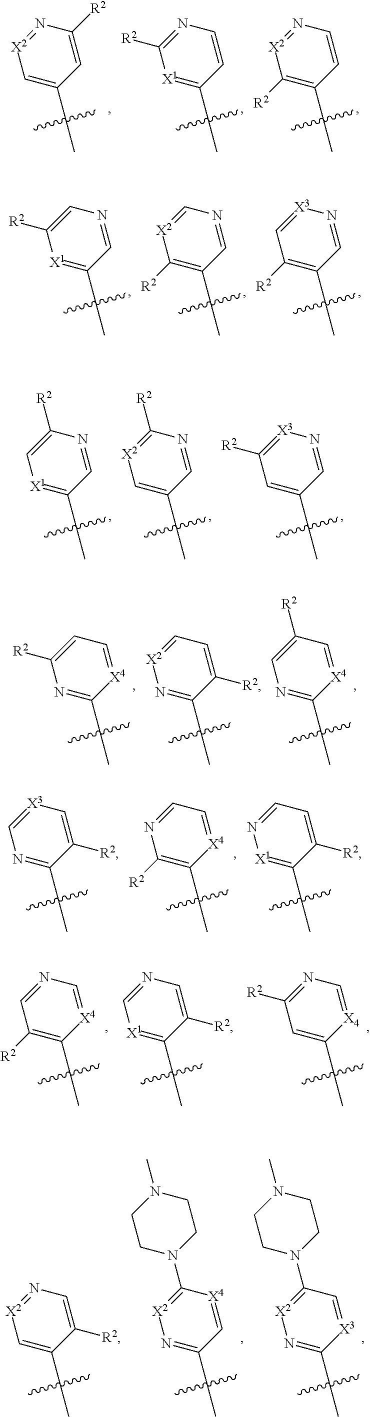

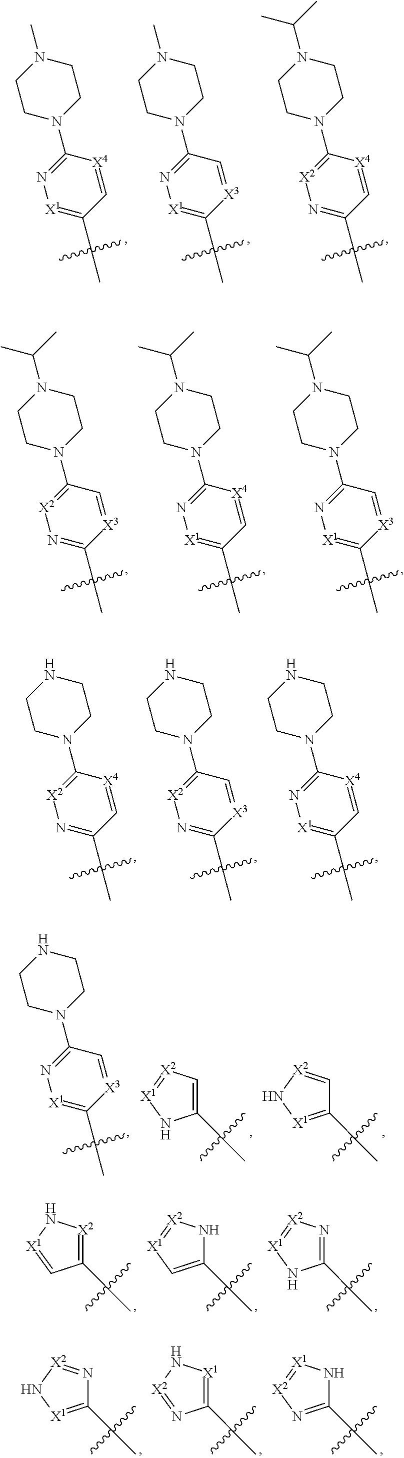

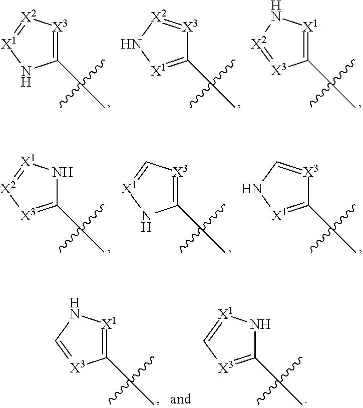

In another embodiment, R⁷ is selected from:

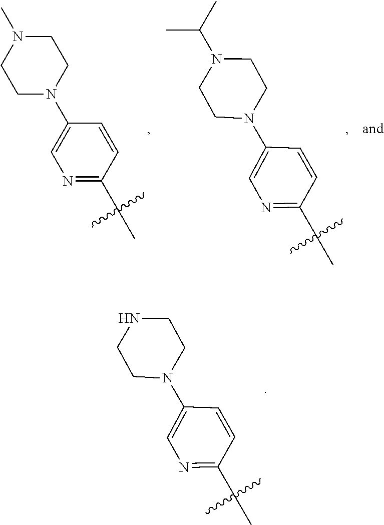 [SMILES: *c1ccc(N2CCN(C)CC2)cn1 ; *c1ccc(N2CCN(C(C)C)CC2)cn1 ; *c1ccc(N2CCNCC2)cn1]

In some aspects, R⁶ is hydrogen.

In an alternative embodiment R⁷ is hydrogen.

In some aspects, R^{x} is not further substituted.

In another embodiment,

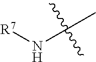

is selected from:

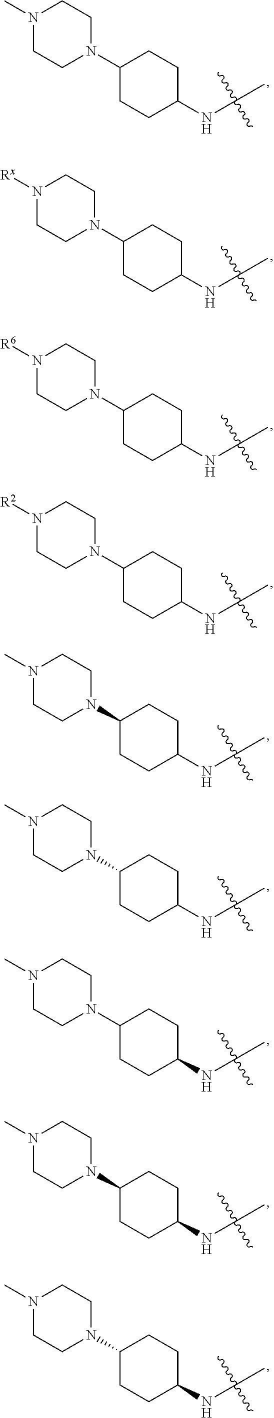

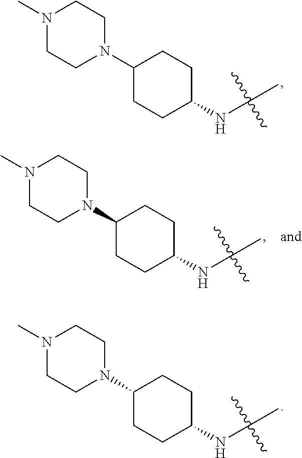

In one embodiment, R⁷ is selected from:

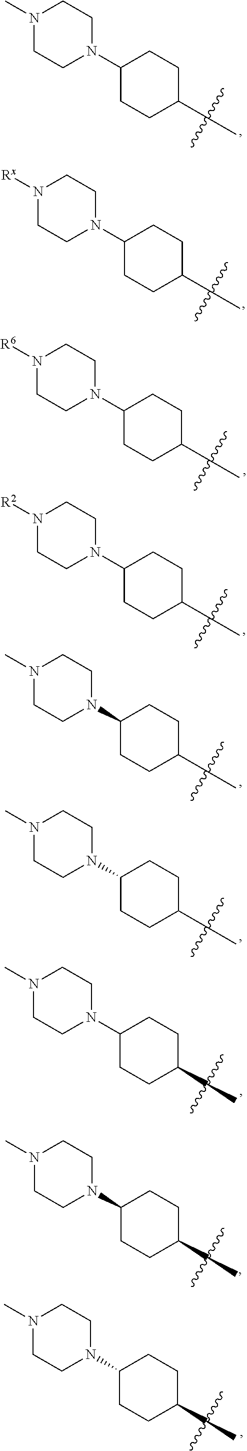

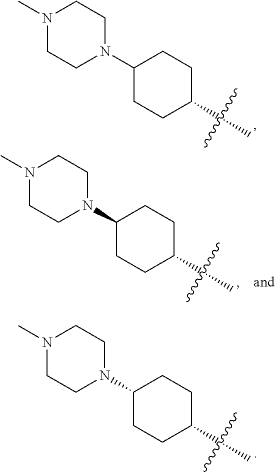

In some embodiments, R⁷ is

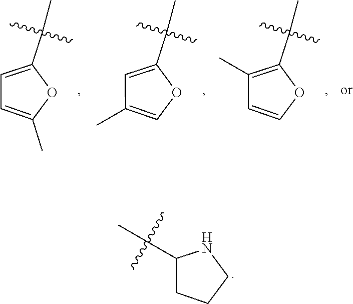

In another embodiment,

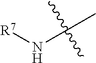

is selected from:

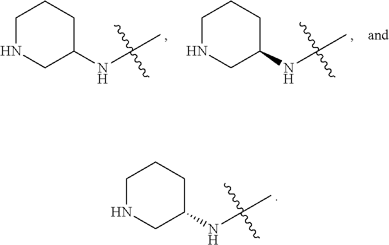 [SMILES: *NC1CCCNC1 ; *N[C@@H]1CCCNC1 ; *N[C@H]1CCCNC1]

In one embodiment, R⁷ is selected from:

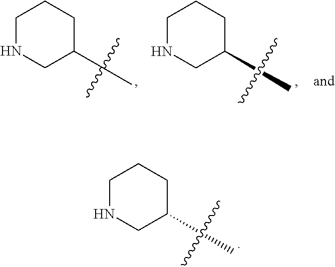

In some embodiments,

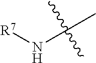

is selected from:

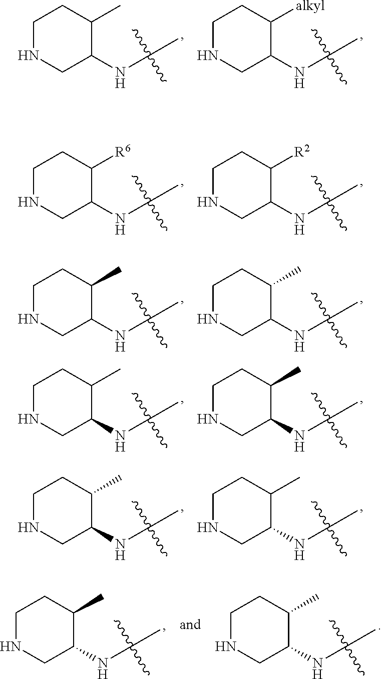

In some embodiments, R⁷ is selected from:

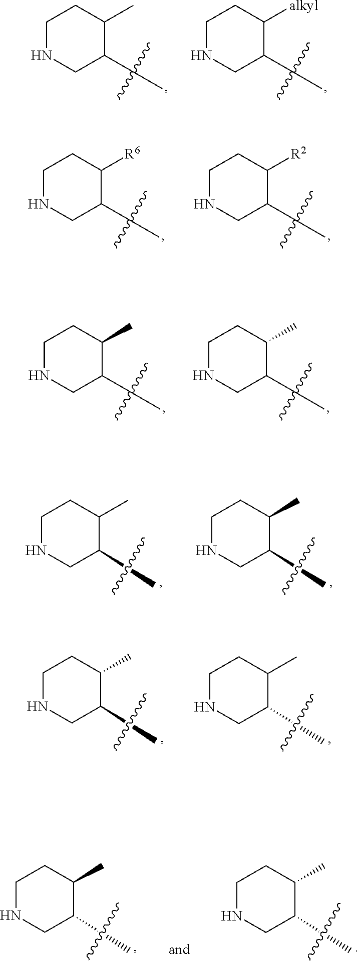

In some embodiments,

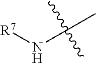

is selected from:

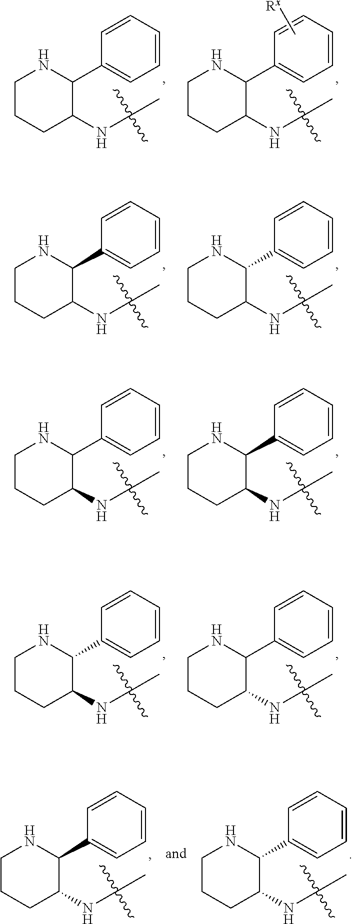

In some embodiments, R⁷ is selected from:

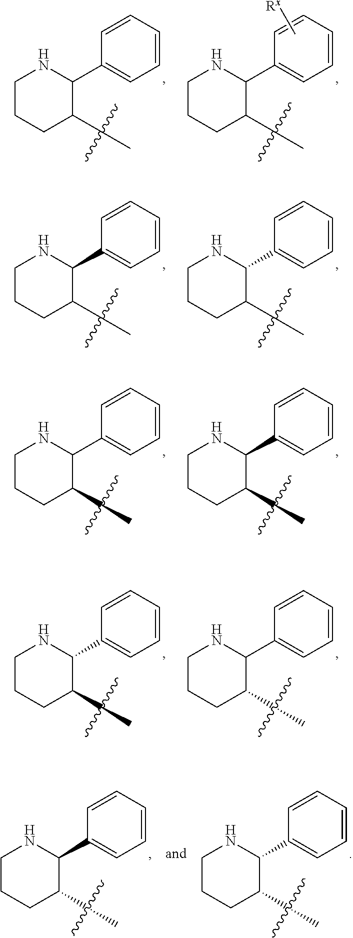

In some embodiments, R⁷ is selected from:

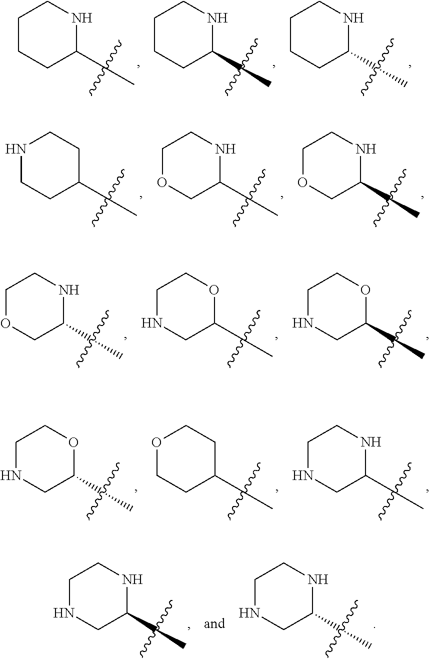

In some embodiments,

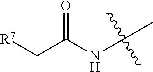

is selected from:

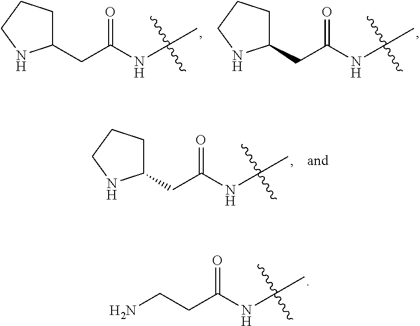 [SMILES: *NC(=O)CC1CCCN1 ; *NC(=O)C[C@@H]1CCCN1 ; *NC(=O)C[C@H]1CCCN1 ; *NC(=O)CCN]

In some embodiments, R⁷ is selected from:

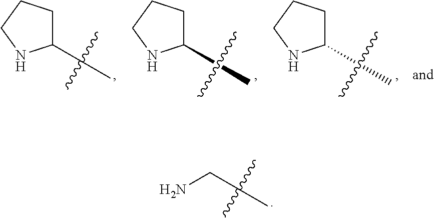

### Embodiments of R¹⁶

In some embodiments, R¹⁶ is selected from:

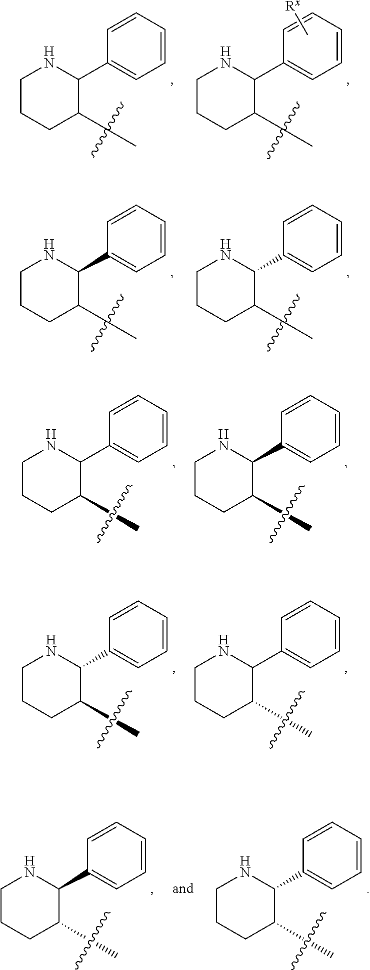

In some embodiments,

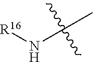

is selected from:

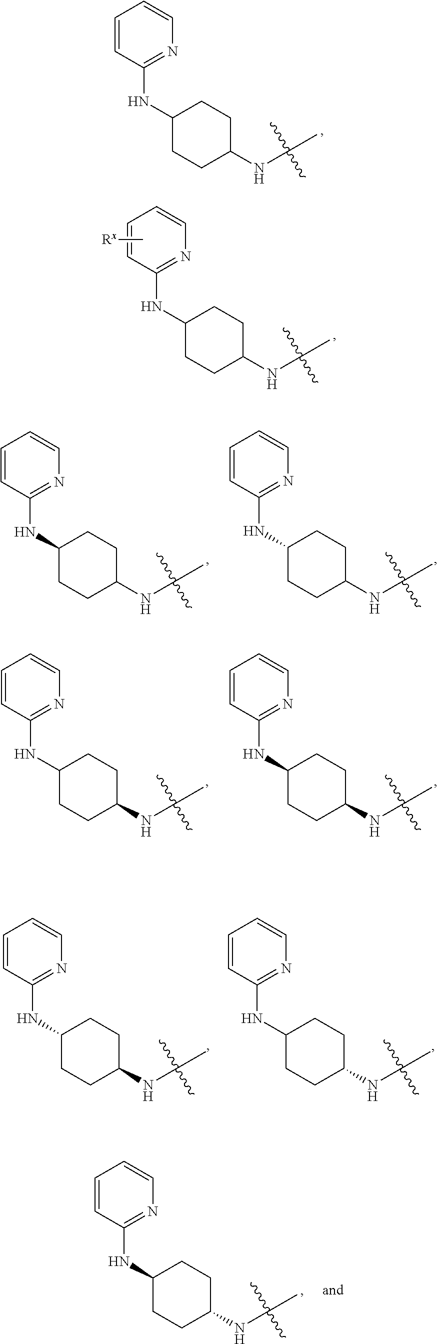

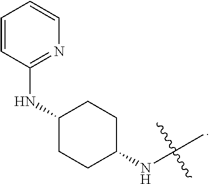 [SMILES: *N[C@H]1CC[C@@H](Nc2ccccn2)CC1]

In some embodiments,

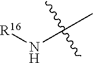

is selected from:

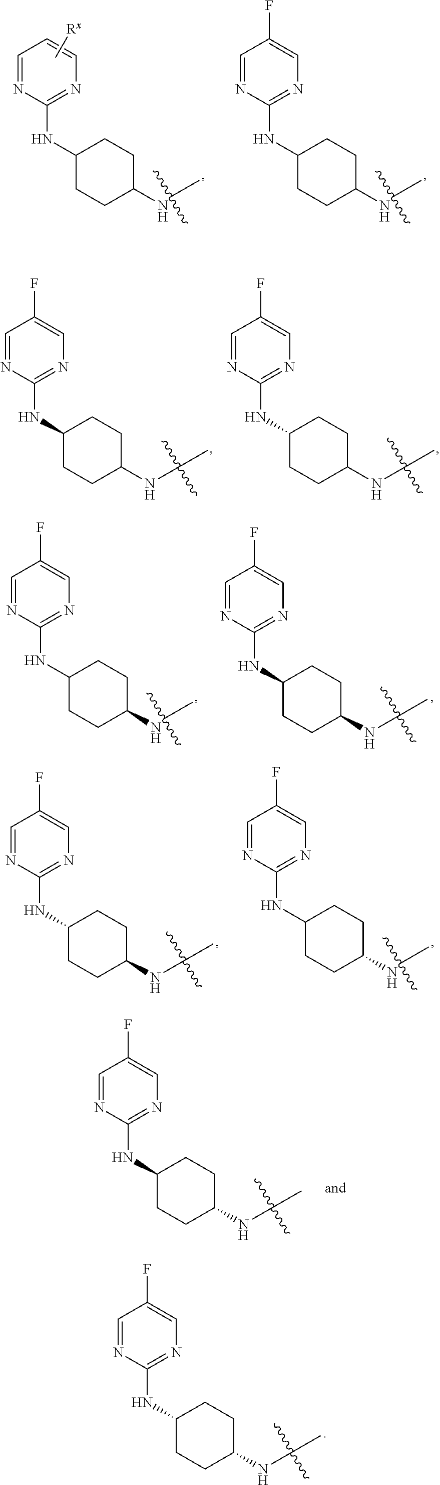

In some embodiments,

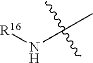

is selected from:

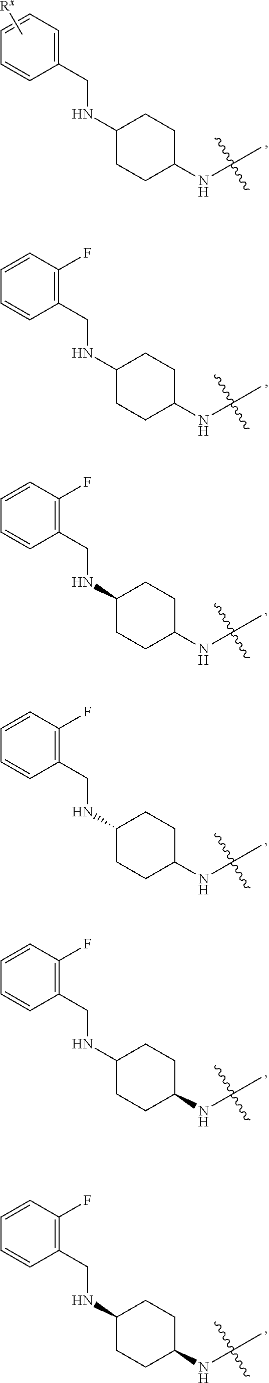

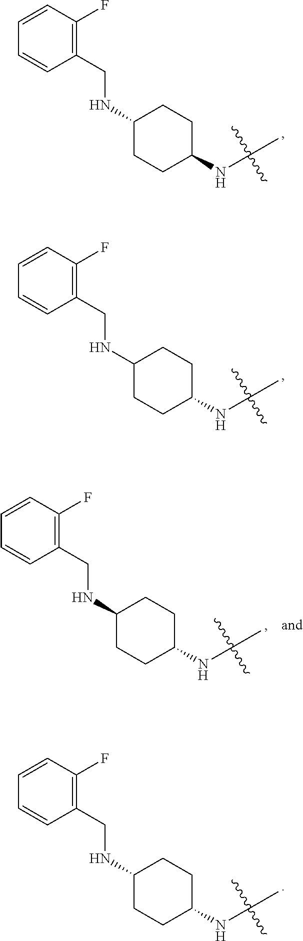

In some embodiments

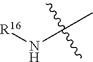

is selected from:

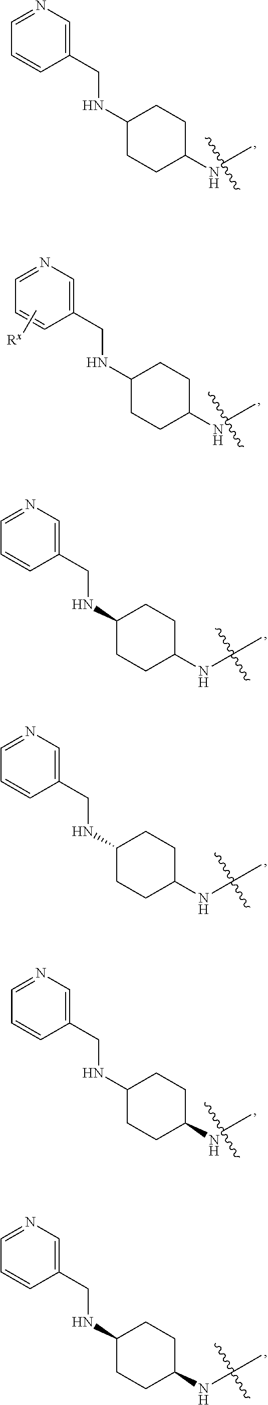

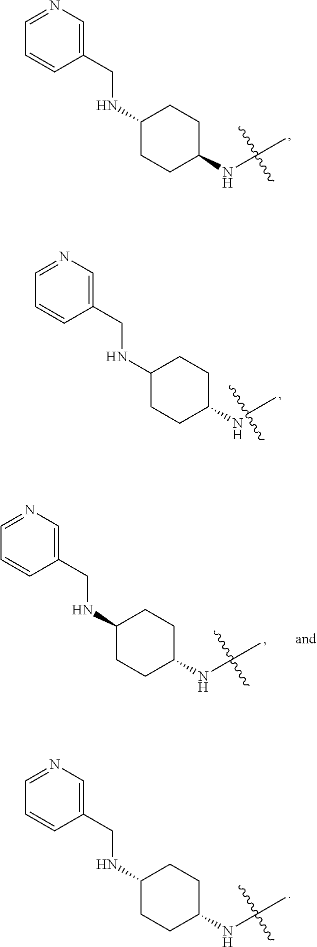

In some embodiments,

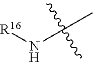

is selected from:

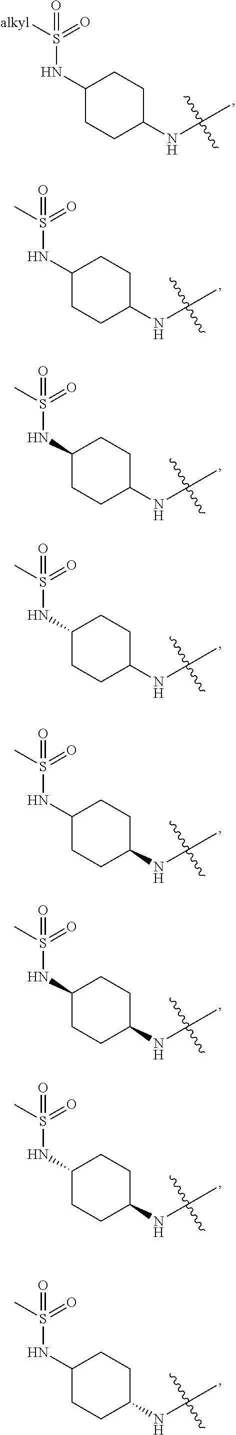

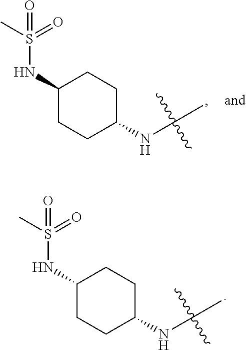 [SMILES: *N[C@H]1CC[C@H](NS(C)(=O)=O)CC1 ; *N[C@H]1CC[C@@H](NS(C)(=O)=O)CC1]

In some embodiments, R¹⁶ is selected from:

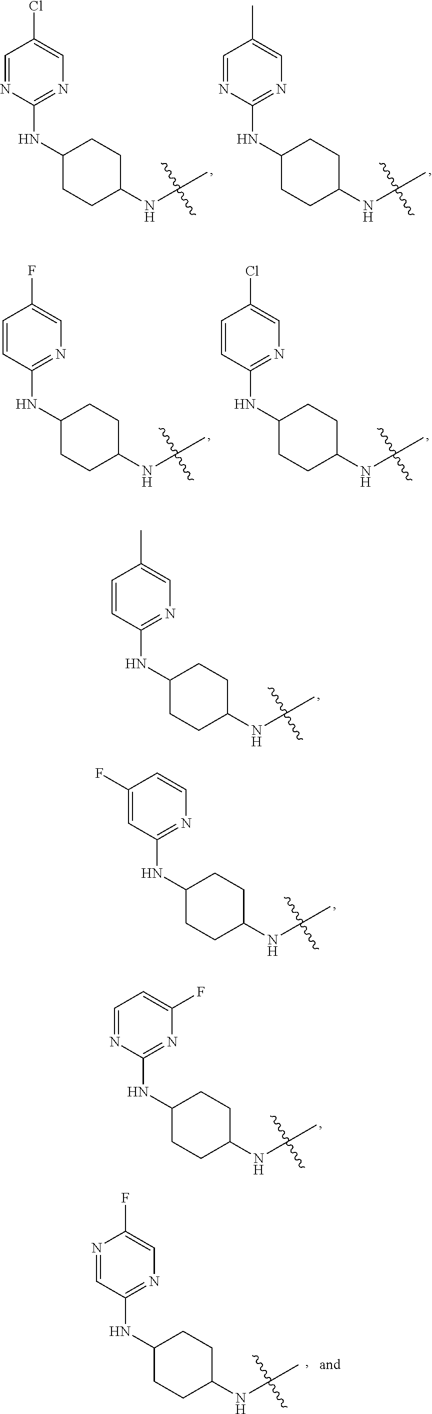

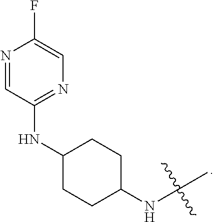 [SMILES: *NC1CCC(Nc2cnc(F)cn2)CC1]

In some embodiments, R¹⁶ is selected from:

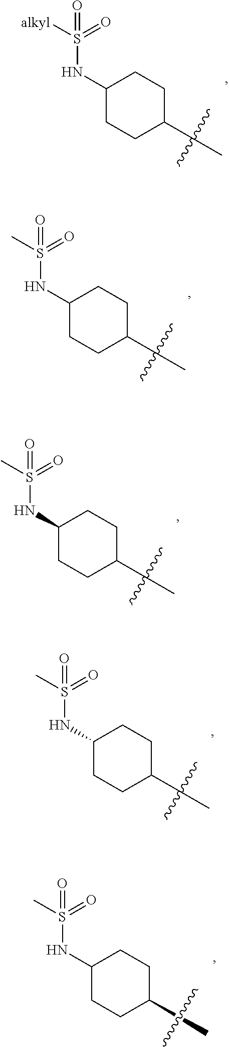

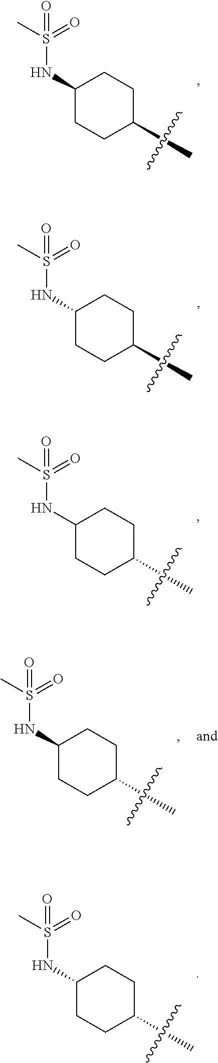

In some embodiments,

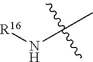

is selected from:

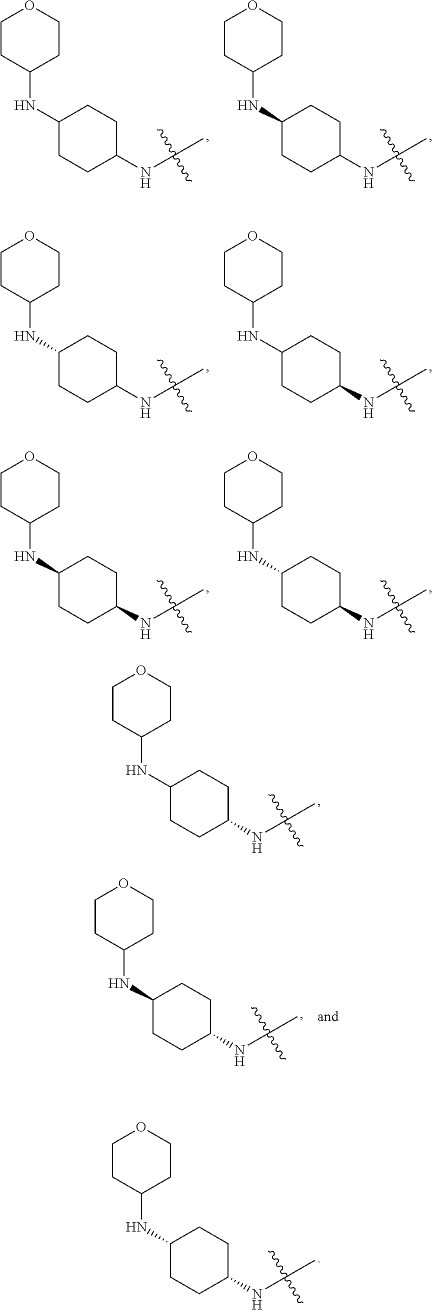

In some embodiments, R¹⁶ is selected from:

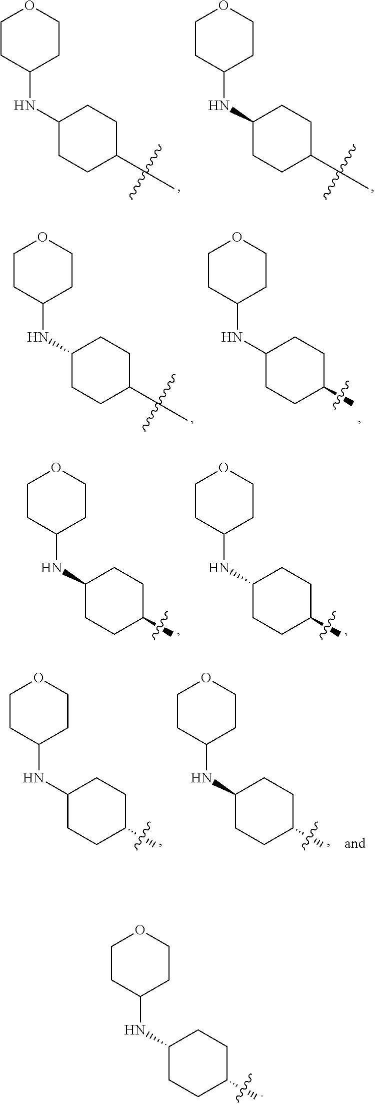

In one embodiment, R¹⁶ is selected from:

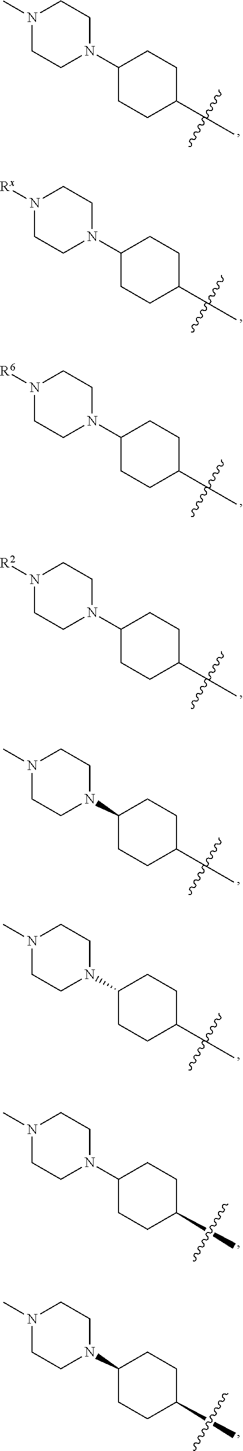

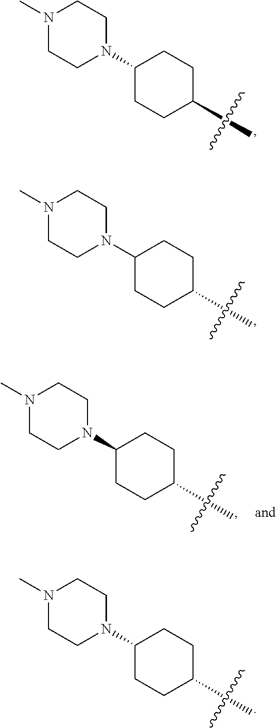

In some embodiments, R¹⁶ is selected from:

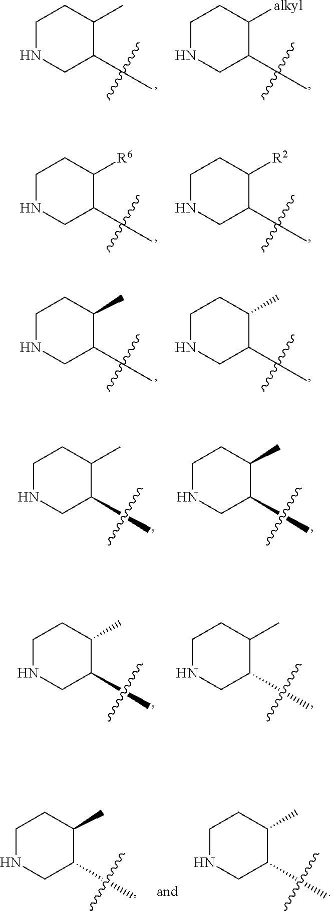

In some embodiments, R¹⁶ is selected from:

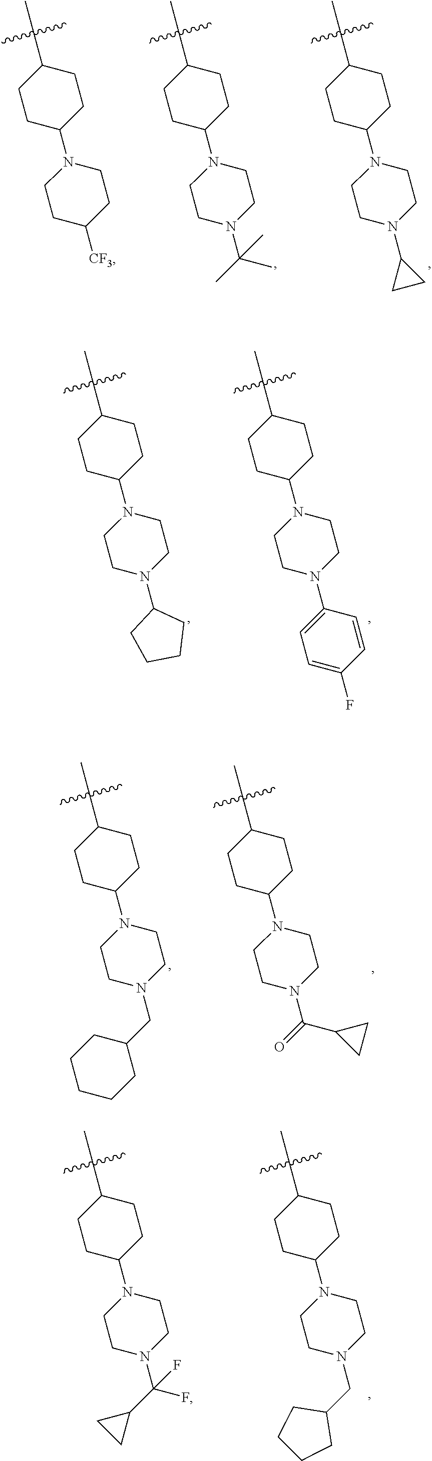

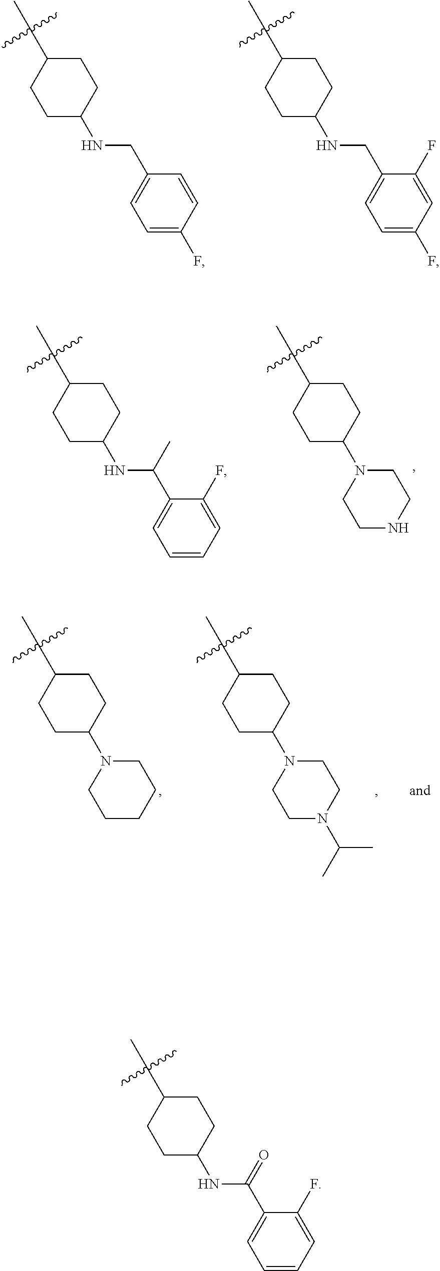

In some embodiments, R¹⁶ is selected from:

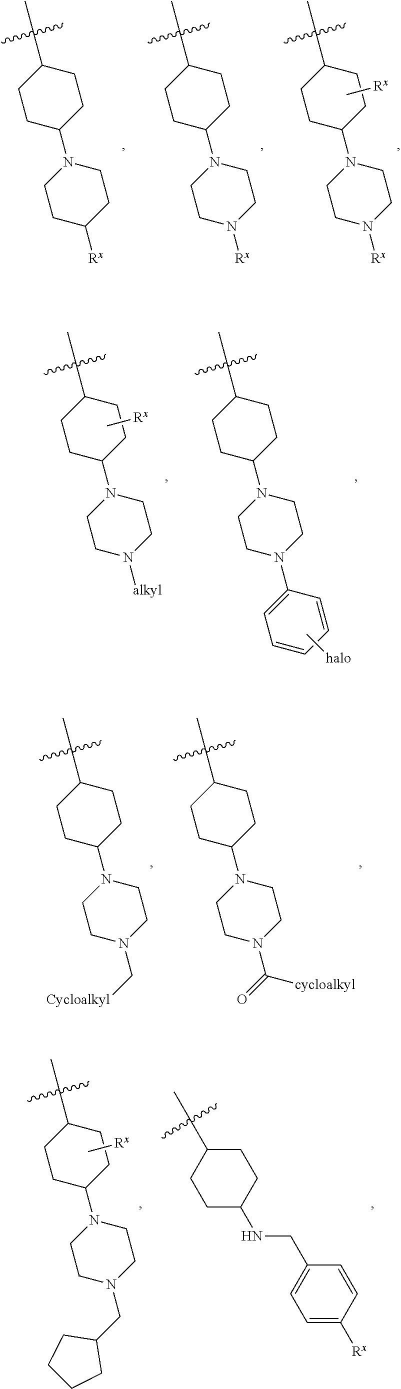

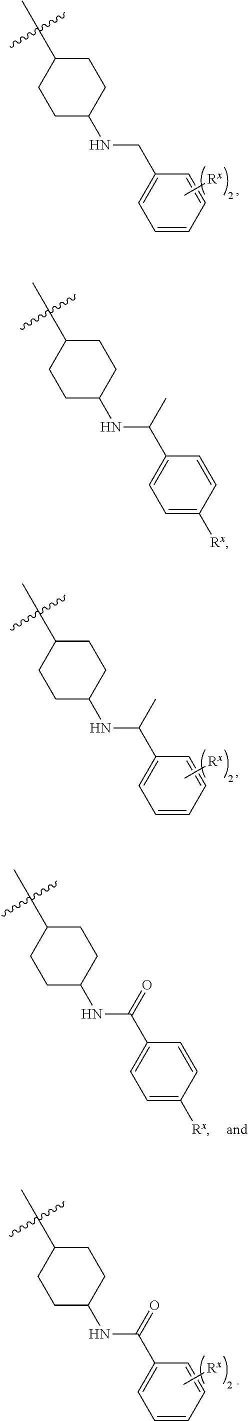

### Embodiments of R²⁰

In some embodiments, R²⁰ is selected from:

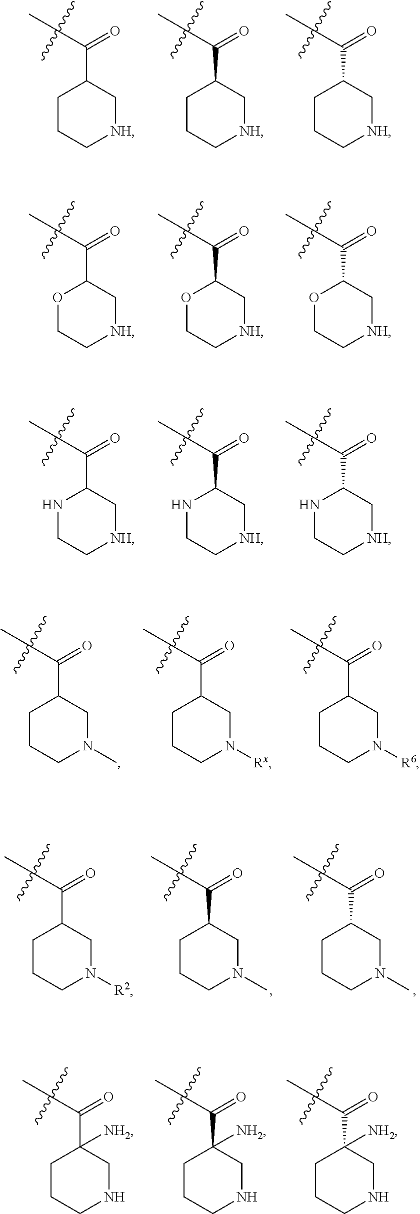

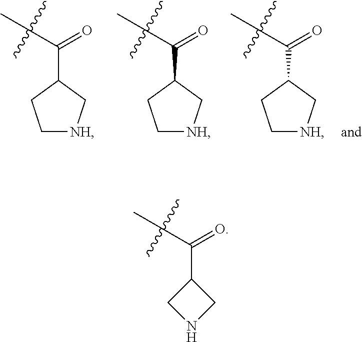 [SMILES: *C(=O)C1CCNC1 ; *C(=O)[C@@H]1CCNC1 ; *C(=O)[C@H]1CCNC1 ; "and" ; *C(=O)C1CNC1]

In some embodiments, R²⁰ is selected from:

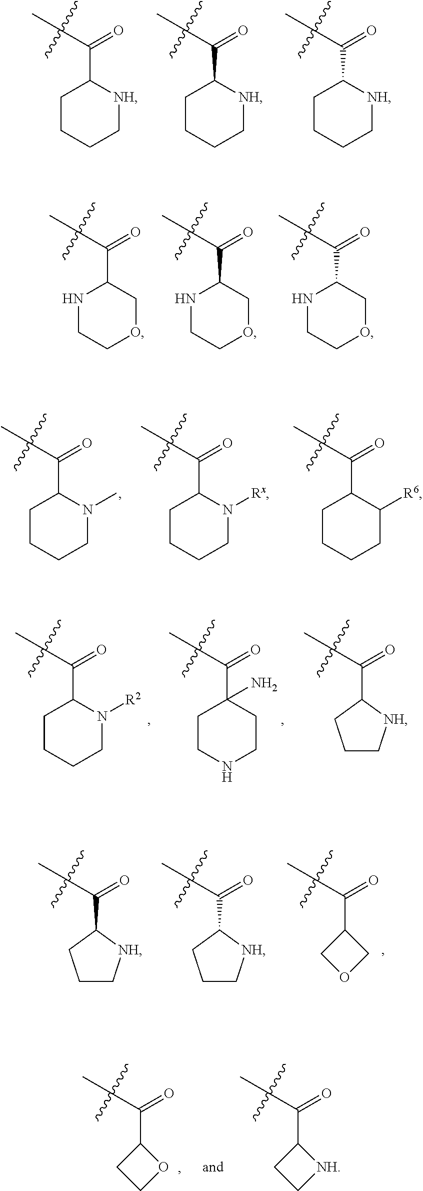

In some embodiments, R²⁰ is selected from:

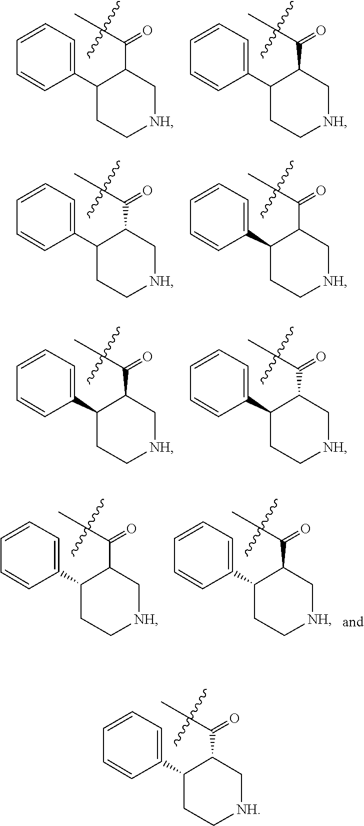

In some embodiments, R²⁰ is selected from:

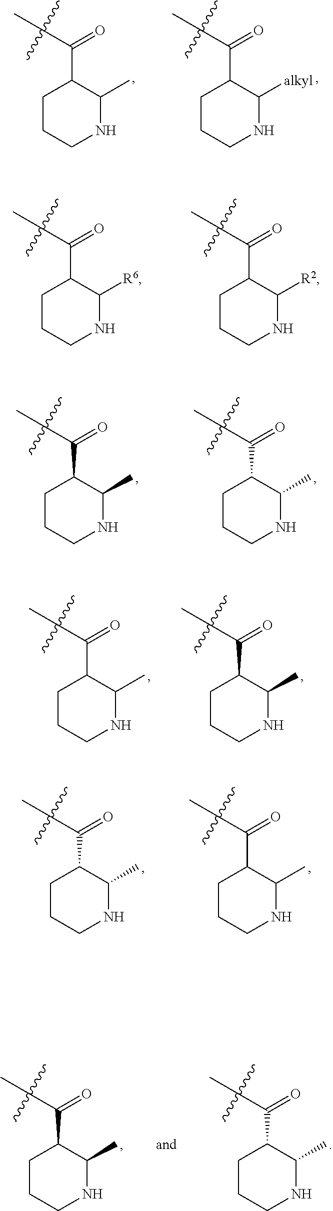

In some embodiments, R²⁰ is selected from:

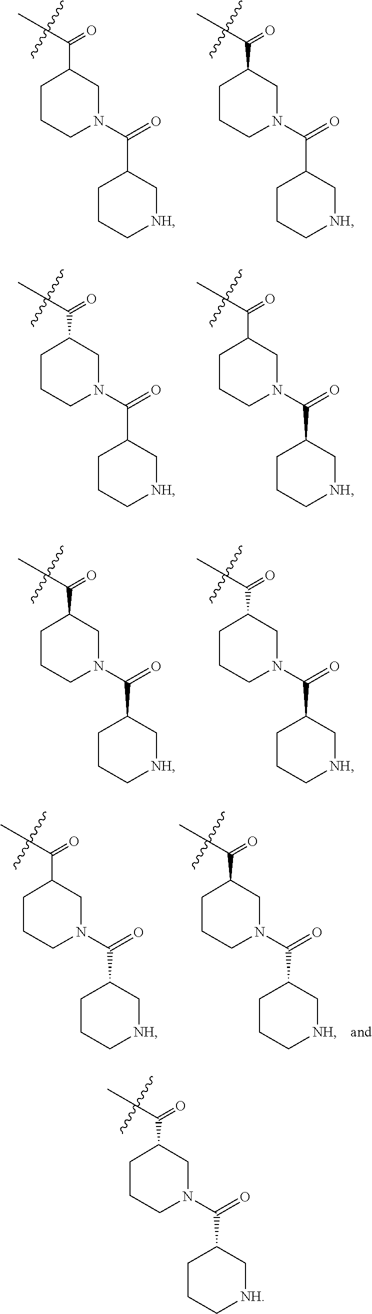

### Embodiments of R²

In some embodiments R²¹ is selected from:

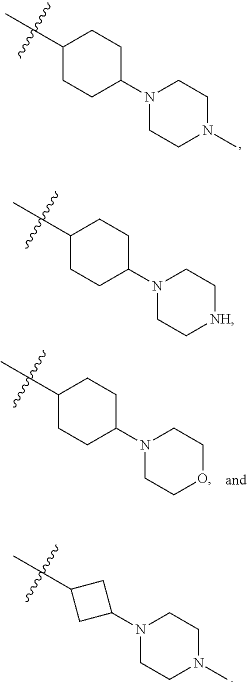 [SMILES: *C1CCC(N2CCN(C)CC2)CC1 ; *C1CCC(N2CCNCC2)CC1 ; *C1CCC(N2CCOCC2)CC1 ; *C1CC(N2CCN(C)CC2)C1]

In some embodiments R²¹ is selected from:

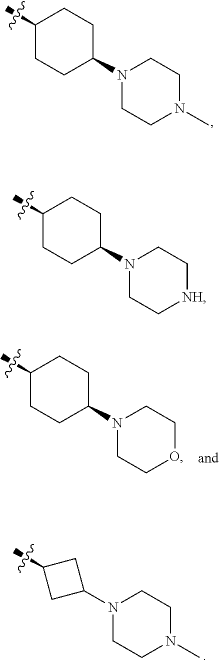

In some embodiments R²¹ is selected from:

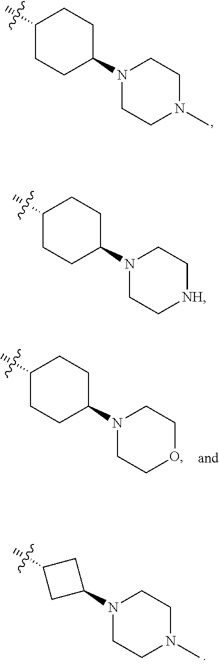

In some embodiments R²¹ is selected from:

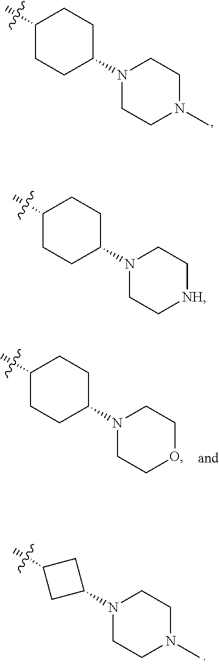

In some embodiments R²¹ is selected from:

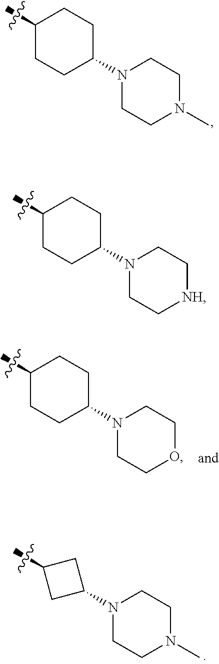

In some embodiments R²¹ is selected from:

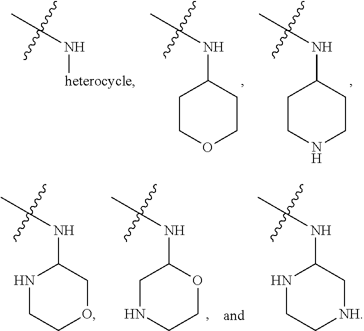

In some embodiments R²¹ is selected from:

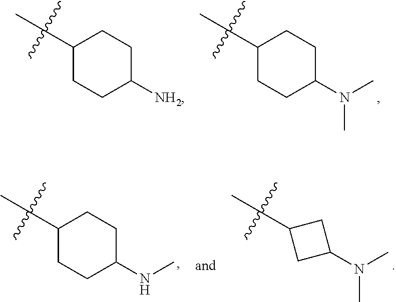

In some embodiments, R²¹ is selected from:

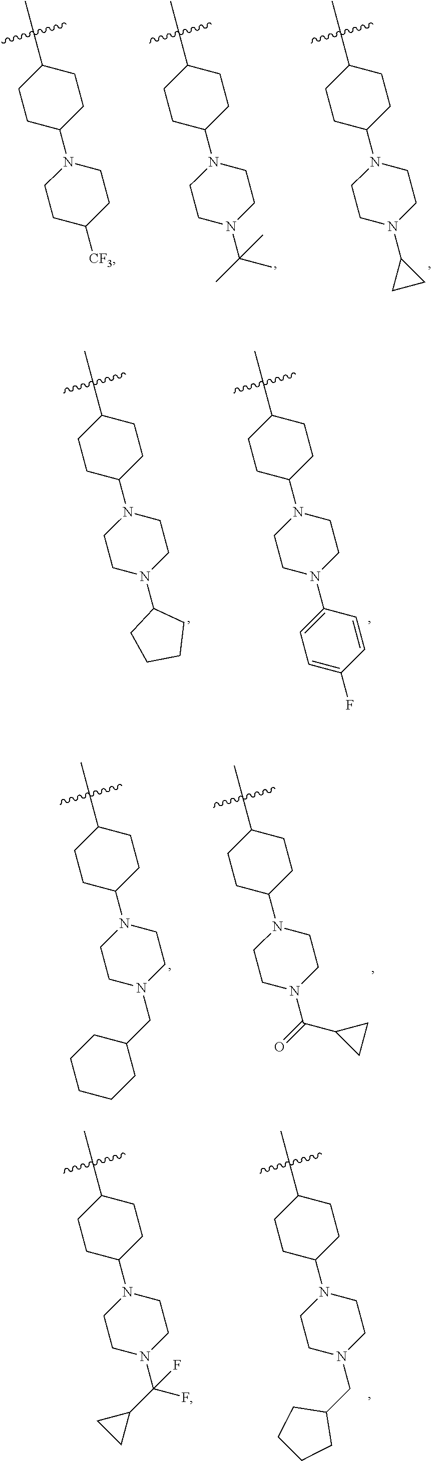

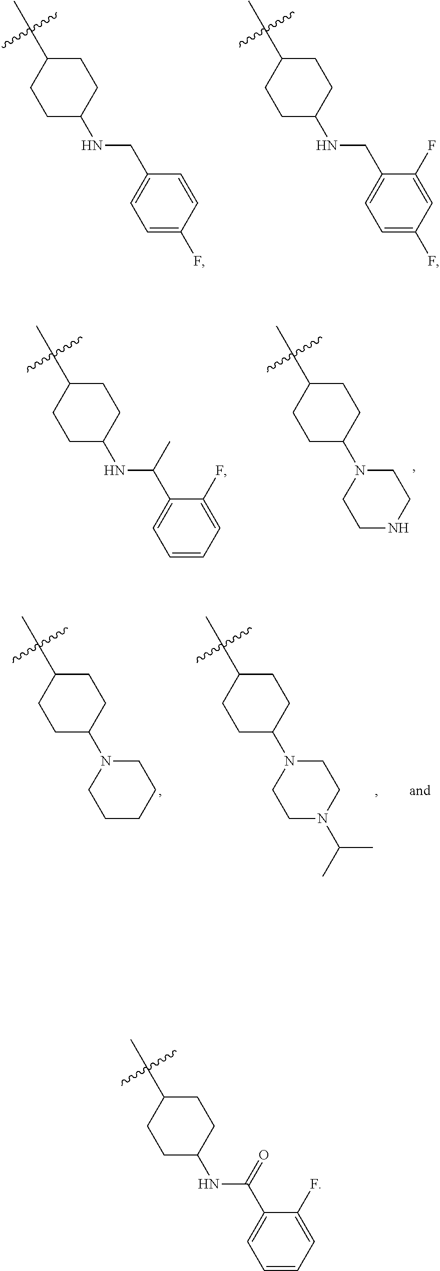

In some embodiments, R²¹ is selected from:

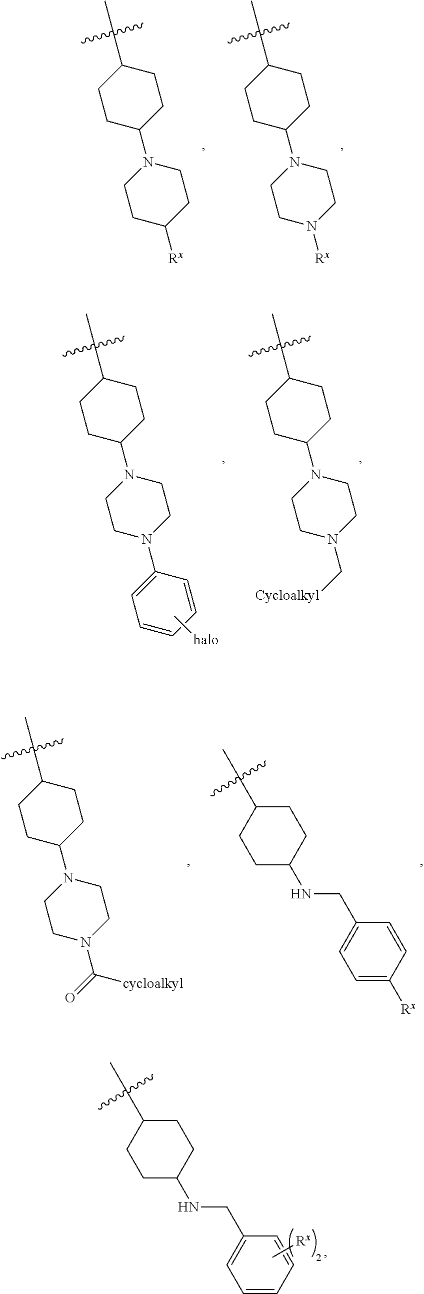

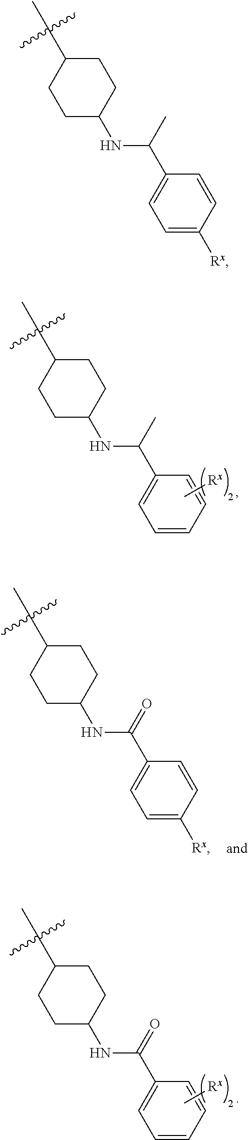

### Embodiments of Formula XI

In one embodiment

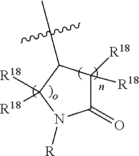

is selected from:

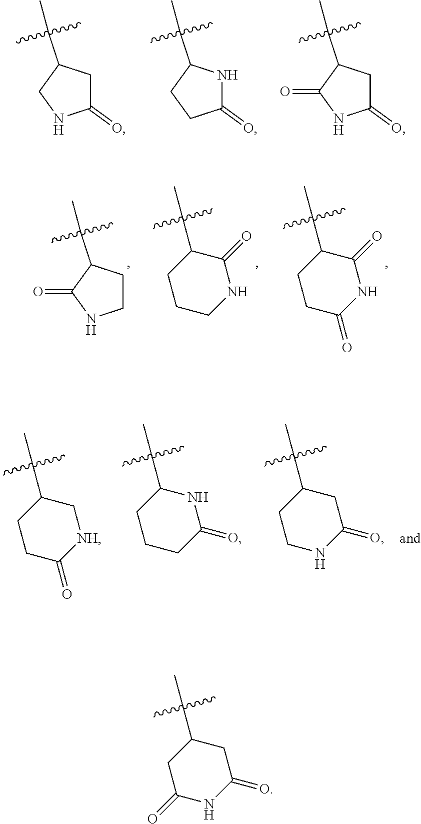

In one embodiment

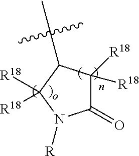

is selected from:

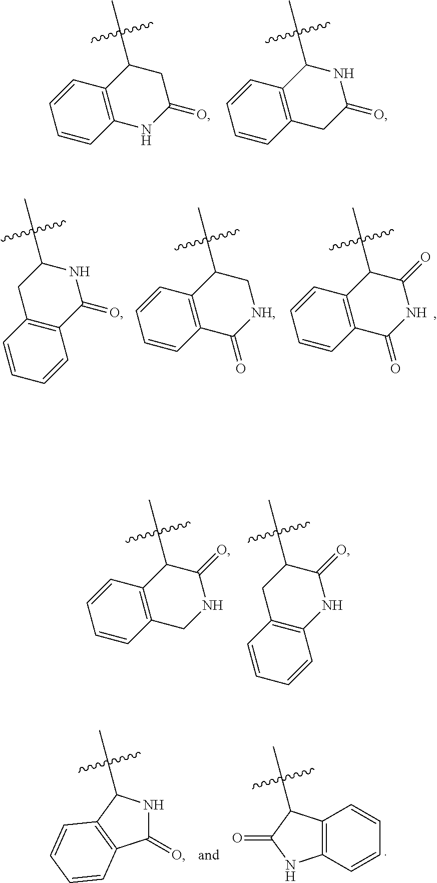

In one embodiment

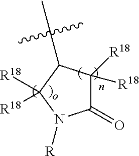

is selected from:

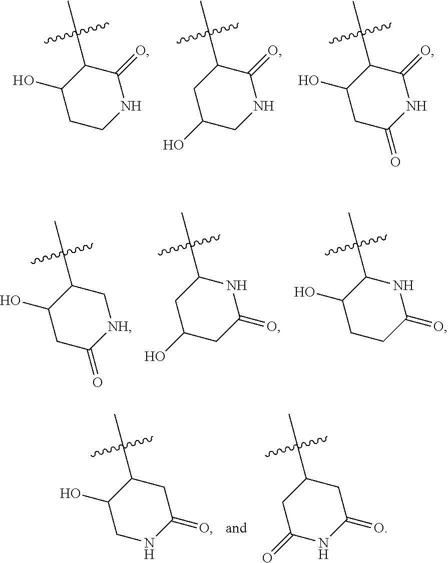

### II. Terminology

Compounds are described using standard nomenclature. Unless defined otherwise, all technical and scientific terms used herein have the same meaning as is commonly understood by one of skill in the art to which this invention belongs.

The compounds in any of the Formulas described herein include racemates, enantiomers, mixtures of enantiomers, diastereomers, mixtures of diastereomers, tautomers, N-oxides, isomers; such as rotamers, as if each is specifically described.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

The present invention includes compounds of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, and Formula XVII with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorous, fluorine, chlorine and iodine such as ²H, ³H, ¹¹C, ¹³C, ¹⁴C, ¹⁵N, ¹⁷O, ¹⁸O, ¹⁸F, ³¹P, ³²P, ³⁵S, ³⁶Cl, and ¹²⁵I respectively. In one non-limiting embodiment, isotopically labelled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (2H) and tritium (3H) may be used anywhere in described structures that achieves the desired result. Alternatively or in addition, isotopes of carbon, e.g., ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one non-limiting embodiment, deuterium is 90, 95 or 99% enriched at a desired location.

In one non-limiting embodiment, the substitution of one or more hydrogen atoms for a deuterium atoms can be provided in any of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII. In one non-limiting embodiment, the substitution of a hydrogen atom for a deuterium atom occurs within a group selected from any of R, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², and R^{x}. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle the unsubstituted carbons may be deuterated.

The compound of the present invention may form a solvate with solvents (including water). Therefore, in one non-limiting embodiment, the invention includes a solvated form of the compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a compound of the invention and water. Pharmaceutically acceptable solvates in accordance with the invention include those wherein the solvent may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C═O)NH₂ is attached through carbon of the keto (C═O) group.

“Alkyl” is a branched or straight chain saturated aliphatic hydrocarbon group. In one non-limiting embodiment, the alkyl group contains from 1 to about 12 carbon atoms, more generally from 1 to about 6 carbon atoms or from 1 to about 4 carbon atoms. In one non-limiting embodiment, the alkyl contains from 1 to about 8 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species. For example, the term C₁-C₄ alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In an alternative embodiment, the alkyl group is optionally substituted. The term “Alkyl” also encompasses cycloalkyl or carbocyclic groups. For example, when a term is used that includes “alk” then “cycloalkyl” or “carbocyclic” can be considered part of the definition, unless unambiguously excluded by the context. For example, and without limitation, the terms alkyl, —O-alkyl, haloalkyl, etc. can all be considered to include the cyclic forms of alkyl, unless unambiguously excluded by context.

As used herein “substituted alkyl” refers to an alkyl group that is substituted with the described substituents. If no substituents are explicitly described “substituted alkyl” refers to an alkyl group that is substituted with 1, 2, 3, or 4 substituents independently selected from F, Cl, Br, I, cyano, hydroxy, —O-alkyl, —SH, —Salkyl, —COOH, —COOalkyl, —COalkyl, —COH, —CONH₂, —CONHalkyl, —CON(alkyl)₂, —OC(O)alkyl, —NHC(O)alkyl, —NalkylC(O)alkyl, nitro, amino, —NHalkyl, N(alkyl)₂, cyano, haloalkyl, aryl, heteroaryl, alkenyl, alkynyl, haloalkyl, cycloalkyl, alkyl-aryl, alkyl-heteroaryl, alkyl-cycloalkyl, alkyl-heterocycle, heterocycle, —COOaryl, —COaryl, —CONHaryl, —CON(alkyl)(aryl), —OC(O)aryl, —NHC(O)aryl, —NalkylC(O)aryl, —COOheteroaryl, —COheteroaryl, —CONHheteroaryl, —CON(alkyl)(heteroaryl), —OC(O)heteroaryl, —NHC(O)heteroaryl, —NalkylC(O)heteroaryl, —COOheterocycle, —COheterocycle, —CONHheterocycle, —CON(alkyl)(heterocycle), —OC(O)heterocycle, —NHC(O)heterocycle, and —NalkyC(O)heterocycle.

“Alkenyl” is a linear or branched aliphatic hydrocarbon groups having one or more carbon-carbon double bonds that may occur at a stable point along the chain. The specified ranges as used herein indicate an alkenyl group having each member of the range described as an independent species, as described above for the alkyl moiety. Examples of alkenyl radicals include, but are not limited to ethenyl, propenyl, allyl, propenyl, butenyl and 4-methylbutenyl. The term “alkenyl” also embodies “cis” and “trans” alkenyl geometry, or alternatively, “E” and “Z” alkenyl geometry. In an alternative embodiment, the alkenyl group is optionally substituted. The term “Alkenyl” also encompasses cycloalkyl or carbocyclic groups possessing at least one point of unsaturation. As used herein “substituted alkenyl” can be substituted with the groups described above for alkyl.

“Alkynyl” is a branched or straight chain aliphatic hydrocarbon group having one or more carbon-carbon triple bonds that may occur at any stable point along the chain. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. In an alternative embodiment, the alkynyl group is optionally substituted. The term “Alkynyl” also encompasses cycloalkyl or carbocyclic groups possessing at least one point of unsaturation. As used herein “substituted alkynyl” can be substituted with the groups described above for alkyl.

“Halo” and “Halogen” is fluorine, chlorine, bromine or iodine.

“Haloalkyl” is a branched or straight-chain alkyl groups substituted with 1 or more halo atoms described above, up to the maximum allowable number of halogen atoms. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perhaloalkyl” means an alkyl group having all hydrogen atoms replaced with halogen atoms. Examples include but are not limited to, trifluoromethyl and pentafluoroethyl.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more cycloalkyl or heterocycle groups wherein the radical or point of attachment is on the aryl ring, and in such instances, the number of carbon atoms continue to designate the number of carbon atoms in the aryl ring system. The one or more fused cycloalkyl or heterocycle groups can be 4 to 7-membered saturated or partially unsaturated cycloalkyl or heterocycle groups. As used herein “substituted aryl” refers to an aryl group that is substituted with the described substituents. If no substituents are explicitly described “substituted aryl” refers to an aryl group that is substituted with 1, 2, 3, or 4 substituents independently selected from F, Cl, Br, I, cyano, hydroxy, —O-alkyl, —SH, —Salkyl, —COOH, —COOalkyl, —COalkyl, —COH, —CONH₂, —CONHalkyl, —CON(alkyl)₂, —OC(O)alkyl, —NHC(O)alkyl, —NalkylC(O)alkyl, nitro, amino, —NHalkyl, N(alkyl)₂, cyano, haloalkyl, aryl, heteroaryl, alkyl, alkenyl, alkynyl, haloalkyl, cycloalkyl, alkyl-aryl, alkyl-heteroaryl, alkyl-cycloalkyl, alkyl-heterocycle, heterocycle, —COOaryl, —COaryl, —CONHaryl, —CON(alkyl)(aryl), —OC(O)aryl, —NHC(O)aryl, —NalkylC(O)aryl, —COOheteroaryl, —COheteroaryl, —CONHheteroaryl, —CON(alkyl)(heteroaryl), —OC(O)heteroaryl, —NHC(O)heteroaryl, —NalkylC(O)heteroaryl, —COOheterocycle, —COheterocycle, —CONHheterocycle, —CON(alkyl)(heterocycle), —OC(O)heterocycle, —NHC(O)heterocycle, and —NalkylC(O)heterocycle.

The terms “heterocyclyl” and “heterocycle” include saturated, and partially saturated heteroatom-containing ring radicals, where the heteroatoms may be selected from nitrogen, sulfur, boron, silicone, and oxygen. Heterocyclic rings comprise monocyclic 3-10 membered rings, as well as 5-16 membered bicyclic ring systems (which can include bridged fused and spiro-fused bicyclic ring systems). It does not include rings containing —O—O—. —O—S— or —S—S-portions. Examples of saturated heterocycle groups include saturated 3- to 6-membered heteromonocyclic groups containing 1 to 4 nitrogen atoms [e.g. pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. As used herein “substituted heterocycle” refers to a heterocycle group that is substituted with the described substituents. If no substituents are explicitly described “substituted heterocycle” refers to a heterocycle group that is substituted with 1, 2, 3, or 4 substituents independently selected from oxo, F, Cl, Br, I, cyano, hydroxy, —O-alkyl, —SH, —Salkyl, —COOH, —COOalkyl, —COalkyl, —COH, —CONH₂, —CONHalkyl, —CON(alkyl)₂, —OC(O)alkyl, —NHC(O)alkyl, —NalkyC(O)alkyl, nitro, amino, —NHalkyl, N(alkyl)₂, cyano, haloalkyl, aryl, heteroaryl, alkyl, alkenyl, alkynyl, haloalkyl, cycloalkyl, alkyl-aryl, alkyl-heteroaryl, alkyl-cycloalkyl, alkyl-heterocycle, heterocycle, —COOaryl, —COaryl, —CONHaryl, —CON(alkyl)(aryl), —OC(O)aryl, —NHC(O)aryl, —NalkylC(O)aryl, —COOheteroaryl, —COheteroaryl, —CONHheteroaryl, —CON(alkyl)(heteroaryl), —OC(O)heteroaryl, —NHC(O)heteroaryl, —NalkylC(O)heteroaryl, —COOheterocycle, —COheterocycle, —CONHheterocycle, —CON(alkyl)(heterocycle), —OC(O)heterocycle, —NHC(O)heterocycle, and -NalkyC(O)heterocycle.

“Heterocycle” also includes groups wherein the heterocyclic radical is fused/condensed with an aryl or carbocycle radical, wherein the point of attachment is the heterocycle ring. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

The term “heteroaryl” denotes stable aromatic ring systems that contain one or more heteroatoms selected from O, N, and S, wherein the ring nitrogen and sulfur atom(s) are optionally oxidized, and nitrogen atom(s) are optionally quarternized. Examples include but are not limited to, unsaturated 5 to 6 membered heteromonocyclyl groups containing 1 to 4 nitrogen atoms, such as pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, IH-1,2,3-triazolyl, 2H-1,2,3-triazolyl]; unsaturated 5- to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]. In one embodiment the “heteroaryl” group is a 8, 9, or 10 membered bicyclic ring system. Examples of 8, 9, or 10 membered bicyclic heteroaryl groups include benzofurazanyl, benzothiophenyl, benzothiazolyl, benzoxazolyl, quinazolinyl, quinoxalinyl, naphthyridinyl, quinolinyl, isoquinolinyl, benzofuranyl, indolyl, indazolyl, and benzotriazolyl. As used herein “substituted heteroaryl” refers to a heteroaryl group that is substituted with the described substituents. If no substituents are explicitly described “substituted heteroaryl” refers to a heteroaryl group that is substituted with 1, 2, 3, or 4 substituents independently selected from F, Cl, Br, I, cyano, hydroxy, —O-alkyl, —SH, —Salkyl, —COOH, —COOalkyl, —COalkyl, —COH, —CONH₂, —CONHalkyl, —CON(alkyl)₂, —OC(O)alkyl, —NHC(O)alkyl, —NalkyC(O)alkyl, nitro, amino, —NHalkyl, N(alkyl)₂, cyano, haloalkyl, aryl, heteroaryl, alkyl, alkenyl, alkynyl, haloalkyl, cycloalkyl, alkyl-aryl, alkyl-heteroaryl, alkyl-cycloalkyl, alkyl-heterocycle, heterocycle, —COOaryl, —COaryl, —CONHaryl, —CON(alkyl)(aryl), —OC(O)aryl, —NHC(O)aryl, —NalkylC(O)aryl, —COOheteroaryl, —COheteroaryl, —CONHheteroaryl, —CON(alkyl)(heteroaryl), —OC(O)heteroaryl, —NHC(O)heteroaryl, —NalkylC(O)heteroaryl, —COOheterocycle, —COheterocycle, —CONHheterocycle, —CON(alkyl)(heterocycle), —OC(O)heterocycle, —NHC(O)heterocycle, and —NalkyC(O)heterocycle.

The term “sulfonyl”, whether used alone or linked to other terms such as alkylsulfonyl, denotes respectively divalent radicals —SO₂—.

“Alkyl-heterocycle” is an alkyl group as defined herein with a heterocycle substituent. Examples include but are not limited to, piperidylmethyl and morpholinylethyl.

“Alkyl-aryl” is an alkyl group as defined herein with an aryl substituent. Non-limiting examples of alkyl-aryl groups include:

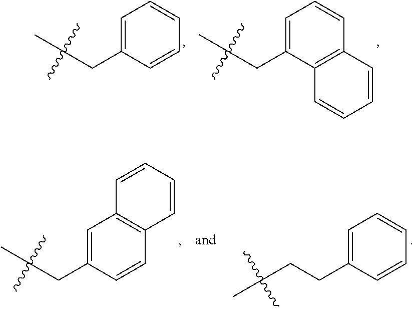 [SMILES: *Cc1ccccc1 ; *Cc1cccc2ccccc12 ; *Cc1ccc2ccccc2c1 ; *CCc1ccccc1]

“Alkyl-heteroaryl” is an alkyl group as defined herein with a heteroaryl substituent. Non-limiting examples of alkyl-heteroaryl groups include:

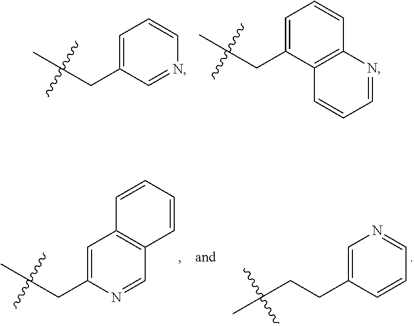 [SMILES: *Cc1cccnc1 ; *Cc1cccc2ncccc12 ; *Cc1cc2ccccc2cn1 ; *CCc1cccnc1]

As used herein, “carbocyclyl”, “carbocyclic”, “carbocycle” or “cycloalkyl” is a saturated or partially unsaturated (i.e., not aromatic) group containing all carbon ring atoms and from 3 to 14 ring carbon atoms (“C₃₋₁₄ cycloalkyl”) and zero heteroatoms in the non-aromatic ring system. In some embodiments, a cycloalkyl group has 3 to 10 ring carbon atoms (“C₃₋₁₀ cycloalkyl”). In some embodiments, a cycloalkyl group has 3 to 9 ring carbon atoms (“C₃₋₉ cycloalkyl”). In some embodiments, a cycloalkyl group has 3 to 8 ring carbon atoms (“C₃₋₈ cycloalkyl”). In some embodiments, a cycloalkyl group has 3 to 7 ring carbon atoms (“C₃₋₇ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Exemplary C₃₋₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ cycloalkyl groups include, without limitation, the aforementioned C₃₋₆ cycloalkyl groups as well as cycloheptyl (C₇), cycloheptenyl (C₇), cycloheptadienyl (C₇), cycloheptatrienyl (C₇), cyclooctyl (C), cyclooctenyl (C), and the like. Exemplary C₃₋₁₀ cycloalkyl groups include, without limitation, the aforementioned C₃₋₈ cycloalkyl groups as well as cyclononyl (C₉), cyclononenyl (C₉), cyclodecyl (C₁₀), cyclodecenyl (C₁₀), and the like. As the foregoing examples illustrate, in certain embodiments, the cycloalkyl group can be saturated or can contain one or more carbon-carbon double or triple bonds. In an alternative embodiment, “cycloalkyl” also includes ring systems wherein the cycloalkyl ring, as defined above, is fused with one heterocycle, aryl or heteroaryl ring wherein the point of attachment is on the cycloalkyl ring, and in such instances, the number of carbons continue to designate the number of carbons in the carbocyclic ring system. In an alternative embodiment, each instance of cycloalkyl is optionally substituted with one or more substituents. In certain embodiments, the cycloalkyl group is an unsubstituted C₃₋₁₄ cycloalkyl.

“Alkyl-cycloalkyl” is an alkyl group as defined herein with a cycloalkyl substituent. Non-limiting examples of alkyl-cycloalkyl groups include:

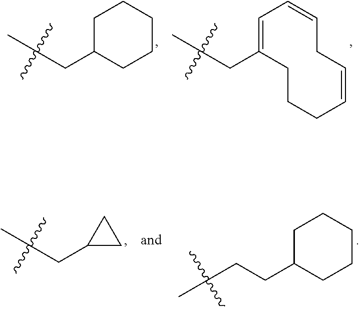 [SMILES: *CC1CCCCC1 ; *C/C1=C/C=C\C/C=C\CCC1 ; *CC1CC1 ; *CCC1CCCCC1]

The term “oxo” as used herein contemplates an oxygen atom attached with a double bond.

### III. Methods of Treatment

In one aspect, a method of treating an abnormal cellular proliferation in a host, including a human, is provided comprising administering an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or its pharmaceutically acceptable salt, N-oxide, deuterated derivative, prodrug, and/or a pharmaceutically acceptable composition thereof as described herein optionally in a pharmaceutically acceptable carrier. Non-limiting examples of abnormal cellular proliferations include tumors, cancers, disorders related to abnormal cellular proliferation, inflammatory disorders, immune disorders, and autoimmune disorders.

More generally, a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII is useful as a therapeutic agent when administered in an effective amount to a host, including a human, to treat a tumor, cancer (solid, non-solid, diffuse, hematological, etc.), abnormal cellular proliferation, immune disorder, inflammatory disorder, blood disorder, a myelo- or lymphoproliferative disorder such as B- or T-cell lymphomas, multiple myeloma, breast cancer, prostate cancer, AML, ALL, ACL, lung cancer, pancreatic cancer, colon cancer, skin cancer, melanoma, Waldenstrom's macroglobulinemia, Wiskott-Aldrich syndrome, or a post-transplant lymphoproliferative disorder; an autoimmune disorder, for example, Lupus, Crohn's Disease, Addison disease, Celiac disease, dermatomyositis, Graves disease, thyroiditis, multiple sclerosis, pernicious anemia, reactive arthritis, or type I diabetes; a disease of cardiologic malfunction, including hypercholesterolemia; an infectious disease, including a viral and/or bacterial infection; an inflammatory condition, including asthma, chronic peptic ulcers, tuberculosis, rheumatoid arthritis, periodontitis, ulcerative colitis, or hepatitis.

In one embodiment the compound of the present invention is used to treat breast cancer. In one embodiment the breast cancer is HR+ and HER2−. In one embodiment the breast cancer is HR− and HER2+. In one embodiment the breast cancer is estrogen-receptor-positive (ER+), progesterone-receptor-negative (PR−) and HER2−. In one embodiment the breast cancer is estrogen-receptor-positive (ER+), progesterone-receptor-negative (PR−) and HER2+. In one embodiment the breast cancer is estrogen-receptor-negative (ER−), progesterone-receptor-positive (PR+) and HER2−. In one embodiment the breast cancer is estrogen-receptor-negative (ER−), progesterone-receptor-positive (PR+) and HER2+.

In one embodiment the compound of the present invention is used to treat non-small cell lung cancer (NSCLC). In one embodiment the NSCLC has an EGFR mutation. In one embodiment the NSCLC has an EGFR mutation and an EGFR inhibitor failed (e.g. 2^{nd} line therapy). In one embodiment an ALK inhibitor failed (e.g. 2^{nd} line therapy). In one embodiment the NSCLC has an KRAS mutation.

In one embodiment the compound of the present invention is used to treat prostate cancer. In one embodiment the prostate cancer is castration resistant. In one embodiment a prior chemotherapeutic agent already failed (e.g. 2^{nd} line therapy).

In one embodiment the compound of the present invention is used to treat lymphoma. In one embodiment the lymphoma is mantel cell lymphoma (MCL), marginal zone lymphoma (MZL), chronic lymphocytic leukemia (CLL), follicular lymphoma (FL), or diffuse large B-cell lymphoma (DLBCL). In one embodiment a prior chemotherapeutic agent already failed (e.g. 2^{nd} line therapy).

In one embodiment the compound of the present invention is used to treat melanoma.

In one embodiment the melanoma is has a BRAF mutation.

In one embodiment the compound of the present invention is used to treat RAS mutated cancer. In one embodiment the RAS mutated cancer is colon cancer (CLC). In one embodiment the RAS mutated cancer is pancreatic cancer. In one embodiment the RAS mutated cancer is cholangiocarcinoma.

In one embodiment the compound of the present invention is used to treat a gastrointestinal stromal tumor (GIST). In one embodiment the treatment with imatinib or sunitinib already failed (e.g. 2^{nd} line therapy).

Exemplary proliferative disorders include, but are not limited to, benign growths, neoplasms, tumors, cancer (Rb positive or Rb negative), autoimmune disorders, inflammatory disorders graft-versus-host rejection, and fibrotic disorders.

Non-limiting examples of cancers that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In another embodiment, the disorder is myelodysplastic syndrome (MDS).

In certain embodiments, the cancer is a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the proliferative disorder is a myeloproliferative neoplasm. In certain embodiments, the myeloproliferative neoplasm (MPN) is primary myelofibrosis (PMF).

In certain embodiments, the cancer is a solid tumor. A solid tumor, as used herein, refers to an abnormal mass of tissue that typically does not contain cysts or liquid areas. Different types of solid tumors are named for the type of cells that form them. Examples of classes of solid tumors include, but are not limited to, sarcomas, carcinomas, and lymphomas, as described above herein. Additional examples of solid tumors include, but are not limited to, squamous cell carcinoma, colon cancer, breast cancer, prostate cancer, lung cancer, liver cancer, pancreatic cancer, and melanoma.

In certain embodiments, the condition treated with a compound Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII is a disorder related to abnormal cellular proliferation.

Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

In certain embodiments, a compound of the present invention and its pharmaceutically acceptable derivatives or pharmaceutically acceptable formulations containing these compounds are also useful in the prevention and treatment of HBV infections and other related conditions such as anti-HBV antibody positive and HBV-positive conditions, chronic liver inflammation caused by HBV, cirrhosis, acute hepatitis, fulminant hepatitis, chronic persistent hepatitis, and fatigue. These compounds or formulations can also be used prophylactically to prevent or retard the progression of clinical illness in individuals who are anti-HBV antibody or HBV-antigen positive or who have been exposed to HBV.

In certain embodiments, the condition is associated with an immune response.

Cutaneous contact hypersensitivity and asthma are just two examples of immune responses that can be associated with significant morbidity. Others include atopic dermatitis, eczema, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, cutaneous lupus erythematosus, scleroderma, vaginitis, proctitis, and drug eruptions. These conditions may result in any one or more of the following symptoms or signs: itching, swelling, redness, blisters, crusting, ulceration, pain, scaling, cracking, hair loss, scarring, or oozing of fluid involving the skin, eye, or mucosal membranes.

In atopic dermatitis, and eczema in general, immunologically mediated leukocyte infiltration (particularly infiltration of mononuclear cells, lymphocytes, neutrophils, and eosinophils) into the skin importantly contributes to the pathogenesis of these diseases. Chronic eczema also is associated with significant hyperproliferation of the epidermis. Immunologically mediated leukocyte infiltration also occurs at sites other than the skin, such as in the airways in asthma and in the tear producing gland of the eye in keratoconjunctivitis sicca.

In one non-limiting embodiment compounds of the present invention are used as topical agents in treating contact dermatitis, atopic dermatitis, eczematous dermatitis, psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, and drug eruptions. The novel method may also be useful in reducing the infiltration of skin by malignant leukocytes in diseases such as mycosis fungoides. These compounds can also be used to treat an aqueous-deficient dry eye state (such as immune mediated keratoconjunctivitis) in a patient suffering therefrom, by administering the compound topically to the eye.

The term “neoplasia” or “cancer” is used throughout the specification to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant hematogenous, ascitic and solid tumors.

Exemplary cancers which may be treated by the present disclosed compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER−2) negative breast cancer, double negative breast cancer (two of estrogen, progesterone and HER−2 are negative), single negative (one of estrogen, progesterone and HER−2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

In another aspect, a method of increasing BIM expression (e.g., BCLC2L11 expression) is provided to induce apoptosis in a cell comprising contacting a compound of the present invention or a pharmaceutically acceptable composition, salt, isotopic analog, or prodrug thereof with the cell. In certain embodiments, the method is an in vitro method. In certain embodiments, the method is an in vivo method. BCL2L11 expression is tightly regulated in a cell. BCL2L11 encodes for BIM, a proapoptotic protein. BCL2L11 is downregulated in many cancers and BIM is inhibited in many cancers, including chronic myelocytic leukemia (CML) and non-small cell lung cancer (NSCLC) and that suppression of BCL2L11 expression can confer resistance to tyrosine kinase inhibitors. See, e.g., Ng et al., Nat. Med. (2012) 18:521-528.

In yet another aspect, a method of treating a condition associated with angiogenesis is provided, such as, for example, a diabetic condition (e.g., diabetic retinopathy), an inflammatory condition (e.g., rheumatoid arthritis), macular degeneration, obesity, atherosclerosis, or a proliferative disorder, comprising administering to a subject in need thereof a compound of the present invention or a pharmaceutically acceptable composition, salt, isotopic analog, or prodrug thereof.

In certain embodiments, the condition associated with angiogenesis is macular degeneration. In certain embodiments, provided is a method of treating macular degeneration comprising administering to a subject in need thereof a compound of the present invention or a pharmaceutically acceptable composition, salt, isotopic analog, or prodrug thereof.

In certain embodiments, the condition associated with angiogenesis is obesity. As used herein, “obesity” and “obese” as used herein, refers to class I obesity, class II obesity, class III obesity and pre-obesity (e.g., being “over-weight”) as defined by the World Health Organization. In certain embodiments, a method of treating obesity is provided comprising administering to a subject in need thereof a compound of the present invention or a pharmaceutically acceptable composition, salt, isotopic analog, or prodrug thereof.

In certain embodiments, the condition associated with angiogenesis is atherosclerosis. In certain embodiments, provided is a method of treating atherosclerosis comprising administering to a subject in need thereof a compound of the present invention or a pharmaceutically acceptable composition, salt, isotopic analog, or prodrug thereof.

In certain embodiments, the condition associated with angiogenesis is a proliferative disorder. In certain embodiments, provided is a method of treating a proliferative disorder comprising administering to a subject in need thereof a compound of the present invention or a pharmaceutically acceptable composition, salt, isotopic analog, or prodrug thereof.

### IV. Methods to Reduce the Side Effects Related to Chemotherapy

In certain embodiments, compounds of the present invention decrease the effect of chemotherapeutic agent toxicity on CDK replication dependent healthy cells, such as hematopoietic stem cells and hematopoietic progenitor cells (together referred to as HSPCs), and/or renal epithelial cells, in subjects, typically humans, that will be, are being, or have been exposed to the chemotherapeutic agent (typically a DNA-damaging agent).

In one embodiment, the subject has been exposed to a chemotherapeutic agent, and, using a compound described herein, the subject's CDK replication dependent healthy cells are placed in G1 arrest following exposure in order to mitigate, for example, DNA damage. In one embodiment, the compound is administered at least 1/2 hour, at least 1 hour, at least 2 hours, at least 3 hours, at least 4 hours, at least 5 hours, at least 6 hours, at least 7 hours, at least 8 hours, at least 10 hours, at least 12 hours, at least 14 hours, at least 16 hours, at least 18 hours, at least 20 hours or more post chemotherapeutic agent exposure.

In one embodiment, the compound can allow for dose intensification (e.g., more therapy can be given in a fixed period of time) in medically related chemotherapies, which will translate to better efficacy. Therefore, the presently disclosed methods can result in chemotherapy regimens that are less toxic and more effective.

In some embodiments, the use of a compound described herein may result in reduced or substantially free of off-target effects, for example, related to inhibition of kinases other than the selective CDK targeted by the compound of the present invention, such as CDK4, CDK6, CDK9, or CDK2. Furthermore, in certain embodiments, the use of the compounds described herein should not induce cell cycle arrest in CDK replication independent cells.

In some embodiments, the use of a compound described herein reduces the risk of undesirable off-target effects including, but not limited to, long term toxicity, anti-oxidant effects, and estrogenic effects. Anti-oxidant effects can be determined by standard assays known in the art. For example, a compound with no significant anti-oxidant effects is a compound that does not significantly scavenge free-radicals, such as oxygen radicals. The anti-oxidant effects of a compound can be compared to a compound with known anti-oxidant activity, such as genistein. Thus, a compound with no significant anti-oxidant activity can be one that has less than about 2, 3, 5, 10, 30, or 100 fold anti-oxidant activity relative to genistein. Estrogenic activities can also be determined via known assays. For instance, a non-estrogenic compound is one that does not significantly bind and activate the estrogen receptor. A compound that is substantially free of estrogenic effects can be one that has less than about 2, 3, 5, 10, 20, or 100 fold estrogenic activity relative to a compound with estrogenic activity, e.g., genistein.

### V. Methods to Treat Abnormal Proliferation of T-Cells, B-Cells and/or NK-Cells

In certain aspects, the invention includes the use of an effective amount of a compound described herein, or its pharmaceutically acceptable salt, prodrug or isotopic variant optionally in a pharmaceutical composition, to treat a host, typically a human, with a selected cancer, tumor, hyperproliferative condition or an inflammatory or immune disorder. Some of the disclosed compounds are highly active against T-cell proliferation. Given the paucity of drugs for T-cell cancers and abnormal proliferation, the identification of such uses represents a substantial improvement in the medical therapy for these diseases.

Abnormal proliferation of T-cells, B-cells, and/or NK-cells can result in a wide range of diseases such as cancer, proliferative disorders and inflammatory/immune diseases. A host, for example a human, afflicted with any of these disorders can be treated with an effective amount of a compound as described herein to achieve a decrease in symptoms (a palliative agent) or a decrease in the underlying disease (a disease modifying agent).

Examples include T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In one embodiment, a compound disclosed herein, or its salt, prodrug, or isotopic variant can be used in an effective amount to treat a host, for example a human, with a lymphoma or lymphocytic or myelocytic proliferation disorder or abnormality. For example, the compounds as described herein can be administered to a host suffering from a Hodgkin Lymphoma or a Non-Hodgkin Lymphoma. For example, the host can be suffering from a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

Alternatively, a compound disclosed herein, or its salt, prodrug, or isotopic variant can be used in an effective amount to treat a host, for example a human, with a Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

Alternatively, a compound disclosed herein, or its salt, prodrug, or isotopic variant can be used in an effective amount to treat a host, for example a human with a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+DLBCL of the elderly; Primary mediastinal (thymic) large B-cell lymphoma; Primary cutaneous DLBCL, leg type; ALK+large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma.

In one embodiment, a compound disclosed herein, or its salt, prodrug, or isotopic variant can be used in an effective amount to treat a host, for example a human with leukemia. For example, the host may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In one embodiment, the patient suffers from an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

### VI. Pharmaceutical Compositions and Dosage Forms

An active compound described herein, or its salt, isotopic analog, or prodrug can be administered in an effective amount to a host, for example human, to treat any of the disorders described herein using any suitable approach which achieves the desired therapeutic result. The amount and timing of active compound administered will, of course, be dependent on the host being treated, the instructions of the supervising medical specialist, on the time course of the exposure, on the manner of administration, on the pharmacokinetic properties of the particular active compound, and on the judgment of the prescribing physician. Thus, because of host to host variability, the dosages given below are a guideline and the physician can titrate doses of the compound to achieve the treatment that the physician considers appropriate for the host. In considering the degree of treatment desired, the physician can balance a variety of factors such as age and weight of the host, presence of preexisting disease, as well as presence of other diseases.

The pharmaceutical composition may be formulated as any pharmaceutically useful form, e.g., as an aerosol, a cream, a gel, a pill, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose.

The therapeutically effective dosage of any active compound described herein will be determined by the health care practitioner depending on the condition, size and age of the patient as well as the route of delivery. In one non-limited embodiment, a dosage from about 0.1 to about 200 mg/kg has therapeutic efficacy, with all weights being calculated based upon the weight of the active compound, including the cases where a salt is employed. In one embodiment, the dosage is at about or greater than 0.1, 0.5, 1, 5, 10, 25, 50, 75, 100, 125, 150, 175, or 200 mg/kg. In some embodiments, the dosage may be the amount of compound needed to provide a serum concentration of the active compound of up to about 10 nM, 50 nM, 100 nM, 200 nM, 300 nM, 400 nM, 500 nM, 600 nM, 700 nM, 800 nM, 900 nM, 1 μM, 5 μM, 10 μM, 20 μM, 30 μM, or 40 μM.

In certain embodiments, the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples of dosage forms with about or at least 5, 10, 15, 20, 25, 50, 100, 200, 250, 300, 400, 500, 600, 700, or 750 mg of active compound, or its salt. The pharmaceutical composition may also include a molar ratio of the active compound and an additional active agent, in a ratio that achieves the desired results.

Compounds disclosed herein or used as described herein may be administered orally, topically, parenterally, by inhalation or spray, sublingually, via implant, including ocular implant, transdermally, via buccal administration, rectally, as an ophthalmic solution, injection, including ocular injection, intravenous, intramuscular, inhalation, intra-aortal, intracranial, subdermal, intraperitoneal, subcutaneous, transnasal, sublingual, or rectal or by other means, in dosage unit formulations containing conventional pharmaceutically acceptable carriers. For ocular delivery, the compound can be administered, as desired, for example, via intravitreal, intrastromal, intracameral, sub-tenon, sub-retinal, retro-bulbar, peribulbar, suprachorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

In accordance with the presently disclosed methods, an oral administration can be in any desired form such as a solid, gel or liquid, including a solution, suspension, or emulsion. In some embodiments, the compounds or salts are administered by inhalation, intravenously, or intramuscularly as a liposomal suspension. When administered through inhalation the active compound or salt may be in the form of a plurality of solid particles or droplets having any desired particle size, and for example, from about 0.01, 0.1 or 0.5 to about 5, 10, 20 or more microns, and optionally from about 1 to about 2 microns. Compounds as disclosed in the present invention have demonstrated good pharmacokinetic and pharmacodynamics properties, for instance when administered by the oral or intravenous routes.

The pharmaceutical formulations can comprise an active compound described herein or a pharmaceutically acceptable salt thereof, in any pharmaceutically acceptable carrier. If a solution is desired, water may sometimes be the carrier of choice for water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and for illustration by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

Thus, the compositions of the disclosure can be administered as pharmaceutical formulations including those suitable for oral (including buccal and sub-lingual), rectal, nasal, topical, pulmonary, vaginal or parenteral (including intramuscular, intra-arterial, intrathecal, subcutaneous and intravenous) administration or in a form suitable for administration by inhalation or insufflation. The preferred manner of administration is intravenous or oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as an continuous infusion system. A formulation provided by the disclosure can be administered using a syringe, injector, pump, or any other device recognized in the art for parenteral administration.

In addition to the active compounds or their salts, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. An antimicrobial preservative is typically employed when the formulations is placed in a vial designed for multi-dose use. The pharmaceutical formulations described herein can be lyophilized using techniques well known in the art.

For oral administration, a pharmaceutical composition can take the form of a solution suspension, tablet, pill, capsule, powder, and the like. Tablets containing various excipients such as sodium citrate, calcium carbonate and calcium phosphate may be employed along with various disintegrants such as starch (e.g., potato or tapioca starch) and certain complex silicates, together with binding agents such as polyvinylpyrrolidone, sucrose, gelatin and acacia. Additionally, lubricating agents such as magnesium stearate, sodium lauryl sulfate, and talc are often very useful for tableting purposes. Solid compositions of a similar type may be employed as fillers in soft and hard-filled gelatin capsules. Materials in this connection also include lactose or milk sugar as well as high molecular weight polyethylene glycols. When aqueous suspensions and/or elixirs are desired for oral administration, the compounds of the presently disclosed host matter can be combined with various sweetening agents, flavoring agents, coloring agents, emulsifying agents and/or suspending agents, as well as such diluents as water, ethanol, propylene glycol, glycerin and various like combinations thereof.

In yet another embodiment of the host matter described herein, there are provided injectable, stable, sterile formulations comprising an active compound as described herein, or a salt thereof, in a unit dosage form in a sealed container. The compound or salt is provided in the form of a lyophilizate, which is capable of being reconstituted with a suitable pharmaceutically acceptable carrier to form liquid formulation suitable for injection thereof into a host. When the compound or salt is substantially water-insoluble, a sufficient amount of emulsifying agent, which is physiologically acceptable, can be employed in sufficient quantity to emulsify the compound or salt in an aqueous carrier. Particularly useful emulsifying agents include phosphatidyl cholines and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound can be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques. The liposomal formulations comprising the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol by inhalation. These formulations comprise a solution or suspension of a desired compound described herein or a salt thereof, or a plurality of solid particles of the compound or salt. The desired formulations can be placed in a small chamber and nebulized. Nebulization can be accomplished by compressed air or by ultrasonic energy to form a plurality of liquid droplets or solid particles comprising the compounds or salts. The liquid droplets or solid particles may for example have a particle size in the range of about 0.5 to about 10 microns, and optionally from about 0.5 to about 5 microns. In one embodiment, the solid particles provide for controlled release through the use of a degradable polymer. The solid particles can be obtained by processing the solid compound or a salt thereof, in any appropriate manner known in the art, such as by micronization. Optionally, the size of the solid particles or droplets can be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

Pharmaceutical formulations also are provided which provide a controlled release of a compound described herein, including through the use of a degradable polymer, as known in the art.

When the pharmaceutical formulations suitable for administration as an aerosol is in the form of a liquid, the formulations can comprise a water-soluble active compound in a carrier that comprises water. A surfactant can be present, which lowers the surface tension of the formulations sufficiently to result in the formation of droplets within the desired size range when hosted to nebulization.

The term “pharmaceutically acceptable salts” as used herein refers to those salts which are, within the scope of sound medical judgment, suitable for use in contact with hosts (e.g., human hosts) without undue toxicity, irritation, allergic response, and the like, commensurate with a reasonable benefit/risk ratio, and effective for their intended use, as well as the zwitterionic forms, where possible, of the compounds of the presently disclosed host matter.

Thus, the term “salts” refers to the relatively non-toxic, inorganic and organic acid addition salts of the presently disclosed compounds. These salts can be prepared during the final isolation and purification of the compounds or by separately reacting the purified compound in its free base form with a suitable organic or inorganic acid and isolating the salt thus formed. Basic compounds are capable of forming a wide variety of different salts with various inorganic and organic acids. Acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents. Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Formulations suitable for rectal administration are typically presented as unit dose suppositories. These may be prepared by admixing the active disclosed compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. In one embodiment, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

Additional non-limiting examples of drug delivery devices and methods include, for example, US20090203709 titled “Pharmaceutical Dosage Form For Oral Administration Of Tyrosine Kinase Inhibitor” (Abbott Laboratories); US20050009910 titled “Delivery of an active drug to the posterior part of the eye via subconjunctival or periocular delivery of a prodrug”, US 20130071349 titled “Biodegradable polymers for lowering intraocular pressure” U.S. Pat. No. 8,481,069 titled “Tyrosine kinase microspheres”, U.S. Pat. No. 8,465,778 titled “Method of making tyrosine kinase microspheres”, U.S. Pat. No. 8,409,607 titled “Sustained release intraocular implants containing tyrosine kinase inhibitors and related methods”, U.S. Pat. No. 8,512,738 and US 2014/0031408 titled “Biodegradable intravitreal tyrosine kinase implants”, US 2014/0294986 titled “Microsphere Drug Delivery System for Sustained Intraocular Release”, U.S. Pat. No. 8,911,768 titled “Methods For Treating Retinopathy With Extended Therapeutic Effect” (Allergan, Inc.); U.S. Pat. No. 6,495,164 titled “Preparation of injectable suspensions having improved injectability” (Alkermes Controlled Therapeutics, Inc.); WO 2014/047439 titled “Biodegradable Microcapsules Containing Filling Material” (Akina, Inc.); WO 2010/132664 titled “Compositions And Methods For Drug Delivery” (Baxter International Inc. Baxter Healthcare SA); US20120052041 titled “Polymeric nanoparticles with enhanced drug loading and methods of use thereof” (The Brigham and Women's Hospital, Inc.); US20140178475, US20140248358, and US20140249158 titled “Therapeutic Nanoparticles Comprising a Therapeutic Agent and Methods of Making and Using Same” (BIND Therapeutics, Inc.); U.S. Pat. No. 5,869,103 titled “Polymer microparticles for drug delivery” (Danbiosyst UK Ltd.); U.S. Pat. No. 8,628,801 titled “Pegylated Nanoparticles” (Universidad de Navarra); US2014/0107025 titled “Ocular drug delivery system” (Jade Therapeutics, LLC); U.S. Pat. No. 6,287,588 titled “Agent delivering system comprised of microparticle and biodegradable gel with an improved releasing profile and methods of use thereof”, U.S. Pat. No. 6,589,549 titled “Bioactive agent delivering system comprised of microparticles within a biodegradable to improve release profiles” (Macromed, Inc.); U.S. Pat. Nos. 6,007,845 and 5,578,325 titled “Nanoparticles and microparticles of non-linear hydrophilic hydrophobic multiblock copolymers” (Massachusetts Institute of Technology); US20040234611, US20080305172, US20120269894, and US20130122064 titled “Ophthalmic depot formulations for periocular or subconjunctival administration (Novartis Ag); U.S. Pat. No. 6,413,539 titled “Block polymer” (Poly-Med, Inc.); US 20070071756 titled “Delivery of an agent to ameliorate inflammation” (Peyman); US 20080166411 titled “Injectable Depot Formulations And Methods For Providing Sustained Release Of Poorly Soluble Drugs Comprising Nanoparticles” (Pfizer, Inc.); U.S. Pat. No. 6,706,289 titled “Methods and compositions for enhanced delivery of bioactive molecules” (PR Pharmaceuticals, Inc.); and U.S. Pat. No. 8,663,674 titled “Microparticle containing matrices for drug delivery” (Surmodics).

### VII. Combination Therapy

The disclosed compounds of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII can be used in an effective amount alone or in combination with another compound of the present invention or another bioactive agent (therapeutic agent) to treat a host such as a human with a disorder as described herein.

The disclosed compounds described herein can be used in an effective amount alone or in combination with another compound of the present invention or another bioactive agent to treat a host such as a human with a disorder as described herein.

The term “bioactive agent” or “therapeutic agent” is used to describe an agent, other than the selected compound according to the present invention, which can be used in combination or alternation with a compound of the present invention to achieve a desired result of therapy. In one embodiment, the compound of the present invention and the bioactive agent are administered in a manner that they are active in vivo during overlapping time periods, for example, have time-period overlapping Cmax, Tmax, AUC or other pharmacokinetic parameter. In another embodiment, the compound of the present invention and the bioactive agent are administered to a host in need thereof that do not have overlapping pharmacokinetic parameter, however, one has a therapeutic impact on the therapeutic efficacy of the other.

In one aspect of this embodiment, the bioactive agent is a chemotherapeutic.

In another aspect of this embodiment, the bioactive agent is a growth factor.

In one aspect of this embodiment, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody.

### Immune Checkpoint Inhibitors

Immune checkpoint inhibitors for use in the methods described herein include, but are not limited to PD-1 inhibitors, PD-L1 inhibitors, PD-L2 inhibitors, CTLA-4 inhibitors, LAG-3 inhibitors, TIM-3 inhibitors, and V-domain Ig suppressor of T-cell activation (VISTA) inhibitors, or combinations thereof.

In one embodiment, the immune checkpoint inhibitor is a PD-1 inhibitor that blocks the interaction of PD-1 and PD-L1 by binding to the PD-1 receptor, and in turn inhibits immune suppression. In one embodiment, the immune checkpoint inhibitor is a PD-1 immune checkpoint inhibitor selected from nivolumab (Opdivo®), pembrolizumab (Keytruda®), pidilizumab, AMP-224 (AstraZeneca and MedImmune), PF-06801591 (Pfizer), MEDI680 (AstraZeneca), PDR001 (Novartis), REGN2810 (Regeneron), MGA012 (MacroGenics), BGB-A317 (BeiGene) SHR-12-1 (Jiangsu Hengrui Medicine Company and Incyte Corporation), TSR-042 (Tesaro), and the PD-L1/VISTA inhibitor CA-170 (Curis Inc.).

In one embodiment, the immune checkpoint inhibitor is the PD-1 immune checkpoint inhibitor nivolumab (Opdivo®) administered in an effective amount for the treatment of Hodgkin lymphoma, melanoma, non-small cell lung cancer, hepatocellular carcinoma, or ovarian cancer. Nivolumab has been FDA approved for the use of metastatic melanoma, non-small cell lung cancer, and renal cell carcinoma. In another aspect of this embodiment, the immune checkpoint inhibitor is the PD-1 immune checkpoint inhibitor pembrolizumab (Keytruda®) administered in an effective amount for the treatment of melanoma, non-small cell lung cancer, small cell lung cancer, head and neck cancer, or urothelial cancer. In an additional aspect of this embodiment, the immune checkpoint inhibitor is the PD-1 immune checkpoint inhibitor pidilizumab (Medivation) administered in an effective amount for refractory diffuse large B-cell lymphoma (DLBCL) or metastatic melanoma.

In one embodiment, the immune checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, atezolizumab, durvalumab, KN035CA-170 (Curis Inc.), and LY3300054 (Eli Lilly). In one embodiment, the PD-L1 inhibitor is atezolizumab. In one embodiment, the PD-L1 inhibitor blocks the interaction between PD-L1 and CD80 to inhibit immune suppression.

In one embodiment, the immune checkpoint inhibitor is the PD-L1 immune checkpoint inhibitor atezolizumab (Tecentriq®) administered in an effective amount for the treatment of metastatic bladder cancer, metastatic melanoma, metastatic non-small cell lung cancer, or metastatic renal cell carcinoma. In another aspect of this embodiment, the immune checkpoint inhibitor is durvalumab (AstraZeneca and MedImmune) administered in an effective amount for the treatment of non-small cell lung cancer or bladder cancer. In yet another aspect of the embodiment, the immune checkpoint inhibitor is KN035 (Alphamab) administered in an effective amount for the treatment of PD-L1 positive solid tumors. An additional example of a PD-L1 immune checkpoint inhibitor is BMS-936559 (Bristol-Myers Squibb), although clinical trials with this inhibitor have been suspended as of 2015.

In one aspect of this embodiment, the immune checkpoint inhibitor is a CTLA-4 immune checkpoint inhibitor that binds to CTLA-4 and inhibits immune suppression. CTLA-4 inhibitors include, but are not limited to, ipilimumab, tremelimumab (AstraZeneca and MedImmune), AGEN1884 and AGEN2041 (Agenus).

In one embodiment, the CTLA-4 immune checkpoint inhibitor is ipilimumab (Yervoy®) administered in an effective amount for the treatment of metastatic melanoma, adjuvant melanoma, or non-small cell lung cancer.

In another embodiment, the immune checkpoint inhibitor is a LAG-3 immune checkpoint inhibitor. Examples of LAG-3 immune checkpoint inhibitors include, but are not limited to, BMS-986016 (Bristol-Myers Squibb), GSK2831781 (GaxoSmithKline), IMP321 (Prima BioMed), LAG525 (Novartis), and the dual PD-1 and LAG-3 inhibitor MGD013 (MacroGenics). In yet another aspect of this embodiment, the immune checkpoint inhibitor is a TIM-3 immune checkpoint inhibitor. A specific TIM-3 inhibitor includes, but is not limited to, TSR-022 (Tesaro).

Other immune checkpoint inhibitors for use in the invention described herein include, but are not limited to, B7-H3/CD276 immune checkpoint inhibitors such as MGA217, indoleamine 2,3-dioxygenase (IDO) immune checkpoint inhibitors such as Indoximod and INCB024360, killer immunoglobulin-like receptors (KIRs) immune checkpoint inhibitors such as Lirilumab (BMS-986015), carcinoembryonic antigen cell adhesion molecule (CEACAM) inhibitors (e.g., CEACAM-1, -3 and/or -5). Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or cross-reacts with CEACAM-1 and CEACAM-5 as described in, e.g., WO 2013/054331 and US 2014/0271618. Still other checkpoint inhibitors can be molecules directed to B and T lymphocyte attenuator molecule (BTLA), for example as described in Zhang et al., Monoclonal antibodies to B and T lymphocyte attenuator (BTLA) have no effect on in vitro B cell proliferation and act to inhibit in vitro T cell proliferation when presented in a cis, but not trans, format relative to the activating stimulus, Clin Exp Immunol. 2011 January; 163(1): 77-87.

### Chemotherapeutic Agents

As contemplated herein, the active compound of the present invention can be administered in combination with any standard chemotherapeutic agent treatment modality, in further combination with an immune checkpoint inhibitor.

In one embodiment, the chemotherapeutic agent is toxic to immune effector cells. In one embodiment the chemotherapeutic agent inhibits cell growth. In one embodiment, the cytotoxic chemotherapeutic agent administered is a DNA damaging chemotherapeutic agent.

In one embodiment, the chemotherapeutic agent is a protein synthesis inhibitor, a DNA-damaging chemotherapeutic, an alkylating agent, a topoisomerase inhibitor, an RNA synthesis inhibitor, a DNA complex binder, a thiolate alkylating agent, a guanine alkylating agent, a tubulin binder, DNA polymerase inhibitor, an anticancer enzyme, RAC1 inhibitor, thymidylate synthase inhibitor, oxazophosphorine compound, integrin inhibitor such as cilengitide, camptothecin or homocamptothecin, antifolate or a folate antimetabolite.

In one embodiment the additional therapeutic agent is trastuzumab. In one embodiment the additional therapeutic agent is lapatinib. In one embodiment the compound of the present invention is dosed with 2, 3, or 4 additional therapeutic agents. In one embodiment there are t2 additional therapeutic agents. In one embodiment the two additional therapeutic agents are lapatinib and trastuzumab.

In one embodiment the additional therapeutic agent is osimertinib.

In one embodiment the additional therapeutic agent is alectinib.

In one embodiment the additional therapeutic agent is a MEK inhibitor.

In one embodiment the additional therapeutic agent is an Androgen Receptor ligand.

In one embodiment the additional therapeutic agent is a BTK inhibitor.

In one embodiment the additional therapeutic agents are a MEK inhibitor and a RAF inhibitor

In one embodiment the additional therapeutic agent is a RAF inhibitor.

In one embodiment the additional therapeutic agent is regorafenib.

### Cytotoxic Chemotherapeutic Agents

Cytotoxic, DNA-damaging chemotherapeutic agents tend to be non-specific and, particularly at high doses, toxic to normal, rapidly dividing cells such as HSPC and immune effector cells. As used herein the term “DNA-damaging” chemotherapy or chemotherapeutic agent refers to treatment with a cytostatic or cytotoxic agent (i.e., a compound) to reduce or eliminate the growth or proliferation of undesirable cells, for example cancer cells, wherein the cytotoxic effect of the agent can be the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect. Such compounds include, but are not limited to, DNA damaging compounds that can kill cells. “DNA damaging” chemotherapeutic agents include, but are not limited to, alkylating agents, DNA intercalators, protein synthesis inhibitors, inhibitors of DNA or RNA synthesis, DNA base analogs, topoisomerase inhibitors, telomerase inhibitors, and telomeric DNA binding compounds. For example, alkylating agents include alkyl sulfonates, such as busulfan, improsulfan, and piposulfan; aziridines, such as a benzodizepa, carboquone, meturedepa, and uredepa; ethylenimines and methylmelamines, such as altretamine, triethylenemelamine, triethylenephosphoramide, triethylenethiophosphoramide, and trimethylol melamine; nitrogen mustards such as chlorambucil, chlornaphazine, cyclophosphamide, estramustine, mechlorethamine, mechlorethamine oxide hydrochloride, melphalan, novembichine, phenesterine, prednimustine, trofosfamide, and uracil mustard; and nitroso ureas, such as carmustine, chlorozotocin, fotemustine, lomustine, nimustine, and ranimustine. Other DNA-damaging chemotherapeutic agents include daunorubicin, doxorubicin, idarubicin, epirubicin, mitomycin, and streptozocin. Chemotherapeutic antimetabolites include gemcitabine, mercaptopurine, thioguanine, cladribine, fludarabine phosphate, fluorouracil (5-FU), floxuridine, cytarabine, pentostatin, methotrexate, azathioprine, acyclovir, adenine β-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycytidine, cytosine β-1-D-arabinoside, diazooxynorleucine, dideoxynucleosides, 5-fluorodeoxycytidine, 5-fluorodeoxyuridine, and hydroxyurea.

Chemotherapeutic protein synthesis inhibitors include abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine. Additional protein synthesis inhibitors include modeccin, neomycin, norvaline, pactamycin, paromomycine, puromycin, ricin, shiga toxin, showdomycin, sparsomycin, spectinomycin, streptomycin, tetracycline, thiostrepton, and trimethoprim.

Inhibitors of DNA synthesis, include alkylating agents such as dimethyl sulfate, nitrogen and sulfur mustards; intercalating agents, such as acridine dyes, actinomycins, anthracenes, benzopyrene, ethidium bromide, propidium diiodide-intertwining; and other agents, such as distamycin and netropsin. Topoisomerase inhibitors, such as irinotecan, teniposide, coumermycin, nalidixic acid, novobiocin, and oxolinic acid; inhibitors of cell division, including colcemide, mitoxantrone, colchicine, vinblastine, and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin, and streptolydigin also can be used as the DNA damaging compound.

In one embodiment the chemotherapeutic agent is a DNA complex binder such as camptothecin, or etoposide; a thiolate alkylating agent such as nitrosourea, BCNU, CCNU, ACNU, or fotesmustine; a guanine alkylating agent such as temozolomide, a tubulin binder such as vinblastine, vincristine, vinorelbine, vinflunine, cryptophycin 52, halichondrins, such as halichondrin B, dolastatins, such as dolastatin 10 and dolastatin 15, hemiasterlins, such as hemiasterlin A and hemiasterlin B, colchicine, combrestatins, 2-methoxyestradiol, E7010, paclitaxel, docetaxel, epothilone, discodermolide; a DNA polymerase inhibitor such as cytarabine; an anticancer enzyme such as asparaginase; a Rac1 inhibitor such as 6-thioguanine; a thymidylate synthase inhibitor such as capecitabine or 5-FU; a oxazophosphorine compound such as Cytoxan; a integrin inhibitor such as cilengitide; an antifolate such as pralatrexate; a folate antimetabolite such as pemetrexed; or a camptothecin or homocamptothecin such as diflomotecan.

In one embodiment the topoisomerase inhibitor is a type I inhibitor. In another embodiment the topoisomerase inhibitor is a type II inhibitor.

Other DNA-damaging chemotherapeutic agents whose toxic effects can be mitigated by the presently disclosed selective CDK4/6 inhibitors include, but are not limited to, cisplatin, hydrogen peroxide, carboplatin, procarbazine, ifosfamide, bleomycin, plicamycin, taxol, transplatinum, thiotepa, oxaliplatin, and the like, and similar acting-type agents. In one embodiment, the DNA damaging chemotherapeutic agent is selected from the group consisting of cisplatin, carboplatin, camptothecin, and etoposide.

Other suitable chemotherapeutic agents include, but are not limited to, radioactive molecules, toxins, also referred to as cytotoxins or cytotoxic agents, which includes any agent that is detrimental to the viability of cells, agents, and liposomes or other vesicles containing chemotherapeutic compounds. General anticancer pharmaceutical agents include: Vincristine (Oncovin®), liposomal vincristine (Marqibo®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Prednisone, and Dexamethasone (Decadron). Examples of additional suitable chemotherapeutic agents include but are not limited to 5-fluorouracil, dacarbazine, alkylating agents, anthramycin (AMC)), anti-mitotic agents, cis-dichlorodiamine platinum (II) (DDP) cisplatin), diamino dichloro platinum, anthracyclines, antibiotics, antimetabolites, asparaginase, BCG live (intravesical), bleomycin sulfate, calicheamicin, cytochalasin B, dactinomycin (formerly actinomycin), daunorubicin HCl, daunorubicin citrate, denileukin diftitox, dihydroxy anthracin dione, Docetaxel, doxorubicin HCl, E. coli L-asparaginase, Erwinia L-asparaginase, etoposide citrovorum factor, etoposide phosphate, gemcitabine HCl, idarubicin HCl, interferon α-2b, irinotecan HCl, maytansinoid, mechlorethamine HCl, melphalan HCl, mithramycin, mitomycin C, mitotane, polifeprosan 20 with carmustine implant, procarbazine HCl, streptozotocin, teniposide, thiotepa, topotecan HCl, valrubicin, vinblastine sulfate, vincristine sulfate, and vinorelbine tartrate.

Additional cytotoxic chemotherapeutic agents for use with the present invention include: epirubicin, abraxane, taxotere, epothilone, tafluposide, vismodegib, azacytidine, doxifluridine, vindesine, and vinorelbine.

In one embodiment the chemotherapeutic agent is not an aromatase inhibitor. In one embodiment the chemotherapeutic agent is not a steroid. In one embodiment the chemotherapeutic agent is not a BCR-ABL inhibitor.

In one embodiment the chemotherapeutic agent is a DNA complex binder. In one embodiment the chemotherapeutic agent is a tubulin binder. In one embodiment the chemotherapeutic agent is an alkylating agent. In one embodiment the chemotherapeutic agent is a thiolate alkylating agent.

### Additional Chemotherapeutic Agents

Additional chemotherapeutic agents that may be used as described herein may include 2-methoxyestradiol or 2ME2, finasunate, etaracizumab (MEDI-522), HLL1, huN901-DM1, atiprimod, saquinavir mesylate, ritonavir, nelfinavir mesylate, indinavir sulfate, plitidepsin, P276-00, tipifarnib, lenalidomide, thalidomide, pomalidomide, simvastatin, and celecoxib. Chemotherapeutic agents useful in the present invention include, but are not limited to, Trastuzumab (Herceptin®), Pertuzumab (Perjeta™), Lapatinib (Tykerb®), Gefitinib (Iressa®), Erlotinib (Tarceva®), Cetuximab (Erbitux®), Panitumumab (Vectibix®), Vandetanib (Caprelsa®), Vemurafenib (Zelboraf®), Vorinostat (Zolinza®), Romidepsin (Istodax®), Bexarotene (Targretin®), Alitretinoin (Panretin®), Tretinoin (Vesanoid®), Carfilzomib (Kyprolis™), Pralatrexate (Folotyn®), Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™).

Additional chemotherapeutic agents contemplated include, but are not limited to, a calcineurin inhibitor, e.g. a cyclosporin or an ascomycin, e.g. Cyclosporin A (Neoral®), FK506 (tacrolimus), pimecrolimus, a mTOR inhibitor, e.g. rapamycin or a derivative thereof, e.g. Sirolimus (Rapamune®), Everolimus (Certican®), temsirolimus, zotarolimus, biolimus-7, biolimus-9, a rapalog, e.g. ridaforolimus, campath 1H, a SIP receptor modulator, a dual mTORC1 and mTORC2 inhibitor, eg. Vistusertib (AZD2014), e.g. fingolimod or an analogue thereof, an antiIL-8 antibody, mycophenolic acid or a salt thereof, e.g. sodium salt, or a prodrug thereof, e.g. Mycophenolate Mofetil (CellCept®), OKT3 (Orthoclone OKT3@), Prednisone, ATGAM®, Thymoglobulin®, Brequinar Sodium, OKT4, T10B9.A-3A, 33B3.1, 15-deoxyspergualin, tresperimus, Leflunomide Arava®, anti-CD25, anti-IL2R, Basiliximab (Simulect®), Daclizumab (Zenapax®), mizoribine, dexamethasone, ISAtx-247, SDZ ASM 981 (pimecrolimus, Elidel®), Abatacept, belatacept, LFA3lg, etanercept (sold as Enbrel® by ImmuneXcite), adalimumab (Humira®), infliximab (Remicade®), an anti-LFA-1 antibody, natalizumab (Antegren®), Enlimomab, gavilimomab, Golimumab, antithymocyte immunoglobulin, siplizumab, Alefacept, efalizumab, Pentasa, mesalazine, asacol, codeine phosphate, benorylate, fenbufen, naprosyn, diclofenac, etodolac, indomethacin, dasatinib (Sprycel®) nilotinib (Tasigna®), bosutinib (Bosulif®), Imatinib mesylate (Gleevec®) and ponatinib (Iclusig™) amifostine, dolasetron mesylate, dronabinol, epoetin-α, etidronate, filgrastim, fluconazole, goserelin acetate, gramicidin D, granisetron, leucovorin calcium, lidocaine, Mesna, ondansetron HCl, pilocarpine HCl, porfimer sodium, vatalanib, 1-dehydrotestosterone, allopurinol sodium, Betamethasone, sodium phosphate and betamethasone acetate, calcium leucovorin, conjugated estrogens, Dexrazoxane, Dibromomannitol, esterified estrogens, estradiol, estramustine phosphate sodium, ethinyl estradiol, flutamide, folinic acid, glucocorticoids, leuprolide acetate, levamisole HCl, medroxyprogesterone acetate, megestrol acetate, methyltestosterone, nilutamide, octreotide acetate, pamidronate disodium, procaine, propranolol, testolactone, tetracaine, toremifene citrate, and sargramostim.

In one embodiment the chemotherapeutic agent is an estrogen receptor ligands such as tamoxifen, raloxifene, fulvestrant, anordrin, bazedoxifene, broparestriol, chlorotrianisene, clomiphene citrate, cyclofenil, lasofoxifene, ormeloxifene, or toremifene; an androgen receptor ligand such as bicalutamide, enzalutamide, apalutamide, cyproterone acetate, chlormadinone acetate, spironolactone, canrenone, drospirenone, ketoconazole, topilutamide, abiraterone acetate, or cimetidine; an aromatase inhibitor such as letrozole, anastrozole, or exemestane; an anti-inflammatory such as prednisone; an oxidase inhibitor such as allopurinol; an anticancer antibody; an anticancer monoclonal antibody; an antibody against CD40 such as lucatumumab or dacetuzumab; an antibody against CD20 such as rituximab; an antibody that binds CD52 such as alemtuzumab; an antibody that binds integrin such as volociximab or natalizumab; an antibody against interleukin-6 receptor such as tocilizumab; an interleukin-2 memetic such as aldesleukin; an antibody that targets IGF1 like figitumumab; an antibody that targets DR4 such as mapatumumab; an antibody that targets TRAIL-R2 such as lexatumumab or dulanermin; a fusion protein such as atacicept; a B cell inhibitor such as atacicept; a proteasome inhibitor such as carfilzomib, bortezomib, or marizomib; a HSP90 inhibitor such as tanespimycin; a HDAC inhibitor such as vorinostat, belinostat or panobinostat; a MAPK ligand such as talmapimod; a PKC inhibitor such as enzastaurin; a HER2 receptor ligand such as trastuzumab, lapatinib, or pertuzumab; an EGFR inhibitor such as gefitinib, erlotinib, cetuximab, panitumumab, or vandetanib; a natural product such as romidepsin; a retinoid such as bexarotene, tretinoin, or alitretinoin; a receptor tyrosine kinase (RTK) inhibitor such as sunitinib, regorafenib, or pazopanib; or a VEGF inhibitor such as ziv-aflibercept, bevacizumab or dovitinib.

In one embodiment, the combinations of a CDK4/6 inhibitor, chemotherapeutic agent, and immune checkpoint inhibitor is further combined with the use of hematopoietic growth factors including, but not limited to, granulocyte colony stimulating factor (G-CSF, for example, sold as Neupogen® (filgrastim), Neulasta® (peg-filgrastim), or lenograstim), granulocyte-macrophage colony stimulating factor (GM-CSF, for example sold as molgramostim and sargramostim (Leukine®)), M-CSF (macrophage colony stimulating factor), Thrombopoietin (megakaryocyte growth development factor (MGDF), for example sold as Romiplostim® and Eltrombopag®) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (sold as for example epoetin-a as Darbepoetin, Epocept, Nanokine, Epofit, Epogen, Eprex, and Procrit; epoetin-P sold as for example NeoRecormon, Recormon and Micera), epoetin-delta (sold as for example Dynepo), epoetin-omega (sold as for example Epomax), epoetin zeta (sold as for example Silapo and Retacrit) as well as for example Epocept, Epotrust, Erypro Safe, Repoitin, Vintor, Epofit, Erykine, Wepox, Espogen, Relipoietin, Shanpoietin, Zyrop and EPIAO).

Additional chemotherapeutic agents contemplated herein, particularly in the treatment of abnormal tissue of the female reproductive system such as breast, ovarian, endometrial, or uterine cancer include an estrogen inhibitor including but not limited to a SERM (selective estrogen receptor modulator), a SERD (selective estrogen receptor degrader), a complete estrogen receptor degrader, or another form of partial or complete estrogen antagonist. Partial anti-estrogens like raloxifene and tamoxifen retain some estrogen-like effects, including an estrogen-like stimulation of uterine growth, and also, in some cases, an estrogen-like action during breast cancer progression which actually stimulates tumor growth. In contrast, fulvestrant, a complete anti-estrogen, is free of estrogen-like action on the uterus and is effective in tamoxifen-resistant tumors. Non-limiting examples of anti-estrogen compounds are provided in WO 2014/19176 assigned to Astra Zeneca, WO2013/090921, WO 2014/203129, WO 2014/203132, and US2013/0178445 assigned to Olema Pharmaceuticals, and U.S. Pat. Nos. 9,078,871, 8,853,423, and 8,703,810, as well as US 2015/0005286, WO 2014/205136, and WO 2014/205138. Additional non-limiting examples of anti-estrogen compounds include: SERMS such as anordrin, bazedoxifene, broparestriol, clomiphene citrate, cyclofenil, lasofoxifene, ormeloxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, delmadinone acetate, dydrogesterone, medroxyprogesterone acetate, megestrol acetate, nomegestrol acetate, norethisterone acetate, progesterone, and spironolactone.

Additional chemotherapeutic agents contemplated herein, particularly in the treatment of abnormal tissue of the male reproductive system such as prostate or testicular cancer, include, but are not limited to, an androgen (such as testosterone) inhibitor including but not limited to a selective androgen receptor modulator, a selective androgen receptor degrader, a complete androgen receptor degrader, or another form of partial or complete androgen antagonist. In one embodiment, the prostate or testicular cancer is androgen-resistant. Non-limiting examples of anti-androgen compounds are provided in WO 2011/156518 and U.S. Pat. Nos. 8,455,534 and 8,299,112. Additional non-limiting examples of anti-androgen compounds include: chlormadinone acetate, spironolactone, canrenone, drospirenone, ketoconazole, topilutamide, abiraterone acetate, and cimetidine.

The chemotherapeutic agent may include a kinase inhibitor, including but not limited to a phosphoinositide 3-kinase (PI3K) inhibitor, a Bruton's tyrosine kinase (BTK) inhibitor, or a spleen tyrosine kinase (Syk) inhibitor, or a combination thereof.

PI3k inhibitors are well known. Examples of PI3 kinase inhibitors include but are not limited to Wortmannin, demethoxyviridin, perifosine, idelalisib, pictilisib, Palomid 529, ZSTK474, PWT33597, CUDC-907, and AEZS-136, duvelisib, GS-9820, GDC-0032 (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), LY3023414, BEZ235(2-Methyl-2-{4-[3-methyl-2-oxo-8-(quinolin-3-yl)-2,3-dihydro-1H-imidazo[4,5-c]quinolin-1-yl]phenyl}propanenitrile), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl] acetate (also known as sonolisib)), and the structure described in WO2014/071109 having the formula:

BTK inhibitors are well known. Examples of BTK inhibitors include ibrutinib (also known as PCI-32765)(Imbruvica™) (1-[(3R)-3-[4-amino-3-(4-phenoxy-phenyl)pyrazolo[3,4-III d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), BGB-3111, and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors are well known, and include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), RO9021 (6-[(1R,2S)-2-Amino-cyclohexylamino]-4-(5,6-dimethyl-pyridin-2-ylamino)-pyridazine-3-carboxylic acid amide), imatinib (Gleevec; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), YM193306(see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

The chemotherapeutic agent can also be a B-cell lymphoma 2 (Bcl-2) protein inhibitor. BCL-2 inhibitors are known in the art, and include, for example, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl] amino]-3-nitrophenyl]sulfonylbenzamide), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), or G3139 (Oblimersen).

Additional chemotherapeutic agents for use in the methods contemplated herein include, but are not limited to, midazolam, MEK inhibitors, RAS inhibitors, ERK inhibitors, ALK inhibitors, HSP inhibitors (for example, HSP70 and HSP 90 inhibitors, or a combination thereof), RAF inhibitors, apoptotic compounds, topoisomerase inhibitors, AKT inhibitors, including but not limited to, MK-2206, GSK690693, Perifosine, (KRX-0401), GDC-0068, Triciribine, AZD5363, Honokiol, PF-04691502, and Miltefosine, or FLT-3 inhibitors, including but not limited to, P406, Dovitinib, Quizartinib (AC220), Amuvatinib (MP-470), Tandutinib (MLN518), ENMD-2076, and KW-2449, or combinations thereof. Examples of MEK inhibitors include but are not limited to trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEAl19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6 carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-,2-oxazinan-2 yl)methyl)benzamide), or AZD8330 (2-((2-fluoro-4-iodophenyl)amino)-N-(2-hydroxyethoxy)-1,5-dimethyl-6-oxo-1,6-dihydropyridine-3-carboxamide). Examples of RAS inhibitors include but are not limited to Reolysin and siG12D LODER. Examples of ALK inhibitors include but are not limited to Crizotinib, AP26113, and LDK378. HSP inhibitors include but are not limited to Geldanamycin or 17-N-Allylamino-17-demethoxygeldanamycin (17AAG), and Radicicol.

Known ERK inhibitors include SCH772984 (Merck/Schering-Plough), VTX-11e (Vertex), DEL-22379, Ulixertinib (BVD-523, VRT752271), GDC-0994, FR 180204, XMD8-92, and ERK5-IN-1.

Raf inhibitors are well known, and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), and Sorafenib N-Oxide (4-[4-[[[[4-Chloro-3(trifluoroMethyl)phenyl]aMino]carbonyl]aMino]phenoxy]-N-Methyl-2pyridinecarboxaMide 1-Oxide).

Known topoisomerase I inhibitors useful in the present invention include (S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride (topotecan), (S)-4-ethyl-4-hydroxy-1H-pyrano[3′,4′:6,7]indolizino[1,2-b]quinoline-3,14-(4H,12H)-dione (camptothecin), (1S,9S)-1-Amino-9-ethyl-5-fluoro-1,2,3,9,12,15-hexahydro-9-hydroxy-4-methyl-10H,13H-benzo(de)pyrano(3′,4′:6,7)indolizino(1,2-b)quinoline-10,13-dione (exatecan), (7-(4-methylpiperazinomethylene)-10,11-ethylenedioxy-20(S)-camptothecin (lurtotecan), or (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate (irinotecan), (R)-5-ethyl-9,10-difluoro-5-hydroxy-4,5-dihydrooxepino[3′,4′:6,7]indolizino[1,2-b]quinoline-3,15(1H,13H)-dione (diflomotecan), (4S)-11-((E)-((1,1-Dimethylethoxy)imino)methyl)-4-ethyl-4-hydroxy-1,12-dihydro-14H-pyrano(3′,4′:6,7)indolizino(1,2-b)quinoline-3,14(4H)-dione (gimatecan), (S)-8-ethyl-8-hydroxy-15-((4-methylpiperazin-1-yl)methyl)-11,14-dihydro-2H-[1,4]dioxino[2,3-g]pyrano[3′,4′:6,7]indolizino[1,2-b]quinoline-9,12(3H,8H)-dione(lurtotecan), (4S)-4-Ethyl-4-hydroxy-11-[2-[(1-methylethyl)amino]ethyl]-1H-pyrano[3?,4?:6,7]indolizino[1,2-b]quinoline-3,14(4H,12H)-dione (belotecan), 6-((1,3-dihydroxypropan-2-yl)amino)-2,10-dihydroxy-12-((2R,3R,4S,5S,6R)-3,4,5-trihydroxy-6-(hydroxymethyl)tetrahydro-2H-pyran-2-yl)-12,13-dihydro-5H-indolo[2,3-a]pyrrolo[3,4-c]carbazole-5,7(6H)-dione (edotecarin), 8,9-dimethoxy-5-(2-N,N-dimethylaminoethyl)-2,3-methylenedioxy-5H-dibenzo(c,h)(1,6)naphthyridin-6-one (topovale), benzo[6,7]indolizino[1,2-b]quinolin-11(13H)-one (rosettacin), (S)-4-ethyl-4-hydroxy-11-(2-(trimethylsilyl)ethyl)-1H-pyrano[3′,4′:6,7]indolizino[1,2-b]quinoline-3,14(4H,12H)-dione (cositecan), tetrakis{(4S)-9-[([1,4′-bipiperidinyl]-1′-carbonyl)oxy]-4,11-diethyl-3,14-dioxo-3,4,12,14-tetrahydro-1H-pyrano[3′,4′:6,7]indolizino[1,2-b]quinolin-4-yl} N,N′,N″,N′″-{methanetetrayltetrakis[methylenepoly(oxyethylene)oxy(1-oxoethylene)]}tetraglycinate tetrahydrochloride (etirinotecan pegol), 10-hydroxy-camptothecin (HOCPT), 9-nitrocamptothecin (rubitecan), SN38 (7-ethyl-10-hydroxycamptothecin), and 10-hydroxy-9-nitrocamptothecin (CPT109), (R)-9-chloro-5-ethyl-5-hydroxy-10-methyl-12-((4-methylpiperidin-1-yl)methyl)-4,5-dihydrooxepino[3′,4′:6,7]indolizino[1,2-b]quinoline-3,15(1H,13H)-dione (elmotecan).

In one embodiment, the chemotherapeutic agent is not an aromatase inhibitor. In one embodiment, the chemotherapeutic agent is not an estrogen or androgen receptor agonist or antagonist.

### Growth Factors

In one embodiment, the combination of one of the active compounds of the present invention, a chemotherapeutic agent, and a checkpoint inhibitor is further combined with the use of hematopoietic growth factors including, but not limited to, granulocyte colony stimulating factor (G-CSF, for example, sold as Neupogen (filgrastim), Neulasta (peg-filgrastim), or lenograstim), granulocyte-macrophage colony stimulating factor (GM-CSF, for example sold as molgramostim and sargramostim (Leukine)), M-CSF (macrophage colony stimulating factor), Thrombopoietin (megakaryocyte growth development factor (MGDF), for example sold as Romiplostim and Eltrombopag) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (sold as for example epoetin-a as Darbepoetin, Epocept, Nanokine, Epofit, Epogen, Eprex, and Procrit; epoetin-0 sold as for example NeoRecormon, Recormon and Micera), epoetin-delta (sold as for example Dynepo), epoetin-omega (sold as for example Epomax), epoetin zeta (sold as for example Silapo and Retacrit) as well as for example Epocept, Epotrust, Erypro Safe, Repoitin, Vintor, Epofit, Erykine, Wepox, Espogen, Relipoietin, Shanpoietin, Zyrop and EPIAO).

### VIII. Examples

### General Methods:

Compounds of the present invention with stereocenters are drawn racemic for convenience. One skilled in the art will recognize that pure enantiomers or enriched or diasteromers can be prepared by methods known in the art. Examples of methods to obtain optically active materials include at least the following.

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme; iv) enzymatic asymmetric synthesis—a synthetic technique whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries; vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including simulated moving bed chromatography, is used in one embodiment. A wide variety of chiral stationary phases are commercially available.

¹H NMR spectra were recorded on a 300 MHz Fourier transform Brücker spectrometer. Spectra were obtained from samples prepared in 5 mm diameter tubes in CDCl₃, CD₃OD or DMSO-d₆. The spin multiplicities are indicated by the symbols s (singlet), d (doublet), t (triplet), m (multiplet) and, br (broad). Coupling constants (J) are reported in Hz. MS spectra were obtained using electrospray ionization (ESI) on an Agilent Technologies 6120 quadrupole MS apparatus. The reactions were generally carried out under a dry nitrogen atmosphere using Sigma-Aldrich anhydrous solvents. All common chemicals were purchased from commercial sources.

### Example 1. Preparation of Substituted 2-aminopyridines

### 1-Methyl-4-(6-nitro-3-pyridyl)piperazine

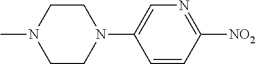 [SMILES: CN1CCN(c2ccc([N+](=O)[O-])nc2)CC1]

To 5-bromo-2-nitropyridine (4.93 g, 24.3 mmole) in DMF (20 mL) was added N-methylpiperazine (2.96 g, 1.1 eq) followed by the addition of DIPEA (4.65 mL, 26.7 mmole). The contents were heated at 90° C. for 24 hrs. After the addition of ethyl acetate (200 mL), water (100 mL) was added and the layers were separated. Drying followed by concentration afforded the crude product which was purified on a silica gel column using (0-10%) DCM/Methanol.

¹H NMR (DMSO-d₆) δ 8.26 (s, 1H), 8.15 (1H, d, J=9.3 Hz), 7.49 (1H, d, J=9.4 Hz), 3.50 (m, 4H), 2.49 (m, 4H), 2.22 (s, 3H).

### 5-(4-Methylpiperazin-1-yl)pyridin-2-amine

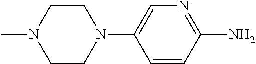

To 1-methyl-4-(6-nitro-3-pyridyl)piperazine 3.4 g in ethyl acetate (100 mL) and ethanol (100 mL) was added 10% Pd/c (400 mg) and then contents stirred under hydrogen (10 psi) overnight. After filtration through Celite®, the solvents were evaporated and the crude product was purified over silica gel using DCM/7N Ammonia in MeOH (0-5%) to afford 5-(4-methylpiperazin-1-yl)pyridin-2-amine (2.2 g).

¹HNMR (DMSO-d₆) δ 7.56 (1H, d, J=3 Hz), 7.13 (1H, m), 6.36 (1H, d, J=8.8 Hz), 5.33 (brs, 2H), 2.88 (m, 4H), 2.47 (m, 4H), 2.16 (s, 3H).

### tert-Butyl 4-(6-amino-3-pyridyl)piperazine-1-carboxylate

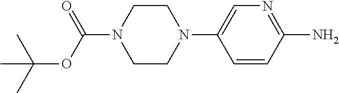 [SMILES: CC(C)(C)OC(=O)N1CCN(c2ccc(N)nc2)CC1]

The compound was prepared as described in WO 2010/020675 A1.

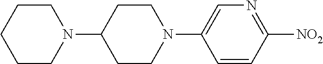 [SMILES: O=[N+]([O-])c1ccc(N2CCC(N3CCCCC3)CC2)cn1]

To 5-bromo-2-nitropyridine (1.2 g, 5.9 mmole) in DMSO (4 mL) was added 1-(4-piperidyl)piperidine (1.0 g, 5.9 mmole) and triethylamine (0.99 mL, 7.1 mmole). The contents were heated to 120° C. in a CEM Discovery microwave system for 3 hours. The crude reaction was then loaded over a silica gel column and eluted with DCM/methanol (0-20%) to afford 2-nitro-5-[4-(1-piperidyl)-1-piperidyl]pyridine as an oil (457 mg).

¹H NMR (600 MHz, DMSO-d₆) δ ppm 1.26-1.36 (m, 2H) 1.43 (m, 6H) 1.76 (m, 2H) 2.37 (m, 5H) 2.94 (t, J=12.74 Hz, 2H) 4.06 (d, J=13.47 Hz, 2H) 7.41 (dd, J=9.37, 2.64 Hz, 1H) 8.08 (d, J=9.37 Hz, 1H) 8.20 (d, J=2.64 Hz, 1H).

### 5-[4-(1-Piperidyl)-1-piperidyl]pyridin-2-amine

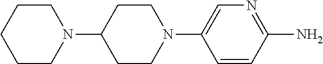 [SMILES: Nc1ccc(N2CCC(N3CCCCC3)CC2)cn1]

5-[4-(1-Piperidyl)-1-piperidyl]pyridin-2-amine was prepared in a manner similar to that used in the synthesis of 5-(4-methylpiperazin-1-yl)pyridin-2-amine.

¹H NMR (600 MHz, DMSO-d₆) δ ppm 1.13-1.37 (m, 6H) 1.40-1.63 (m, 6H) 1.71 (m, 2H), 2.24 (m, 1H) 2.43 (m, 2H) 3.33 (d, J=12.30 Hz, 2H) 5.31 (s, 2H) 6.33 (d, J=8.78 Hz, 1H) 7.10 (dd, J=8.78, 2.93 Hz, 1H) 7.55 (d, J=2.64 Hz, 1H). LCMS (ESI) 261 (M+H).

### 4-[1-(6-Nitro-3-pyridyl)-4-piperidyl] morpholine

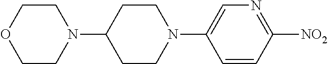 [SMILES: O=[N+]([O-])c1ccc(N2CCC(N3CCOCC3)CC2)cn1]

4-[1-(6-Nitro-3-pyridyl)-4-piperidyl]morpholine was synthesized in a manner similar to that used in the synthesis of 2-nitro-5-[4-(1-piperidyl)-1-piperidyl]pyridine.

1H NMR (600 MHz, DMSO-d₆) δ ppm 1.41 (m, 2H) 1.82 (m, 2H) 2.42 (m, 5H) 2.98 (t, J=12.44 Hz, 2H) 3.52 (s, 4H) 4.04 (d, J=12.88 Hz, 2H) 7.42 (d, J=9.37 Hz, 1H) 8.08 (d, J=9.08 Hz, 1H) 8.21 (s, 1H).

### 5-(4-Morpholino-1-piperidyl) pyridin-2-amine

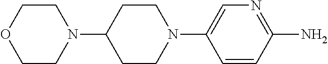 [SMILES: Nc1ccc(N2CCC(N3CCOCC3)CC2)cn1]

5-(4-Morpholino-1-piperidyl)pyridin-2-amine was prepared in a manner similar to that used in the synthesis of 5-(4-methylpiperazin-1-yl)pyridin-2-amine.

¹H NMR (600 MHz, DMSO-d₆) δ ppm 1.34-1.52 (m, 2H) 1.78 (m, 2H) 2.14 (m, 1H) 2.43 (m, 4H) 3.32 (d, J=12.30 Hz, 4H) 3.47-3.59 (m, 4H) 5.32 (s, 2H) 6.34 (d, J=8.78 Hz, 1H) 7.11 (dd, J=8.93, 2.78 Hz, 1H) 7.47-7.62 (m, 1H). LCMS (ESI) 263 (M+H).

### 4-[1-(6-Nitro-3-pyridyl)-4-piperidyl] thiomorpholine

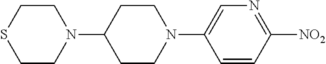 [SMILES: O=[N+]([O-])c1ccc(N2CCC(N3CCSCC3)CC2)cn1]

4-[1-(6-Nitro-3-pyridyl)-4-piperidyl] thiomorpholine was synthesized in a manner similar to that used in the synthesis of 2-nitro-5-[4-(1-piperidyl)-1-piperidyl]pyridine.

¹H NMR (600 MHz, DMSO-d₆) δ ppm 1.40-1.52 (m, 2H) 1.71 (m, 2H) 2.49-2.55 (m, 4H) 2.56-2.63 (m, 1H) 2.68-2.75 (m, 4H) 2.88-2.98 (m, 2H) 4.09 (d, J=13.18 Hz, 2H) 7.42 (dd, J=9.22, 3.07 Hz, 1H) 8.08 (d, J=9.37 Hz, 1H) 8.20 (d, J=3.22 Hz, 1H).

### 5-(4-Thiomorpholino-1-piperidyl)pyridin-2-amine

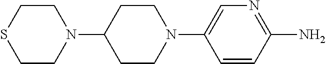 [SMILES: Nc1ccc(N2CCC(N3CCSCC3)CC2)cn1]

5-(4-Thiomorpholino-1-piperidyl) pyridin-2-amine was prepared in a manner similar to that used in the synthesis of 5-(4-methylpiperazin-1-yl)pyridin-2-amine.

¹H NMR (600 MHz, DMSO-d₆) δ ppm 1.47-1.59 (m, 2H) 1.65 (m, 2H) 2.22-2.38 (m, 1H) 2.50-2.59 (m, 6H) 2.68-2.82 (m, 4H) 3.33 (d, J=12.00 Hz, 2H) 5.31 (s, 2H) 6.33 (d, J=9.08 Hz, 1H) 7.10 (dd, J=8.78, 2.93 Hz, 1H) 7.55 (d, J=2.64 Hz, 1H). LCMS (ESI) 279 (M+H).

### 2-Nitro-5-(1-piperidyl)pyridine

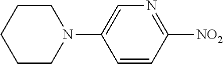 [SMILES: O=[N+]([O-])c1ccc(N2CCCCC2)cn1]

2-Nitro-5-(1-piperidyl) pyridine was synthesized in a manner similar to that used in the synthesis of 2-nitro-5-[4-(1-piperidyl)-1-piperidyl]pyridine.

¹H NMR (600 MHz, DMSO-d₆) δ ppm 1.56 (m, 6H) 3.49 (d, J=4.39 Hz, 4H) 7.30-7.47 (m, 1H) 8.02-8.12 (m, 1H) 8.15-8.26 (m, 1H).

### 5-(1-Piperidyl)pyridin-2-amine

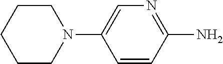 [SMILES: Nc1ccc(N2CCCCC2)cn1]

5-(1-Piperidyl) pyridin-2-amine was prepared in a manner similar to that used in the synthesis of 5-(4-methylpiperazin-1-yl)pyridin-2-amine.

¹H NMR (600 MHz, DMSO-d₆) δ ppm 1.39-1.46 (m, 2H) 1.51-1.62 (m, 4H) 2.75-2.92 (m, 4H) 5.30 (s, 2H) 6.34 (d, J=8.78 Hz, 1H) 7.09 (dd, J=8.78, 2.93 Hz, 1H) 7.54 (d, J=2.93 Hz, 1H). LCMS (ESI) 178 (M+H).

### 4-(6-Nitro-3-pyridyl) thiomorpholine

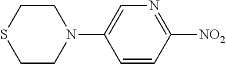 [SMILES: O=[N+]([O-])c1ccc(N2CCSCC2)cn1]

4-(6-nitro-3-pyridyl) thiomorpholine was synthesized in a manner similar to that used in the synthesis of 2-nitro-5-[4-(1-piperidyl)-1-piperidyl]pyridine.

¹H NMR (600 MHz, DMSO-d₆) δ ppm 2.56-2.69 (m, 4H) 3.79-3.92 (m, 4H) 7.43 (dd, J=9.22, 3.07 Hz, 1H) 8.10 (d, J=9.37 Hz, 1H) 8.20 (d, J=2.93 Hz, 1H).

### 5-Thiomorpholinopyridin-2-amine

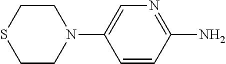 [SMILES: Nc1ccc(N2CCSCC2)cn1]

5-Thiomorpholinopyridin-2-amine was prepared in a manner similar to that used in the synthesis of 5-(4-methylpiperazin-1-yl) pyridin-2-amine.

¹H NMR (600 MHz, DMSO-d₆) δ ppm 2.59-2.73 (m, 4H) 3.04-3.20 (m, 4H) 5.41 (s, 2H) 6.35 (d, J=8.78 Hz, 1H) 7.10 (dd, J=8.78, 2.93 Hz, 1H) 7.57 (d, J=2.64 Hz, 1H). LCMS (ESI) 196 (M+H).

### tert-Butyl (4R)-5-(6-nitro-3-pyridyl)-2,5-diazabicyclo[2.2.1]heptane-2-carboxylate

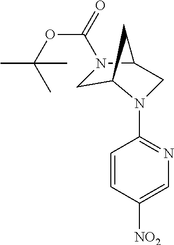 [SMILES: CC(C)(C)OC(=O)N1C[C@H]2CC1CN2c1ccc([N+](=O)[O-])cn1]

tert-Butyl (4R)-5-(6-nitro-3-pyridyl)-2,5-diazabicyclo[2.2.1]heptane-2-carboxylate was synthesized in a manner similar to that used in the synthesis of 2-nitro-5-[4-(1-piperidyl)-1-piperidyl]pyridine.

¹H NMR (600 MHz, DMSO-d₆) δ ppm 1.33 (d, J=32.21 Hz, 11H) 1.91 (m, 2H) 3.15 (d, J=10.25 Hz, 1H) 3.58 (m, 1H) 4.46 (m, 1H) 4.83 (s, 1H) 7.16 (s, 1H) 7.94 (s, 1H) 8.05-8.16 (m, 1H).

### tert-Butyl(4R)-5-(6-amino-3-pyridyl)-2,5-diazabicyclo[2.2.1]heptane-2-carboxylate

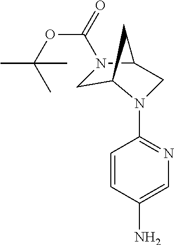 [SMILES: CC(C)(C)OC(=O)N1C[C@H]2CC1CN2c1ccc(N)cn1]

tert-Butyl (4R)-5-(6-amino-3-pyridyl)-2,5-diazabicyclo[2.2.1]heptane-2-carboxylate was prepared in a manner similar to that used in the synthesis of 5-(4-methylpiperazin-1-yl)pyridin-2-amine.

¹H NMR (600 MHz, DMSO-d₆) δ ppm 1.31 (d, J=31.91 Hz, 11H) 1.83 (m, 2H) 2.71-2.82 (m, 1H) 3.44 (m, 1H) 4.30 (d, 2H) 5.08 (s, 2H) 6.35 (d, J=8.78 Hz, 1H) 6.77-6.91 (m, 1H) 7.33 (s, 1H). LCMS (ESI) 291 (M+H).

### N,N-dimethyl-1-(6-nitro-3-pyridyl) piperidin-4-amine

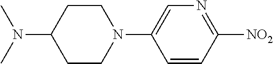 [SMILES: CN(C)C1CCN(c2ccc([N+](=O)[O-])nc2)CC1]

N,N-dimethyl-1-(6-nitro-3-pyridyl)piperidin-4-amine was synthesized in a manner similar to that used in the synthesis of 2-nitro-5-[4-(1-piperidyl)-1-piperidyl]pyridine.

¹H NMR (600 MHz, DMSO-d₆) δ ppm 1.30-1.45 (m, 2H) 1.79 (m, 2H) 2.14 (s, 6H) 2.33 (m, 1H) 2.92-3.04 (m, 2H) 4.03 (d, J=13.76 Hz, 2H) 7.42 (dd, J=9.22, 3.07 Hz, 1H) 8.04-8.11 (m, 1H) 8.21 (d, J=2.93 Hz, 1H).

### 5-[4-(Dimethylamino)-1-piperidyl]pyridin-2-amine

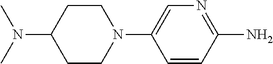 [SMILES: CN(C)C1CCN(c2ccc(N)nc2)CC1]

5-[4-(dimethylamino)-1-piperidyl]pyridin-2-amine was prepared in a manner similar to that used in the synthesis of 5-(4-methylpiperazin-1-yl)pyridin-2-amine.

¹H NMR (600 MHz, DMSO-d₆) δ ppm 1.35-1.50 (m, 2H) 1.69-1.81 (m, 2H) 2.00-2.10 (m, 1H) 2.11-2.22 (s, 6H) 3.17-3.36 (m, 4H) 5.19-5.38 (s, 2H) 6.34 (d, J=8.78 Hz, 1H) 7.10 (dd, J=8.78, 2.93 Hz, 1H) 7.55 (d, J=2.63 Hz, 1H). LCMS (ESI) 221 (M+H).

### 4-(6-Nitro-3-pyridyl) morpholine

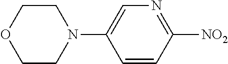 [SMILES: O=[N+]([O-])c1ccc(N2CCOCC2)cn1]

4-(6-Nitro-3-pyridyl) morpholine was synthesized in a manner similar to that used in the synthesis of 2-nitro-5-[4-(1-piperidyl)-1-piperidyl] pyridine.

### 5-Morpholinopyridin-2-amine

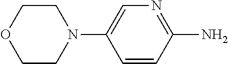 [SMILES: Nc1ccc(N2CCOCC2)cn1]

5-Morpholinopyridin-2-amine was prepared in a manner similar to that used in the synthesis of 5-(4-methylpiperazin-1-yl) pyridin-2-amine.

¹H NMR (600 MHz, CHCl₃-d) δ ppm 2.91-3.00 (m, 4H) 3.76-3.84 (m, 4H) 4.19 (br. s., 2H) 6.45 (d, J=8.78 Hz, 1H) 7.12 (dd, J=8.78, 2.93 Hz, 1H) 7.72 (d, J=2.93 Hz, 1H).

### 5-(4-Isobutylpiperazin-1-yl) pyridin-2-amine

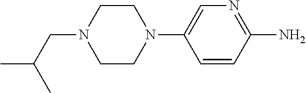 [SMILES: CC(C)CN1CCN(c2ccc(N)nc2)CC1]

1-Isobutyl-4-(6-nitro-3-pyridyl)piperazine was synthesized in a manner similar to that used in the synthesis of 2-nitro-5-[4-(1-piperidyl)-1-piperidyl]pyridine which was then converted 5-(4-isobutylpiperazin-1-yl)pyridin-2-amine in a manner similar to that used in the synthesis of 5-(4-methylpiperazin-1-yl)pyridin-2-amine.

¹H NMR (600 MHz, CHCl₃-d) δ ppm 0.88 (d, J=6.73 Hz, 6H) 1.71-1.84 (m, 1H) 2.10 (d, J=7.32 Hz, 2H) 2.46-2.58 (m, 4H) 2.97-3.07 (m, 4H) 4.12 (s, 2H) 6.45 (d, J=8.78 Hz, 1H) 7.14 (dd, J=8.78, 2.93 Hz, 1H) 7.75 (d, J=2.93 Hz, 1H). LCMS (ESI) 235 (M+H).

### 5-(4-Isopropylpiperazin-1-yl) pyridin-2-amine

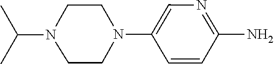 [SMILES: CC(C)N1CCN(c2ccc(N)nc2)CC1]

1-Isopropyl-4-(6-nitro-3-pyridyl)piperazine was synthesized in a manner similar to that used in the synthesis of 2-nitro-5-[4-(1-piperidyl)-1-piperidyl]pyridine which was then converted to 5-(4-isopropylpiperazin-1-yl)pyridin-2-amine in a manner similar to that used in the synthesis of 5-(4-methylpiperazin-1-yl)pyridin-2-amine.

¹H NMR (600 MHz, CHCl₃-d) δ ppm 1.06 (d, J=6.44 Hz, 6H) 2.59-2.75 (m, 5H) 2.97-3.10 (m, 4H) 4.13 (s, 2H) 6.45 (d, J=8.78 Hz, 1H) 7.15 (dd, J=9.08, 2.93 Hz, 1H) 7.76 (d, J=2.93 Hz, 1H). LCMS (ESI) 221 (M+H).

### 5-[(2R,6S)-2,6-Dimethylmorpholin-4-yl]pyridin-2-amine

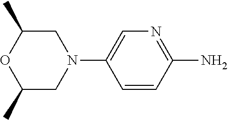

(2S,6R)-2,6-Dimethyl-4-(6-nitro-3-pyridyl)morpholine was synthesized in a manner similar to that used in the synthesis of 2-nitro-5-[4-(1-piperidyl)-1-piperidyl]pyridine which was then converted to 5-[(2R,6S)-2,6-dimethylmorpholin-4-yl]pyridin-2-amine in a manner similar to that used in the synthesis of 5-(4-methylpiperazin-1-yl)pyridin-2-amine. ¹H NMR (600 MHz, CHCl₃-d) δ ppm 1.20 (d, J=6.44 Hz, 6H) 2.27-2.39 (m, 2H) 3.11-3.21 (m, 2H) 3.70-3.84 (m, 2H) 4.15 (s, 2H) 6.45 (d, J=8.78 Hz, 1H) 7.12 (dd, J=8.78, 2.93 Hz, 1H) 7.72 (d, J=2.63 Hz, 1H). LCMS (ESI) 208 (M+H).

### 5-[(3R,5S)-3,5-Dimethylpiperazin-1-yl]pyridin-2-amine

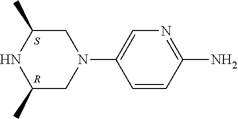

(3S,5R)-3,5-Dimethyl-1-(6-nitro-3-pyridyl)piperazine was synthesized in a manner similar to that used in the synthesis of 2-nitro-5-[4-(1-piperidyl)-1-piperidyl]pyridine which was then converted to 5-[(3R,5S)-3,5-dimethylpiperazin-1-yl]pyridin-2-amine in a manner similar to that used in the synthesis of 5-(4-methylpiperazin-1-yl)pyridin-2-amine. ¹H NMR (600 MHz, CHCl₃-d) δ ppm 1.09 (d, J=6.44 Hz, 6H) 2.20 (t, J=10.83 Hz, 2H) 2.95-3.08 (m, 2H) 3.23 (dd, J=11.71, 2.05 Hz, 2H) 4.13 (s, 2H) 6.45 (d, J=8.78 Hz, 1H) 7.14 (dd, J=8.78, 2.93 Hz, 1H) 7.73 (d, J=2.63 Hz, 1H). LCMS (ESI) 207 (M+H).

### Example 2: Preparation of Final Compounds

### Synthesis of 2′-(((3S,4R)-4-methylpiperidin-3-yl)amino)-7′,8′-dihydro-6′H-spiro[cyclohexane-1,9′-pyrazino[1′,2′:1,5]pyrrolo[2,3-d]pyrimidin]-6′-one (COMPOUND 1) and 2′-(((3S,4S)-4-methylpiperidin-3-yl)amino)-7′,8′-dihydro-6′H-spiro[cyclohexane-1,9′-pyrazino[1′,2′:1,5]pyrrolo[2,3-d]pyrimidin]-6′-one (COMPOUND 2)

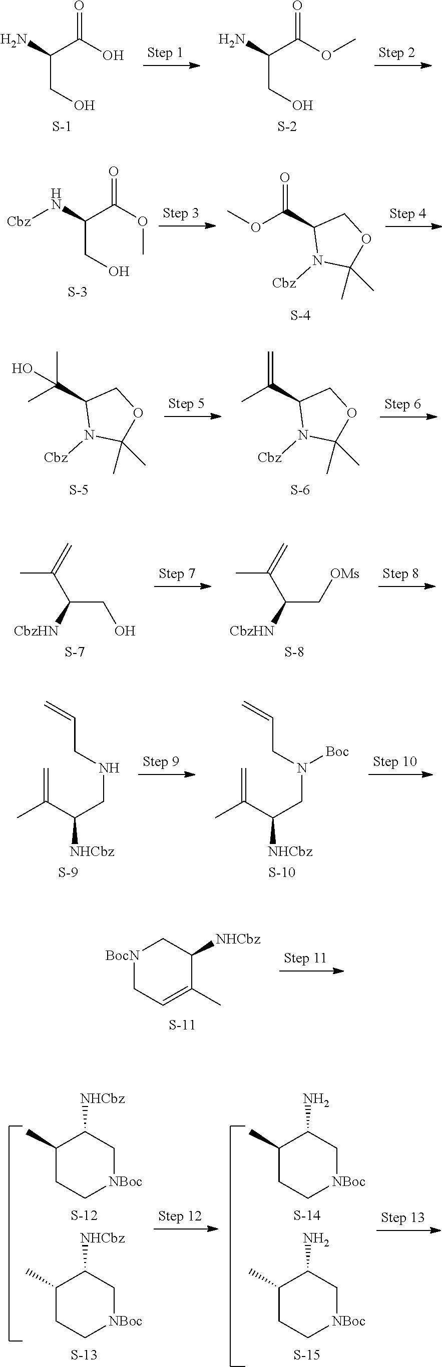

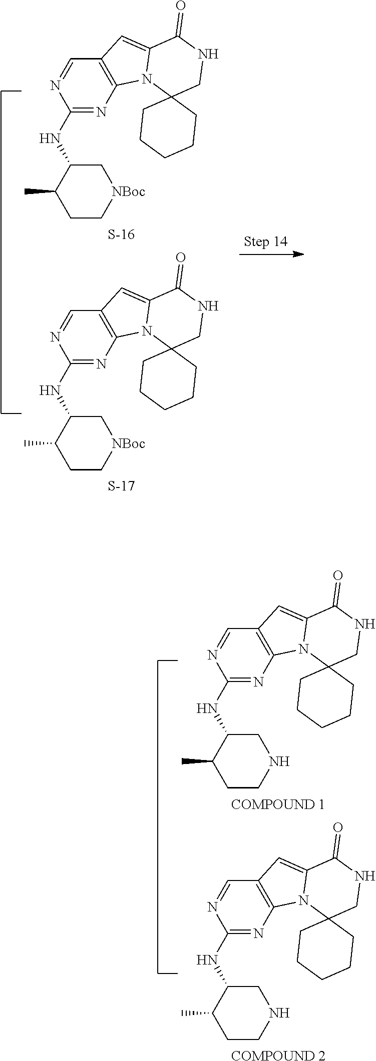

In Step 1, 20 g of S-1 is reacted with thionyl chloride in methanol to provide 22 g of S-2. In Step 2, 20 g of S-2 was reacted with benzyl chloroformate and sodium bicarbonate in a mixture of THE and water to provide 27 g of S-3. In Step 3, 27 g of S-3 was reacted with 2,2-dimethoxypropane and boron trifluoride diethyletherate in acetone to provide 30 g of S-4. In Step 4, 3.0 g of S-4 was reacted with methylmagnesium bromide in THE at 0° C. to provide 2.0 g of S-5. In Step 5, 6.0 g of S-5 was reacted with methanesulfonyl chloride and triethylamine in dichloromethane to provide 3.0 g of S-6. In Step 6, 0.4 g of S-6 was reacted with 4-toluenesulfonic acid in methanol to provide 300 mg of S-7. In Step 7, 2.2 g of S-7 was reacted with methanesulfonyl chloride and triethylamine in dichloromethane to provide 2.4 g of S-8. In Step 8, 2.4 g of S-8 was reacted with allylamine at 80° C. to provide 1.8 g of S-9. In Step 9, 1.8 g of S-9 was reacted with triethylamine in dichloromethane to provide 1.8 g of S-10. In Step 10, 1.8 g of S-10 was reacted with Grubbs-II catalyst in dichloromethane at 40° C. to provide 1.2 g of S-11. In Step 11, 1.2 g of S-11 was reacted with PtO₂ in methanol to provide 400 mg of S-12 and 180 mg of S-13.

In one run of Step 12 to Step 14, S-12 was used. In Step 12, 400 mg of S-12 was reacted with Pd/C under hydrogen gas in methanol to provide 260 mg of S-14. In Step 13, 50 mg of S-14 reacted with triethylamine in ethanol in a microwave reactor at 120° C. to provide 6 mg of S-16 after purification by preparatory thin layer chromatography. In Step 14, 6 mg of S-16 was reacted with trifluoroacetic acid in dichloromethane. Upon combination and purification with a prior batch, 3.3 mg of COMPOUND 1 was obtained as confirmed by HPLC, LC-MS, and ¹H-NMR.

In another run of Step 12 to Step 14, S-13 was used. In Step 12, 180 mg of S-13 was reacted with Pd/C in methanol under hydrogen gas to provide 100 mg of S-15. In Step 13, 100 mg of S-15 was reacted with N,N-diisopropylethylamine in ethanol in a microwave reactor at 120° C. to provide 10 mg of S-17. In Step 14, 10 mg of S-17 was reacted with trifluoroacetic acid in dichloromethane at room temperature for two hours. Upon purification, 2.4 mg of COMPOUND 2 was obtained as confirmed by HPLC, LC-MS, and ¹H-NMR.

### Synthesis of (S)—N-(6′-oxo-7′,8′-dihydro-6′H-spiro[cyclohexane-1,9′-pyrazino[1′,2′:1,5]pyrrolo[2,3-d]pyrimidin]-2′-yl)piperidine-3-carboxamide (COMPOUND 3)

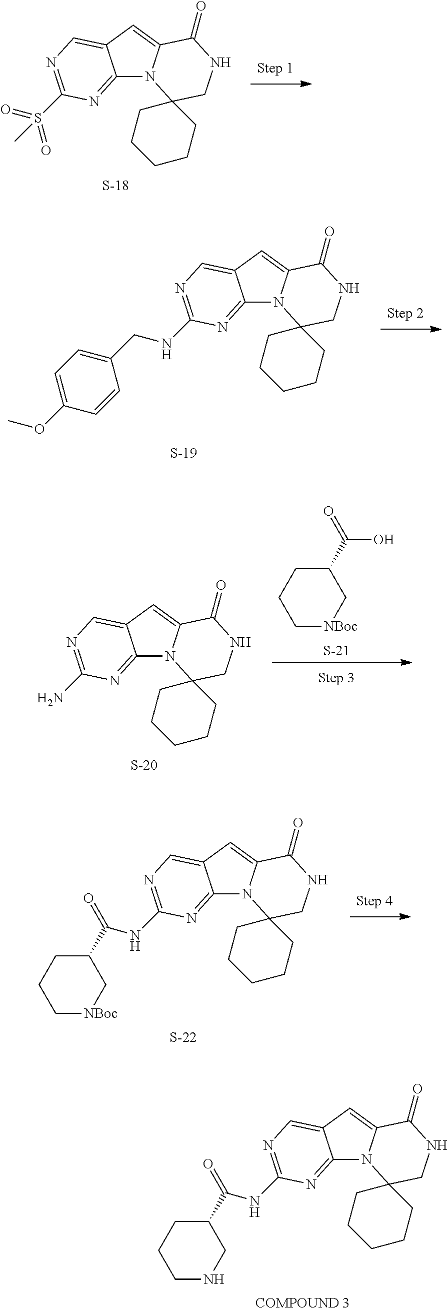

In Step 1, 5 g of S-18 was reacted in N-methyl-2-pyrrolidone at 120° C. to provide 6.1 g of S-19. In Step 2, 6.1 g of S-19 was reacted with trifluoroacetic acid to provide 5.4 g of S-20.

### Synthesis of (R)-1-methyl-N-(6′-oxo-7′,8′-dihydro-6′H-spiro[cyclohexane-1,9′-pyrazino[1′,2′:1,5]pyrrolo[2,3-d]pyrimidin]-2′-yl)piperidine-3-carboxamide (COMPOUND 4)

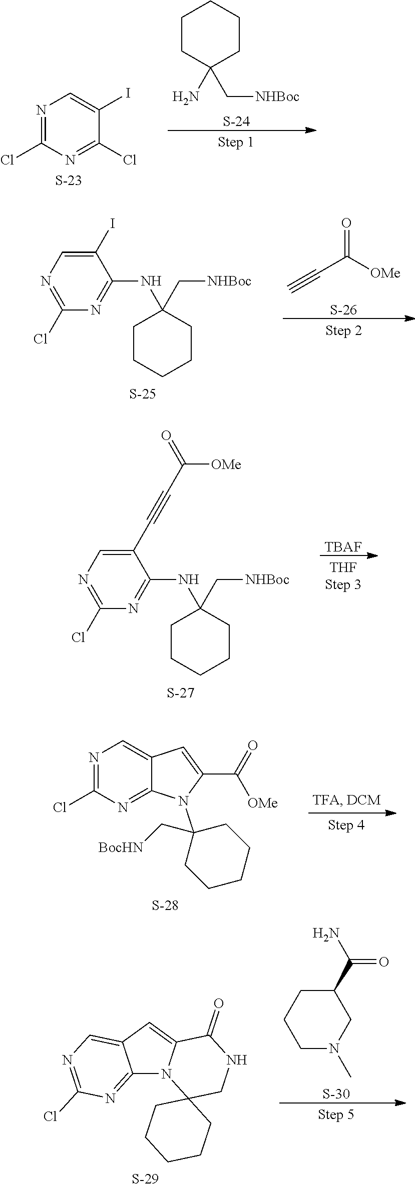

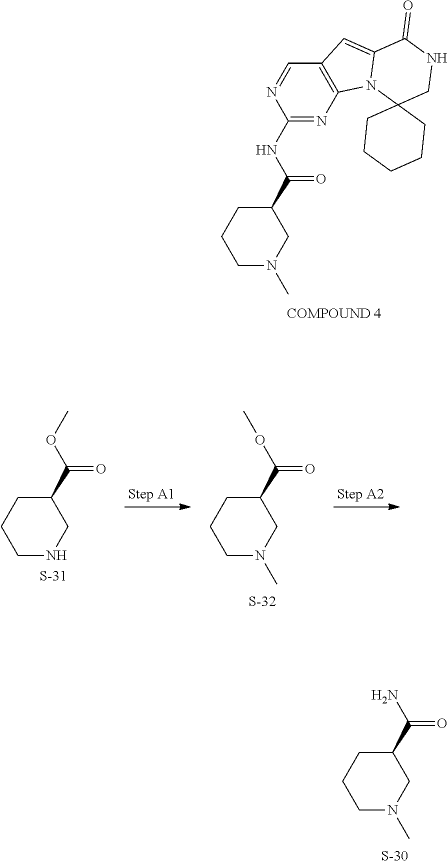

In Step 1, 20 g of S-23 was reacted with S-24 and potassium carbonate in N,N-dimethylacetamide at 80° C. to provide 25 g of crude S-25. In Step A1, 200 mg S-31 was reacted with methyl iodide and sodium bicarbonate in THE to provide 150 mg of S-32.

### Synthesis of (R)—N-(6′-oxo-7′,8′-dihydro-6′H-spiro[cyclohexane-1,9′-pyrazino[1′,2′:1,5]pyrrolo[2,3-d]pyrimidin]-2′-yl)-1-((R)-piperidine-3-carbonyl)piperidine-3-carboxamide (COMPOUND 5)

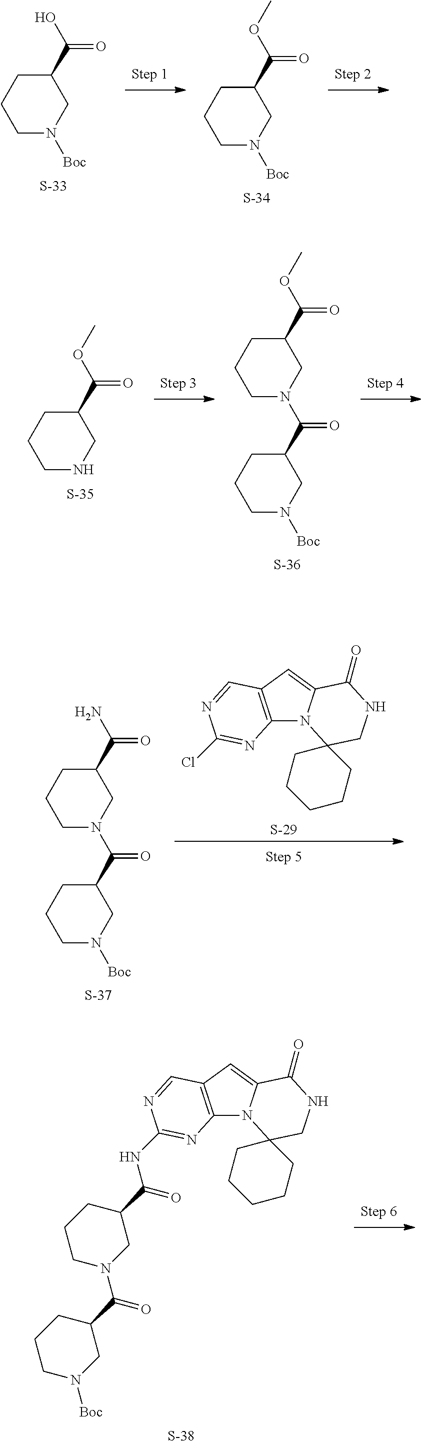

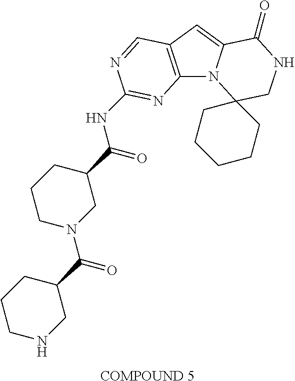 [SMILES: O=C1NCC2(CCCCC2)n2c1cc1cnc(NC(=O)[C@@H]3CCCN(C(=O)[C@@H]4CCCNC4)C3)nc12 ; "COMPOUND 5"]

In Step 1, 1 g of S-33 was reacted with methyl iodide and potassium carbonate in N,N-dimethylacetamide at 40° C. to provide 1.0 g of S-34. In Step 2, 1.0 g of S-34 reacted with trifluoroacetic acid in dichloromethane to provide 450 mg of S-35. In Step 3, 300 mg of S-33 was reacted with isobutyl chloroformate and N-methylmorpholine in N,N-dimethylformamide at room temperature for one hour. S-35 was then added, and the reaction mixture was stirred for an additional hour. Upon purification, 220 mg of S-36 was obtained.

### Synthesis of 3-amino-N-(6′-oxo-7′,8′-dihydro-6′H-spiro[cyclohexane-1,9′-pyrazino[1′,2′:1,5]pyrrolo[2,3-d]pyrimidin]-2′-yl)propanamide (COMPOUND 6)

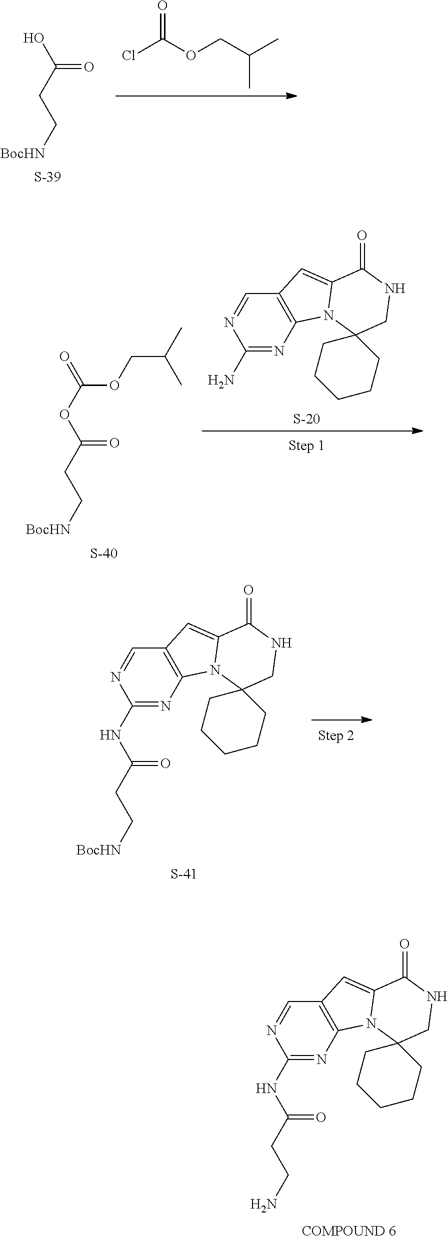

In Step 1, S-39 was reacted with isobutyl chloroformate and N-methylmorpholine in N,N-dimethylacetamide to provide crude S-40. S-20 was then added directly, and the reaction mixture was stirred at 60° C. for 48 hours. Upon purification by preparatory HPLC, 5 mg of S-41 was obtained. In Step 2, 5 mg of S-41 was reacted with trifluoroacetic acid in dichloromethane at room temperature for one hour to provide COMPOUND 6.

### Synthesis of 2′-(((3S,5R)-5-hydroxy-2-oxopiperidin-3-yl)amino)-7′,8′-dihydro-6′H-spiro[cyclohexane-1,9′-pyrazino[1′,2′:1,5]pyrrolo[2,3-d]pyrimidin]-6′-one (COMPOUND 7) and 2′-(((3R,5R)-5-hydroxy-2-oxopiperidin-3-yl)amino)-7′,8′-dihydro-6′H-spiro[cyclohexane-1,9′-pyrazino[1′,2′:1,5]pyrrolo[2,3-d]pyrimidin]-6′-one (COMPOUND 8)

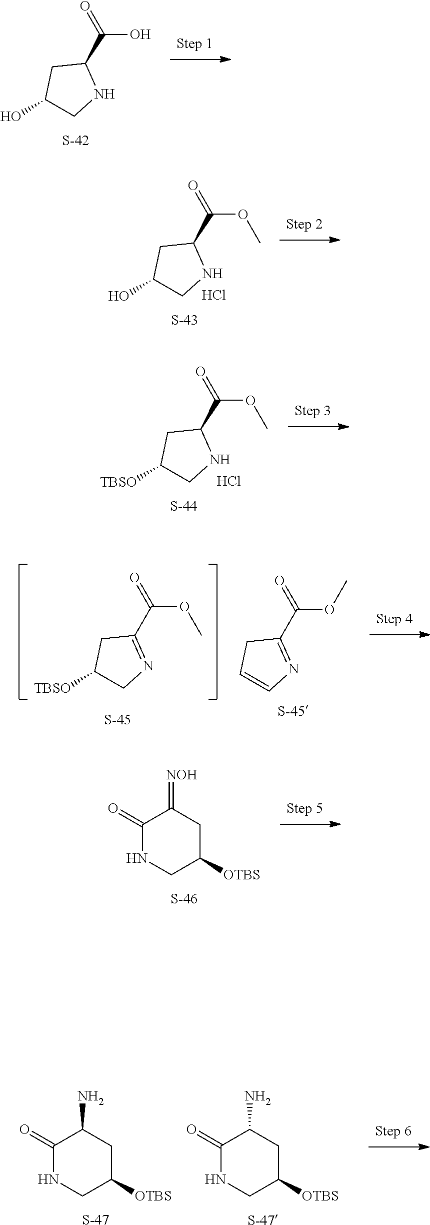

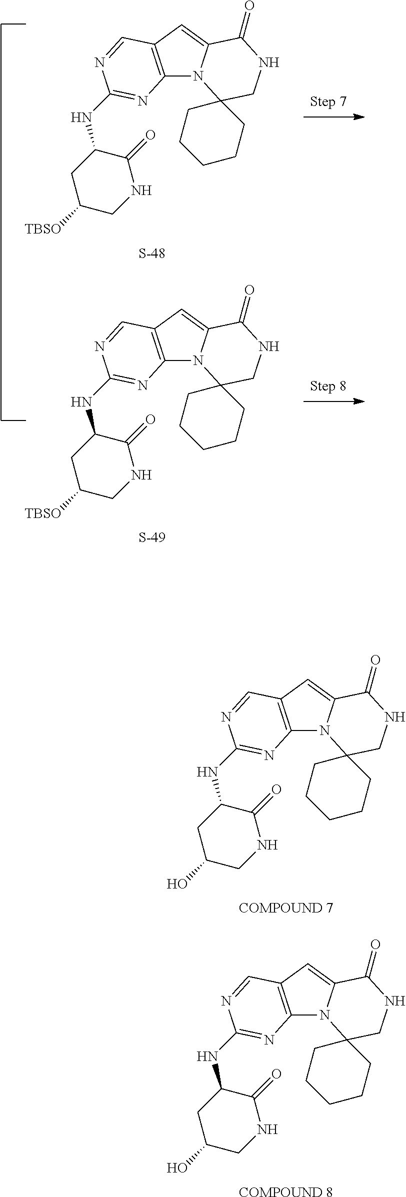

In Step 1, 10 g of S-42 was reacted with thionyl chloride in methanol to provide 11.2 g of S-43. In Step 2, 9.6 g of S-43 was reacted with TBSCl and imidazole in dichloromethane to provide 9.9 g of S-44. In Step 3, 9.3 g of S-44 was reacted with tert-butyl hypochlorite and triethylamine in diethyl ether at 0° C. to provide crude S-45 in addition to 10% S-45′. In step 4, crude S-45 was directly reacted with hydroxylamine hydrochloride and sodium bicarbonate in ethanol to provide 6.1 g of S-46 over two steps. In step 5, 2 g of S-46 was reacted with zinc in acetic acid to provide 600 mg of S-47 and 510 mg of S-47′. In one run of step 6, 25 mg of S-47 was reacted with N,N-diisopropylethylamine in N,N-dimethylacetamide at 110° C. to provide 11 mg of S-48. In another run of step 6, 25 mg of S-47′ was reacted with N,N-diisopropylethylamine in N,N-dimethylacetamide at 110° C. to provide 8 mg of S-49. In Step 7, 11 mg of S-48 was reacted with tetrabutylammonium fluoride in THF to provide COMPOUND 7 as confirmed by LC-MS. In step 8, 8 mg of S-49 was reacted with tetrabutylammonium fluoride in THF to provide COMPOUND 8 as confirmed by LC-MS.

### Synthesis of 2′-(((2R,3R)-2-phenylpiperidin-3-yl)amino)-7′,8′-dihydro-6′H-spiro[cyclohexane-1,9′-pyrazino[1′,2′:1,5]pyrrolo[2,3-d]pyrimidin]-6′-one (COMPOUND 9) and 2′-(((2R,3S)-2-phenylpiperidin-3-yl)amino)-7′,8′-dihydro-6′H-spiro[cyclohexane-1,9′-pyrazino[1′,2′:1,5]pyrrolo[2,3-d]pyrimidin]-6′-one (COMPOUND 10)

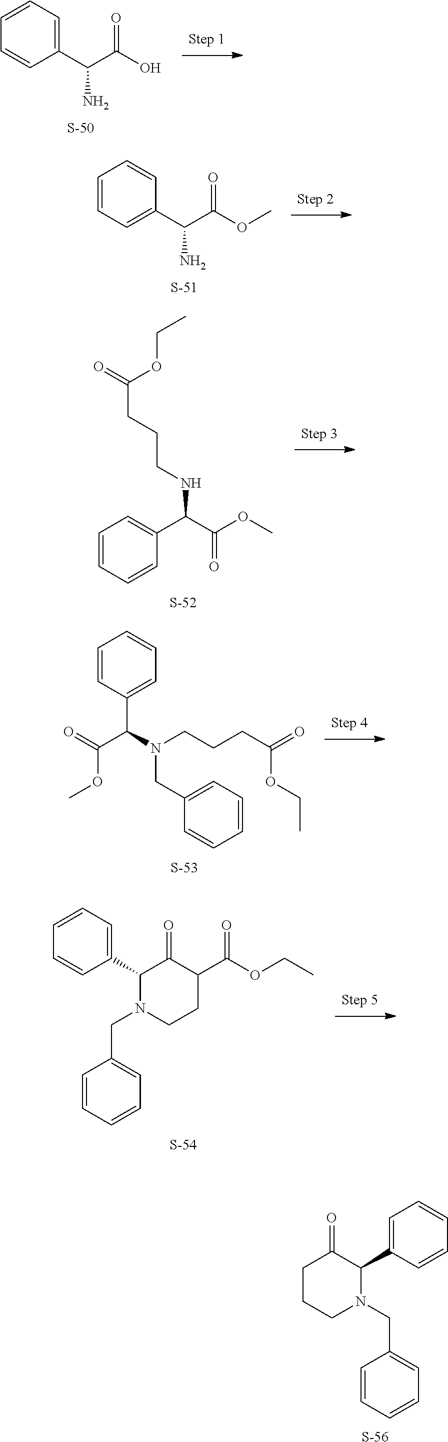

In step 1, 25 g of S-50 was reacted with thionyl chloride in methanol at 60° C. to provide 26 g of S-51. In step 2, 20 g of S-51 was reacted with potassium carbonate in acetonitrile at 50° C. to provide 24 g of S-52. In step 3, 5 g of S-52 was reacted with benzyl bromide and potassium carbonate in acetonitrile at 70° C. to provide 4.2 g of S-53. In step 4, S-54 was reacted in concentrated HCl at 85° C. to provide 2.1 g of S-56.

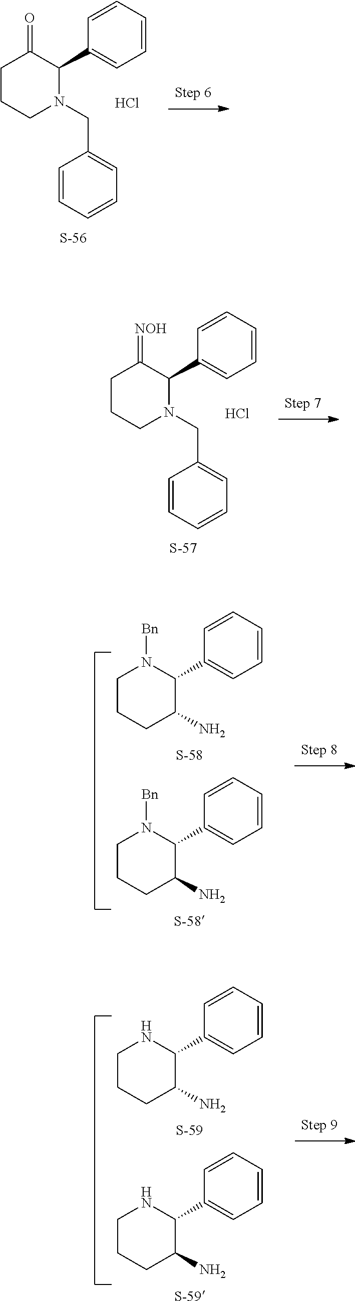

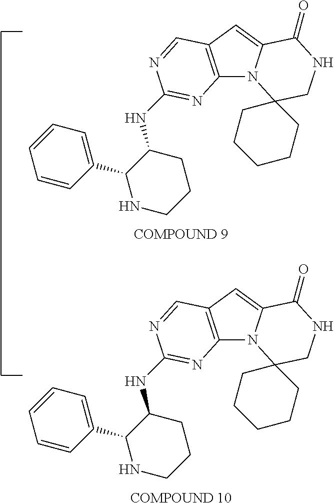

In step 6, 600 mg of S-56 was reacted with potassium acetate in an ethanol/water mixture at 75° C. to provide 500 mg of S-57. In step 7, 500 mg of S-57 was reacted with Raney nickel in ethanol under a hydrogen atmosphere at 55° C. to provide 130 mg of a mixture of S-58 and S-58′. In step 8, 50 mg of S-58/S-58′ was reacted with Pd/C in methanol at 55° C. to provide 20 mg of a mixture of S-59 and S-59′. In Step 9, 10 mg of S-59/S-59′ was reacted with triethylamine in ethanol in a microwave reactor at 140° C. to provide COMPOUND 9 and COMPOUND 10.

### Synthesis of (R)—N-(6′-oxo-7′,8′-dihydro-6′H-spiro[cyclohexane-1,9′-pyrazino[1′,2′:1,5]pyrrolo[2,3-d]pyrimidin]-2′-yl)piperidine-3-carboxamide (COMPOUND 11)

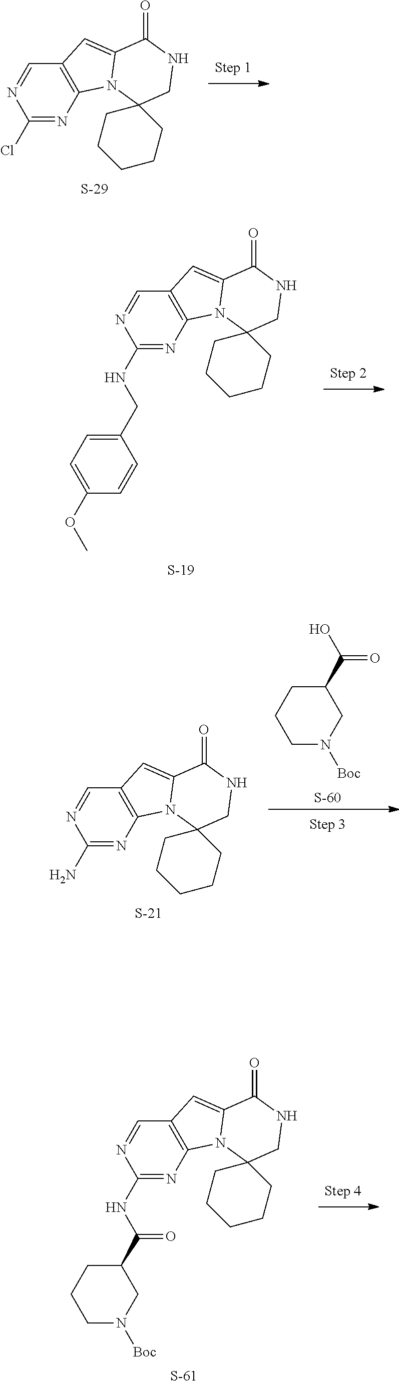

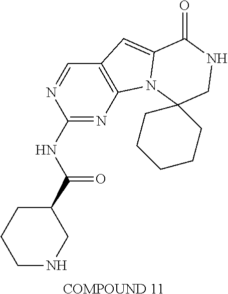

In Step 1, 100 mg of S-29was reacted with 4-methoxybenzylamine and N-diisopropylethylamine in N,N-dimethylacetamide at 130° C. to provide 105 mg of S-19. In step 2, 100 mg of S-19 was reacted with trifluoroacetic acid to provide 85 mg of S-21. In Step 3, 80 mg of S-21 was reacted with the acyl chloride derivative of S-60 and triethylamine in dichloromethane to provide 20 mg of S-61. In Step 4, 20 mg of S-61 was reacted with trifluoroacetic acid in dichloromethane to provide 7.1 mg of COMPOUND 11.

### Synthesis of 2′-((5-(4-methylpiperazin-1-yl)pyridin-2-yl)amino)-8′H-spirocyclohexane-1,9′-pyrazino[1′,2′:1,5]pyrrolo[2,3-d]pyrimidin-8′-one (COMPOUND 12)

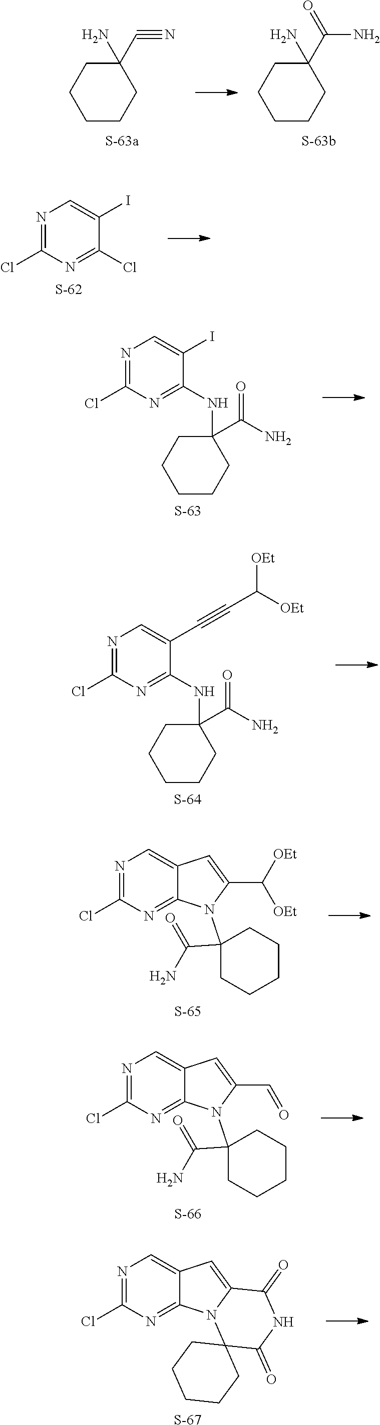

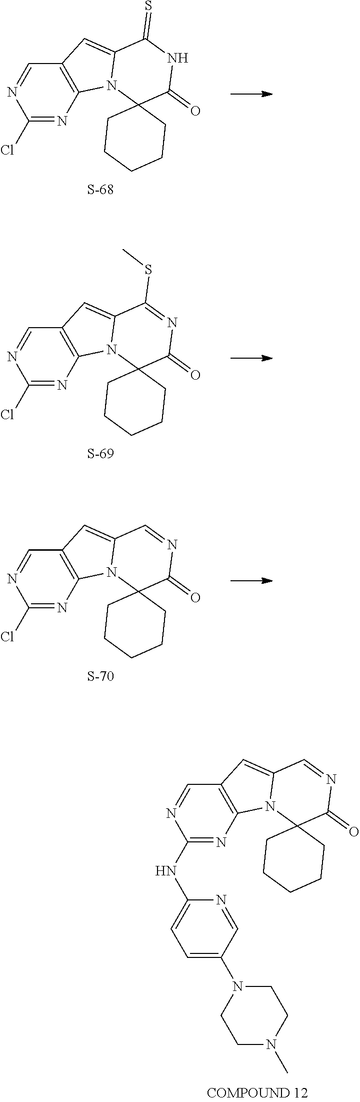

S-63a (40 g) was converted to S-63b using H₂SO₄ in DCM and stirring at 25° C. for 2 hours. After purification, 20.6 g of S-63b was obtained. S-62 (20 g) was converted to S-63 by reaction S-63b and S-62 in the presence of NaHCO₃ in DMA at 80° C. for 12 h. After purification, 12 g of S-63 was obtained. S-63 (1.1 g) was converted to S-64 using (PPh₃)₂PdCl₂, CuI, and TEA. The reaction was stirred in TH at room temperature overnight. Purification afford 750 mg of S-64. ¹HNMR confirmed the structure of S-64. S-64 (700 mg) was converted to S-65 using TBAF in THE at 65° C. for 2 hours. After purification, 450 mg of S-65 was obtained. S-65 (400 mg) was converted to S-66 using HOAc in THE and H₂O. The reaction was stirred at 60° C. for 2 hours. After purification, 80 mg of S-66 was obtained. (Alternatively, S-66 (80 mg) was obtained by subjecting S-65 (450 mg) to NaClO₂ and NaH₂PO₄ and stirring at room temperature overnight). S-66 (40 mg) was converted to S-67 using NaClO₂ and NaH₂PO₄ in t-BuOH. The reaction stirred at room temperature for 12 hours. After purification, 35 mg of S-67 was obtained. S-67 (80 mg) was converted to S-68 using P₂S₅ in toluene. The reaction stirred at 110° C. for 2 hours at which point S-68 was observed by LCMS. Purification afforded 58 mg of S-68. S-68 (48 mg) was converted to S-69 using CH₃I and K₂CO₃ in acetone. The reaction stirred at room temperature for 12 hours at which point S-69 was observed by LCMS. After purification, 30 mg of S-69 was obtained and the structure was confirmed by ¹HNMR and LCMS.

### Synthesis of 2′-(((1s,4s)-4-(4-methylpiperazin-1-yl)cyclohexyl)amino)-7′,8′-dihydro-6′H-spiro[cyclohexane-1,9′-pyrazino[1,2-e]purine]-6′-thione (COMPOUND 13)

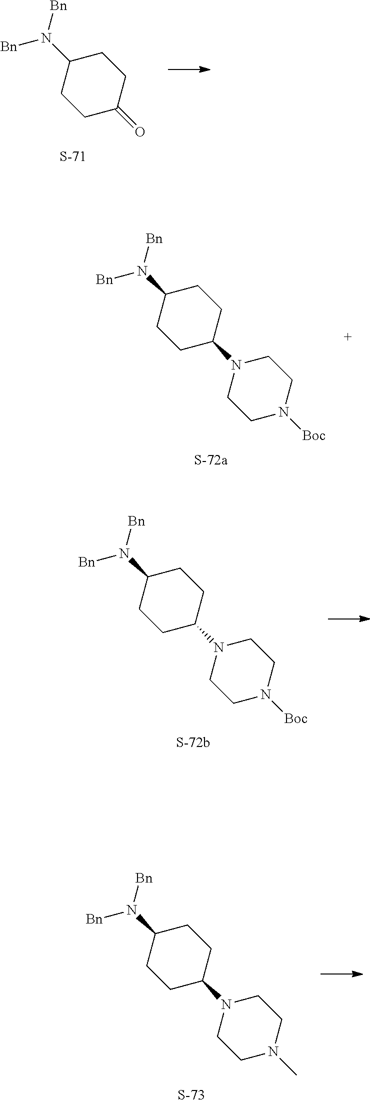

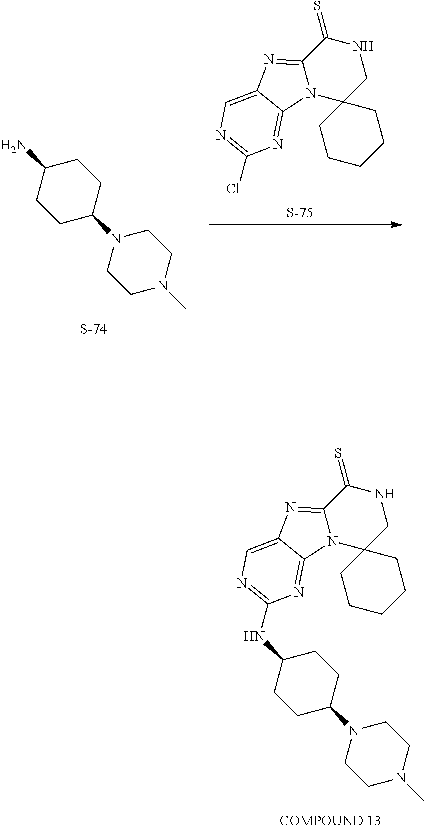

S-71(5 g) was converted to S-72a and S-72b using NaHBCN in acetonitrile and stirring the reaction at room temperature overnight. After column purification, 1.5 g of S-72 and 0.6 g of S-73 were obtained. S-72a (1.2 g) was converted to S-73 using LAH in THF. The reaction was refluxed for 3 hours and afforded 0.73 g of S-73. S-73 (0.7 g) was converted to S-74 using Pd/C in i-PrOH at a pressure of 2 MPa H2 at 40° C. for 6 hours. After work-up, 265 mg of S-74 was obtained. Following a reaction with S-74 in the presence of S-75, COMPOUND 13 was 5 obtained (2 mg, >95% purity).

### Synthesis of (S)-2′-(piperidin-3-ylamino)-7′,8′-dihydro-6′H-spiro[cyclohexane-1,9′-pyrazino[1,2-e]purine]-6′-thione (COMPOUND 14)

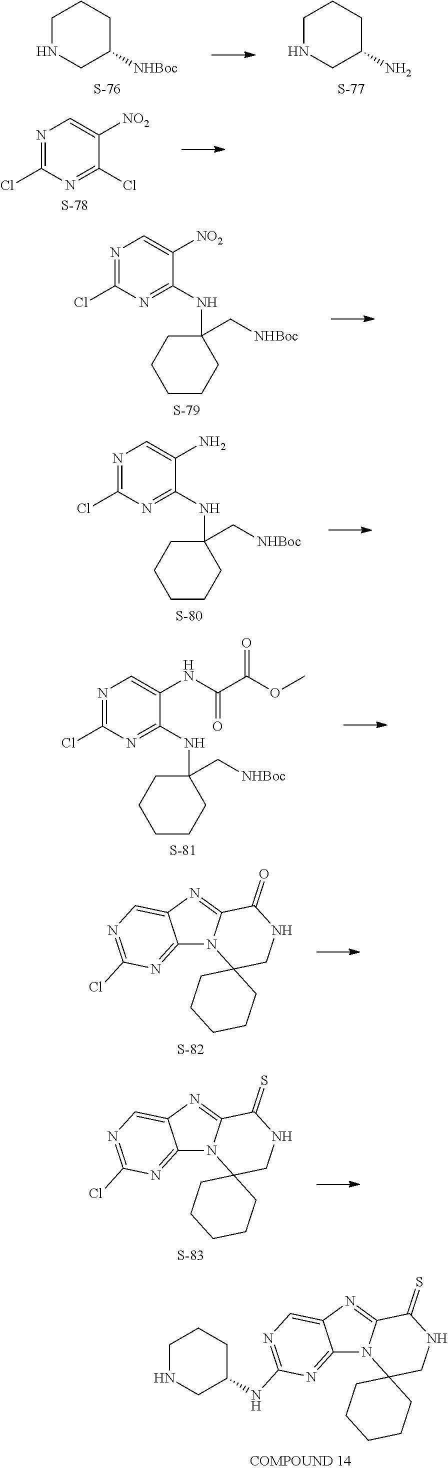

S-78 (10.0 g) was converted to S-79 using NaHCO₃ in THE and stirring at 20° C. for 12 hours. After purification, 12.0 g of S-79 was obtained. S-79 (12.0 g) was converted to S-80 using Fe powder and NH₄Cl in EtOH at 50° C. for 12 h. After purification, 8.0 g of S-80 was obtained. S-80 (8 g) was converted to S-81 using NaHCO₃ in THE and stirring at room temperature for 4 hours. After purification, 6.0 g of pure S-81 (and 2.5 g of impure S-81) was obtained. S-81 (4 g) was converted to S-82 using diphenyl ethers. The reaction was heated to approximately 200° C. and stirred for 1 hour. After purification, 520 mg of pure S-82 (and 500 mg of impure S-82) was obtained. S-82 (30 mg) was converted to S-83 using Lawesson's reagent in THE at 80° C. for 12 hours. S-83 was isolated and the molecular weight was confirmed by LC-MS. Alternatively, S-82 (20 mg) was converted to S-83 using P₂S₅ in dioxane at 110° C. for 12 hours. S-82 is also converted to S-83 using Lawesson's reagent in pyridine at 90° C. for 12 h and P₂S₅ in pyridine at 90° C. for 12 hours.

### Synthesis of 2′-(((1r,4r)-4-((tetrahydro-2H-pyran-4-yl)amino)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 16)

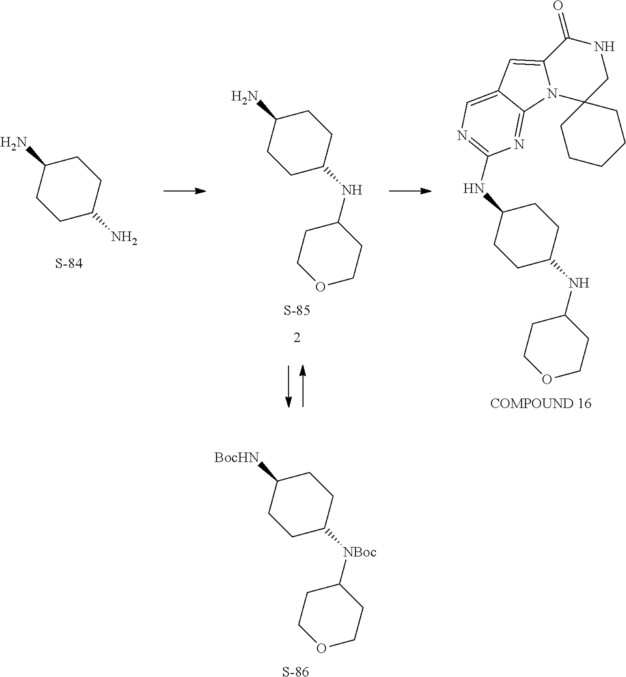

S-84 (1 g) was converted to S-85 using NaBH₃CN in the presence of HOAc and DCM. The reaction was stirred overnight at room temperature. S-85 was observed by LC-MS, but was difficult to purify due to its high polarity. Therefore, S-85 was converted to S-86 and purified to afford pure S-86 (410 mg). S-86 (410 mg) was converted to S-85 using TFA in DCM and stirring at room temperature for 3 hours to afford 380 mg of S-85 as a TFA salt. S-85 TFA salt (50 mg) was converted to COMPOUND 16 using DIEA in EtOH and refluxing overnight. After purification, 5.9 mg of COMPOUND 16 was obtained and the product was confirmed via ¹H-NMR and HPLC.

### Synthesis of 2′-(((5-methylfuran-2-yl)methyl)amino)-7′,8′-dihydro-6′H-spiro[cyclohexane-1,9′-pyrazino[1′,2′:1,5]pyrrolo[2,3-d]pyrimidin]-6′-one (COMPOUND 17)

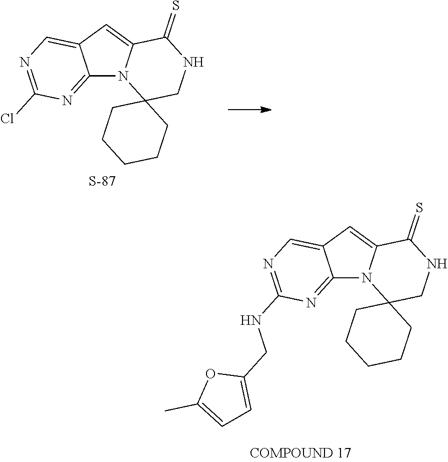

20 mg of S-87 was converted to COMPOUND 17 using TEA in EtOH at 120° C. for 12 hours. After purification, 6.4 mg of COMPOUND 17 was obtained.

### Synthesis of 2′-(((1,4-trans)-4-(pyridin-2-ylamino)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 18)

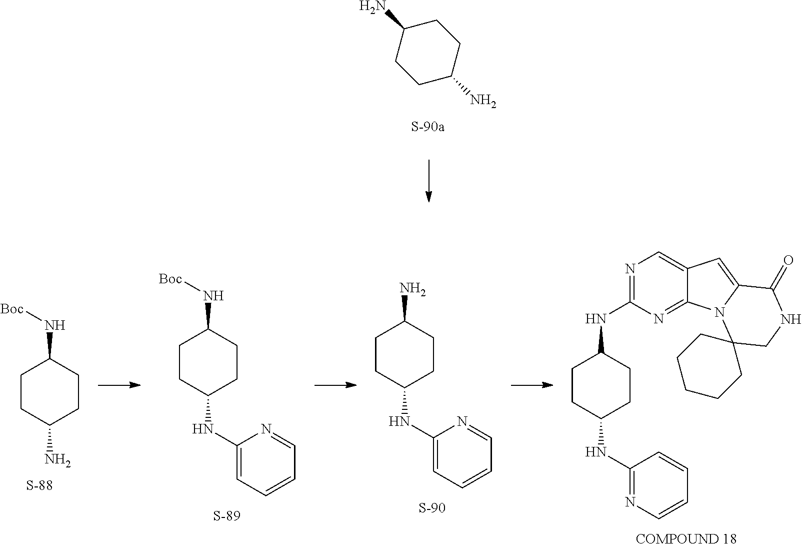

S-88 (4 g) was converted to S-89 using Boc₂O in THE and NaHCO₃ at room temperature overnight. After purification, 2.5 g of 2-89 was obtained. Alternatively, S-88 (100 mg) was converted to S-89 using K₂CO₃ in NMP. The reaction was heated to 120° C. in a microwave for 0.5 hours to afford 5 mg of S-89. ¹HNMR confirmed the structure. S-90a (100 mg) was converted to S-90 using K₂CO₃ and 2-fluoropyridine in NMP and the reaction was heated to 140° C. for 12 hours. After purification, 20 mg of S-90 was obtained. S-90 (20 mg) was converted to COMPOUND 18 using TEA in EtOH. The reaction was heated to 140° C. in a microwave reactor for 30 minutes. After preparative TLC purification, 4.1 mg of COMPOUND 18 was obtained and confirmed via ¹HNMR and HPLC.

Alternatively, S-91 is converted to COMPOUND 18:

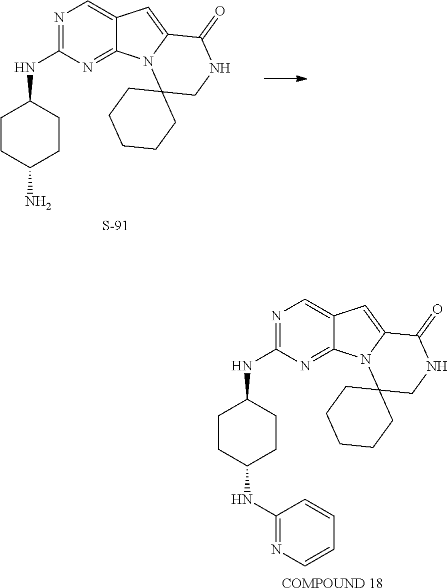

### Synthesis of 2′-(((1,4-trans)-4-((5-fluoropyrimidin-2-yl)amino)cyclohexyl)-12-azaneyl)-7′,8′-dihydro-6′H-spiro[cyclohexane-1,9′-pyrazino[1′,2′:1,5]pyrrolo[2,3-d]pyrimidin]-6′-one (COMPOUND 19)

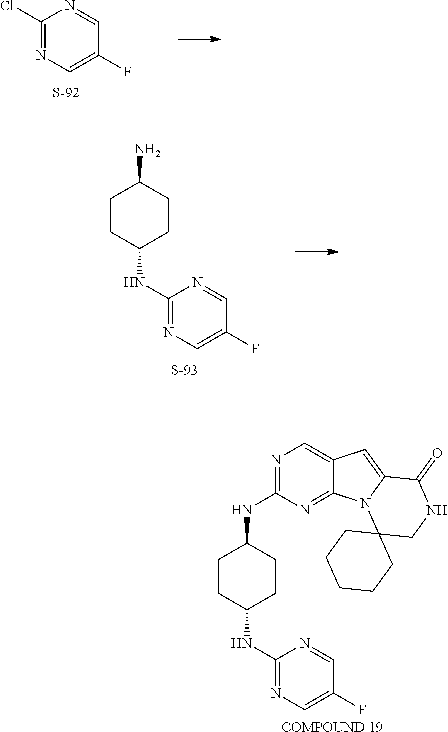

S-92 (130 mg) was converted to S-93 using DIEA in i-PrOH. The reaction was heated in a microwave reactor for 140° C. for 20 minutes. After purification, 60 mg of S-93 was obtained. S-93 (40 mg) was converted to COMPOUND 19 using DIEA in i-PrOH. The reaction was heated to 130° C. in a microwave reactor for 30 minutes. After purification, 6.1 mg of COMPOUND 19 was obtained and confirmed via HPLC and ¹HNMR. Alternative approaches to the synthesis of COMPOUND 19 include:

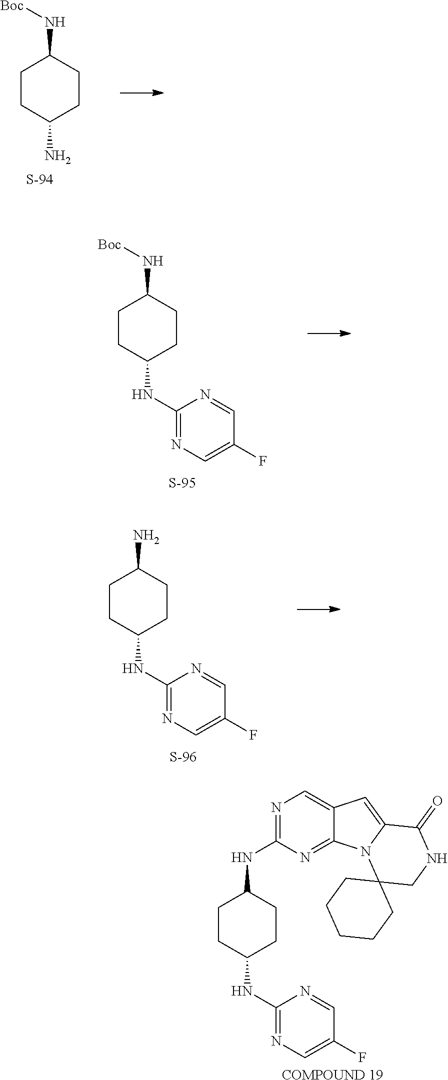

S-94 (200 mg) was converted to S-95 using TEA in DMAc at 120° C. overnight. After purification, 10 mg of S-95 was obtained Alternatively, COMPOUND 19 is synthesized as shown below:

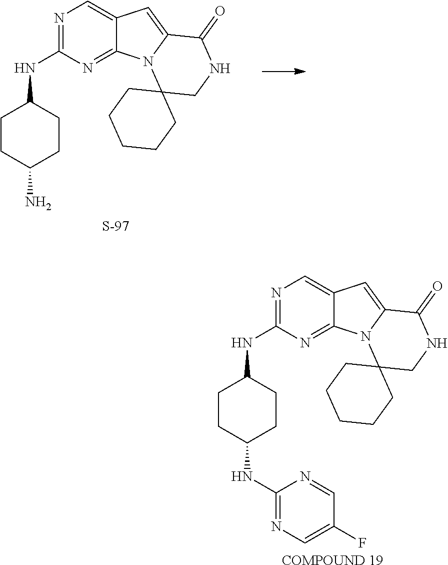

### Synthesis of 2′-(((1,4-trans)-4-((pyridin-3-ylmethyl)amino)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 21)

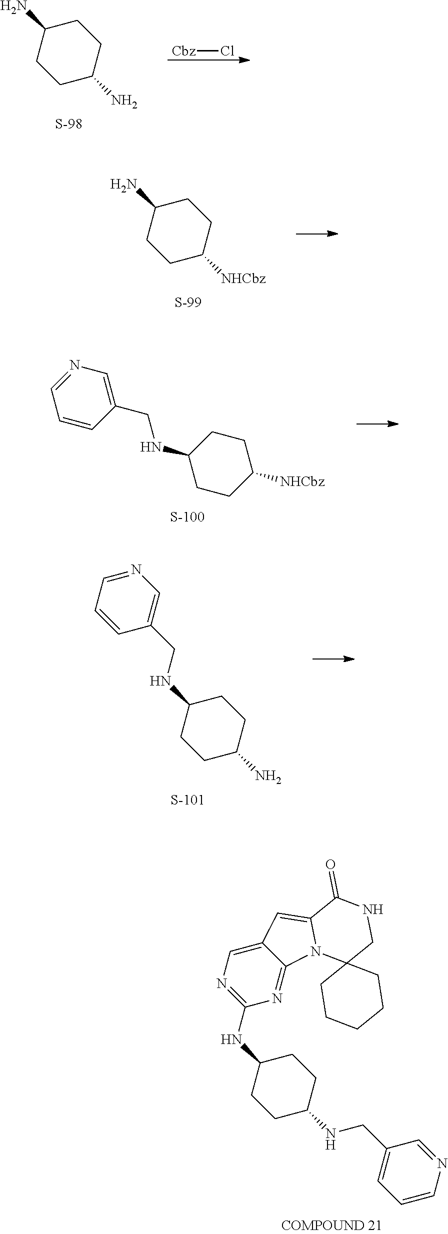

S-98 (2.28 g) was converted to S-99 using CBzCl in the presence of NaHCO₃ in THE at room temperature for 2 hours. After workup, 2.1 g of S-99 was obtained. S-99 is converted to S-100 using NaBH₃CN in DCM and HOAc. The reaction is stirred at room temperature overnight.

### Synthesis of N-((1,4-trans)-4-((6′-oxo-7′,8′-dihydro-6′H-spiro[cyclohexane-1,9′-pyrazino[1′,2′:1,5]pyrrolo[2,3-d]pyrimidin]-2′-yl)amino)cyclohexyl)methanesulfonamide (COMPOUND 22)

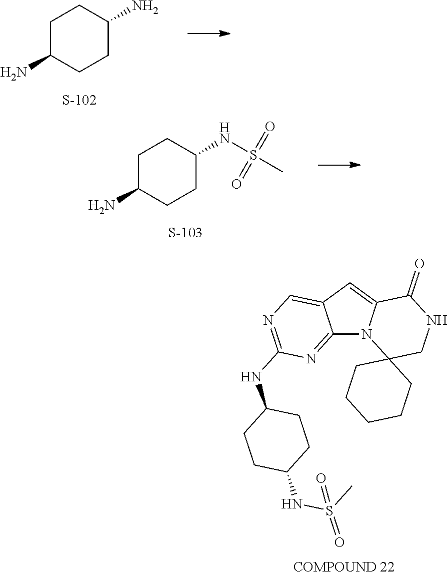 [SMILES: N[C@H]1CC[C@H](N)CC1 ; CS(=O)(=O)N[C@H]1CC[C@H](N)CC1 ; CS(=O)(=O)N[C@H]1CC[C@H](Nc2ncc3cc4n(c3n2)C2(CCCCC2)CNC4=O)CC1]

S-102 (200 mg) was converted to S-103 using MsCl in DCM at 0° C. for 1 hour. After purification, 250 mg of S-103 was obtained. 50 mg of S-103 is converted to COMPOUND 22 using TEA in EtOH. The reaction was heated to 140° C. in a microwave reactor for 15 minutes.

### Synthesis of (S)-2′-((2-oxopiperidin-3-yl)amino)-7′,8′-dihydro-6′H-spiro[cyclohexane-1,9′-pyrazino[1′,2′:1,5]pyrrolo[2,3-d]pyrimidin]-6′-one (COMPOUND 23)

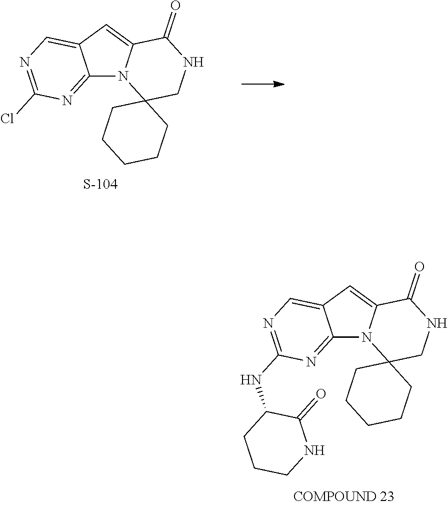 [SMILES: O=C1NCC2(CCCCC2)n2c1cc1cnc(Cl)nc12 ; O=C1NCC2(CCCCC2)n2c1cc1cnc(N[C@H]3CCCNC3=O)nc12]

S-104 (20 mg) was converted to COMPOUND 23 using TEA in EtOH. The reaction was heated to 140° C. in a microwave for 20 minutes. After purification by preparative TLC, 6.4 mg of COMPOUND 23 was obtained and confirmed via H-NMR and HPLC.

### Synthesis of (S)-2′-((6-oxopiperidin-3-yl)amino)-7′,8′-dihydro-6′H-spiro[cyclohexane-1,9′-pyrazino[1′,2′:1,5]pyrrolo[2,3-d]pyrimidin]-6′-one (COMPOUND 24)

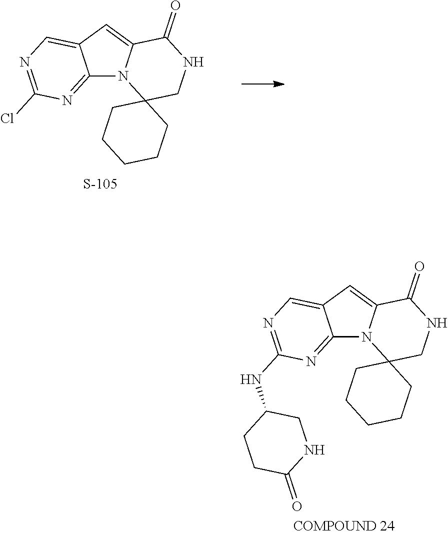 [SMILES: O=C1NCC2(CCCCC2)n2c1cc1cnc(Cl)nc12 ; O=C1CC[C@H](Nc2ncc3cc4n(c3n2)C2(CCCCC2)CNC4=O)CN1]

S-105 (20 mg) was converted to COMPOUND 24 using TEA in EtOH. The reaction was heated to 140° C. in a microwave reactor for 30 minutes. After purification by preparative TLC, 8.0 mg of COMPOUND 24 was obtained and confirmed via H-NMR and HPLC.

### Synthesis of (S)-3-((6′-oxo-7′,8′-dihydro-6′H-spiro[cyclohexane-1,9′-pyrazino[1′,2′:1,5]pyrrolo[2,3-d]pyrimidin]-2′-yl)amino)piperidine-2,6-dione (COMPOUND 25)

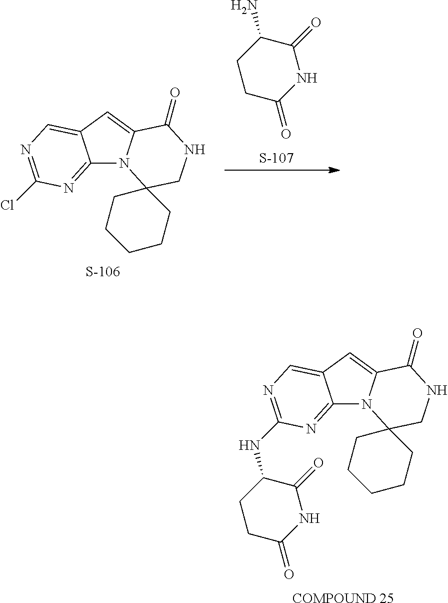

S-106 is converted to COMPOUND 25 in the presence of S-107 and NaHCO₃ in NMP at 100° C. for 2 hours. Alternatively, S-106 is converted to COMPOUND 25 in the presence of S-107 using TEA in EtOH. The reaction is heated to 140° C. in a microwave reactor for 15 minutes. Alternatively, S-106 is converted to COMPOUND 25 using LHMDS.

### Synthesis of (S)-2′-((2-oxo-1,2,3,4-tetrahydroquinolin-3-yl)amino)-7′,8′-dihydro-6′H-spiro[cyclohexane-1,9′-pyrazino[1′,2′:1,5]pyrrolo[2,3-d]pyrimidin]-6′-one (COMPOUND 27)

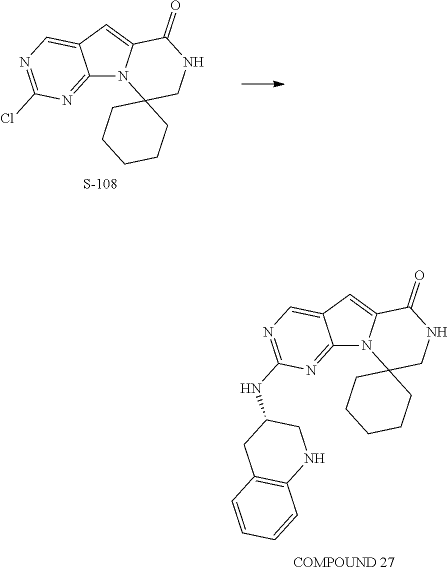 [SMILES: O=C1NCC2(CCCCC2)n2c1cc1cnc(Cl)nc12 ; O=C1NCC2(CCCCC2)n2c1cc1cnc(N[C@@H]3CNc4ccccc4C3)nc12]

S-108 (30 mg) was converted to COMPOUND 27 using TEA in EtOH. The reaction was heated in a microwave reactor at 140° C. for 15 minutes. After purification, 2.8 mg of COMPOUND 27 was obtained and confirmed via ¹HNMR and HPLC.

### Synthesis of (2R,3R)-2-methyl-N-(6′-oxo-7′,8′-dihydro-6′H-spiro[cyclohexane-1,9′-pyrazino[1′,2′:1,5]pyrrolo[2,3-d]pyrimidin]-2′-yl)piperidine-3-carboxamide (COMPOUND 30)

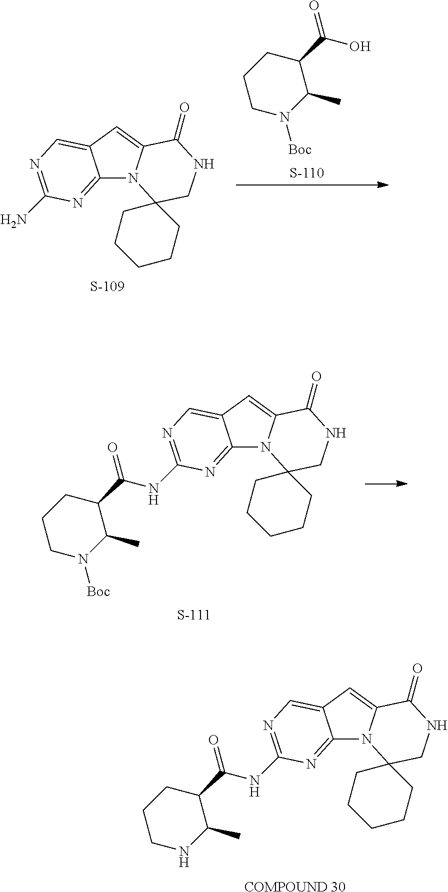

S-109 (50 mg) was converted to S-111 using S-110 in the presence of isobutyl chloroformate and NMM in DMAc. The reaction was stirred at room temperature for 12 hours. After workup, 100 mg of crude S-111 was obtained and used directly in the next step. Crude S-111 is converted to COMPOUND 30 using TFA in DCM at room temperature for 2 hours.

### Synthesis of N-(6′-Oxo-7′,8′-dihydro-6′H-spiro[cyclohexane-1,9′-pyrazino[1′,2′:1,5]pyrrolo[2,3-d]pyrimidin]-2′-yl)azetidine-3-carboxamide (COMPOUND 31)

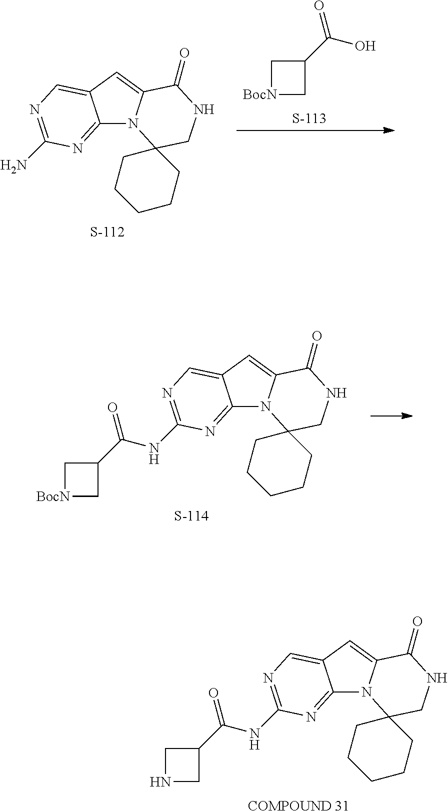

S-112 (100 mg) was reacted with S-113 using i-BuCO₂Cl in NMM and DMAc. The reaction was stirred at room temperature for 12 hours to afford S-114. After purification, 30 mg of S-114 was obtained. S-114 (10 mg) was converted to COMPOUND 31 using TFA in DCM. The reaction was stirred for 2 hours at room temperature. After purification by preparative HPLC, 6.4 mg of COMPOUND 31 as the TFA salt was obtained and confirmed via ¹H-NMR and LC-MS (HPLC purity of 93%).

### Synthesis of (R)—N-(6′-Oxo-7′,8′-dihydro-6′H-spiro[cyclohexane-1,9′-pyrazino[1′,2′:1,5]pyrrolo[2,3-d]pyrimidin]-2′-yl)pyrrolidine-3-carboxamide (COMPOUND 32)

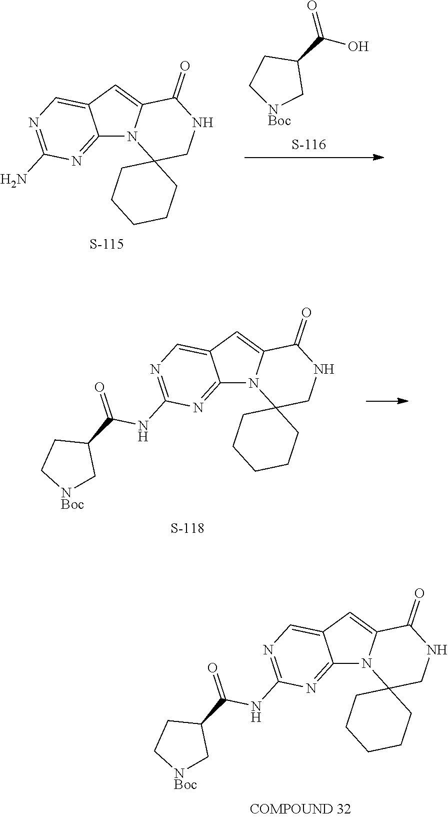 [SMILES: Nc1ncc2cc3n(c2n1)C1(CCCCC1)CNC3=O ; CC(C)(C)OC(=O)N1CC[C@@H](C(=O)O)C1 ; CC(C)(C)OC(=O)N1CC[C@@H](C(=O)Nc2ncc3cc4n(c3n2)C2(CCCCC2)CNC4=O)C1 ; CC(C)(C)OC(=O)N1CC[C@@H](C(=O)Nc2ncc3cc4n(c3n2)C2(CCCCC2)CNC4=O)C1]

S-115 (100 mg) and S-116 were converted to S-118 using i-BuCO₂Cl in NMM and DMAc. The reaction was stirred at room temperature for 12 hours. After purification, 120 mg of crude S-118 was obtained and used directly in the next step. Crude S-118 (30 mg) was converted to COMPOUND 32 using TFA in DCM. The reaction was stirred at room temperature for 2 hours. After purification, 8.1 mg of COMPOUND 32 was obtained.

### Synthesis of (S)—N-(6′-Oxo-7′,8′-dihydro-6′H-spiro[cyclohexane-1,9′-pyrazino[1′,2′:1,5]pyrrolo[2,3-d]pyrimidin]-2′-yl)pyrrolidine-3-carboxamide (COMPOUND 33)

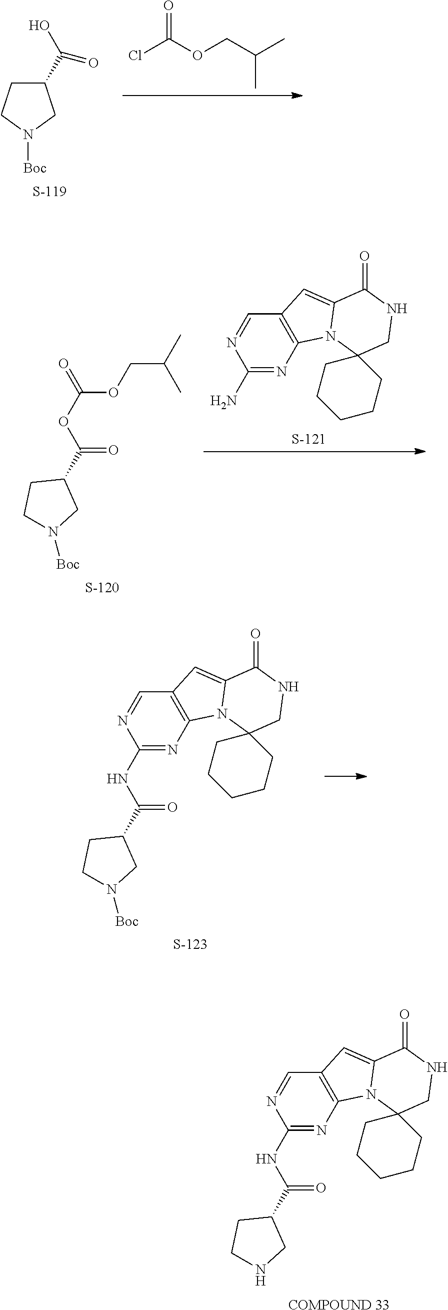

S-119 (200 mg) was converted to S-120 in the presence of isobutyl carbonochloridate using i-BuCO₂Cl in NMM and DMAc. The reaction was stirred at room temperature for 1 hour. S-121 was added and the reaction was stirred at 60° C. for 72 hours. After purification, 15 mg of S-122 was obtained. S-122 (15 mg) was converted to COMPOUND 33 using TFA in DCM. The reaction stirred at room temperature for 1 hour. After purification by preparative TLC and washing with DCM, 3.3 mg of COMPOUND 33 was obtained and the structure was confirmed via ¹H-NMR, HPLC and LC-MS.

### Synthesis of (S)—N-(6′-Oxo-7′,8′-dihydro-6′H-spiro[cyclohexane-1,9′-pyrazino[1′,2′:1,5]pyrrolo[2,3-d]pyrimidin]-2′-yl)-2-(pyrrolidin-2-yl)acetamide (COMPOUND 34)

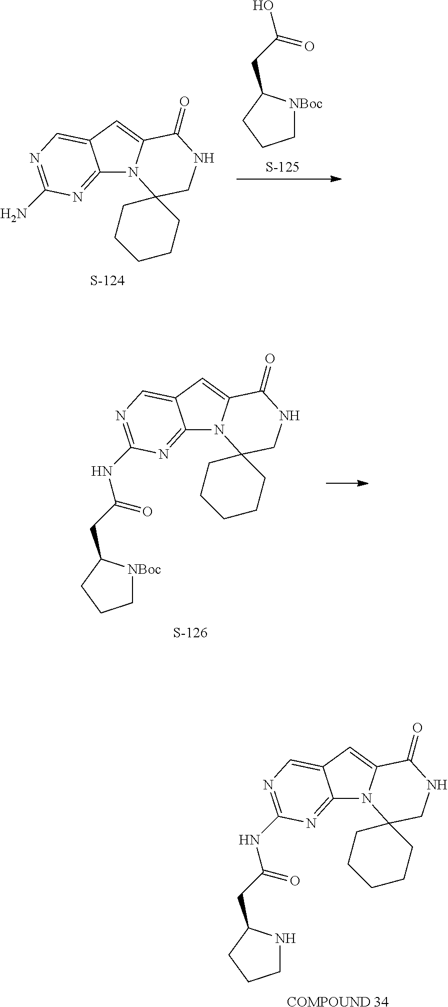

S-124 (100 mg) and S-125 were converted to S-126 using isobutyl chloroformate in NMM and DMAc. The reaction was stirred at room temperature for 12 hours. After workup, 120 mg of crude S-126 was obtained that was converted to COMPOUND 34 using TFA in DCM. The reaction stirred at room temperature for 12 hours. After purification, 19.0 mg of COMPOUND 34 was obtained and the structure was confirmed via ¹H-NMR, HPLC and LC-MS.

### Synthesis of (R)—N-(6′-Oxo-7′,8′-dihydro-6′H-spiro[cyclohexane-1,9′-pyrazino[1′,2′:1,5]pyrrolo[2,3-d]pyrimidin]-2′-yl)piperazine-2-carboxamide (COMPOUND 36)

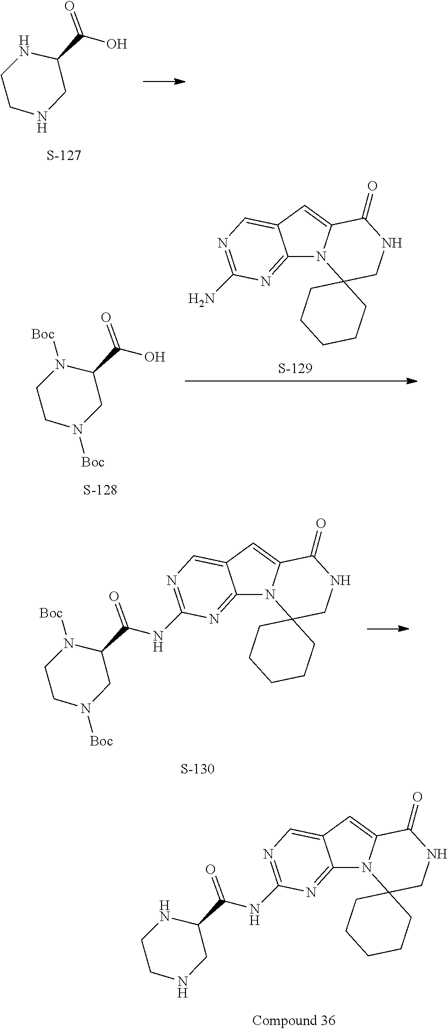 [SMILES: O=C(O)[C@H]1CNCCN1 ; CC(C)(C)OC(=O)N1CCN(C(=O)OC(C)(C)C)[C@@H](C(=O)O)C1 ; Nc1ncc2cc3n(c2n1)C1(CCCCC1)CNC3=O ; CC(C)(C)OC(=O)N1CCN(C(=O)OC(C)(C)C)[C@@H](C(=O)Nc2ncc3cc4n(c3n2)C2(CCCCC2)CNC4=O)C1 ; O=C1NCC2(CCCCC2)n2c1cc1cnc(NC(=O)[C@H]3CNCCN3)nc12]

S-127 (200 mg) was converted to S-128 using Boc₂O in the presence of NaOH in dioxane and H₂O. The reaction was stirred at room temperature for 12 hours. After purification, 400 mg of S-128 was obtained. S-128 is coupled to S-129 using isobutyl chloroformate in NMM and DMAc. The reaction stirred at room temperature for 12 hours.

### Synthesis of (R)—N-(6′-Oxo-7′,8′-dihydro-6′H-spiro[cyclohexane-1,9′-pyrazino[1′,2′:1,5]pyrrolo[2,3-d]pyrimidin]-2′-yl)-2-(pyrrolidin-2-yl)acetamide (COMPOUND 37)

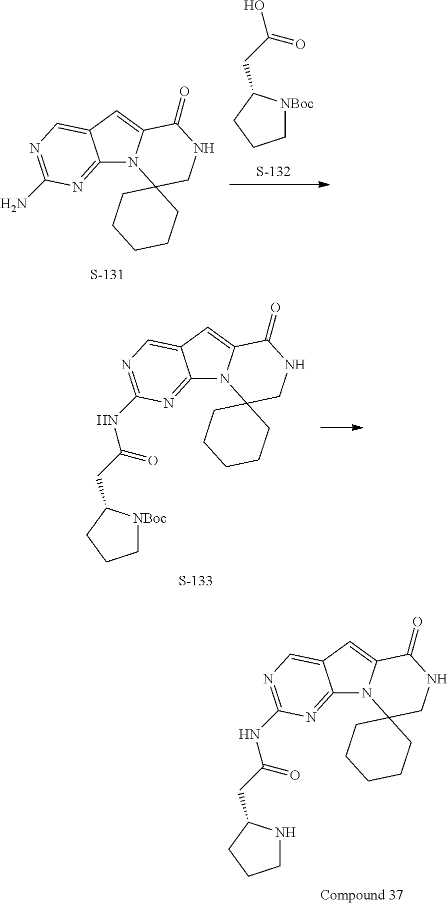

S-131 (100 mg) was coupled to S-132 using i-BuCO₂Cl in NMM and DMAc at 20° C. The reaction stirred for 12 hours. After workup, 120 mg of crude S-133 was obtained. Crude S-133 (120 mg) was converted to COMPOUND 37 using TFA in DCM. The reaction stirred at 20° C. for 2 hours. After purification, 11.3 mg of COMPOUND 37 was obtained and the structure was confirmed via ¹H-NMR, HPLC and LC-MS.

### Synthesis of 3-((6′-Oxo-7′,8′-dihydro-6′H-spiro[cyclohexane-1,9′-pyrazino[1′,2′:1,5]pyrrolo[2,3-d]pyrimidin]-2′-yl)amino)benzenesulfonamide (COMPOUND 42)

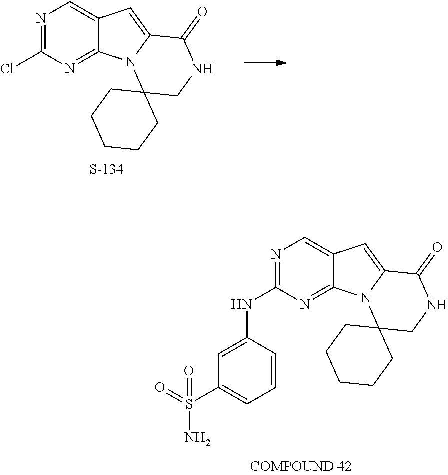 [SMILES: O=C1NCC2(CCCCC2)n2c1cc1cnc(Cl)nc12 ; NS(=O)(=O)c1cccc(Nc2ncc3cc4n(c3n2)C2(CCCCC2)CNC4=O)c1]

S-134 (30 mg) was converted to COMPOUND 42 using HCl in i-PrOH. The reaction was heated in a microwave reactor to 140° C. for 30 minutes. After purification and washing with DCM/MeOH (5/1), 6.8 mg of COMPOUND 52 was obtained and the structure was confirmed via ¹HNMR, LC-MS and HPLC. Alternatively, S-134 can be converted to COMPOUND 42 using Pd(OAc)₂/x-phos in the presence of CS₂CO₃ in dioxane at 100° C. for 4 hours. Alternatively, S-134 can be converted to COMPOUND 42 using TFA in i-PrOH. The reaction is refluxed for 48 hours. Alternatively, S-134 can be converted to COMPOUND 42 using 2 drops of concentrated. HCl in i-PrOH. The reaction is heated in a microwave reactor to 80° C. for 20 minutes. Alternatively, S-134 can be converted to COMPOUND 42 by the scheme below:

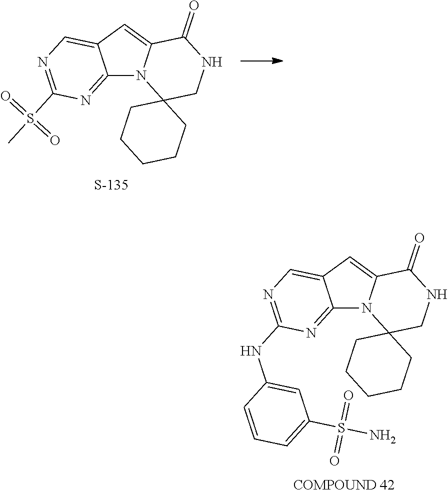 [SMILES: CS(=O)(=O)c1ncc2cc3n(c2n1)C1(CCCCC1)CNC3=O ; NS(=O)(=O)c1cccc(Nc2ncc3cc4n(c3n2)C2(CCCCC2)CNC4=O)c1]

S-135 (10 mg) was converted to COMPOUND 42 using Pd(OAc)₂/x-phos in the presence of Cs₂CO₃ in dioxane. The reaction was heated in a microwave reactor at 100° C. for 30 minutes. Alternatively, S-135 is converted to COMPOUND 42 using TFA in i-PrOH. The reaction is refluxed for 48 hours.

### Synthesis of (3-((6′-Oxo-7′,8′-dihydro-6′H-spiro[cyclohexane-1,9′-pyrazino[1′,2′:1,5]pyrrolo[2,3-d]pyrimidin]-2′-yl)amino)phenyl)methanesulfonamide (COMPOUND 43)

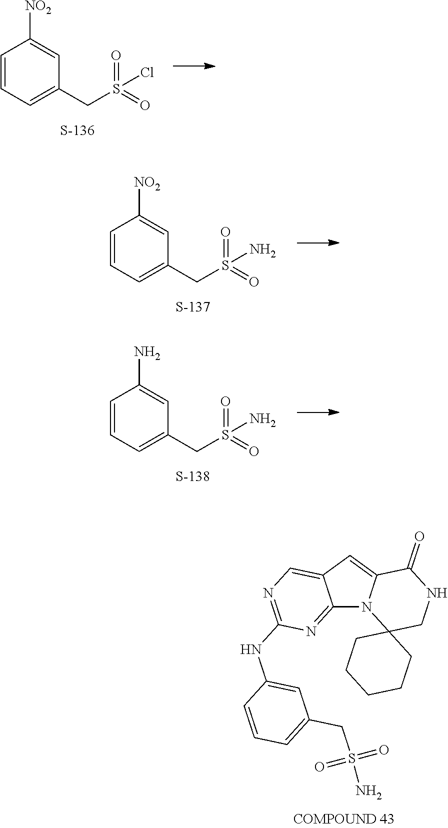 [SMILES: O=[N+]([O-])c1cccc(CS(=O)(=O)Cl)c1 ; NS(=O)(=O)Cc1cccc([N+](=O)[O-])c1 ; Nc1cccc(CS(N)(=O)=O)c1 ; NS(=O)(=O)Cc1cccc(Nc2ncc3cc4n(c3n2)C2(CCCCC2)CNC4=O)c1]

S-136 (850 mg) was converted to S-137 using NH₃.H₂O in THE at 20° C. for 12 hours. After purification, 620 mg of S-137 was obtained. S-137 (100 mg) was converted to S-138 using Pd/C in EtOH and AcOH at 30° C. for 12 hours. After purification, 60 mg of S-138 was obtained. S-138 was converted to COMPOUND 43 using HCl in EtOH. The reaction was heated in a microwave reactor at 70° C. for 30 minutes. After the purification, 4.1 mg of COMPOUND 43 was obtained and the structure was confirmed via ¹HNMR. Alternatively, S-138 is converted to COMPOUND 43 using TEA in EtOH. The reaction is heated in a microwave reactor at 140° C. for 15 minutes.

### Synthesis of 2′-(((1,4-Trans)-4-morpholinocyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 44)

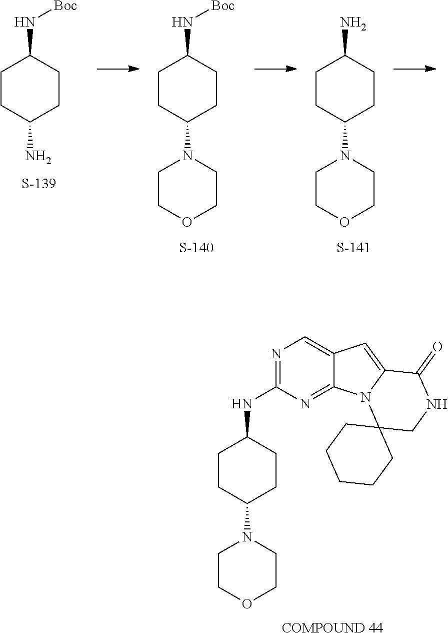 [SMILES: CC(C)(C)OC(=O)N[C@H]1CC[C@H](N)CC1 ; CC(C)(C)OC(=O)N[C@H]1CC[C@H](N2CCOCC2)CC1 ; N[C@H]1CC[C@H](N2CCOCC2)CC1 ; O=C1NCC2(CCCCC2)n2c1cc1cnc(N[C@H]3CC[C@H](N4CCOCC4)CC3)nc12]

S-139 (200 mg) was converted to S-140 using 3-oxa-1,5-dichloropentane in the presence of K₂CO₃ and heating the reaction at 100° C. in DMF overnight. After purification, 100 mg of S-140 was obtained. S-140 (100 mg) was converted to S-141 using TFA in DCM and stirring the reaction at room temperature for 1 hour. After purification, 50 mg of S-141 was obtained. S-141 (50 mg) is converted to COMPOUND 44 using TEA in DMAc and heating to 120° C. overnight.

### Synthesis of 2′-(((1,4-Trans)-4-(dimethylamino)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 45)

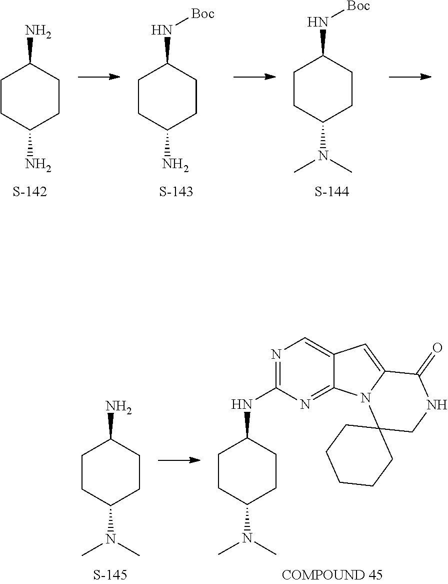

S-142 (1 g) was converted to S-143 using (Boc)₂O in the presence of NaHCO₃ in THE and stirring at room temperature overnight. After purification, 1.4 g of S-143 was obtained. S-143 (200 mg) was converted to S-144 using NaBH₃CN in the presence of HOAc in MeOH and stirring at room temperature overnight. The purification afforded 100 mg of S-144. S-144 (100 mg) was converted to S-145 using TFA in DCM and stirring the reaction at room temperature overnight. After workup, 60 mg of S-145 was obtained. S-145 (60 mg) was converted to COMPOUND 45 using TEA in DMAc and stirring at 120° C. overnight. After purification by preparative TLC, 15.4 mg of COMPOUND 45 was obtained and the structure was confirmed via ¹H-NMR and HPLC.

### Synthesis of 2′-(((1,4-Trans)-4-(piperidin-1-yl)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 46)

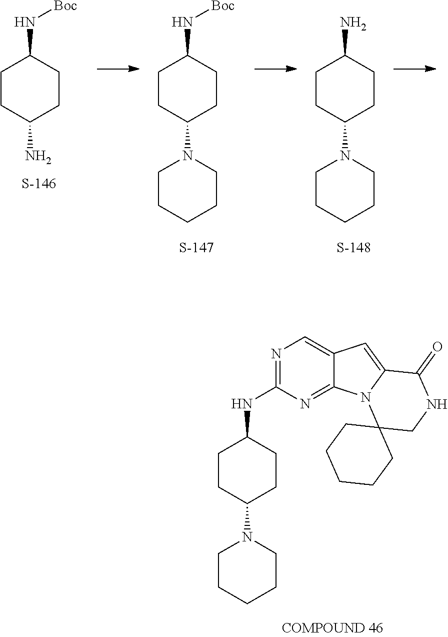 [SMILES: CC(C)(C)OC(=O)N[C@H]1CC[C@H](N)CC1 ; CC(C)(C)OC(=O)N[C@H]1CC[C@H](N2CCCCC2)CC1 ; N[C@H]1CC[C@H](N2CCCCC2)CC1 ; O=C1NCC2(CCCCC2)n2c1cc1cnc(N[C@H]3CC[C@H](N4CCCCC4)CC3)nc12]

S-146 (200 mg) was converted to S-147 using K₂CO₃ in EtOH at 80° C. overnight. After purification, 100 mg of S-147 was obtained. S-147 (100 mg) was converted to S-148 using TFA in DCM and stirring at room temperature for 1 hour. After purification, 50 mg of S-148 was obtained. S-148 (50 mg) was converted to COMPOUND 46 using TEA in DMAc for 120° C. overnight. The purification afforded 6.7 mg of COMPOUND 46.

### Synthesis of (S)—N-(Piperidin-3-yl)-8′H-spiro[cyclohexane-1,9′-pyrazino[1′,2′:1,5]pyrrolo[2,3-d]pyrimidin]-2′-amine (COMPOUND 50)

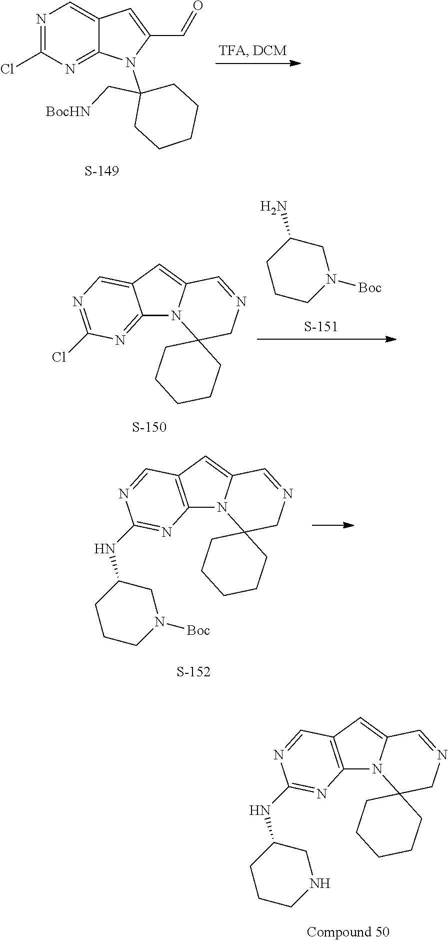

S-149 (1.0 g) was converted to S-150 using TFA in DCM and stirring at room temperature for 1 hour. After purification, 350 mg of S-150 was obtained. S-150 is coupled to S-151 using TEA in EtOH and refluxing for 48 hours to afford S-152.

### Synthesis of (R)—N-(Piperidin-3-yl)-8′H-spiro[cyclohexane-1,9′-pyrazino[1′,2′:1,5]pyrrolo[2,3-d]pyrimidin]-2′-amine (COMPOUND 51)

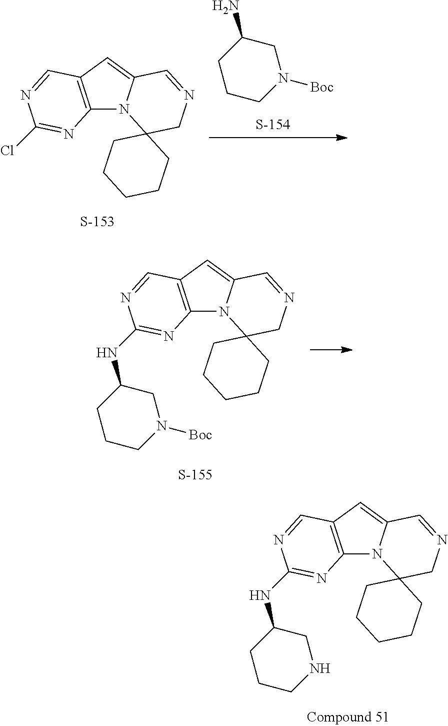

S-153 (25 mg) was coupled to S-154 to afford S-155 using TEA in EtOH and refluxing for 48 hours. After purification, 3.2 mg of S-155 was obtained. S-155 (3.2 mg) is converted to COMPOUND 51 using TFA in DCM and stirring at room temperature for 2 hours.

### Synthesis of N-(Tetrahydro-2H-pyran-4-yl)-8′H-spiro[cyclohexane-1,9′-pyrazino[1′,2′:1,5]pyrrolo[2,3-d]pyrimidin]-2′-amine (COMPOUND 52)

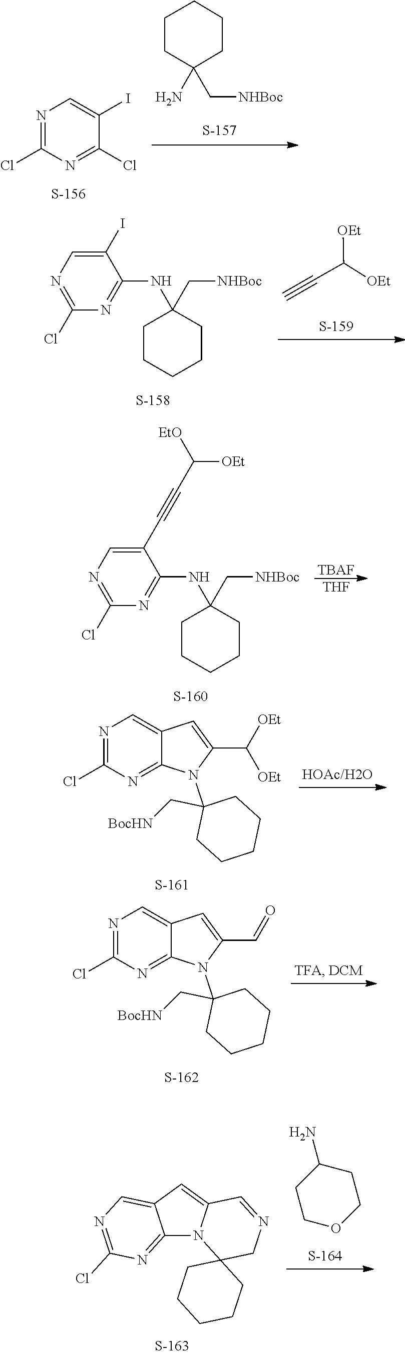

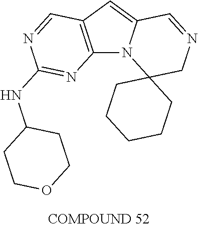 [SMILES: C1=NCC2(CCCCC2)n2c1cc1cnc(NC3CCOCC3)nc12]

S-156 (20 g) was coupled to S-157 in the presence of K₂CO₃ in DMAc at 80° C. overnight to afford S-158. After purification, 25 g of crude S-158 was obtained. S-158 (15 g) was converted to S-160 in the presence of S-159 using (PPh₃)₂PdCl₂, CuI and TEA in THE at 40° C. for 4 hours. After purification, 9.3 g of crude S-160 was obtained. S-160 (9.3 g) was converted to S-161 using TBAF in THE at 60° C. for 4 hours. S-161 (5.6 g) was converted to S-162 using HOAc in THF/H₂O at 60° C. for 6 hours. After purification, 3.5 g of S-162 was obtained. S-162 (1.2 g) was converted to S-163 using TFA in DCM and stirring at room temperature for 1 hour. After purification, 410 mg of S-163 was obtained. S-163 (25 mg) was coupled to S-164 to afford COMPOUND 52 using TEA in EtOH and refluxing for 12 hours. After purification by preparative TLC, 5.8 mg of COMPOUND 52 was obtained and the structure was confirmed via H-NMR, HPLC and LC-MS.

### Synthesis of N-((1s,4s)-4-Methylcyclohexyl)-8′H-spiro[cyclohexane-1,9′-pyrazino[1′,2′:1,5]pyrrolo[2,3-d]pyrimidin]-2′-amine (COMPOUND 53)

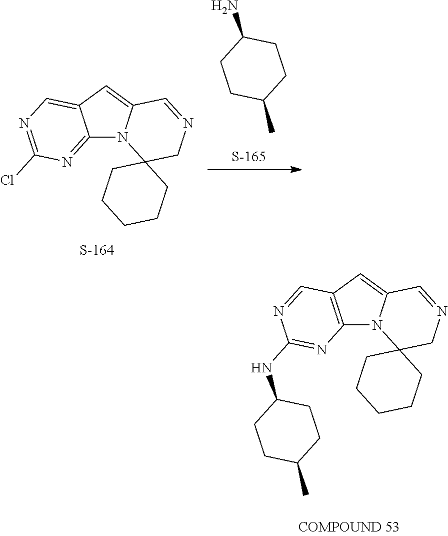

S-164 (30 mg) is coupled to S-165 using TEA in EtOH and refluxing for 48 hours to afford COMPOUND 53.

### Synthesis of N-((1r,4r)-4-Methylcyclohexyl)-8′H-spiro[cyclohexane-1,9′-pyrazino[1′,2′:1,5]pyrrolo[2,3-d]pyrimidin]-2′-amine (COMPOUND 54)

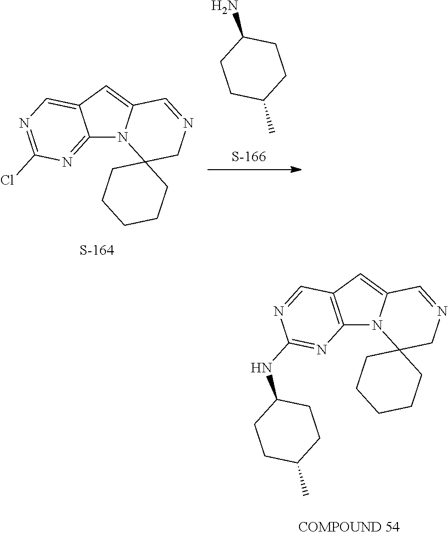

S-164 (25 mg) is coupled to S-166 using TEA in EtOH and refluxing at 100° C. for 48 hours to afford COMPOUND 54.

### Synthesis of (1s,4s)-N1-(8′H-Spiro[cyclohexane-1,9′-pyrazino[1′,2′:1,5]pyrrolo[2,3-d]pyrimidin]-2′-yl)cyclohexane-1,4-diamine (COMPOUND 55)

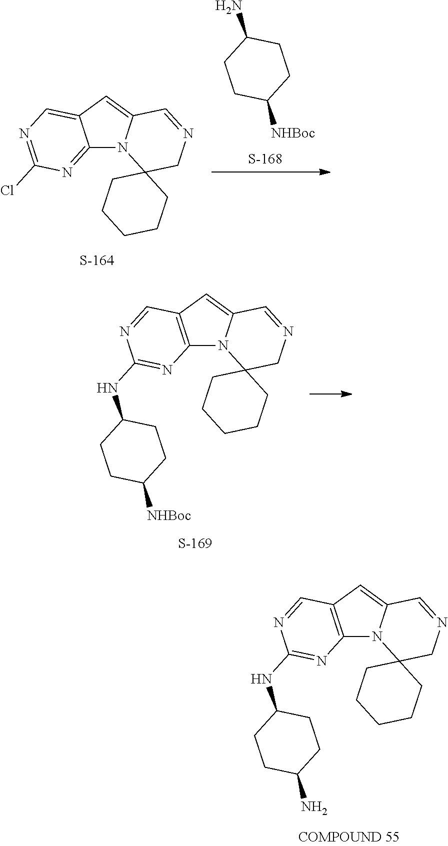 [SMILES: Clc1ncc2cc3n(c2n1)C1(CCCCC1)CN=C3 ; CC(C)(C)OC(=O)N[C@H]1CC[C@@H](N)CC1 ; CC(C)(C)OC(=O)N[C@H]1CC[C@@H](Nc2ncc3cc4n(c3n2)C2(CCCCC2)CN=C4)CC1 ; N[C@H]1CC[C@@H](Nc2ncc3cc4n(c3n2)C2(CCCCC2)CN=C4)CC1]

S-167 S-169 COMPOUND 55 S-167 (25 mg) was coupled to S-168 using TEA in EtOH and refluxing for 48 hours to afford S-169. After purification, 5.3 mg of S-169 was obtained. S-169 is converted to COMPOUND 55 using TFA in DCM and stirring at room temperature for 2 hours.

### Synthesis of 2′-(((1r,4r)-4-(4-Cyclopropylpiperazin-1-yl)cyclohexyl)amino)-7′,8′-dihydrospiro[cyclohexane-1,9′-pyrido[3′,4′:4,5]cyclopenta[1,2-d]pyrimidin]-6′(9a¹H)-one (COMPOUND 58)

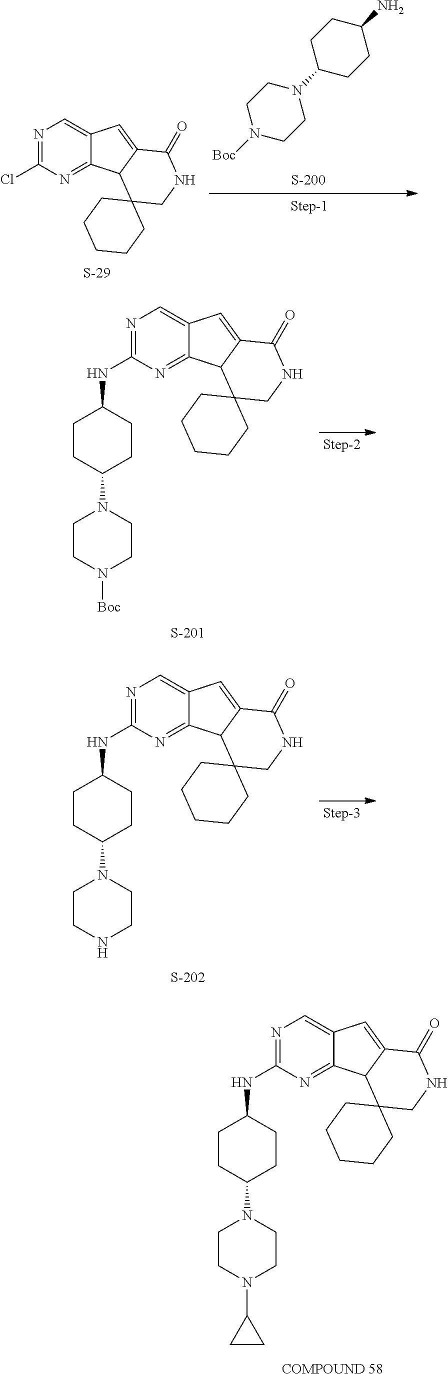

In Step 1, 100 mg of S-29 was reacted with S-200 to provide S-201. In Step 2, 50 mg of S-201 was reacted with trifluoroacetic acid to provide 45 mg of S-202. In Step 3, 30 mg of S-202 was reacted with the (1-ethoxycyclopropoxy)trimethylsilane in the presence of acetic acid and NaBH₃CN in methanol overnight to provide COMPOUND 58.

### Synthesis of 2′-(((1r,4r)-4-(4-Cyclopentylpiperazin-1-yl)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 59)

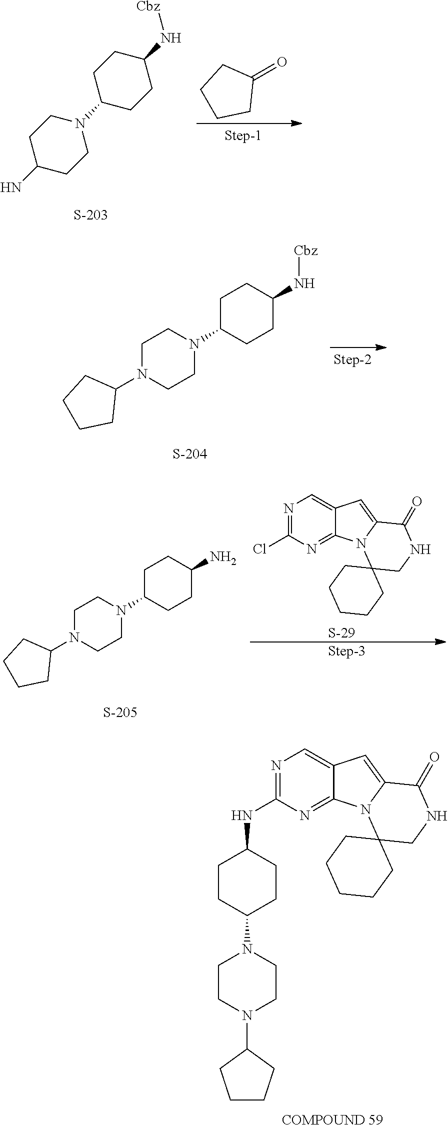

In Step 1, 300 mg of S-203 was reacted with cyclohexanone in the presence of sodium triacetoxyborohydride and acetic acid in DCM to provide 246 mg of S-204. In Step 2, 246 mg of S-204 was hydrogenated with Palladium on carbon to provide 190 mg of S-205. In Step 3, 50 mg of S-205 was reacted with the S-29 in the presence of sodium bicarbonate at 125 C in DMAc overnight to provide COMPOUND 59.

### Synthesis of 2′-(((1r,4r)-4-(4-(4-Fluorophenyl)piperazin-1-yl)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 60)

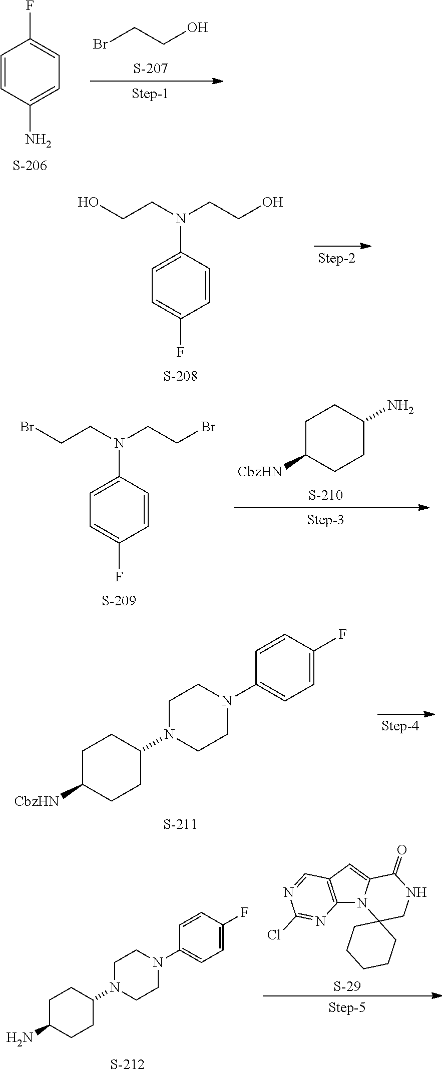

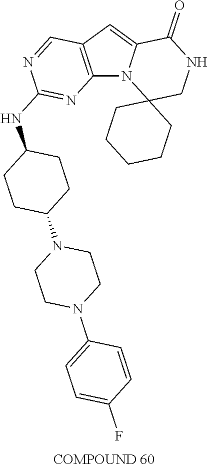 [SMILES: O=C1NCC2(CCCCC2)n2c1cc1cnc(N[C@H]3CC[C@H](N4CCN(c5ccc(F)cc5)CC4)CC3)nc12]

In Step 1, 3 g of S-206 was reacted with S-207 in the presence of calcium carbonate in water at 100 C overnight to provide 5 5 g of S-208 after purification. In Step 2, 2.7 g of S-208 was reacted with PBr₃ in diethyl ether at 0 C to provide 1.8 g of S-209. In Step 3, 1.6 g of S-209 was reacted with S-210 in the presence of DIEA at 100 C in DMF overnight to provide 455 mg of S-211. In Step 4, 455 mg of S-211 was hydrogenated with palladium on carbon in methanol and ethylacetate to afford 206 mg of S-212. In Step 5, 50 mg of S-212 was reacted with S-29 at 140 C in NMP to afford 4.6 mg of COMPOUND 60.

### Synthesis of 2′-(((1r,4r)-4-(4-(Cyclohexylmethyl)piperazin-1-yl)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 61)

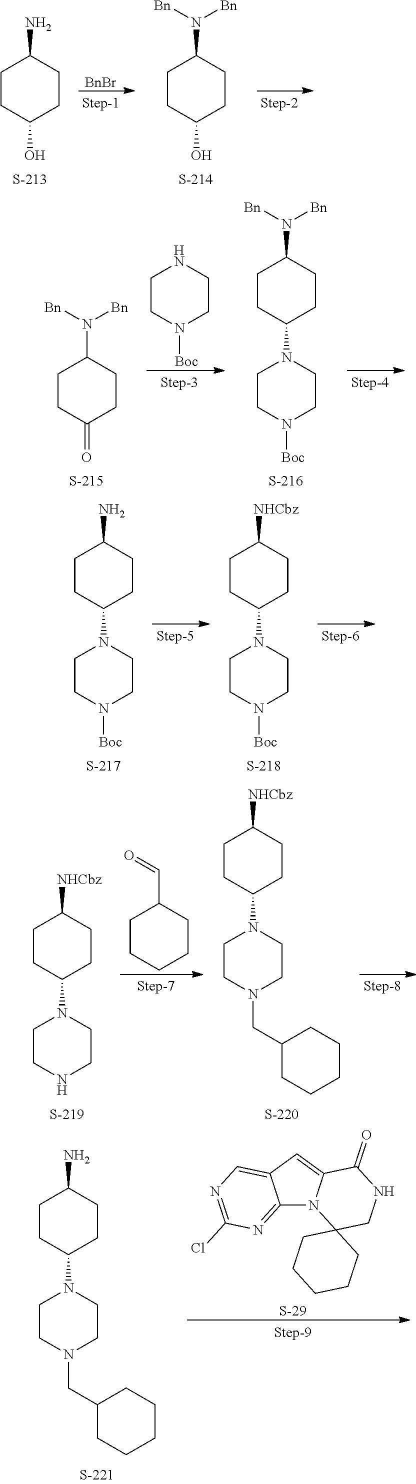

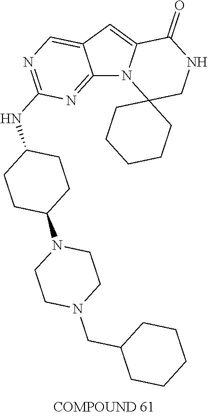 [SMILES: O=C1NCC2(CCCCC2)n2c1cc1cnc(N[C@H]3CC[C@H](N4CCN(CC5CCCCC5)CC4)CC3)nc12]

In Step 1, 30 g of S-213 was reacted with benzyl bromide in the presence of potassium carbonate in DMF overnight to provide 60 g of S-214. In Step 2, 56 g of S-214 was reacted with (COCl)₂(1.5 eq) in DMSO in the presence of triethyl amine (3.2 eq) to provide 43.8 g of S-215 after purification. In Step 3, 1 g of S-215 was reacted with mon-Boc protected piperazine (1.2 eq) in the presence of TsOH (0.05 eq) at 120 C in Toluene to provide 780 mg of S-216. In Step 4, 700 mg of S-216 was hydrogenated with palladium on carbon in isopropyl alcohol with acetic acid at 40 C to afford 500 mg of S-217. In Step 5, 500 mg of S-217 was reacted with CbzCl in DCM to afford 100 mg of S-218. In Step 6, 100 mg of S-218 is deprotected with trifluoroacetic acid to afford 80 mg of S-219. In Step 7, 80 mg of S-219 is converted to 50 mg of S-220 by reductive amination. In Step 8, 50 mg of S-220 is converted to 30 mg of S-221 by hydrogenation with palladium on carbon. In Step 9, 30 mg of S-221 was reacted with S-29 at 125 C in the presence of triethylamine in DMAc to afford 2.5 mg of COMPOUND 61.

### Synthesis of 2′-(((1r,4r)-4-(4-(Cyclopropanecarbonyl)piperazin-1-yl)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 62)

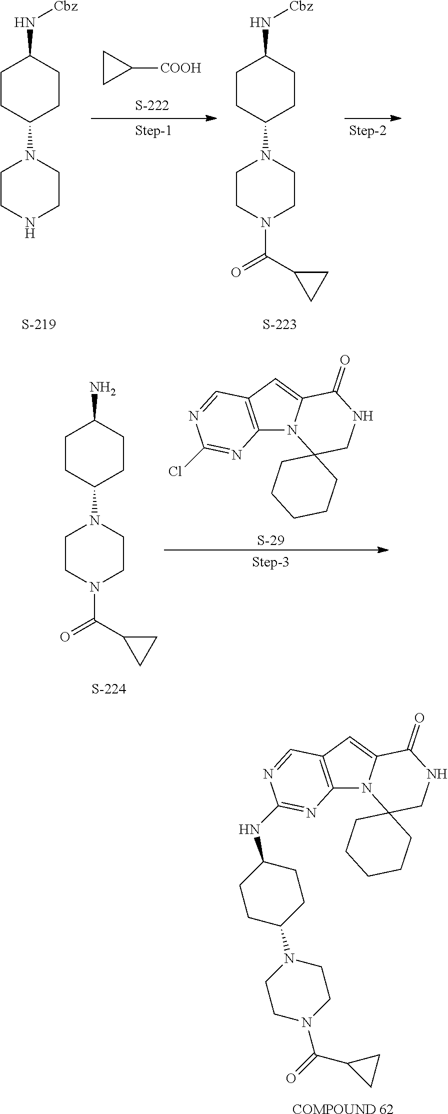

In Step 1, 600 mg of S-219 was reacted with S-222, EDCI, HOBT (0.5 eq), and trimethylamine (2 eq) in DCM overnight to provide 500 mg of S-223. In Step 2, 100 mg of S-223 was hydrogenated with palladium on carbon (15%) in methanol for 3 hours to provide 50 mg of S-224 after purification. In Step 3, 50 mg of S-224 was reacted with S-29 at 120 C in the presence of sodium bicarbonate (10 eq) in DMAc to afford 3.7 mg of COMPOUND 62.

### Synthesis of 2′-(((1r,4r)-4-((4-Fluorobenzyl)amino)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 69)

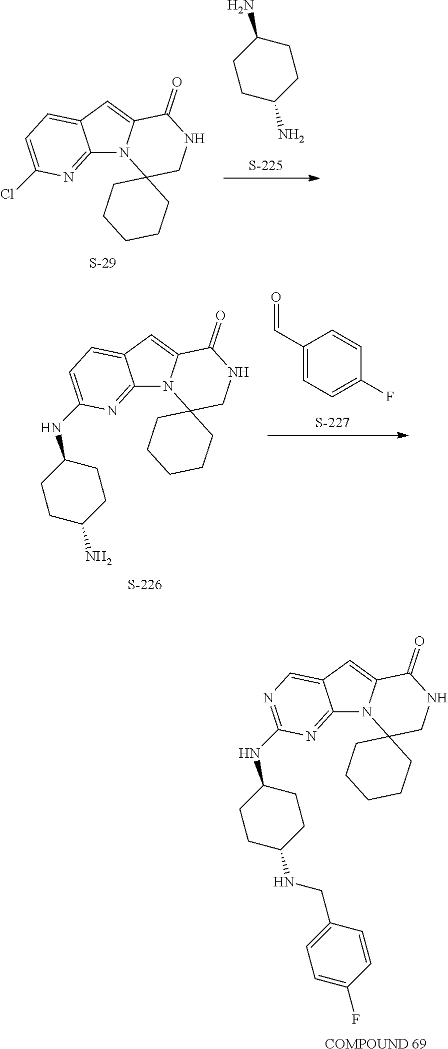

In Step 1, 425 mg of S-29 was reacted with S-225 in the presence of sodium bicarbonate in DMAc at 110 C overnight to provide 115 mg of S-226. In Step 2, 50 mg of S-226 was reacted with S-227 with sodium triacetoxy borohydride and triethyl amine in DCM and methanol to afford 12.4 mg of COMPOUND 69.

### Synthesis of 2′-(((r,4r)-4-((2,4-Difluorobenzyl)amino)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 70)

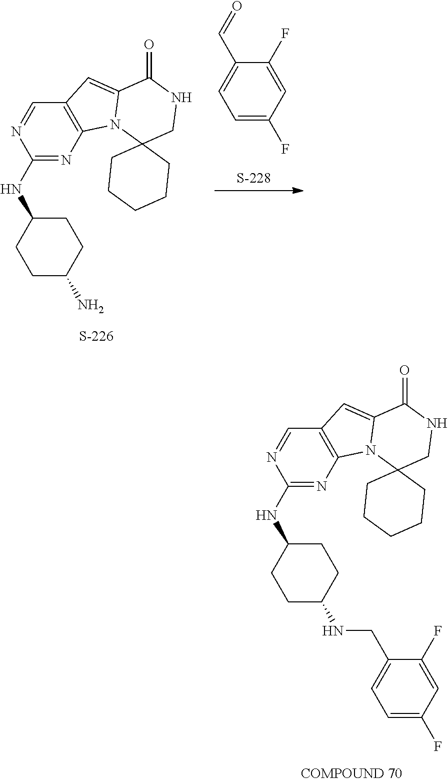

In Step 1, 20 mg of S-226 was reacted with S-228 with sodium triacetoxy borohydride and triethyl amine in methanol to afford 8.2 mg of COMPOUND 70.

### Synthesis of 2′-(((1r,4r)-4-((1-(2-Fluorophenyl)ethyl)amino)cyclohexyl)amino)-7′,8′-dihydro-6′H-spiro[cyclohexane-1,9′-pyrazino[1′,2′:1,5]pyrrolo[2,3-d]pyrimidin]-6′-one (COMPOUND 71)

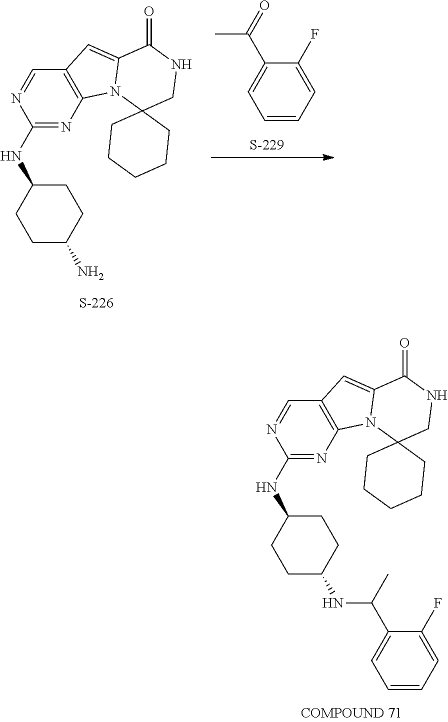

In Step 1, 20 mg of S-226 was reacted with S-229 with sodium triacetoxy borohydride and formic acid in methanol to afford COMPOUND 71.

### Synthesis of 2-Fluoro-N-((1r,4r)-4-((6′-oxo-7′,8′-dihydro-6′H-spiro[cyclohexane-1,9′-pyrazino[1′,2′:1,5]pyrrolo[2,3-d]pyrimidin]-2′-yl)amino)cyclohexyl)benzamide (COMPOUND 72)

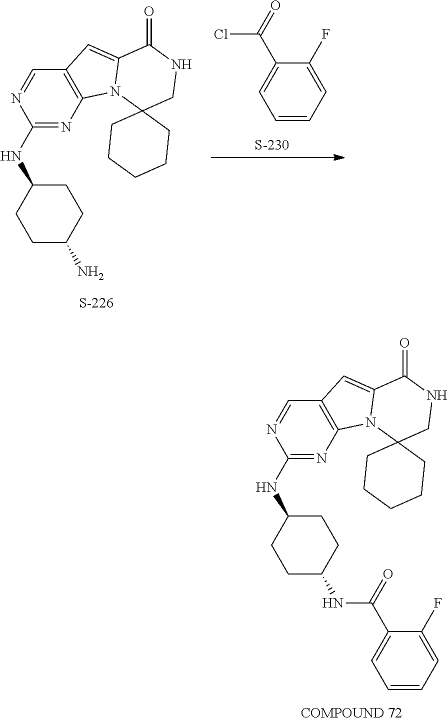 [SMILES: N[C@H]1CC[C@H](Nc2ncc3cc4n(c3n2)C2(CCCCC2)CNC4=O)CC1 ; O=C(Cl)c1ccccc1F ; O=C(N[C@H]1CC[C@H](Nc2ncc3cc4n(c3n2)C2(CCCCC2)CNC4=O)CC1)c1ccccc1F]

In Step 1, 18 mg of S-226 was reacted with S-230 in the presence of pyridine in DCM to afford 4.4 mg of COMPOUND 72.

### Example 3. Representative Compounds of the Present Invention

Table 1, Table 2, and Table 3 provide non-limiting examples of compounds of the present invention which can be made according to the procedures above.

TABLE 1

| Entry | Compound Structure | Name |
|---|---|---|
| 1 | 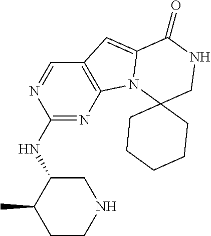 [SMILES: C[C@@H]1CCNC[C@H]1Nc1ncc2cc3n(c2n1)C1(CCCCC1)CNC3=O] | 2′-(((3S,4R)-4-methylpiperidin-3- yl)amino)-7′,8′-dihydro-6′H- spiro[cyclohexane-1,9′- pyrazino[1′,2′:1,5]pyrrolo[2,3- d]pyrimidin]-6′-one |
| 2 | 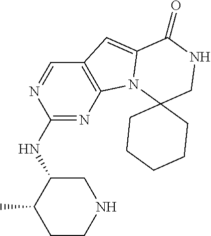 [SMILES: C[C@H]1CCNC[C@H]1Nc1ncc2cc3n(c2n1)C1(CCCCC1)CNC3=O] | 2′-(((3S,4S)-4-methylpiperidin-3- yl)amino)-7′,8′-dihydro-6′H- spiro[cyclohexane-1,9′- pyrazino[1′,2′:1,5]pyrrolo[2,3- d]pyrimidin]-6′-one |
| 3 | 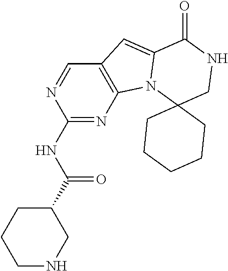 [SMILES: O=C1NCC2(CCCCC2)n2c1cc1cnc(NC(=O)[C@H]3CCCNC3)nc12] | (S)-N-(6′-oxo-7′,8′-dihydro-6′H- spiro[cyclohexane-1,9′- pyrazino[1′,2′:1,5]pyrrolo[2,3- d]pyrimidin]-2′-yl)piperidine-3- carboxamide |
| 4 | 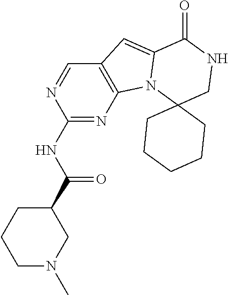 [SMILES: CN1CCC[C@@H](C(=O)Nc2ncc3cc4n(c3n2)C2(CCCCC2)CNC4=O)C1] | (R)-1-methyl-N-(6′-oxo-7′,8′-dihydro-6′H- spiro[cyclohexane-1,9′- pyrazino[1′,2′:1,5]pyrrolo[2,3- d]pyrimidin]-2′-yl)piperidine-3- carboxamide |
| 5 | 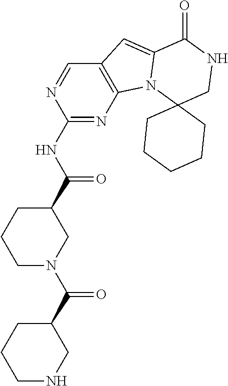 [SMILES: O=C1NCC2(CCCCC2)n2c1cc1cnc(NC(=O)[C@@H]3CCCN(C(=O)[C@@H]4CCCNC4)C3)nc12] | (R)-N-(6′-oxo-7′,8′-dihydro-6′H- spiro[cyclohexane-1,9′- pyrazino[1′,2′:1,5]pyrrolo[2,3- d]pyrimidin]-2′-yl)-1-((R)-piperidine-3- carbonyl)piperidine-3-carboxamide |
| 6 | 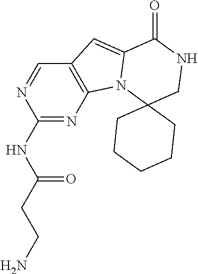 [SMILES: NCCC(=O)Nc1ncc2cc3n(c2n1)C1(CCCCC1)CNC3=O] | 3-amino-N-(6′-oxo-7′,8′-dihydro-6′H- spiro[cyclohexane-1,9′- pyrazino[1′,2′:1,5]pyrrolo[2,3- d]pyrimidin]-2′-yl)propanamide |
| 7 | 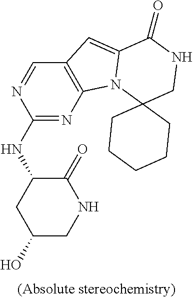 [SMILES: O=C1NCC2(CCCCC2)n2c1cc1cnc(N[C@H]3C[C@@H](O)CNC3=O)nc12] | 2′-(((3S,5R)-5-hydroxy-2-oxopiperidin-3- yl)amino)-7′,8′-dihydro-6′H- spiro[cyclohexane-1,9′- pyrazino[1′,2′:1,5]pyrrolo[2,3- d]pyrimidin]-6′-one |
| 8 | 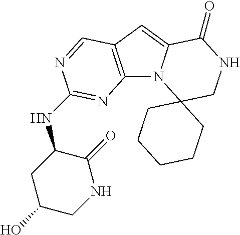 [SMILES: O=C1NCC2(CCCCC2)n2c1cc1cnc(N[C@@H]3C[C@@H](O)CNC3=O)nc12] | 2′-(((3R,5R)-5-hydroxy-2-oxopiperidin-3- yl)amino)-7′,8′-dihydro-6′H- spiro[cyclohexane-1,9′- pyrazino[1′,2′:1,5]pyrrolo[2,3- d]pyrimidin]-6′-one |
| 9 | 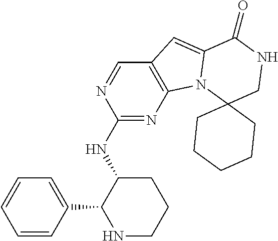 [SMILES: O=C1NCC2(CCCCC2)n2c1cc1cnc(N[C@@H]3CCCN[C@@H]3c3ccccc3)nc12] | 2′-(((2R,3R)-2-phenylpiperidin-3- yl)amino)-7′,8′-dihydro-6′H- spiro[cyclohexane-1,9′- pyrazino[1′,2′:1,5]pyrrolo[2,3- d]pyrimidin]-6'-one |
| 10 | 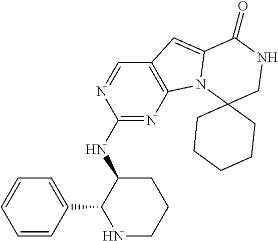 [SMILES: O=C1NCC2(CCCCC2)n2c1cc1cnc(N[C@H]3CCCN[C@@H]3c3ccccc3)nc12] | 2′-(((2R,3S)-2-phenylpiperidin-3- yl)amino)-7′,8′-dihydro-6′H- spiro[cyclohexane-1,9′- pyrazino[1′,2′:1,5]pyrrolo[2,3- d]pyrimidin]-6′-one |
| 11 | 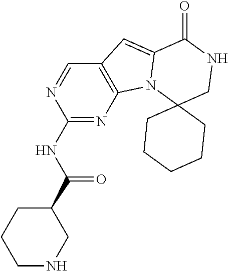 [SMILES: O=C1NCC2(CCCCC2)n2c1cc1cnc(NC(=O)[C@@H]3CCCNC3)nc12] | (R)-N-(6′-oxo-7′,8′-dihydro-6′H- spiro[cyclohexane-1,9′- pyrazino[1′,2′:1,5]pyrrolo[2,3- d]pyrimidin]-2′-yl)piperidine-3- carboxamide |
| 12 | 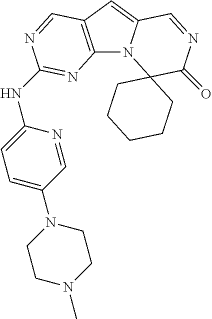 [SMILES: CN1CCN(c2ccc(Nc3ncc4cc5n(c4n3)C3(CCCCC3)C(=O)N=C5)nc2)CC1] | 2′-((5-(4-methylpiperazin-1-yl)pyridin-2- yl)amino)-8′H-spiro[cyclohexane-1,9′- pyrazino[1′,2′:1,5]pyrrolo[2,3- d]pyrimidin]-8′-one |
| 13 | 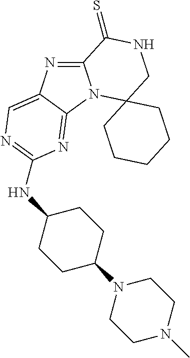 [SMILES: CN1CCN([C@H]2CC[C@@H](Nc3ncc4nc5n(c4n3)C3(CCCCC3)CNC5=S)CC2)CC1] | 2′-(((1s,4s)-4-(4-methylpiperazin-1- yl)cyclohexyl)amino)-7′,8′-dihydro-6′H- spiro[cyclohexane-1,9′-pyrazino[1,2- e]purine]-6-thione |
| 14 | 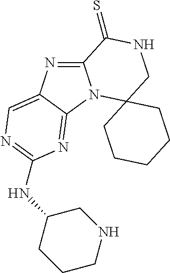 [SMILES: S=C1NCC2(CCCCC2)n2c1nc1cnc(N[C@H]3CCCNC3)nc12] | (S)-2′-(piperidin-3-ylamino)-7′,8′-dihydro- 6′H-spiro[cyclohexane-1,9′-pyrazino[1,2- e]purine]-6′-thione |
| 15 | 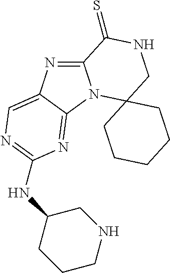 [SMILES: S=C1NCC2(CCCCC2)n2c1nc1cnc(N[C@@H]3CCCNC3)nc12] | (R)-2′-(piperidin-3-ylamino)-7′,8′-dihydro- 6′H-spiro[cyclohexane-1,9′-pyrazino[1,2- e]purine]-6′-thione |
| 16 | 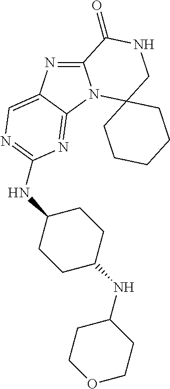 | 2′-(((1r,4r)-4-((tetrahydro-2H-pyran-4- yl)amino)cyclohexyl)amino)-7′,8′- dihydro-6′H-spiro[cyclohexane-1,9′- pyrazino[1′,2′:1,5]pyrrolo[2,3- d]pyrimidin]-6′-one |
| 17 | 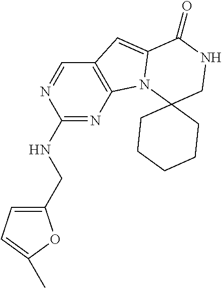 [SMILES: Cc1ccc(CNc2ncc3cc4n(c3n2)C2(CCCCC2)CNC4=O)o1] | 2′-(((5-methylfuran-2-yl)methyl)amino)- 7′,8′-dihydro-6′H-spiro[cyclohexane-1,9′- pyrazino[1′,2′:1,5]pyrrolo[2,3- d]pyrimidin]-6′-one |
| 18 | 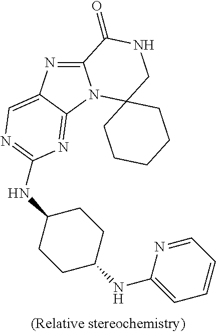 [SMILES: O=C1NCC2(CCCCC2)n2c1nc1cnc(N[C@H]3CC[C@H](Nc4ccccn4)CC3)nc12] | 2′-(((1,4-trans)-4-(pyridin-2- ylamino)cyclohexyl)amino)-7′,8′-dihydro- 6′H-spiro[cyclohexane-1,9′- pyrazino[1′,2′:1,5]pyrrolo[2,3- d]pyrimidin]-6′-one |
| 19 | 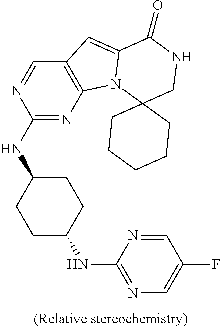 [SMILES: O=C1NCC2(CCCCC2)n2c1cc1cnc(N[C@H]3CC[C@H](Nc4ncc(F)cn4)CC3)nc12] | 2′-(((1,4-trans)-4-((5-fluoropyrimidin-2- yl)amino)cyclohexyl)-12-azaneyl)-7′,8′- dihydro-6′H-spiro[cyclohexane-1,9′- pyrazino[1′,2′:1,5]pyrrolo[2,3- d]pyrimidin]-6′-one |
| 20 | 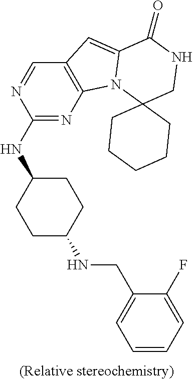 [SMILES: O=C1NCC2(CCCCC2)n2c1cc1cnc(N[C@H]3CC[C@H](NCc4ccccc4F)CC3)nc12] | 2′-(((1,4-trans)-4-((2- fluorobenzyl)amino)cyclohexyl)amino)- 7′,8′-dihydro-6′H-spiro[cyclohexane-1,9′- pyrazino[1′,2′:1,5]pyrrolo[2,3- d]pyrimidin]-6′-one |
| 21 | 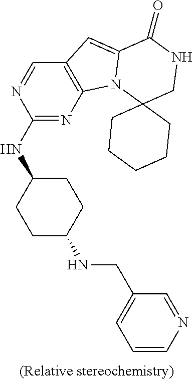 [SMILES: O=C1NCC2(CCCCC2)n2c1cc1cnc(N[C@H]3CC[C@H](NCc4cccnc4)CC3)nc12] | 2′-(((1,4-trans)-4-((pyridin-3- ylmethyl)amino)cyclohexyl)amino)-7′,8′- dihydro-6′H-spiro[cyclohexane-1,9′- pyrazino[1′,2′:1,5]pyrrolo[2,3- d]pyrimidin]-6′-one |
| 22 | 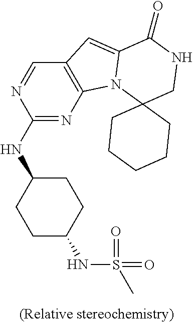 [SMILES: CS(=O)(=O)N[C@H]1CC[C@H](Nc2ncc3cc4n(c3n2)C2(CCCCC2)CNC4=O)CC1] | N-((1,4-trans)-4-((6′-oxo-7′,8′-dihydro- 6′H-spiro[cyclohexane-1,9′- pyrazino[1′,2′:1,5]pyrrolo[2,3- d]pyrimidin]-2′- yl)amino)cyclohexyl)methanesulfonamide |
| 23 | 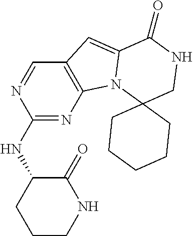 [SMILES: O=C1NCC2(CCCCC2)n2c1cc1cnc(N[C@H]3CCCNC3=O)nc12] | (S)-2′-((2-oxopiperidin-3-yl)amino)-7′,8′- dihydro-6′H-spiro[cyclohexane-1,9′- pyrazino[1′,2′:1,5]pyrrolo[2,3- d]pyrimidin]-6′-one |
| 24 | 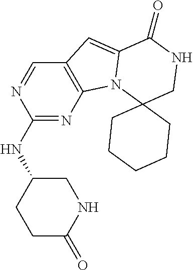 [SMILES: O=C1CC[C@H](Nc2ncc3cc4n(c3n2)C2(CCCCC2)CNC4=O)CN1] | (S)-2′-((6-oxopiperidin-3-yl)amino)-7′,8′- dihydro-6′H-spiro[cyclohexane-1,9′- pyrazino[1′,2′:1,5]pyrrolo[2,3- d]pyrimidin]-6′-one |
| 25 | 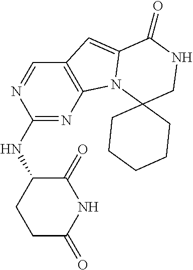 [SMILES: O=C1CC[C@H](Nc2ncc3cc4n(c3n2)C2(CCCCC2)CNC4=O)C(=O)N1] | (S)-3-((6′-oxo-7′,8′-dihydro-6′H- spiro[cyclohexane-1,9′- pyrazino[1′,2′:1,5]pyrrolo[2,3- d]pyrimidin]-2′-yl)amino)piperidine-2,6- dione |
| 26 | 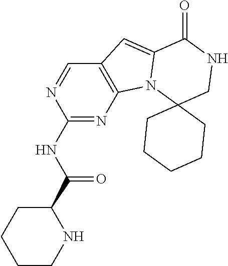 [SMILES: O=C1NCC2(CCCCC2)n2c1cc1cnc(NC(=O)[C@@H]3CCCCN3)nc12] | (S)-N-(6′-oxo-7′,8′-dihydro-6′H- spiro[cyclohexane-1,9′- pyrazino[1′,2′:1,5]pyrrolo[2,3- d]pyrimidin]-2′-yl)piperidine-2- carboxamide |
| 27 | 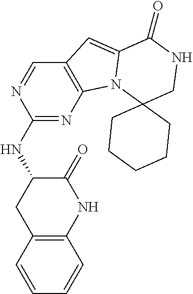 [SMILES: O=C1NCC2(CCCCC2)n2c1cc1cnc(N[C@H]3Cc4ccccc4NC3=O)nc12] | (S)-2′-((2-oxo-1,2,3,4-tetrahydroquinolin- 3-yl)amino)-7′,8′-dihydro-6′H- spiro[cyclohexane-1,9′- pyrazino[1′,2′:1,5]pyrrolo[2,3- d]pyrimidin]-6′-one |
| 28 | 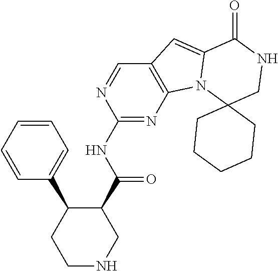 [SMILES: O=C1NCC2(CCCCC2)n2c1cc1cnc(NC(=O)[C@H]3CNCC[C@H]3c3ccccc3)nc12] | (3R,4R)-N-(6′-oxo-7′,8′-dihydro-6′H- spiro[cyclohexane-1,9′- pyrazino[1′,2′:1,5]pyrrolo[2,3- d]pyrimidin]-2′-yl)-4-phenylpiperidine-3- carboxamide |
| 29 | 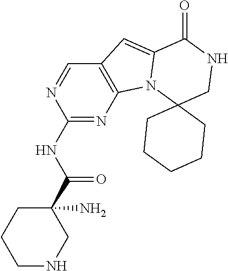 [SMILES: N[C@@]1(C(=O)Nc2ncc3cc4n(c3n2)C2(CCCCC2)CNC4=O)CCCNC1] | (S)-3-amino-N-(6′-oxo-7′,8′-dihydro-6′H- spiro[cyclohexane-1,9′- pyrazino[1′,2′:1,5]pyrrolo[2,3- d]pyrimidin]-2′-yl)piperidine-3- carboxamide |
| 30 | 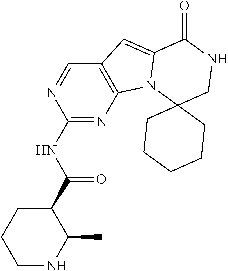 [SMILES: C[C@H]1NCCC[C@H]1C(=O)Nc1ncc2cc3n(c2n1)C1(CCCCC1)CNC3=O] | (2R,3R)-2-methyl-N-(6′-oxo-7′,8′-dihydro- 6′H-spiro[cyclohexane-1,9′- pyrazino[1′,2′:1,5]pyrrolo[2,3- d]pyrimidin]-2′-yl)piperidine-3- carboxamide |
| 31 | 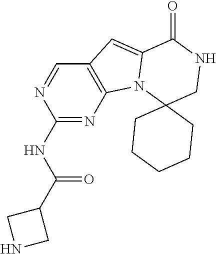 [SMILES: O=C1NCC2(CCCCC2)n2c1cc1cnc(NC(=O)C3CNC3)nc12] | N-(6′-oxo-7′,8′-dihydro-6′H- spiro[cyclohexane-1,9′- pyrazino[1′,2′:1,5]pyrrolo[2,3- d]pyrimidin]-2′-yl)azetidine-3- carboxamide |
| 32 | 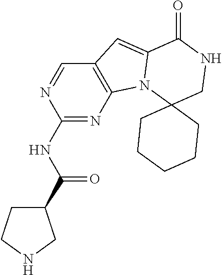 [SMILES: O=C1NCC2(CCCCC2)n2c1cc1cnc(NC(=O)[C@@H]3CCNC3)nc12] | (R)-N-(6′-oxo-7′,8′-dihydro-6′H- spiro[cyclohexane-1,9′- pyrazino[1′,2′:1,5]pyrrolo[2,3- d]pyrimidin]-2′-yl)pyrrolidine-3- carboxamide |
| 33 | 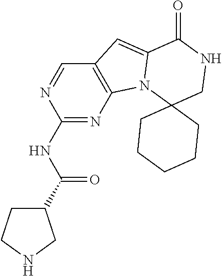 | (S)-N-(6′-oxo-7′,8′-dihydro-6′H- spiro[cyclohexane-1,9′- pyrazino[1′,2′:1,5]pyrrolo[2,3- d]pyrimidin]-2′-yl)pyrrolidine-3- carboxamide |
| 34 | 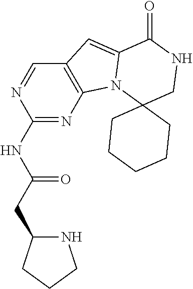 [SMILES: O=C(C[C@@H]1CCCN1)Nc1ncc2cc3n(c2n1)C1(CCCCC1)CNC3=O] | (S)-N-(6′-oxo-7′,8′-dihydro-6′H- spiro[cyclohexane-1,9′- pyrazino[1′,2′:1,5]pyrrolo[2,3- d]pyrimidin]-2′-yl)-2-(pyrrolidin-2- yl)acetamide |
| 35 | 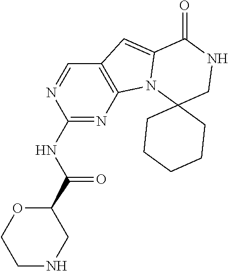 [SMILES: O=C1NCC2(CCCCC2)n2c1cc1cnc(NC(=O)[C@H]3CNCCO3)nc12] | (R)-N-(6′-oxo-7′,8′-dihydro-6′H- spiro[cyclohexane-1,9′- pyrazino[1′,2′:1,5]pyrrolo[2,3- d]pyrimidin]-2′-yl)morpholine-2- carboxamide |
| 36 | 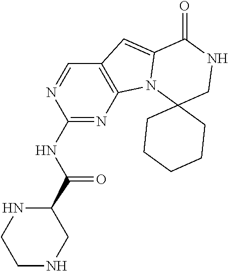 [SMILES: O=C1NCC2(CCCCC2)n2c1cc1cnc(NC(=O)[C@H]3CNCCN3)nc12] | (R)-N-(6′-oxo-7′,8′-dihydro-6′H- spiro[cyclohexane-1,9′- pyrazino[1′,2′:1,5]pyrrolo[2,3- d]pyrimidin]-2′-yl)piperazine-2- carboxamide |
| 37 | 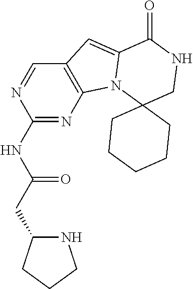 [SMILES: O=C(C[C@H]1CCCN1)Nc1ncc2cc3n(c2n1)C1(CCCCC1)CNC3=O] | (R)-N-(6′-oxo-7′,8′-dihydro-6′H- spiro[cyclohexane-1,9′- pyrazino[1′,2′:1,5]pyrrolo[2,3- d]pyrimidin]-2′-yl)-2-(pyrrolidin-2- yl)acetamide |

TABLE 2

| Entry | Compound Structure | Name |
|---|---|---|
| 38 | 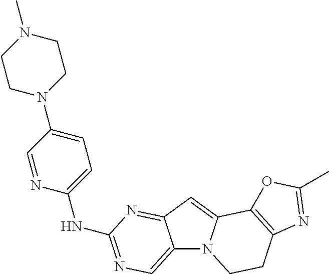 [SMILES: Cc1nc2c(o1)-c1cc3nc(Nc4ccc(N5CCN(C)CC5)cn4)ncc3n1CC2] | 2-methyl-N-(5-(4-methylpiperazin-1- yl)pyridin-2-yl)-4,5-dihydrooxazolo[4,5- g]pyrimido[4,5-b]indolizin-9-amine |
| 39 | 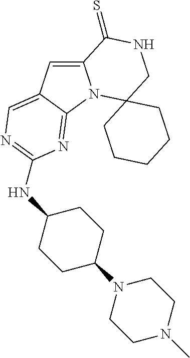 [SMILES: CN1CCN([C@H]2CC[C@@H](Nc3ncc4cc5n(c4n3)C3(CCCCC3)CNC5=S)CC2)CC1] | 2′-(((1s,4s)-4-(4-methylpiperazin-1- yl)cyclohexyl)amino)-7′,8′-dihydro-6′H- spiro[cyclohexane-1,9′- pyrazino[1′,2′:1,5]pyrrolo[2,3- d]pyrimidine]-6′-thione |
| 40 | 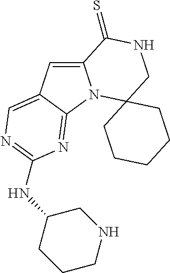 [SMILES: S=C1NCC2(CCCCC2)n2c1cc1cnc(N[C@H]3CCCNC3)nc12] | (S)-2′-(piperidin-3-ylamino)-7′,8′-dihydro- 6′H-spiro[cyclohexane-1,9′- pyrazino[1′,2′:1,5]pyrrolo[2,3- d]pyrimidine]-6′-thione |
| 41 | 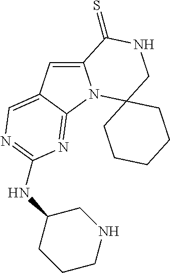 [SMILES: S=C1NCC2(CCCCC2)n2c1cc1cnc(N[C@@H]3CCCNC3)nc12] | (R)-2′-(piperidin-3-ylamino)-7′,8′-dihydro- 6′H-spiro[cyclohexane-1,9′- pyrazino[1′,2′:1,5]pyrrolo[2,3- d]pyrimidine]-6′-thione |
| 42 | 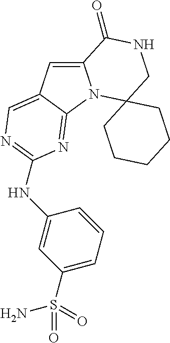 [SMILES: NS(=O)(=O)c1cccc(Nc2ncc3cc4n(c3n2)C2(CCCCC2)CNC4=O)c1] | 3-((6′-oxo-7′,8′-dihydro-6′H- spiro[cyclohexane-1,9′- pyrazino[1′,2′:1,5]pyrrolo[2,3- d]pyrimidin]-2′- yl)amino)benzenesulfonamide |
| 43 | 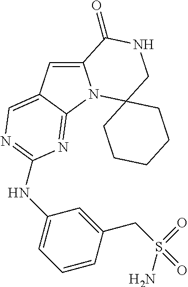 [SMILES: NS(=O)(=O)Cc1cccc(Nc2ncc3cc4n(c3n2)C2(CCCCC2)CNC4=O)c1] | (3-((6′-oxo-7′,8′-dihydro-6′H- spiro[cyclohexane-1,9′- pyrazino[1′,2′:1,5]pyrrolo[2,3- d]pyrimidin]-2′- yl)amino)phenyl)methanesulfonamide |
| 44 | 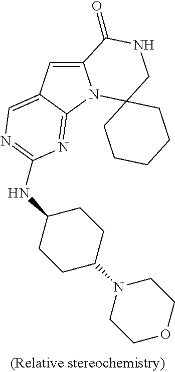 | 2′-(((1,4-trans)-4- morpholinocyclohexyl)amino)-7′,8′- dihydro-6′H-spiro[cyclohexane-1,9′- pyrazino[1′,2′:1,5]pyrrolo[2,3- d]pyrimidin]-6′-one |
| 45 | 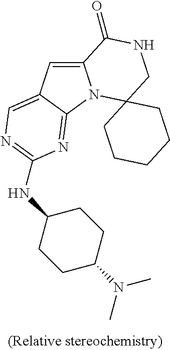 [SMILES: CN(C)[C@H]1CC[C@H](Nc2ncc3cc4n(c3n2)C2(CCCCC2)CNC4=O)CC1] | 2′-(((1,4-trans)-4- (dimethylamino)cyclohexyl)amino)-7′,8′- dihydro-6′H-spiro[cyclohexane-1,9′- pyrazino[1′,2′:1,5]pyrrolo[2,3- d]pyrimidin]-6′-one |
| 46 | 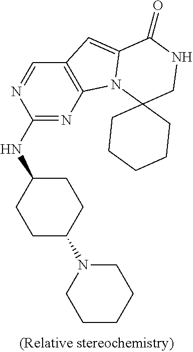 [SMILES: O=C1NCC2(CCCCC2)n2c1cc1cnc(N[C@H]3CC[C@H](N4CCCCC4)CC3)nc12] | 2′-(((1,4-trans)-4-(piperidin-1- yl)cyclohexyl)amino)-7′,8′-dihydro-6′H- spiro[cyclohexane-1,9′- pyrazino[1′,2′:1,5]pyrrolo[2,3- d]pyrimidin]-6′-one |
| 47 | 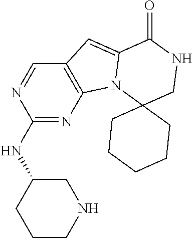 [SMILES: O=C1NCC2(CCCCC2)n2c1cc1cnc(N[C@H]3CCCNC3)nc12] | (S)-2′-(piperidin-3-ylamino)-7′,8′-dihydro- 6′H-spiro[cyclohexane-1,9′- pyrazino[1′,2′:1,5]pyrrolo[2,3- d]pyrimidin]-6′-one |
| 48 | 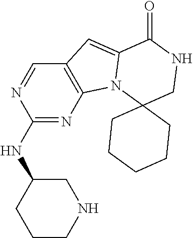 [SMILES: O=C1NCC2(CCCCC2)n2c1cc1cnc(N[C@@H]3CCCNC3)nc12] | (R)-2′-(piperidin-3-ylamino)-7′,8′-dihydro- 6′H-spiro[cyclohexane-1,9′- pyrazino[1′,2′:1,5]pyrrolo[2,3- d]pyrimidin]-6′-one |
| 49 | 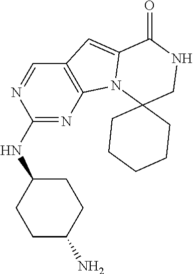 [SMILES: N[C@H]1CC[C@H](Nc2ncc3cc4n(c3n2)C2(CCCCC2)CNC4=O)CC1] | 2′-(((1r,4r)-4-aminocyclohexyl)amino)- 7′,8′-dihydro-6′H-spiro[cyclohexane-1,9′- pyrazino[1′,2′:1,5]pyrrolo[2,3- d]pyrimidin]-6′-one |
| 50 | 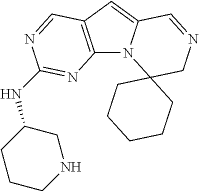 [SMILES: C1=NCC2(CCCCC2)n2c1cc1cnc(N[C@H]3CCCNC3)nc12] | (S)-N-(piperidin-3-yl)-8′H- spiro[cyclohexane-1,9′- pyrazino[1′,2′:1,5]pyrrolo[2,3- d]pyrimidin]-2′-amine |
| 51 | 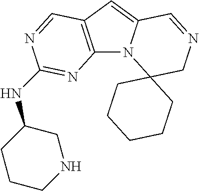 [SMILES: C1=NCC2(CCCCC2)n2c1cc1cnc(N[C@@H]3CCCNC3)nc12] | (R)-N-(piperidin-3-yl)-8′H- spiro[cyclohexane-1,9′- pyrazino[1′,2′:1,5]pyrrolo[2,3- d]pyrimidin]-2′-amine |
| 52 | 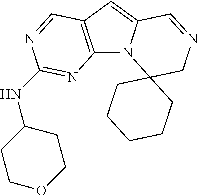 [SMILES: C1=NCC2(CCCCC2)n2c1cc1cnc(NC3CCOCC3)nc12] | N-(tetrahydro-2H-pyran-4-yl)-8′H- spiro[cyclohexane-1,9′- pyrazino[1′,2′:1,5]pyrrolo[2,3- d]pyrimidin]-2′-amine |
| 53 | 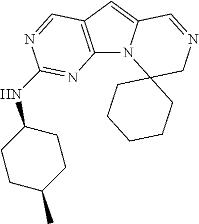 [SMILES: C[C@H]1CC[C@@H](Nc2ncc3cc4n(c3n2)C2(CCCCC2)CN=C4)CC1] | N-((1s,4s)-4-methylcyclohexyl)-8′H- spiro[cyclohexane-1,9′- pyrazino[1′,2′:1,5]pyrrolo[2,3- d]pyrimidin]-2′-amine |
| 54 | 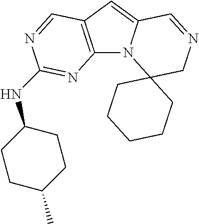 [SMILES: C[C@H]1CC[C@H](Nc2ncc3cc4n(c3n2)C2(CCCCC2)CN=C4)CC1] | N-((1r,4r)-4-methylcyclohexyl)-8′H- spiro[cyclohexane-1,9′- pyrazino[1′,2′:1,5]pyrrolo[2,3- d]pyrimidin]-2′-amine |
| 55 | 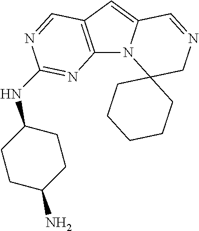 [SMILES: N[C@H]1CC[C@@H](Nc2ncc3cc4n(c3n2)C2(CCCCC2)CN=C4)CC1] | (1s,4s)-N¹-(8′H-spiro[cyclohexane-1,9′- pyrazino[1′,2′:1,5]pyrrolo[2,3- d]pyrimidin]-2′-yl)cyclohexane-1,4- diamine |

TABLE 3

| Entry | Compound Structure | Name |
|---|---|---|
| 56 | 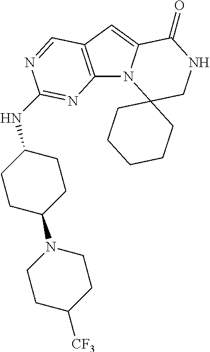 [SMILES: O=C1NCC2(CCCCC2)n2c1cc1cnc(N[C@H]3CC[C@H](N4CCC(C(F)(F)F)CC4)CC3)nc12] | 2′-(((1r,4r)-4-(4- (trifluoromethyl)piperidin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 57 | 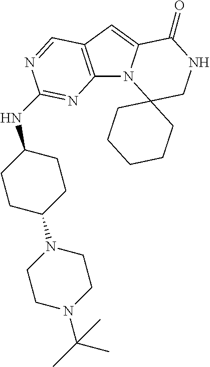 [SMILES: CC(C)(C)N1CCN([C@H]2CC[C@H](Nc3ncc4cc5n(c4n3)C3(CCCCC3)CNC5=O)CC2)CC1] | 2′-(((1r,4r)-4-(4-(tert- butyl)piperazin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 58 | 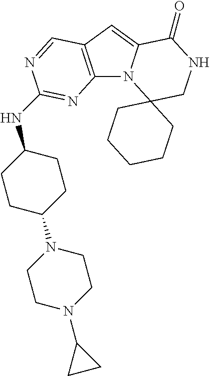 [SMILES: O=C1NCC2(CCCCC2)n2c1cc1cnc(N[C@H]3CC[C@H](N4CCN(C5CC5)CC4)CC3)nc12] | 2′-(((1r,4r)-4-(4- cyclopropylpiperazin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 59 | 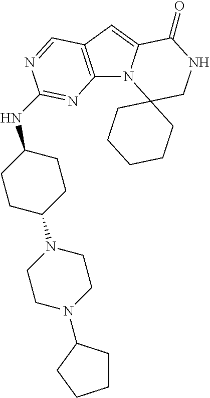 [SMILES: O=C1NCC2(CCCCC2)n2c1cc1cnc(N[C@H]3CC[C@H](N4CCN(C5CCCC5)CC4)CC3)nc12] | 2′-(((1r,4r)-4-(4- cyclopentylpiperazin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 60 | 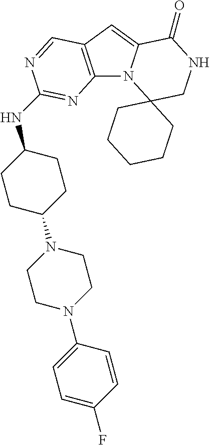 [SMILES: O=C1NCC2(CCCCC2)n2c1cc1cnc(N[C@H]3CC[C@H](N4CCN(c5ccc(F)cc5)CC4)CC3)nc12] | 2′-(((1r,4r)-4-(4-(4- fluorophenyl)piperazin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 61 | 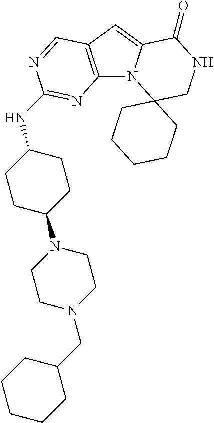 [SMILES: O=C1NCC2(CCCCC2)n2c1cc1cnc(N[C@H]3CC[C@H](N4CCN(CC5CCCCC5)CC4)CC3)nc12] | 2′-(((1r,4r)-4-(4- (cyclohexylmethyl) piperazin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 62 | 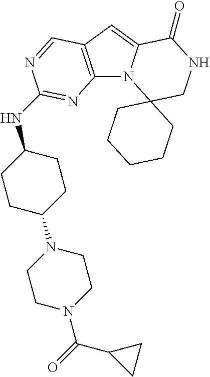 [SMILES: O=C1NCC2(CCCCC2)n2c1cc1cnc(N[C@H]3CC[C@H](N4CCN(C(=O)C5CC5)CC4)CC3)nc12] | 2′-(((1r,4r)-4-(4- (cyclopropanecarbonyl) piperazin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 63 | 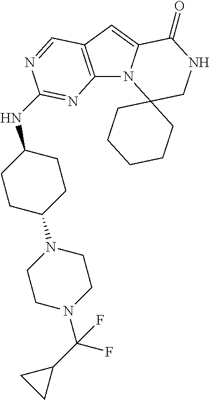 [SMILES: O=C1NCC2(CCCCC2)n2c1cc1cnc(N[C@H]3CC[C@H](N4CCN(C(F)(F)C5CC5)CC4)CC3)nc12] | 2′-(((1r,4r)-4-(4- (cyclopropyldifluoromethyl) piperazin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 64 | 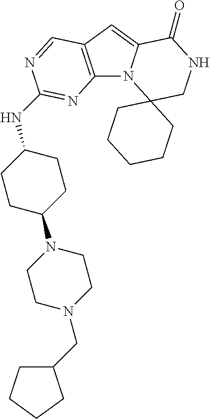 [SMILES: O=C1NCC2(CCCCC2)n2c1cc1cnc(N[C@H]3CC[C@H](N4CCN(CC5CCCC5)CC4)CC3)nc12] | 2′-(((1r,4r)-4-(4- (cyclopentylmethyl) piperazin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 69 | 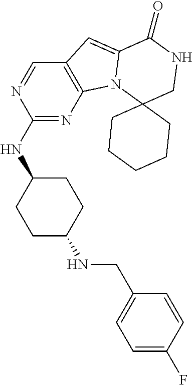 [SMILES: O=C1NCC2(CCCCC2)n2c1cc1cnc(N[C@H]3CC[C@H](NCc4ccc(F)cc4)CC3)nc12] | 2′-(((1r,4r)-4-((4- fluorobenzyl)amino) cyclohexyl)amino)- 7′,8′-dihydro-6′H-spiro [cyclohexane-1,9′- pyrazino[1′,2′:1,5] pyrrolo[2,3- d]pyrimidin]-6′-one |
| 70 | 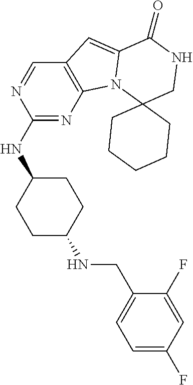 [SMILES: O=C1NCC2(CCCCC2)n2c1cc1cnc(N[C@H]3CC[C@H](NCc4ccc(F)cc4F)CC3)nc12] | 2′-(((1r,4r)-4-((2,4- difluorobenzyl)amino) cyclohexyl)amino)- 7′,8′-dihydro-6′H-spiro [cyclohexane-1,9′- pyrazino[1′,2′:1,5] pyrrolo[2,3- d]pyrimidin]-6′-one |
| 71 | 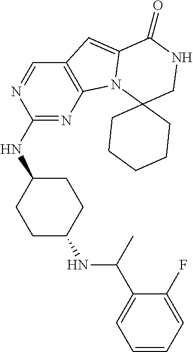 [SMILES: CC(N[C@H]1CC[C@H](Nc2ncc3cc4n(c3n2)C2(CCCCC2)CNC4=O)CC1)c1ccccc1F] | 2′-(((1r,4r)-4-((1-(2- fluorophenyl)ethyl) amino)cyclohexyl) amino)-7′,8′-dihydro- 6′H-spiro [cyclohexane-1,9′-pyrazino [1′,2′:1,5]pyrrolo [2,3-d]pyrimidin]-6′-one |
| 72 | 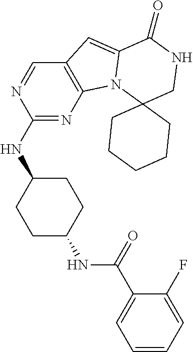 [SMILES: O=C(N[C@H]1CC[C@H](Nc2ncc3cc4n(c3n2)C2(CCCCC2)CNC4=O)CC1)c1ccccc1F] | 2′-fluoro-N-((1r,4r)- 4-((6′-oxo-7′,8′- dihydro-6′H-spiro [cyclohexane-1,9′- pyrazino[1′,2′:1,5] pyrrolo[2,3- d]pyrimidin]-2′- yl)amino)cyclohexyl) benzamide |
| 73 | 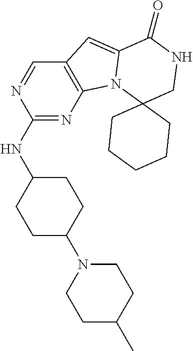 [SMILES: CC1CCN(C2CCC(Nc3ncc4cc5n(c4n3)C3(CCCCC3)CNC5=O)CC2)CC1] | 2′-((4-(4- methylpiperidin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 74 | 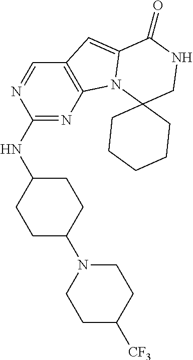 [SMILES: O=C1NCC2(CCCCC2)n2c1cc1cnc(NC3CCC(N4CCC(C(F)(F)F)CC4)CC3)nc12] | 2′-((4-(4-(trifluoromethyl) piperidin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 79 | 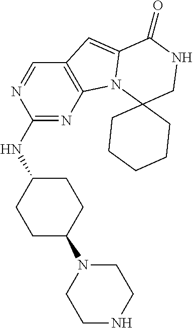 [SMILES: O=C1NCC2(CCCCC2)n2c1cc1cnc(N[C@H]3CC[C@H](N4CCNCC4)CC3)nc12] | 2′-(((1r,4r)-4-(piperazin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 80 | 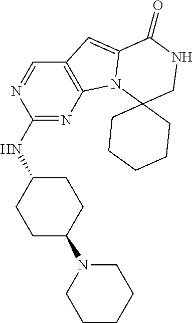 [SMILES: O=C1NCC2(CCCCC2)n2c1cc1cnc(N[C@H]3CC[C@H](N4CCCCC4)CC3)nc12] | 2′-(((1r,4r)-4-(piperidin-1- yl)cyclohexyl)amino)- 7′,8′-dihydro-6′H- spiro[cyclohexane-1,9′- pyrazino[1′,2′:1,5] pyrrolo[2,3- d]pyrimidin]-6′-one |
| 82 | 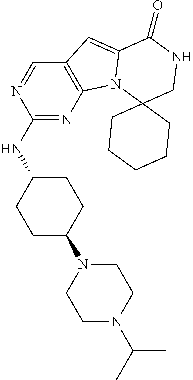 [SMILES: CC(C)N1CCN([C@H]2CC[C@H](Nc3ncc4cc5n(c4n3)C3(CCCCC3)CNC5=O)CC2)CC1] | 2′-((5-(piperidin-1-yl) pyridin-2-yl)amino)- 7′,8′-dihydro-6′H-spiro [cyclohexane-1,9′- pyrazino[1′,2′:1,5] pyrrolo[2,3- d]pyrimidin]-6′-one |

### Example 4: CDK Inhibition In Vitro Assays

Selected compounds disclosed herein were tested in kinase assays by Nanosyn (Santa Clara, Calif.) to determine their inhibitory effect on selected CDKs. The assays were performed using microfluidic kinase detection technology (Caliper Assay Platform). The compounds were tested in 12-point dose-response format in singlicate at Kmfor ATP. Phosphoacceptor substrate peptide concentration used was 1 μM for all assays and Staurosporine was used as the reference compound for all assays. Specifics of each assay are as described below:

CDK1/Cyclin B1: Enzyme concentration: 0.08 nM; ATP concentration: 40 μM; Incubation time: 3 hr.

CDK2/Cyclin A: Enzyme concentration: 0.1 nM; ATP concentration: 50 μM; Incubation time: 3 hr.

CDK2/Cyclin E: Enzyme concentration: 0.15 nM; ATP concentration: 100 μM; Incubation time: 3 hr.

CDK3/Cyclin E: Enzyme concentration: 1 nM; ATP concentration: 400 μM; Incubation time: 3 hr.

CDK4/Cyclin D1: Enzyme concentration: 1 nM; ATP concentration: 200 μM; Incubation time: 3 hr.

CDK5/p35: Enzyme concentration: 0.05 nM; ATP concentration: 20 μM; Incubation time: 3 hr.

CDK5/p25: Enzyme concentration: 0.1 nM; ATP concentration: 20 μM; Incubation time: 3 hr.

CDK6/Cyclin D3: Enzyme concentration: 2 nM; ATP concentration: 300 μM; Incubation time: 3 hr.

CDK7/Cyclin H: Enzyme concentration: 10 nM; ATP concentration: 50 μM; Incubation time: 17 hr.

CDK7/Cyclin Ti: Enzyme concentration: 5 nM; ATP concentration: 10 μM; Incubation time: 17 hr.

TABLE 4
Biological Data

| | CDK1/ | CDK2/ | CDK2/ | CDK3/ | CDK4/ | CDK5/ |
|---|---|---|---|---|---|---|
| | Cyclin | Cyclin | Cyclin E | Cyclin E | Cyclin | p35 |
| Compd # | B1 (μM) | A (μM) | (μM) | (μM) | D1 (μM) | (μM) |
| 7 | >100 | 65 | >100 | >100 | 0.82 | >100 |
| 8 | 27 | 11 | 24 | 18 | 0.10 | 26 |
| 9 | 82 | 71 | >100 | >100 | 0.12 | >100 |
| 10 | 8.8 | 3.6 | 5.7 | 16 | 0.036 | 9.7 |
| 11 | 4.4 | 3.1 | 13 | 7.2 | 2.9 | 30 |
| 16 | 0.091 | 0.11 | 0.19 | 0.24 | 0.013 | 0.47 |
| 18 | 0.080 | 0.049 | 0.078 | 0.19 | 0.0092 | 0.099 |
| 19 | 0.096 | 0.071 | 0.11 | 0.22 | 0.0086 | 0.12 |
| 20 | 0.36 | 0.18 | 0.31 | 0.40 | 0.014 | 0.37 |
| 21 | 0.31 | 0.14 | 0.22 | 0.29 | 0.014 | 0.35 |
| 22 | 0.41 | 0.078 | 0.19 | 0.51 | 0.069 | 0.17 |
| 23 | 39 | 28 | 35 | 56 | 0.17 | 47 |
| 24 | 0.56 | 0.22 | 0.24 | 0.29 | 0.25 | 0.33 |
| 27 | 8.9 | 8.0 | 11 | 26 | 0.27 | 10 |
| 28 | 93 | 67 | >100 | >100 | 9.0 | >100 |
| 30 | 3.8 | 1.9 | 5.4 | 6.9 | 1.5 | 8.0 |
| 32 | 5.6 | 3.1 | 12 | 8.6 | 2.3 | 16 |
| 33 | 16 | 6.1 | 26 | 23 | 3.3 | 34 |
| 34 | 18 | 12 | 27 | 33 | 2.9 | 29 |
| 35 | 31 | 10 | 26 | 54 | 27 | 40 |
| 36 | >100 | 88 | >100 | >100 | 9.4 | >100 |
| 37 | 27 | 16 | 29 | 38 | 6.3 | 28 |
| 39 | 1.2 | 0.14 | 0.30 | 0.42 | 0.0095 | 1.5 |
| 40 | 0.061 | 0.10 | 0.11 | 0.017 | 0.023 | 0.051 |
| 41 | 0.33 | 0.15 | 0.31 | 0.21 | 0.0067 | 1.1 |
| 42 | 0.0088 | 0.0043 | 0.0078 | 0.012 | 0.0089 | 0.0075 |
| 43 | >100 | 99 | >100 | >100 | 47 | 94 |
| 44 | 0.49 | 0.17 | 0.29 | 0.47 | 0.024 | 0.32 |
| 45 | 0.27 | 0.14 | 0.26 | 0.24 | 0.011 | 0.31 |
| 46 | 0.51 | 0.25 | 0.47 | 0.52 | 0.021 | 0.71 |
| 47 | 0.17 | 0.35 | 0.55 | 0.11 | 0.13 | 0.25 |
| 48 | 0.51 | 0.39 | 1.1 | 1.0 | 0.051 | 1.3 |
| 49 | 0.14 | 0.033 | 0.072 | 0.11 | 0.0068 | 0.16 |
| 50 | 0.28 | 0.28 | 0.19 | 0.064 | 0.31 | 0.18 |
| 51 | 1.6 | 0.90 | 0.49 | 0.61 | 0.29 | 1.5 |
| 52 | 0.078 | 0.016 | 0.0087 | 0.024 | 0.067 | 0.032 |
| 53 | 1.5 | 0.67 | 0.48 | 0.85 | 2.9 | 1.5 |
| 54 | 1.7 | 0.42 | 0.26 | 0.64 | 0.67 | 0.74 |
| 55 | 1.4 | 0.81 | 0.33 | 0.46 | 0.092 | 1.7 |

TABLE 5
Additional Biological Data

| | CDK5/ | CDK6/ | CDK7/ | CDK9/ |
|---|---|---|---|---|
| Compd | p25 | Cyclin D3 | Cyclin | Cyclin T |
| # | (μM) | (μM) | H (μM) | (μM) |
| 7 | >100 | 5.6 | >100 | 3.4 |
| 8 | 25 | 0.53 | 7.2 | 0.44 |
| 9 | >100 | 12 | 20 | 1.6 |
| 10 | 11 | 1.2 | 1.8 | 0.08 |
| 11 | 35 | 28 | 13 | 0.0055 |
| 16 | 0.48 | 0.072 | 0.17 | 0.0027 |
| 18 | 0.087 | 0.059 | 0.14 | 0.0023 |
| 19 | 0.12 | 0.075 | 0.74 | 0.0021 |
| 20 | 0.37 | 0.078 | 0.23 | 0.0047 |
| 21 | 0.32 | 0.071 | 0.11 | 0.0021 |
| 22 | 0.23 | 0.35 | 3.4 | 0.056 |
| 23 | 55 | 0.94 | 61 | 0.69 |
| 24 | 0.26 | 1.0 | 0.47 | 0.025 |
| 27 | 9.6 | 0.97 | 14 | 0.56 |
| 28 | >100 | >100 | >100 | 0.24 |
| 30 | 8.4 | 15 | 4.5 | 0.045 |
| 32 | 19 | 14 | 12 | 0.012 |
| 33 | 34 | 12 | 20 | 0.035 |
| 34 | 52 | 8.4 | 17 | 0.19 |
| 35 | 43 | >100 | 61 | 0.088 |
| 36 | >100 | 82 | >100 | 0.24 |
| 37 | 31 | 14 | 17 | 0.12 |
| 39 | 1.6 | 0.048 | 0.50 | 0.013 |
| 40 | 0.040 | 0.079 | 0.010 | 0.0034 |
| 41 | 0.97 | 0.023 | 0.085 | 0.0043 |
| 42 | 0.0055 | 0.022 | 0.24 | 0.0012 |
| 43 | 99 | >100 | >100 | 10 |
| 44 | 0.27 | 0.10 | 0.27 | 0.0062 |
| 45 | 0.36 | 0.047 | 0.086 | 0.0033 |
| 46 | 0.76 | 0.078 | 0.28 | 0.0064 |
| 47 | 0.23 | 0.37 | 0.027 | 0.0026 |
| 48 | 1.2 | 0.23 | 0.33 | 0.009 |
| 49 | 0.14 | 0.040 | 0.10 | 0.0022 |
| 50 | 0.19 | 1.1 | 0.042 | 0.023 |
| 51 | 1.5 | 1.1 | 0.23 | 0.20 |
| 52 | 0.031 | 0.25 | 0.28 | 0.058 |
| 53 | 1.5 | 9.0 | 4.6 | 0.89 |
| 54 | 0.66 | 2.0 | 3.6 | 0.60 |
| 55 | 2.0 | 0.68 | 0.12 | 0.022 |

TABLE 6
Additional Biological Data

| Compd | CDK4/Cyclin | CDK9/Cyclin |
|---|---|---|
| # | D1 (nM) | T (nM) |
| 56 | 27 | 4 |
| 57 | 5 | 3 |
| 58 | 33 | 5 |
| 59 | 4 | 2 |
| 60 | 13 | 3 |
| 61 | 13 | 5 |
| 62 | 20 | 5 |

All publications and patent applications cited in this specification are herein incorporated by reference as if each individual publication or patent application were specifically and individually indicated to be incorporated by reference.

The descriptions herein are described by way of illustration and example for purposes of clarity of understanding for embodiments only. It will be readily apparent to one of ordinary skill in the art in light of the teachings of this invention that certain changes and modifications may be made thereto without departing from the spirit or scope of the invention as defined in the appended claims.

We claim:

1. A compound selected from the group consisting of: 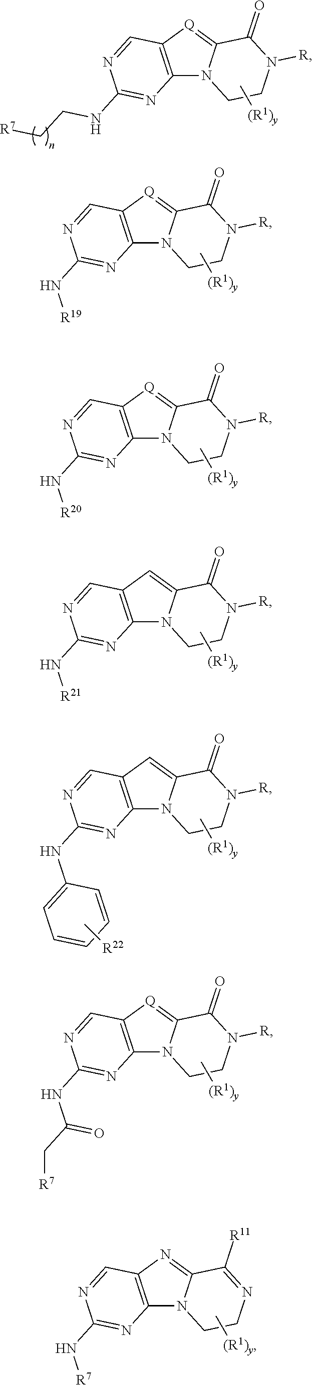 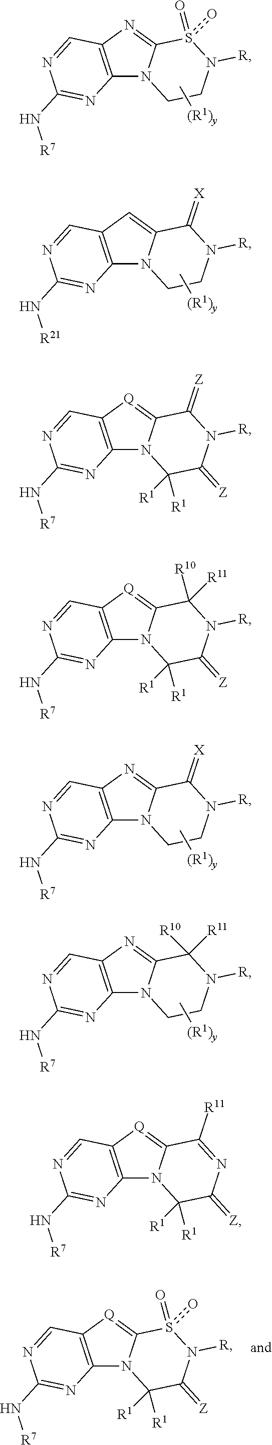 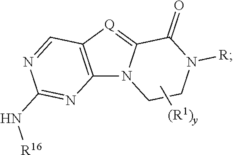
or a pharmaceutically acceptable salt thereof,
wherein:
each y is independently 0, 1, 2, 3, or 4;
X is S, CH₂, CHR¹², CR¹²R¹³, NH, or NR¹²;
Z is independently O, S, CH₂, CHR¹², CR¹²R¹³, NH, or NR¹²;
Q is CH or N; [symbol]
represents the presence or absence of a double bond;
each R is independently hydrogen, C₁-C₆alkyl, —(C₀-C₂alkyl)(C₃-C₆cycloalkyl), —(C₀-C₂alkyl)(C₃-C₈ heterocycle), —(C₀-C₂alkyl)(aryl), —(C₀-C₂alkyl)(heteroaryl), —COOalkyl, —COOalkyl-aryl, or —COOH;
each R¹ is independently hydrogen, alkyl, aryl, cycloalkyl, haloalkyl, heteroaryl, or heterocycle; wherein two R¹ groups on adjacent ring atom(s) or on the same ring atom may come together with the ring atom(s) to which they are attached to optionally constitute a 3, 4, 5, 6, 7, or 8-membered cycloalkyl or heterocycle that has 1, 2, or 3 heteroatoms selected from N, O, and S; wherein the 3, 4, 5, 6, 7, or 8-membered cycloalkyl or heterocycle formed by combining two R¹s with the atom(s) to which they are attached can be optionally substituted with 1, 2, 3, or 4 substituents independently selected from R⁵⁰;
R⁵⁰ is selected from the group consisting of hydrogen, amino, —NHR¹⁴, —NR¹⁴R¹⁵, hydroxyl, OR¹⁴, R⁶, and R²;
R⁷ is selected from the group consisting of aryl, heteroaryl, cycloalkyl, heterocycle, alkyl, —C(O)aryl, —C(O)heteroaryl, —C(O)cycloalkyl, —C(O)heterocycle, —C(O)alkyl, and —C(O)heterocycle; each of which R⁷ is optionally substituted with 1, 2, 3, or 4 substituents independently selected from the group consisting of amino, halogen, alkyl, —NHR¹⁴, —NR¹⁴R¹⁵, hydroxyl, OR¹⁴, R⁶, and R²;
R² is independently selected from the group consisting of -(alkylene)ₘ-heterocycle, -(alkylene)ₘ-heteroaryl, -(alkylene)ₘ-NR³R⁴, -(alkylene)ₘ-C(O)—NR³R⁴; (alkylene)ₘ-C(O)—O-alkyl; -(alkylene)ₘ-O—R⁵, -(alkylene)ₘ-S(O)ₙ—R⁵, and -(alkylene)ₘ-S(O)ₙ—NR³R⁴; any of which may be optionally independently substituted with 1, 2, 3, or 4 R^{x} groups as allowed by valance, and wherein two R^{x} groups bound to the same or adjacent atom may optionally combine to form a ring;
m is 0 or 1;
n is independently 0, 1, or 2;
R³ and R⁴ at each occurrence are independently selected from the group consisting of hydrogen, alkyl, cycloalkyl, heterocycle, aryl, heteroaryl, alkyl-cycloalkyl, alkyl-heterocycle, alkyl-aryl, and alkyl-heteroaryl; each of which R³ and R⁴ except hydrogen may be optionally independently substituted with 1, 2, 3, or 4 R^{x} groups as allowed by valance; or
R³ and R⁴ together with the nitrogen atom to which they are attached may combine to form a heterocycle ring optionally independently substituted with 1, 2, 3, or 4 R^{x} groups as allowed by valance, and wherein two R^{x}s bound to the same or adjacent atom(s) may optionally combine to form a 3, 4, 5, 6, 7, or 8-membered cycloalkyl or heterocycle that has 1, 2, or 3 heteroatoms selected from N, O, and S;
R⁵ is independently selected at each occurrence from the group consisting of hydrogen, alkyl, alkenyl, alkynyl, cycloalkyl, heterocycle, aryl, heteroaryl, alkyl-cycloalkyl, alkyl-heterocycle, alkyl-aryl, and alkyl-heteroaryl; each of which R⁵ except hydrogen may be optionally independently substituted with 1, 2, 3, or 4 R^{x} groups as allowed by valance;
R^{x} at each occurrence is independently selected from the group consisting of hydroxy, —O-alkyl, halogen, cyano, nitro, oxo, alkyl, haloalkyl, alkenyl, alkynyl, cycloalkyl, heterocycle, aryl, heteroaryl, alkyl-aryl, alkyl-heteroaryl, alkyl-cycloalkyl, amino, —C(O)N(R⁶)₂, —C(O)OR⁶, and alkyl-heterocycle; each of which R^{x} groups except halogen, cyano, nitro, and oxo may be further independently substituted with 1, 2, 3, or 4 substituents independently selected from the group consisting of hydroxy, —O-alkyl, halo, cyano, nitro, oxo, alkyl, haloalkyl, alkenyl, alkynyl, cycloalkyl, heterocycle, aryl, heteroaryl, alkyl-aryl, alkyl-heteroaryl, alkyl-cycloalkyl, and alkyl-heterocycle; or
R^{x} is selected from the group consisting of —C(O)alkyl and —C(O)cycloalkyl;
R⁶ is selected independently at each instance from the group consisting of alkyl, alkenyl, alkynyl, cycloalkyl, heterocycle, aryl, heteroaryl, alkyl-cycloalkyl, alkyl-heterocycle, alkyl-aryl, and alkyl-heteroaryl;
R¹⁰ and R¹¹ are independently selected from the group consisting of hydrogen, alkyl, —NH₂, —NHR¹², —NR¹²R¹³, —S(O)alkyl, —SO₂alkyl, cycloalkyl, heterocycle, aryl, heteroaryl, alkyl-aryl, and alkyl-heteroaryl; each of which R¹⁰ and R¹¹ except hydrogen is optionally substituted with 1, 2, 3, or 4 substituents selected from the group consisting of amino, —NHR¹⁴, —NR¹⁴R¹⁵, hydroxyl, OR¹⁴, R⁶, and R²;
R¹² is selected from the group consisting of hydrogen, alkyl, alkenyl, alkynyl, —C(O)R⁶, —C(O)alkyl, —C(S)alkyl, aryl, —SO₂alkyl, heteroaryl, alkyl-aryl, cycloalkyl, heterocycle, and alkyl-heteroaryl; each of which R¹² except hydrogen is optionally substituted with 1, 2, 3, or 4 substituents selected from the group consisting of amino, —NHR¹⁴, —NR¹⁴R¹⁵, hydroxyl, OR¹⁴, R⁶, and R²;
R¹³ is independently selected from the group consisting of hydrogen, alkyl, alkenyl, alkynyl, —C(O)R⁶, —C(O)alkyl, —C(S)alkyl, aryl, —SO₂alkyl, heteroaryl, alkyl-aryl, cycloalkyl, heterocycle, and alkyl-heteroaryl; each of which R¹³ except hydrogen is optionally substituted with 1, 2, 3, or 4 substituents selected from the group consisting of amino, —NHR¹⁴, —NR¹⁴R¹⁵, hydroxyl, OR¹⁴, R⁶, and R²;
R¹⁴ and R¹⁵ are independently selected from the group consisting of hydrogen, alkyl, alkenyl, alkynyl, —C(O)R⁶, —C(O)alkyl, —C(S)alkyl, aryl, —SO₂alkyl, heteroaryl, heterocycle, alkyl-aryl, and alkyl-heteroaryl;
R¹⁶ is cycloalkyl substituted with at least one substituent selected from R¹⁷ and optionally substituted with 1, 2, 3, or 4 substituents independently selected from R^{x}; or
R¹⁶ is cycloalkyl-heterocycle-R^{x} optionally substituted with 1, 2, 3, or 4 additional substituents independently selected from R^{x};
R¹⁷ is independently selected from the group consisting of —NR—S(O)alkyl, —NR—S(O)₂alkyl, —NR-heterocycle, —NR-heteroaryl, —NR-aryl, —NR-alkyl-heteroaryl, and —NR-alkyl-aryl, each of which R¹⁷ is optionally substituted with 1, 2, 3, or 4 substituents selected from the group consisting of R^{x};
R¹⁹ is a heterocycle substituted with at least one substituent independently selected from the group consisting of amino, halogen, alkyl, —NHR¹⁴, —NR¹⁴R¹⁵, hydroxyl, OR¹⁴, R⁶, oxo, and R²;
R²⁰ is selected from the group consisting of —C(O)alkyl, —C(O)aryl, —C(O)heteroaryl, —C(O)cycloalkyl, and —C(O)heterocycle; each of which R²⁰ is optionally substituted with 1, 2, 3, or 4 substituents independently selected from the group consisting of amino, halogen, alkyl, —NHR¹⁴, —NR¹⁴R¹⁵ hydroxyl, OR¹⁴, R⁶, —C(O)R⁶, and R²;
R²¹ is selected from the group consisting of 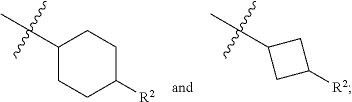
and
R²² is selected from the group consisting of

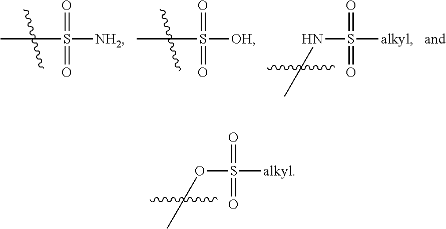

2. The compound of claim 1 wherein R is hydrogen.

3. The compound of claim 2, wherein the compound is of formula: 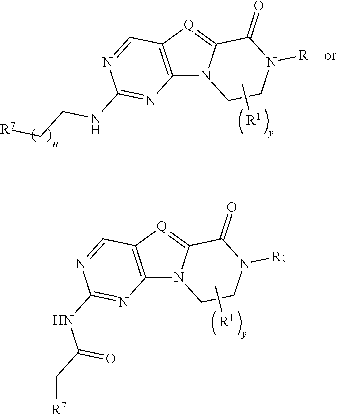
or a pharmaceutically acceptable salt thereof.

4. The compound of claim 3, wherein R⁷ is selected from the group consisting of:

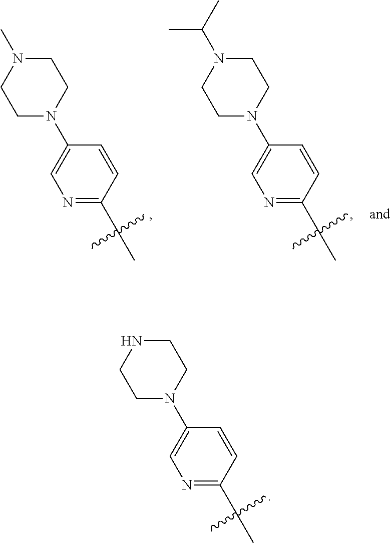 [SMILES: *c1ccc(N2CCN(C)CC2)cn1 ; *c1ccc(N2CCN(C(C)C)CC2)cn1 ; *c1ccc(N2CCNCC2)cn1]

5. The compound of claim 4, wherein n is 0.

6. The compound of claim 5, wherein Q is CH.

7. The compound of claim 2, wherein the compound is of formula: 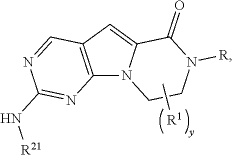
or a pharmaceutically acceptable salt thereof.

8. The compound of claim 7, wherein R²¹ is

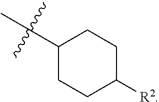

9. The compound of claim 2, wherein the compound is of formula: 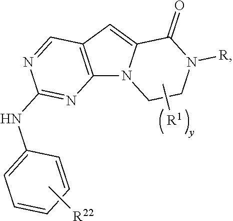
or a pharmaceutically acceptable salt thereof.

10. The compound of claim 9, wherein R²² is

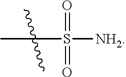 [SMILES: *S(N)(=O)=O]

11. The compound of claim 9, wherein R²² is

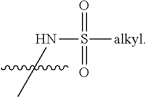

12. The compound of claim 9, wherein R²² is

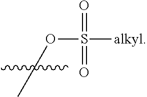

13. The compound claim 1, wherein R¹ is hydrogen, alkyl, or aryl.

14. The compound claim 1, wherein y is 0, 1, or 2.

15. The compound of claim 1, wherein two R¹ groups on the same ring atom come together with the ring atom to which they are attached to constitute a 3, 4, 5, 6, 7, or 8-membered cycloalkyl or heterocycle that has 1, 2, or 3 heteroatoms selected from N, O, and S.

16. The compound of claim 1, wherein two R¹ groups on the same ring atom come together with the ring atom to which they are attached to constitute a 6-membered cycloalkyl.

17. The compound of claim 1 selected from the group consisting of: 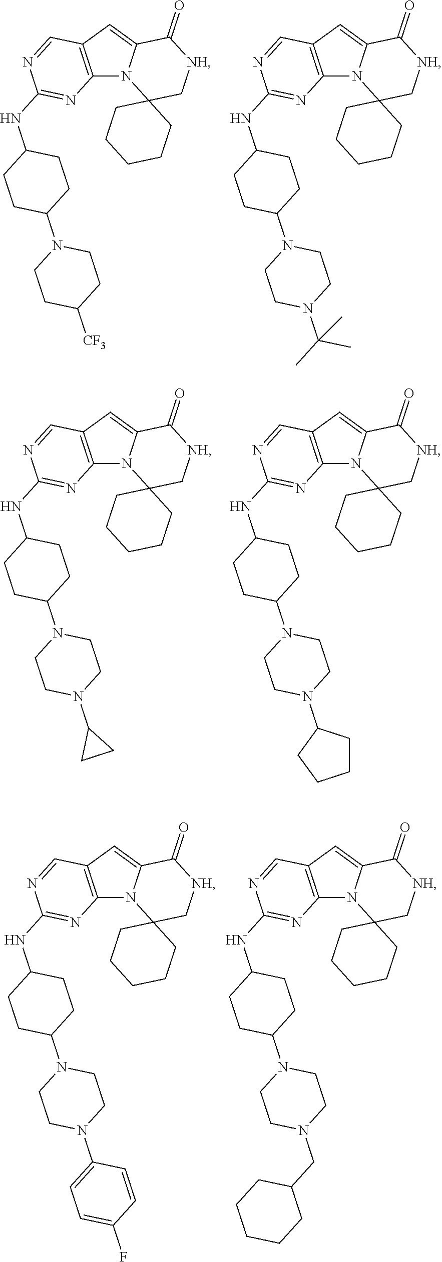 [SMILES: O=C1NCC2(CCCCC2)n2c1cc1cnc(NC3CCC(N4CCC(C(F)(F)F)CC4)CC3)nc12 ; CC(C)(C)N1CCN(C2CCC(Nc3ncc4cc5n(c4n3)C3(CCCCC3)CNC5=O)CC2)CC1 ; O=C1NCC2(CCCCC2)n2c1cc1cnc(NC3CCC(N4CCN(C5CC5)CC4)CC3)nc12 ; O=C1NCC2(CCCCC2)n2c1cc1cnc(NC3CCC(N4CCN(C5CCCC5)CC4)CC3)nc12 ; O=C1NCC2(CCCCC2)n2c1cc1cnc(NC3CCC(N4CCN(c5ccc(F)cc5)CC4)CC3)nc12 ; O=C1NCC2(CCCCC2)n2c1cc1cnc(NC3CCC(N4CCN(CC5CCCCC5)CC4)CC3)nc12] 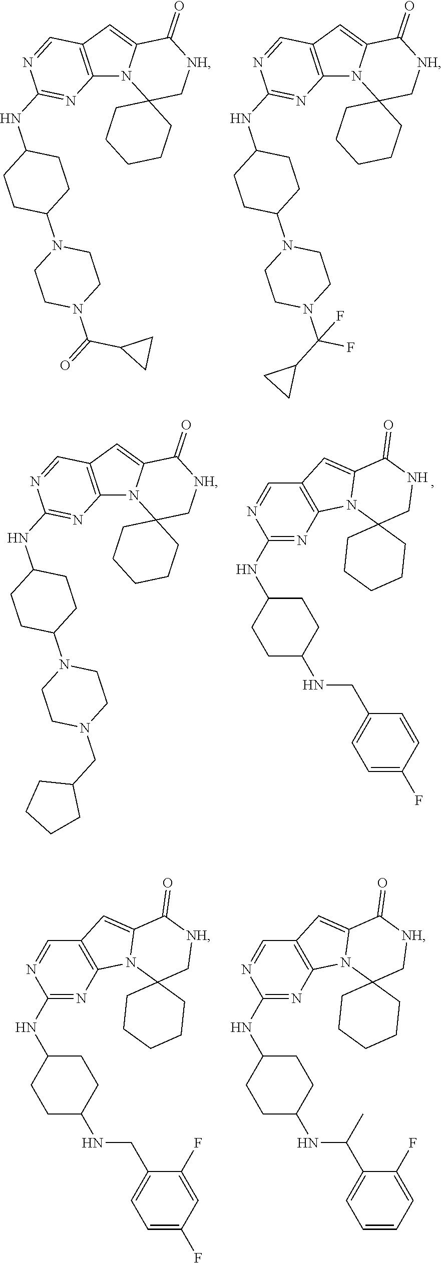 [SMILES: O=C1NCC2(CCCCC2)n2c1cc1cnc(NC3CCC(N4CCN(C(=O)C5CC5)CC4)CC3)nc12 ; O=C1NCC2(CCCCC2)n2c1cc1cnc(NC3CCC(N4CCN(C(F)(F)C5CC5)CC4)CC3)nc12 ; O=C1NCC2(CCCCC2)n2c1cc1cnc(NC3CCC(N4CCN(CC5CCCC5)CC4)CC3)nc12 ; O=C1NCC2(CCCCC2)n2c1cc1cnc(NC3CCC(NCc4ccc(F)cc4)CC3)nc12 ; O=C1NCC2(CCCCC2)n2c1cc1cnc(NC3CCC(NCc4ccc(F)cc4F)CC3)nc12 ; CC(NC1CCC(Nc2ncc3cc4n(c3n2)C2(CCCCC2)CNC4=O)CC1)c1ccccc1F] 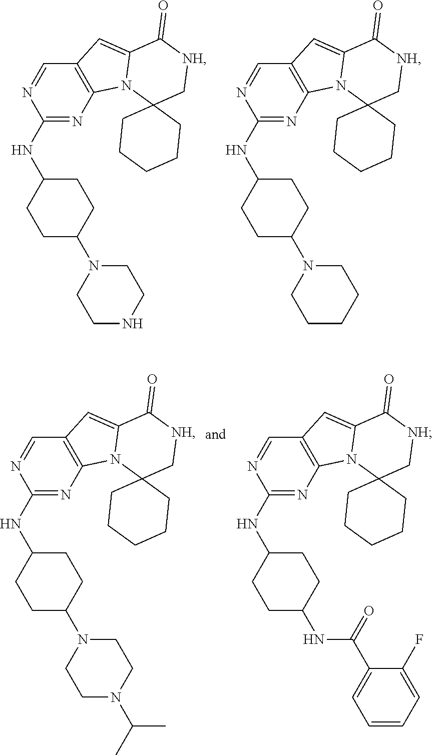 [SMILES: O=C1NCC2(CCCCC2)n2c1cc1cnc(NC3CCC(N4CCNCC4)CC3)nc12 ; O=C1NCC2(CCCCC2)n2c1cc1cnc(NC3CCC(N4CCCCC4)CC3)nc12 ; CC(C)N1CCN(C2CCC(Nc3ncc4cc5n(c4n3)C3(CCCCC3)CNC5=O)CC2)CC1 ; O=C(NC1CCC(Nc2ncc3cc4n(c3n2)C2(CCCCC2)CNC4=O)CC1)c1ccccc1F]
or a pharmaceutically acceptable salt thereof.

18. The compound of claim 1 selected from the group consisting of 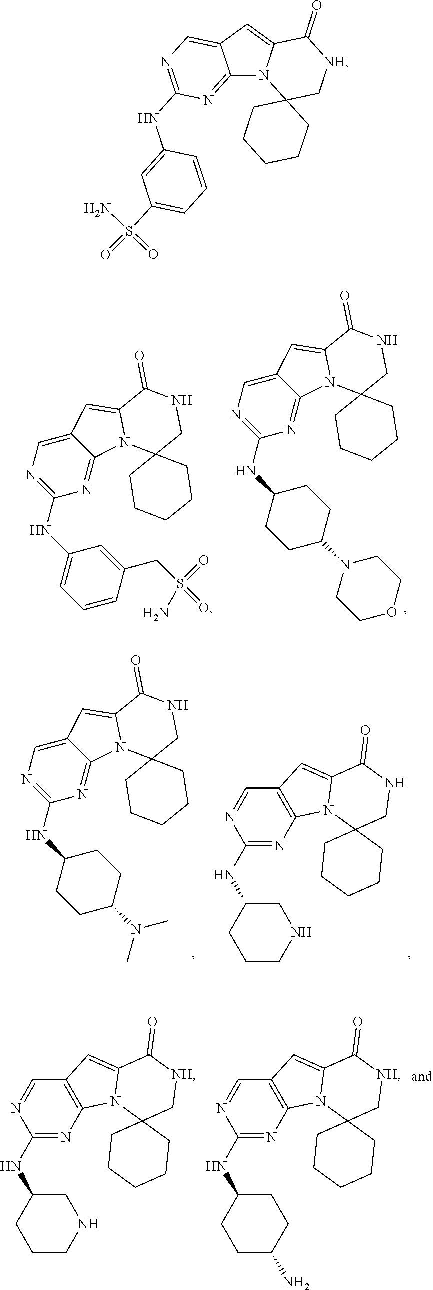 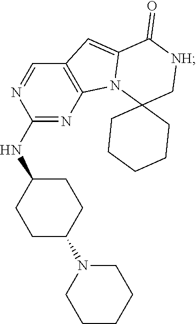 [SMILES: O=C1NCC2(CCCCC2)n2c1cc1cnc(N[C@H]3CC[C@H](N4CCCCC4)CC3)nc12]
or a pharmaceutically acceptable salt thereof.

19. A pharmaceutical composition comprising a compound of claim 1 and a pharmaceutically acceptable excipient.

20. A method for reducing the effect of chemotherapy on healthy cells in a human being treated for cancer or abnormal cell proliferation comprising administering an effective amount of a compound of claim 1 to a host in need thereof, or a pharmaceutically acceptable salt thereof, optionally in a pharmaceutically acceptable carrier.

21. A method for the treatment of a disorder associated with abnormal cellular proliferation comprising administering an effective amount of a compound of claim 1 to a host in need thereof, or a pharmaceutically acceptable salt thereof, optionally in a pharmaceutically acceptable carrier.
